(19) United States
(12) Patent Application Publication
(10) Pub. No.: US 2025/0263385 A1
(43) Pub. Date: Aug. 21, 2025

(54) "Good" buffer-based cationic lipids

(71) Applicants: SANOFI, Paris (FR); TRANSLATE BIO, INC., Waltham, MA (US)

(72) Inventors: Frank DEROSA, Cambridge, MA (US); Hongfeng DENG, Cambridge, MA (US); Rebecca GOLDMAN, Cambridge, MA (US); Shrirang KARVE, Cambridge, MA (US); Saswata KARMAKAR, Cambridge, MA (US); Ramesh DASARI, Cambridge, MA (US); Ryan LANDIS, Cambridge, MA (US)

(73) Assignees: SANOFI, Paris (FR); TRANSLATE BIO, INC., Waltham, MA (US)

(21) Appl. No.: 18/856,198

(22) PCT Filed: Apr. 13, 2023
(86) PCT No.: PCT/EP2023/059726

§ 371 Date: Oct. 11, 2024

(30) Foreign Application Priority Data
Apr. 13, 2022 (AR) P20220100953
Apr. 14, 2022 (TW) 111114318
Apr. 15, 2022 (WO) PCT/US2022/025067
Apr. 4, 2023 (EP) 23305491.5

(51) Int. Cl. C07D 295/088; A61K 9/1272; A61K 9/51; A61K 45/00

(52) U.S. Cl. CPC C07D 295/088; A61K 9/1272; A61K 9/5123; A61K 45/00

(57) ABSTRACT

The present invention provides, in part, second generation “good” buffer-based cationic lipids of Formula (I), and subformulas thereof: Formula (I), (t), or a pharmaceutically acceptable salt thereof. The compounds provided herein can be useful for delivery and expression of mRNA and encoded protein, e.g., as a component of liposomal delivery vehicle, and accordingly can be useful for treating various diseases, disorders and conditions, such as those associated with deficiency of one or more proteins.

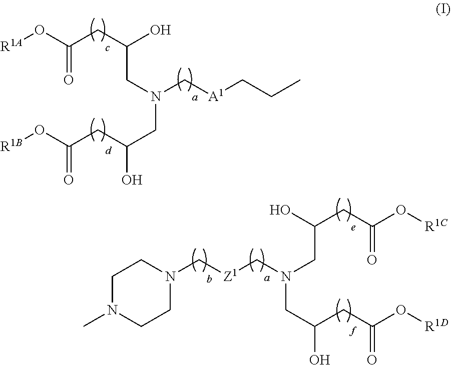

### CROSS-REFERENCE TO RELATED APPLICATIONS

The present application claims benefit of Argentina Patent Application with Serial No. P 22 01 00953, filed 13 Apr. 2022, Taiwan Patent Application No. 111114318, filed 14 Apr. 2022, International Patent Application No. PCT/US2022/025067, filed 15 Apr. 2022, and European Patent Application No. EP23305491.5, filed 4 Apr. 2023, each of which is incorporated by reference in its entirety.

### BACKGROUND

Delivery of nucleic acids has been explored extensively as a potential therapeutic option for certain disease states. In particular, messenger RNA (mRNA) therapy has become an increasingly important option for the prevention and treatment of various diseases (e.g. in the use of vaccines).

Efficient delivery of liposome-encapsulated nucleic acids remains an active area of research. The cationic lipid component of a liposome plays an important role in facilitating effective encapsulation of the nucleic acid during the loading of liposomes. In addition, cationic lipids may play an important role in the efficient release of the nucleic acid cargo from the liposome into the cytoplasm of a target cell. Various cationic lipids suitable for in vivo use have been discovered. However, there remains a need to identify cationic lipids that can be synthesized efficiently and cheaply without the formation of potentially toxic by-products. There also remains a need to identify cationic lipids that exhibit improved biodegradability.

“Good” buffers (or Good's buffers) are buffering agents for biochemical and biological research that were first selected and described by Norman Good and his colleagues (Good, N. E., et al. (1966) Hydrogen Ion Buffers for Biological Research. Biochemistry 5(2), 467-477). Most biological reactions take place near-neutral pH between 6 and 8. Good therefore reasoned that an ideal buffer for biochemical or biological applications would have a pKa value in this region to provide maximum buffering capacity. Additional selection criteria included high solubility, lack of toxicity, limited interference with biochemical reactions, very low absorbance between 240 nm and 700 nm, enzymatic and hydrolytic stability, minimal changes due to temperature and concentration, limited effects due to ionic or salt composition of the solution, limited interaction with mineral cations, and limited permeability of biological membranes.

### BRIEF DESCRIPTION OF DRAWINGS

FIG. 1 shows that lipid nanoparticles comprising the lipids described herein are highly effective in delivering hEPO mRNA and show high levels of hEPO protein expression at 6 hours post-IM injection dose.

### SUMMARY OF THE INVENTION

The foregoing characteristics make “Good” buffers exceptionally good starting points for the synthesis of cationic lipids for use in in vivo settings. Many “Good” buffers remain crucial tools in modern biochemistry and biology laboratories and are therefore readily available at low cost.

The inventors of the present invention have surprisingly found that lipid nanoparticles comprising a second generation of cationic lipids derived from “Good” buffers which contain an ester moiety in the lipid tails and short (C₃-C₆)alkyl tails, such as butyl, isopropyl and pentan-3-yl, after the ester moiety exhibit improved properties relative to lipid nanoparticles comprising other cationic lipids derived from “Good” buffers, such as in WO 2022/221688 A1 and WO 2022/066916 A1, both incorporated herein by reference. For example, it is contemplated that lipid nanoparticles comprising the second generation of cationic lipids derived from “Good” buffers may exhibit improved degradation in vivo. It is also contemplated that the lipid nanoparticles comprising the second generation of cationic lipids derived from “Good” buffers may also exhibit higher generalized polarization (GP) values from the laurdan assay. A lower generalized polarization (GP) value is associated with a hydrated and fluid membrane while a higher generalized polarization (GP) value typically means less water molecules and more ordered lipid packing. It is thought that the additional esters and/or carbon branches in the lipid tails of the second generation of cationic lipids derived from “Good” buffers could result in tighter packed membranes compared to lipid nanoparticles comprising other cationic lipids derived from “Good” buffers, such as in WO 2022/221688 A1 and WO 2022/066916 A1. It is thought that lipid nanoparticles with tighter bilayer packing may perform better in vivo by increasing lipid nanoparticle stability under physiological pH conditions. The “Good” HEPES, HEPPS, and HEPBS buffers form the cores of some of the cationic lipids of the invention and were used to synthesize unique ionizable lipids containing different degradable moieties and carbon tails. The core structure with a hydroxyl and sulfonic acid group on either side allows for the ionizable lipids to contain both ester and disulfide degradable moieties. Preferably the compounds also feature asymmetric lipids tails on either arm of the final molecule and in the lipids of the invention, those tails contain ester moieties with the aim of achieving higher degradability.

The present invention provides, among other things, cationic lipid compounds for in vivo delivery of therapeutic agents, such as nucleic acids. The cationic lipids of the present invention can be synthesized from readily available starting reagents, such as “Good's” buffers (see Table 1). The cationic lipids of the present invention also comprise cleavable groups (e.g., esters and disulphides) that are contemplated to improve biodegradability and thus contribute to their favorable safety profile. It is contemplated that lipid nanoparticles comprising these cationic lipid compounds are capable of highly effective in vivo delivery while maintaining a favorable safety profile. It is also contemplated that lipid nanoparticles comprising these cationic lipid compounds may exhibit improved degradation in vivo. It is further contemplated that lipid nanoparticles comprising these cationic lipid compounds may exhibit higher generalized polarization (GP) values.

In an aspect, provided herein are cationic lipids having a structure according to Formula (I):

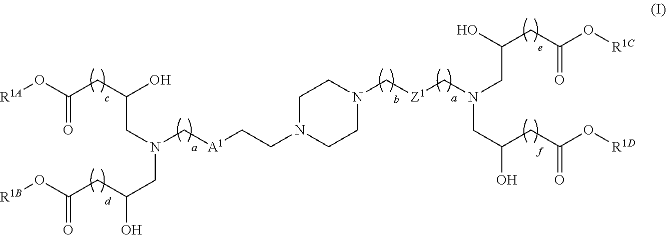

or a pharmaceutically acceptable salt thereof, wherein:
A¹ is selected from

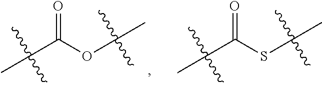 [SMILES: *OC(*)=O ; *SC(*)=O]

and —S—S—, wherein the left hand side of each depicted structure is bound to the —(CH₂)a-;
Z¹ is selected from

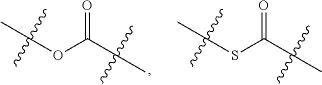 [SMILES: *OC(*)=O ; *SC(*)=O]

and —S—S—, wherein the right hand side of each depicted structure is bound to the —(CH₂)a-;
each a is independently selected from 3 or 4;
b is 1, 2, 3, 4 or 5;
each c, d, e and f is independently selected from 3, 4, 5 or 6; and
each R^{1A}, R^{1B}, R^{1C} and R^{1D} is independently selected from optionally substituted (C₃-C₆)alkyl.

In an aspect, provided herein are cationic lipids that are pharmaceutically acceptable salts of Formula (I).

In an aspect, provided herein are compositions comprising the cationic lipid of the present invention or a pharmaceutically acceptable salt thereof, and further comprising:
(i) one or more non-cationic lipids (e.g. a phospholipid, such as DOPE),
(ii) one or more cholesterol-based lipids (e.g. cholesterol) and
(iii) one or more PEG-modified lipid.

In an aspect, the composition is a lipid nanoparticle, optionally a liposome.

In an aspect, the compositions comprising the cationic lipids of the present invention may be used in therapy.

In an aspect, the compositions of the invention are administered by intramuscular injection.

### DETAILED DESCRIPTION OF CERTAIN EMBODIMENTS

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Amino acid: As used herein, the term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an I-amino acid. “Standard amino acid” refers to any of the twenty standard I-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, a bovine, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the term “biologically active” refers to a characteristic of any agent that has activity in a biological system, and particularly in an organism. For instance, an agent that, when administered to an organism, has a biological effect on that organism, is considered to be biologically active.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery”).

Expression: As used herein, “expression” of a nucleic acid sequence refers to translation of an mRNA into a polypeptide, assemble multiple polypeptides into an intact protein (e.g., enzyme) and/or post-translational modification of a polypeptide or fully assembled protein (e.g., enzyme). In this application, the terms “expression” and “production,” and grammatical equivalents thereof, are used interchangeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Helper lipid: The term “helper lipid” as used herein refers to any neutral or zwitterionic lipid material including cholesterol. Without wishing to be held to a particular theory, helper lipids may add stability, rigidity, and/or fluidity within lipid bilayers/nanoparticles.

Improve, increase, or reduce: As used herein, the terms “improve,” “increase,” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Liposome: As used herein, the term “liposome” refers to any lamellar, multilamellar, or solid nanoparticle vesicle. Typically, a liposome as used herein can be formed by mixing one or more lipids or by mixing one or more lipids and polymer(s). In some embodiments, a liposome suitable for the present invention contains a cationic lipid(s) and optionally further comprises:
(i) non-cationic lipid(s),
(ii) cholesterol-based lipid(s), and/or
(iii) PEG-modified lipid(s).

messenger RNA (mRNA): As used herein, the term “messenger RNA (mRNA)” or “mRNA” refers to a polynucleotide that encodes at least one polypeptide. mRNA as used herein encompasses both modified and unmodified RNA. The term “modified mRNA” related to mRNA comprising at least one chemically modified nucleotide. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C5-propynyl-cytidine, C5-propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. In some embodiments, “nucleic acid” encompasses ribonucleic acids (RNA), including but not limited to any one or more of interference RNAs (RNAi), small interfering RNA (siRNA), short hairpin RNA (shRNA), antisense RNA (aRNA), messenger RNA (mRNA), modified messenger RNA (mmRNA), long non-coding RNA (lncRNA), micro-RNA (miRNA) multimeric coding nucleic acid (MCNA), polymeric coding nucleic acid (PCNA), guide RNA (gRNA) and CRISPR RNA (crRNA). In some embodiments, “nucleic acid” encompasses deoxyribonucleic acid (DNA), including but not limited to any one or more of single-stranded DNA (ssDNA), double-stranded DNA (dsDNA) and complementary DNA (cDNA). In some embodiments, “nucleic acid” encompasses both RNA and DNA. In embodiments, DNA may be in the form of antisense DNA, plasmid DNA, parts of a plasmid DNA, pre-condensed DNA, a product of a polymerase chain reaction (PCR), vectors (e.g., P1, PAC, BAC, YAC, artificial chromosomes), expression cassettes, chimeric sequences, chromosomal DNA, or derivatives of these groups. In embodiments, RNA may be in the form of messenger RNA (mRNA), ribosomal RNA (rRNA), signal recognition particle RNA (7 SL RNA or SRP RNA), transfer RNA (tRNA), transfer-messenger RNA (tmRNA), small nuclear RNA (snRNA), small nucleolar RNA (snoRNA), SmY RNA, small Cajal body-specific RNA (scaRNA), guide RNA (gRNA), ribonuclease P (RNase P), Y RNA, telomerase RNA component (TERC), spliced leader RNA (SL RNA), antisense RNA (aRNA or asRNA), cis-natural antisense transcript (cis-NAT), CRISPR RNA (crRNA), long noncoding RNA (lncRNA), micro-RNA (miRNA), piwi-interacting RNA (piRNA), small interfering RNA (siRNA), transacting siRNA (tasiRNA), repeat associated siRNA (rasiRNA), 73K RNA, retrotransposons, a viral genome, a viroid, satellite RNA, or derivatives of these groups. In some embodiments, a nucleic acid is a mRNA encoding a protein such as an enzyme.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable,” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, non-toxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quarternization of an amine using an appropriate electrophile, e.g., an alkyl halide, to form a quarternized alkylated amino salt.

Systemic distribution or delivery: As used herein, the terms “systemic distribution” or “systemic delivery,” or grammatical equivalents thereof, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### Chemical Definitions

Acyl: As used herein, the term “acyl” refers to R^{z}—(C═O)—, wherein R^{z} is, for example, any alkyl, alkenyl, alkynyl, heteroalkyl or heteroalkylene.

Aliphatic: As used herein, the term aliphatic refers to (C₁-C₅₀) hydrocarbons and includes both saturated and unsaturated hydrocarbons. An aliphatic may be linear, branched, or cyclic. For example, (C₁-C₂₀)aliphatics can include (C₁-C₂₀)alkyls (e.g., linear or branched (C₁-C₂₀) saturated alkyls), (C₂-C₂₀) alkenyls (e.g., linear or branched (C₄-C₂₀) dienyls, linear or branched (C₆-C₂₀) trienyls, and the like), and (C₂-C₂₀) alkynyls (e.g., linear or branched (C₂-C₂₀) alkynyls). (C₁-C₂₀) aliphatics can include (C₃-C₂₀) cyclic aliphatics (e.g., (C₃-C₂₀) cycloalkyls, (C₄-C₂₀) cycloalkenyls, or (C₈-C₂₀) cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In embodiments, the aliphatic is unsubstituted. In embodiments, the aliphatic does not include any heteroatoms. Alkyl: As used herein, the term “alkyl” means acyclic linear and branched hydrocarbon groups, e.g. “(C₁-C₃₀) alkyl” refers to alkyl groups having 1-30 carbons. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In embodiments, the alkyl is substituted (e.g., with 1, 2, 3, 4, 5, or 6 substituent groups as described herein). In embodiments, an alkyl group is substituted with a —OH group and may also be referred to herein as a “hydroxyalkyl” group, where the prefix denotes the —OH group and “alkyl” is as described herein.

As used herein, “alkyl” also refers to a radical of a straight-chain or branched saturated hydrocarbon group having from 1 to 50 carbon atoms (“(C₁-C₅₀) alkyl”). In some embodiments, an alkyl group has 1 to 40 carbon atoms (“(C₁-C₄₀) alkyl”). In some embodiments, an alkyl group has 1 to 30 carbon atoms (“(C₁-C₃₀) alkyl”). In some embodiments, an alkyl group has 1 to 20 carbon atoms (“(C₁-C₂₀) alkyl”). In some embodiments, an alkyl group has 1 to 10 carbon atoms (“(C₁-C₁₀) alkyl”). In some embodiments, an alkyl group has 1 to 9 carbon atoms (“(C₁-C₉) alkyl”). In some embodiments, an alkyl group has 1 to 8 carbon atoms (“(C₁-C₈) alkyl”). In some embodiments, an alkyl group has 1 to 7 carbon atoms (“(C₁-C₇) alkyl”). In some embodiments, an alkyl group has 1 to 6 carbon atoms (“(C₁-C₆) alkyl”). In some embodiments, an alkyl group has 1 to 5 carbon atoms (“(C₁-C₅) alkyl”). In some embodiments, an alkyl group has 1 to 4 carbon atoms (“(C₁-C₄) alkyl”). In some embodiments, an alkyl group has 1 to 3 carbon atoms (“(C₁-C₃) alkyl”). In some embodiments, an alkyl group has 1 to 2 carbon atoms (“(C₁-C₂) alkyl”). In some embodiments, an alkyl group has 1 carbon atom (“C₁ alkyl”). In some embodiments, an alkyl group has 2 to 6 carbon atoms (“(C₂-C₆) alkyl”). In some embodiments, an alkyl group has 3 to 6 carbon atoms (“(C₃-C₆) alkyl”). Examples of (C₁-C₆) alkyl groups include, without limitation, methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted (C₁-C₅₀) alkyl. In certain embodiments, the alkyl group is a substituted (C₁-C₅₀) alkyl.

Affixing the suffix “-ene” to a group indicates the group is a divalent moiety, e.g., arylene is the divalent moiety of aryl, and heteroarylene is the divalent moiety of heteroaryl.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In certain embodiments, an alkylene, alkenylene, or alkynylene is unsubstituted. In certain embodiments, an alkylene, alkenylene, or alkynylene does not include any heteroatoms. Alkenyl: As used herein, “alkenyl” means any linear or branched hydrocarbon chains having one or more unsaturated carbon-carbon double bonds that may occur in any stable point along the chain, e.g. “(C₂-C₃₀) alkenyl” refers to an alkenyl group having 2-30 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In embodiments, the alkenyl comprises 1, 2, or 3 carbon-carbon double bond. In embodiments, the alkenyl comprises a single carbon-carbon double bond. In embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In embodiments, the alkenyl is unsubstituted. In embodiments, the alkenyl is substituted (e.g., with 1, 2, 3, 4, 5, or 6 substituent groups as described herein). In embodiments, an alkenyl group is substituted with a-OH group and may also be referred to herein as a “hydroxyalkenyl” group, where the prefix denotes the —OH group and “alkenyl” is as described herein.

As used herein, “alkenyl” also refers to a radical of a straight-chain or branched hydrocarbon group having from 2 to 50 carbon atoms and one or more carbon-carbon double bonds (e.g., 1, 2, 3, or 4 double bonds) (“(C₂-C₅₀) alkenyl”). In some embodiments, an alkenyl group has 2 to 40 carbon atoms (“(C₂-C₄₀) alkenyl”). In some embodiments, an alkenyl group has 2 to 30 carbon atoms (“(C₂-C₃₀) alkenyl”). In some embodiments, an alkenyl group has 2 to 20 carbon atoms (“(C₂-C₂₀) alkenyl”). In some embodiments, an alkenyl group has 2 to 10 carbon atoms (“(C₂-C₁₀) alkenyl”). In some embodiments, an alkenyl group has 2 to 9 carbon atoms (“(C₂—C) alkenyl”). In some embodiments, an alkenyl group has 2 to 8 carbon atoms (“(C₂—C) alkenyl”). In some embodiments, an alkenyl group has 2 to 7 carbon atoms (“(C₂-C₇) alkenyl”). In some embodiments, an alkenyl group has 2 to 6 carbon atoms (“(C₂-C₆) alkenyl”). In some embodiments, an alkenyl group has 2 to 5 carbon atoms (“(C₂-C₅) alkenyl”). In some embodiments, an alkenyl group has 2 to 4 carbon atoms (“(C₂-C₄) alkenyl”). In some embodiments, an alkenyl group has 2 to 3 carbon atoms (“(C₂-C₃) alkenyl”). In some embodiments, an alkenyl group has 2 carbon atoms (“(C₂) alkenyl”). The one or more carbon-carbon double bonds can be internal (such as in 2-butenyl) or terminal (such as in 1-butenyl). Examples of (C₂-C₄) alkenyl groups include, without limitation, ethenyl (C₂), 1-propenyl (C₃), 2-propenyl (C₃), 1-butenyl (C₄), 2-butenyl (C₄), butadienyl (C₄), and the like. Examples of (C₂-C₆) alkenyl groups include the aforementioned (C₂-C₄) alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted (C₂-C₅₀) alkenyl. In certain embodiments, the alkenyl group is a substituted (C₂-C₅₀) alkenyl.

Alkynyl: As used herein, “alkynyl” means any hydrocarbon chain of either linear or branched configuration, having one or more carbon-carbon triple bonds occurring in any stable point along the chain, e.g., “(C₂-C₃₀) alkynyl”, refers to an alkynyl group having 2-30 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR″, —CO₂H, —CO₂R″, —CN, —OH, —OR″, —OCOR″, —OCO₂R″, —NH₂, —NHR″, —N(R″)₂, —SR″ or —SO₂R″, wherein each instance of R″ independently is (C₁-C₂₀) aliphatic (e.g., (C₁-C₂₀) alkyl, (C₁-C₁₅) alkyl, (C₁-C₁₀) alkyl, or (C₁-C₃) alkyl). In embodiments, R″ independently is an unsubstituted alkyl (e.g., unsubstituted (C₁-C₂₀) alkyl, (C₁-C₁₅) alkyl, (C₁-C₁₀) alkyl, or (C₁-C₃) alkyl). In embodiments, R″ independently is unsubstituted (C₁-C₃) alkyl. In embodiments, the alkynyl is unsubstituted. In embodiments, the alkynyl is substituted (e.g., with 1, 2, 3, 4, 5, or 6 substituent groups as described herein).

As used herein, “alkynyl” also refers to a radical of a straight-chain or branched hydrocarbon group having from 2 to 50 carbon atoms and one or more carbon-carbon triple bonds (e.g., 1, 2, 3, or 4 triple bonds) and optionally one or more double bonds (e.g., 1, 2, 3, or 4 double bonds) (“(C₂-C₅₀) alkynyl”). An alkynyl group that has one or more triple bonds, and one or more double bonds is also referred to as an “ene-yne”. In some embodiments, an alkynyl group has 2 to 40 carbon atoms (“(C₂-C₄₀) alkynyl”). In some embodiments, an alkynyl group has 2 to 30 carbon atoms (“(C₂-C₃₀) alkynyl”). In some embodiments, an alkynyl group has 2 to 20 carbon atoms (“(C₂-C₂₀) alkynyl”). In some embodiments, an alkynyl group has 2 to 10 carbon atoms (“(C₂-C₁₀) alkynyl”). In some embodiments, an alkynyl group has 2 to 9 carbon atoms (“(C₂-C₉) alkynyl”). In some embodiments, an alkynyl group has 2 to 8 carbon atoms (“(C₂-C₈) alkynyl”). In some embodiments, an alkynyl group has 2 to 7 carbon atoms (“(C₂-C₇) alkynyl”). In some embodiments, an alkynyl group has 2 to 6 carbon atoms (“(C₂-C₆) alkynyl”). In some embodiments, an alkynyl group has 2 to 5 carbon atoms (“(C₂-C₅) alkynyl”). In some embodiments, an alkynyl group has 2 to 4 carbon atoms (“(C₂-C₄) alkynyl”). In some embodiments, an alkynyl group has 2 to 3 carbon atoms (“(C₂-C₃) alkynyl”). In some embodiments, an alkynyl group has 2 carbon atoms (“(C₂) alkynyl”). The one or more carbon--carbon triple bonds can be internal (such as in 2-butynyl) or terminal (such as in 1-butynyl). Examples of (C₂-C₄) alkynyl groups include, without limitation, ethynyl (C₂), 1-propynyl (C₃), 2-propynyl (C₃), 1-butynyl (C₄), 2-butynyl (C₄), and the like. Examples of (C₂-C₆) alkenyl groups include the aforementioned (C₂-C₄) alkynyl groups as well as pentynyl (C₅), hexynyl (C₅), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted (C₂-C₅₀) alkynyl. In certain embodiments, the alkynyl group is a substituted (C₂-C₅₀) alkynyl.

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 4 to 7 ring members. In embodiments, an aryl group has 6 ring carbon atoms (“(C₆) aryl,” e.g., phenyl). In some embodiments, an aryl group has 10 ring carbon atoms (“(C₁₀) aryl,” e.g., naphthyl such as 1-naphthyl and 2-naphthyl). In some embodiments, an aryl group has 14 ring carbon atoms (“(C₁₄) aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

As used herein, “aryl” also refers to a radical of a monocyclic or polycyclic (e.g., bicyclic or tricyclic) 4n+2 aromatic ring system (e.g., having 6, 10, or 14 T electrons shared in a cyclic array) having 6-14 ring carbon atoms and zero heteroatoms provided in the aromatic ring system (“(C₆-C₁₄) aryl”). In some embodiments, an aryl group has 6 ring carbon atoms (“(C₅) aryl”; e.g., phenyl). In some embodiments, an aryl group has 10 ring carbon atoms (“(C₁₀) aryl”; e.g., naphthyl such as 1-naphthyl and 2-naphthyl). In some embodiments, an aryl group has 14 ring carbon atoms (“(C₁₄) aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted (C₆-C₁₄) aryl. In certain embodiments, the aryl group is a substituted (C₆-C₁₄) aryl.

Arylene: The term “arylene” as used herein refers to an aryl group that is divalent (that is, having two points of attachment to the molecule). Exemplary arylenes include phenylene (e.g., unsubstituted phenylene or substituted phenylene).

Carbocyclyl: As used herein, “carbocyclyl” or “carbocyclic” refers to a radical of a non-aromatic cyclic hydrocarbon group having from 3 to 10 ring carbon atoms (“(C₃-C₁₀) carbocyclyl”) and zero heteroatoms in the non-aromatic ring system. In some embodiments, a carbocyclyl group has 3 to 8 ring carbon atoms (“(C₃-C₈) carbocyclyl”). In some embodiments, a carbocyclyl group has 3 to 7 ring carbon atoms (“(C₃-C₇) carbocyclyl”). In some embodiments, a carbocyclyl group has 3 to 6 ring carbon atoms (“(C₃-C₆) carbocyclyl”). In some embodiments, a carbocyclyl group has 4 to 6 ring carbon atoms (“(C₄-C₆) carbocyclyl”). In some embodiments, a carbocyclyl group has 5 to 6 ring carbon atoms (“(C₅-C₆) carbocyclyl”). In some embodiments, a carbocyclyl group has 5 to 10 ring carbon atoms (“(C₅-C₁₀) carbocyclyl”). Exemplary (C₃-C₆) carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₅), cyclohexadienyl (C₅), and the like. Exemplary (C₃-C₈) carbocyclyl groups include, without limitation, the aforementioned (C₃-C₆) carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary (C₃-C₁₀) carbocyclyl groups include, without limitation, the aforementioned (C₃-C₈) carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃-C₁₀ carbocyclyl. In certain embodiments, the carbocyclyl group is a substituted (C₃-C₁₀) carbocyclyl.

In some embodiments, “carbocyclyl” or “carbocyclic” is referred to as a “cycloalkyl”, i.e., a monocyclic, saturated carbocyclyl group having from 3 to 10 ring carbon atoms (“(C₃-C₁₀) cycloalkyl”). In some embodiments, a cycloalkyl group has 3 to 8 ring carbon atoms (“(C₃-C₈) cycloalkyl”). In some embodiments, a cycloalkyl group has 3 to 6 ring carbon atoms (“(C₃-C₆), cycloalkyl”). In some embodiments, a cycloalkyl group has 4 to 6 ring carbon atoms (“(C₄-C₆) cycloalkyl”). In some embodiments, a cycloalkyl group has 5 to 6 ring carbon atoms (“(C₅-C₆) cycloalkyl”). In some embodiments, a cycloalkyl group has 5 to 10 ring carbon atoms (“(C₅-C₁₀) cycloalkyl”). Examples of (C₅-C₆) cycloalkyl groups include cyclopentyl (C₅) and cyclohexyl (C₅). Examples of (C₃-C₆) cycloalkyl groups include the aforementioned (C₅-C₆) cycloalkyl groups as well as cyclopropyl (C₃) and cyclobutyl (C₄). Examples of (C₃-C₈) cycloalkyl groups include the aforementioned (C₃-C₆) cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted (C₃-C₁₀) cycloalkyl. In certain embodiments, the cycloalkyl group is a substituted (C₃-C₁₀) cycloalkyl.

Halogen: As used herein, the term “halogen” means fluorine, chlorine, bromine, or iodine.

Heteroalkyl: The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl.

Heteroalkylene: The term “heteroalkylene,” as used herein, represents a divalent form of a heteroalkyl group as described herein.

Heteroaryl: The term “heteroaryl,” as used herein, is fully unsaturated heteroatom-containing ring wherein at least one ring atom is a heteroatom such as, but not limited to, nitrogen and oxygen.

As used herein, “heteroaryl” also refers to a radical of a 5-14 membered monocyclic or polycyclic (e.g., bicyclic or tricyclic) 4n+2 aromatic ring system (e.g., having 6, 10, or 14 T electrons shared in a cyclic array) having ring carbon atoms and 1 or more (e.g., 1, 2, 3, or 4 ring heteroatoms) ring heteroatoms provided in the aromatic ring system, wherein each heteroatom is independently selected from oxygen, sulfur, nitrogen, boron, silicon, and phosphorus (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-10 membered heteroaryl”). In some embodiments, a heteroaryl group is a 5-8 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-8 membered heteroaryl”). In some embodiments, a heteroaryl group is a 5-6 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-6 membered heteroaryl”). In some embodiments, the 5-6 membered heteroaryl has 1 or more (e.g., 1, 2, or 3) ring heteroatoms selected from oxygen, sulfur, nitrogen, boron, silicon, and phosphorus. In some embodiments, the 5-6 membered heteroaryl has 1 or 2 ring heteroatoms selected from oxygen, sulfur, nitrogen, boron, silicon, and phosphorus. In some embodiments, the 5-6 membered heteroaryl has 1 ring heteroatom selected from oxygen, sulfur, nitrogen, boron, silicon, and phosphorus. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As used herein, “heterocyclyl” or “heterocyclic” refers to a radical of a 3- to 14-membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)) and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-10 membered heterocyclyl”). In some embodiments, a heterocyclyl group is a 5-8 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-8 membered heterocyclyl”). In some embodiments, a heterocyclyl group is a 5-6 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-6 membered heterocyclyl”). In some embodiments, the 5-6 membered heterocyclyl has 1 or more (e.g., 1, 2, or 3) ring heteroatoms selected from oxygen, sulfur, nitrogen, boron, silicon, and phosphorus. In some embodiments, the 5-6 membered heterocyclyl has 1 or 2 ring heteroatoms selected from oxygen, sulfur, nitrogen, boron, silicon, and phosphorus. In some embodiments, the 5-6 membered heterocyclyl has 1 ring heteroatom selected from oxygen, sulfur, nitrogen, boron, silicon, and phosphorus.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno [3,2-b]pyridinyl, 1,2,3,4-tetrahydro-1,6-naphthyridinyl, and the like.

Heterocycloalkyl: The term “heterocycloalkyl,” as used herein, is a non-aromatic ring wherein at least one atom is a heteroatom such as, but not limited to, nitrogen, oxygen, sulfur, or phosphorus, and the remaining atoms are carbon. The heterocycloalkyl group can be substituted or unsubstituted.

As understood from the above, alkyl, alkenyl, alkynyl, acyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valences of the heteroatoms and results in the formation of a stable moiety.

Exemplary carbon atom substituents include, but are not limited to, halogen, —CN, —NO₂, —N₃, —SO₂, —SO₃H, —OH, —OR^{aa}, —ON(R^{bb})₂, —N(R^{bb})₂, —N(R^{bb})₃+X⁻, —N(OR″)R^{bb}, —SeH, —SeR^{aa}, —SH, —SR^{aa}, —SSR^{cc}, —C(═O)R^{aa}, —CO₂H, —CHO, —C(OR″)₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃—OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR″)₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(ORR″)₂, —NR^{bb}P(═O)(NR^{bb})₂, —P(R″)₂, —P(R″)₃, —OP(R″)₂, —OP(R″)₃, —B(R^{aa})₂, —B(ORR″)₂, —BR^{aa}(OR″), (C₁-C₅₀) alkyl, (C₂-C₅₀) alkenyl, (C₂-C₅₀) alkynyl, (C₃-C₁₄) carbocyclyl, 3-14 membered heterocyclyl, (C₆-C₁₄) aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, =NNR^{bb}C(═O)R^{aa}, =NNR^{bb}C(═O)OR^{aa}, =NNR^{bb}S(═O)₂R^{aa}, =NR^{bb}, or =NOR^{cc};
each instance of R^{aa} is, independently, selected from (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{bb} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{cc} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃+X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NRC(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, (C₁-C₅₀) alkyl, (C₂-C₅₀) alkenyl, (C₂-C₅₀) alkynyl, (C₃-C₁₀) carbocyclyl, 3-10 membered heterocyclyl, (C₆-C₁₀) aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or =S;
each instance of R^{ee} is, independently, selected from (C₁-C₅₀) alkyl, (C₂-C₅₀) alkenyl, (C₂-C₅₀) alkynyl, (C₃-C₁₀) carbocyclyl, (C₆-C₁₀) aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{f} is, independently, selected from hydrogen, (C₁-C₅₀) alkyl, (C₂-C₅₀) alkenyl, (C₂-C₅₀) alkynyl, (C₃-C₁₀) carbocyclyl, 3-10 membered heterocyclyl, (C₆-C₁₀) aryl and 5-10 membered heteroaryl, or two R^{f} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —O(C₁-C₅₀) alkyl, —ON((C₁-C₅₀) alkyl)₂, —N((C₁-C₅₀) alkyl)₂, —N((C₁-C₅₀) alkyl)₃+X⁻, —NH((C₁-C₅₀) alkyl)₂+X⁻, —NH₂((C₁-C₅₀) alkyl)+X⁻, —NH₃+X⁻, —N(O(C₁-C₅₀) alkyl)((C₁-C₅₀) alkyl), —N(OH)((C₁-C₅₀) alkyl), —NH(OH), —SH, —S(C₁-C₅₀) alkyl, —SS((C₁-C₅₀) alkyl), —C(═O)((C₁-C₅₀) alkyl), —CO₂H, —CO₂((C₁-C₅₀) alkyl), —OC(═O)((C₁-C₅₀) alkyl), —OCO₂((C₁-C₅₀) alkyl), —C(═O)NH₂, —C(═O)N((C₁-C₅₀) alkyl)₂, —OC(═O)NH((C₁-C₅₀) alkyl), —NHC(═O)((C₁-C₅₀) alkyl), —N((C₁-C₅₀) alkyl)C(═O)((C₁-C₅₀) alkyl), —NHCO₂((C₁-C₅₀) alkyl), —NHC(═O)N((C₁-C₅₀) alkyl)₂, —NHC(═O)NH((C₁-C₅₀) alkyl), —NHC(═O)NH₂, —C(═NH)O((C₁-C₅₀) alkyl), —OC(═NH)((C₁-C₅₀) alkyl), —OC(═NH)O(C₁-C₅₀) alkyl, —C(═NH)N((C₁-C₅₀) alkyl)₂, —C(═NH)NH((C₁-C₅₀) alkyl), —C(═NH)NH₂, —OC(═NH)N((C₁-C₅₀)alkyl)₂, —OC(NH)NH((C₁-C₅₀) alkyl), —OC(NH)NH₂, —NHC(NH)N((C₁-C₅₀) alkyl)₂, —NHC(═NH)NH₂, —NHSO₂((C₁-C₅₀) alkyl), —SO₂N((C₁-C₅₀) alkyl)₂, —SO₂NH((C₁-C₅₀) alkyl), —SO₂NH₂, —SO₂((C₁-C₅₀) alkyl), —SO₂O((C₁-C₅₀) alkyl), —OSO₂((C₁-C₆) alkyl), —SO((C₁-C₆) alkyl), —Si((C₁-C₅₀) alkyl)₃, —OSi((C₁-C₆) alkyl)₃, —C(═S)N((C₁-C₅₀) alkyl)₂, C(═S)NH((C₁-C₅₀) alkyl), C(═S)NH₂, —C(═O)S((C₁-C₆) alkyl), —C(═S)S((C₁-C₆) alkyl), —SC(═S)S((C₁-C₆) alkyl), —P(═O)₂((C₁-C₅₀) alkyl), —P(═O)((C₁-C₅₀) alkyl)₂, —OP(═O)((C₁-C₅₀) alkyl)₂, —OP(═O)(O(C₁-C₅₀) alkyl)₂, (C₁-C₅₀) alkyl, (C₂-C₅₀) alkenyl, (C₂-C₅₀) alkynyl, (C₃-C₁₀) carbocyclyl, (C₆-C₁₀) aryl, 3-10 membered heterocyclyl, 5-10 membered heteroaryl; or two geminal R^{gg} substituents can be joined to form ═O or =S; wherein X⁻ is a counterion.

As used herein, the term “halo” or “halogen” refers to fluorine (fluoro, —F), chlorine (chloro, —Cl), bromine (bromo, —Br), or iodine (iodo, —I).

As used herein, a “counterion” is a negatively charged group associated with a positively charged quarternary amine in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-l-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, (C₁-C₅₀) alkyl, (C₂-C₅₀) alkenyl, (C₂-C₅₀) alkynyl, (C₃-C₁₀) carbocyclyl, 3-14 membered heterocyclyl, (C₆-C₁₄) aryl, and 5-14 membered heteroaryl, or two R^{cc} groups, together with the N atom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (lpaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-l-cyclopropylmethyl carbamate, 1-methyl-1(3,5-dimethoxyphenyl)ethyl carbamate, 1-methyl-1-(p-phenylazophenyl)ethyl carbamate, 1-methyl-I-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary sulfur protecting groups include, but are not limited to, alkyl, benzyl, p-methoxybenzyl, 2,4,6-trimethylbenzyl, 2,4,6-trimethoxybenzyl, o-hydroxybenzyl, p-hydroxybenzyl, o-acetoxybenzyl, p-acetoxybenzyl, p-nitrobenzyl, 4-picolyl, 2-quinolinylmethyl, 2-picolyl N-oxido, 9-anthrylmethyl, 9-fluorenylmethyl, xanthenyl, ferrocenylmethyl, diphenylmethyl, bis(4-methoxyphenyl)methyl, 5-dibenzosuberyl, triphenylmethyl, diphenyl-4-pyridylmethyl, phenyl, 2,4-dinitrophenyl, t-butyl, 1-adamantyl, methoxymethyl (MOM), isobutoxymethyl, benzyloxymethyl, 2-tetrahydropyranyl, benzylthiomethyl, phenylthiomethyl, thiazolidino, acetamidomethyl, trimethylacetamidomethyl, benzamidomethyl, allyloxycarbonylaminomethyl, phenylacetamidomethyl, phthalimidomethyl, acetylmethyl, carboxymethyl, cyanomethyl, (2-nitro-1-phenyl)ethyl, 2-(2,4-dinitrophenyl)ethyl, 2-cyanoethyl, 2-(Trimethylsilyl)ethyl, 2,2-bis(carboethoxy)ethyl, (1-m-nitrophenyl-2-benzoyl)othyl, 2-phenylsulfonylethyl, 2-(4-methylphenylsulfonyl)-2-methylprop-2-yl, acetyl, benzoyl, trifluoroacetyl, N-[[(p-biphenylyl)isopropoxy]carbonyl]-N-methyl]-γ-aminothiobutyrate, 2,2,2-trichloroethoxycarbonyl, t-butoxycarbonyl, benzyloxycarbonyl, p-methoxybenzyloxycarbonyl, N-ethyl, N-methoxymethyl, sulfonate, sulfenylthiocarbonate, 3-nitro-2-pyridinesulfenyl sulfide, oxathiolone.

### Compounds of the Invention

Liposomal-based vehicles are considered as an attractive carrier for therapeutic agents and remain subject to continued development efforts. While liposomal-based vehicles that comprise certain lipid components have shown promising results with regard to encapsulation, stability and site localization, there remains a great need for improvement of liposomal-based delivery systems. For example, a significant drawback of liposomal delivery systems relates to the construction of liposomes that have sufficient cell culture or in vivo stability to reach desired target cells and/or intracellular compartments, and the ability of such liposomal delivery systems to efficiently release their encapsulated materials to such target cells.

In particular, there remains a need for cationic lipids that are effective for intramuscular delivery of mRNA. There also remains a need for improved lipid compounds that demonstrate improved pharmacokinetic properties, and which are capable of delivering macromolecules, such as nucleic acids, to a wide variety cell types and tissues with enhanced efficiency. Importantly, there also remains a particular need for novel lipid compounds that are characterized as having improved safety profiles and are capable of efficiently delivering encapsulated nucleic acids and polynucleotides to targeted cells, tissues and organs.

Described herein is a novel class of cationic lipid compounds for improved in vivo delivery of therapeutic agents, such as nucleic acids. In particular, a cationic lipid described herein may be used, optionally with other lipids, to formulate a lipid-based nanoparticle (e.g., liposome) for encapsulating therapeutic agents, such as nucleic acids (e.g., DNA, siRNA, mRNA, microRNA) for therapeutic use, such as disease treatment and prevention (vaccine) purposes.

In embodiments, compounds of the invention as described herein can provide one or more desired characteristics or properties. That is, in certain embodiments, compounds of the invention as described herein can be characterized as having one or more properties that afford such compounds advantages relative to other similarly classified lipids. For example, compounds disclosed herein can allow for the control and tailoring of the properties of liposomal compositions (e.g., lipid nanoparticles) of which they are a component. In particular, compounds disclosed herein can be characterized by enhanced transfection efficiencies and their ability to provoke specific biological outcomes. Such outcomes can include, for example enhanced cellular uptake, endosomal/lysosomal disruption capabilities and/or promoting the release of encapsulated materials (e.g., polynucleotides) intracellularly. The compounds disclosed herein can also be characterized by achieving high levels of peptide or protein expression when delivering mRNA encoding for said peptide or protein by intravenous, intrathecal or intramuscular administration, or by pulmonary delivery, optionally through nebulization. Additionally, the compounds disclosed herein have advantageous pharmacokinetic properties, biodistribution, and efficiency.

The present application demonstrates that not only are the cationic lipids of the present invention synthetically tractable from readily available starting materials, but they also have unexpectedly high encapsulation efficiencies.

Additionally, the cationic lipids of the present invention have cleavable groups such as ester groups. These cleavable groups (e.g. esters, disulphides) are contemplated to improve biodegradability and thus contribute to the lipids' favorable safety profiles.

Provided herein are compounds which are cationic lipids. For example, the cationic lipids of the present invention include compounds having a structure according to Formula (I):

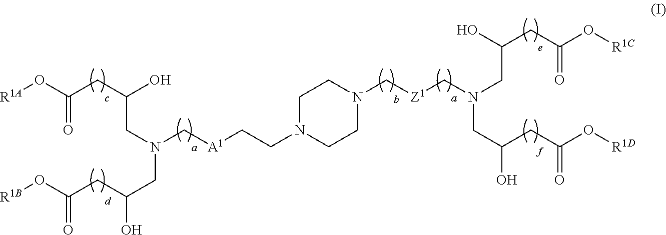

or a pharmaceutically acceptable salt thereof, wherein:
A¹ is selected from

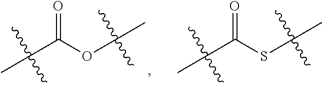 [SMILES: *OC(*)=O ; *SC(*)=O]

and —S—S—, wherein the left hand side of each depicted structure is bound to the —(CH₂)a-;
Z¹ is selected from

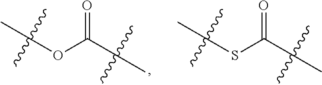 [SMILES: *OC(*)=O ; *SC(*)=O]

and —S—S—, wherein the right hand side of each depicted structure is bound to the —(CH₂)a-;
each a is independently selected from 3 or 4;
b is 1, 2, 3, 4 or 5;
each c, d, e and f is independently selected from 3, 4, 5 or 6; and
each R^{1A}, R^{1B}, R^{1C} and R^{1D} is independently selected from optionally substituted (C₃-C₆)alkyl.

In embodiments, the cationic lipid has a structure according to Formula (Ia):

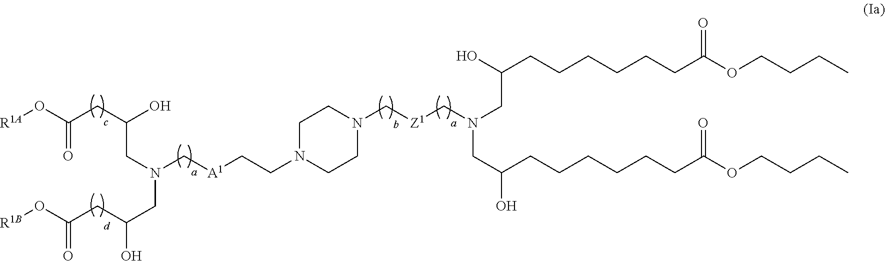

or a pharmaceutically acceptable salt thereof, optionally wherein:
(a) b is 2;
(b) b is 2, A¹ is

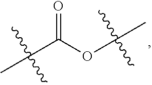 [SMILES: *OC(*)=O]

wherein the left hand side of the depicted structure is bound to the —(CH₂)a- and Z¹ is —S—S—; or
(c) b is 2, A¹ is

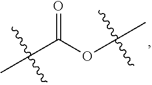 [SMILES: *OC(*)=O]

wherein the left hand side of the depicted structure is bound to the —(CH₂)a-, Z¹ is —S—S— and each c and d is independently selected from 3, 4, or 6.

In embodiments, the cationic lipid has a structure according to Formula (Ib):

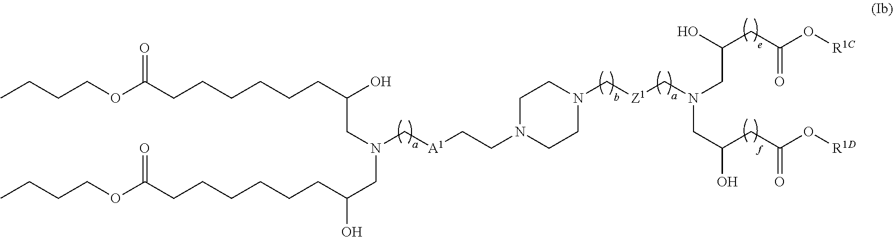

or a pharmaceutically acceptable salt thereof, optionally wherein:
(a) b is 2;
(b) b is 2, A¹ is

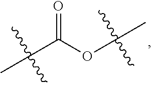 [SMILES: *OC(*)=O]

wherein the left hand side of the depicted structure is bound to the —(CH₂)a- and Z¹ is —S—S—; or
(c) b is 2, A¹ is

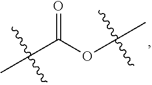 [SMILES: *OC(*)=O]

wherein the left hand side of the depicted structure is bound to the —(CH₂)a-, Z¹ is —S—S— and each e and f is independently selected from 3, 4, or 6.

In embodiments, the cationic lipid has a structure according to Formula (Ic):

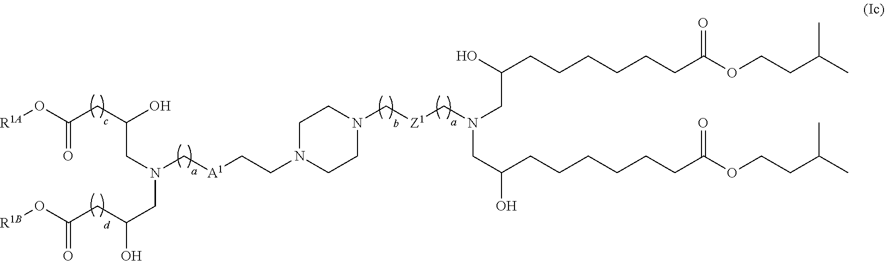

or a pharmaceutically acceptable salt thereof, optionally wherein:
(a) b is 2;
(b) b is 2, A¹ is

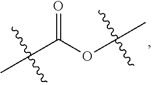 [SMILES: *OC(*)=O]

wherein the left hand side of the depicted structure is bound to the —(CH₂)a- and Z¹ is —S—S—; or
(c) b is 2, A¹ is

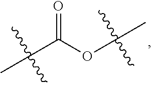 [SMILES: *OC(*)=O]

wherein the left hand side of the depicted structure is bound to the —(CH₂)a-, Z¹ is —S—S— and each c and d is independently selected from 3, 4, or 6.

In embodiments, the cationic lipid has a structure according to Formula (Id):

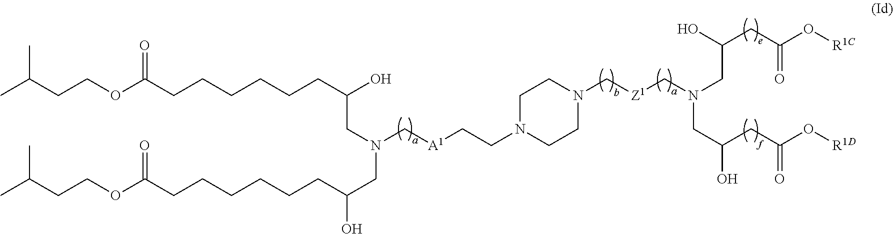

or a pharmaceutically acceptable salt thereof, optionally wherein:
(a) b is 2;
(b) b is 2, A¹ is

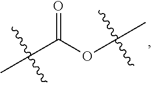 [SMILES: *OC(*)=O]

wherein the left hand side of the depicted structure is bound to the —(CH₂)a- and Z¹ is —S—S—; or
(c) b is 2, A¹ is

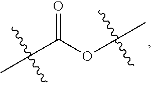 [SMILES: *OC(*)=O]

wherein the left hand side of the depicted structure is bound to the —(CH₂)a-, Z¹ is —S—S— and each e and f is independently selected from 3, 4, or 6.

In embodiments, the cationic lipid has a structure according to Formula (Ie):

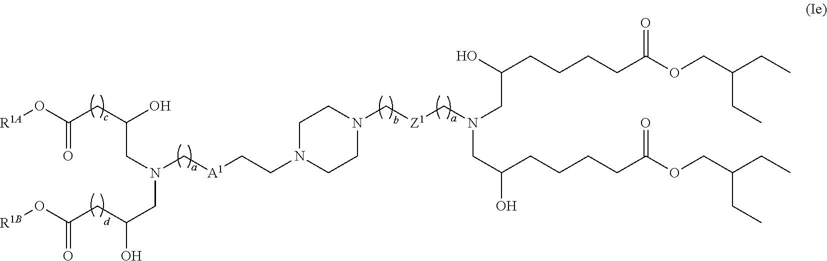

or a pharmaceutically acceptable salt thereof, optionally wherein:
(a) b is 2;
(b) b is 2, A¹ is

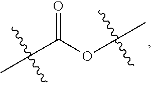 [SMILES: *OC(*)=O]

wherein the left hand side of the depicted structure is bound to the —(CH₂)a- and Z¹ is —S—S—; or
(c) b is 2, A¹ is

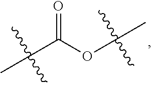 [SMILES: *OC(*)=O]

wherein the left hand side of the depicted structure is bound to the —(CH₂)a-, Z¹ is —S—S— and each c and d is independently selected from 3, 4, or 6.

In embodiments, the cationic lipid has a structure according to Formula (if):

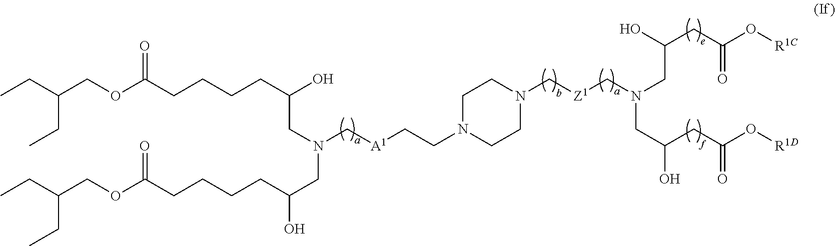

or a pharmaceutically acceptable salt thereof, optionally wherein:
(a) b is 2;
(b) b is 2, A¹ is

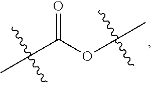 [SMILES: *OC(*)=O]

wherein the left hand side of the depicted structure is bound to the —(CH₂)a- and Z¹ is —S—S—; or
(c) b is 2, A¹ is

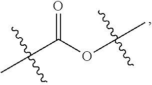 [SMILES: *OC(*)=O]

wherein the left hand side of the depicted structure is bound to the —(CH₂)a-, Z¹ is —S—S— and each e and f is independently selected from 3, 4, or 6.

In embodiments, the cationic lipid has a structure according to Formula (Ig):

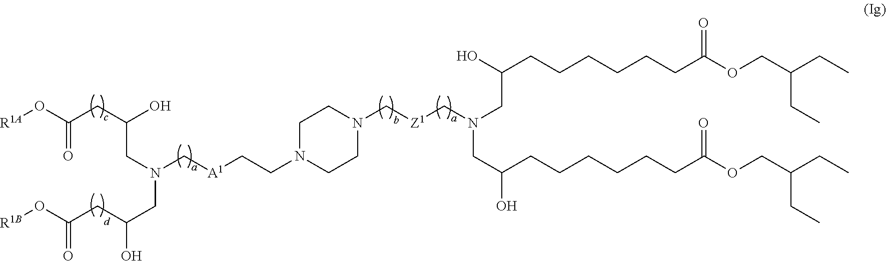

or a pharmaceutically acceptable salt thereof, optionally wherein:
(a) b is 2;
(b) b is 2, A¹ is

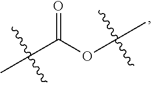 [SMILES: *OC(*)=O]

wherein the left hand side of the depicted structure is bound to the —(CH₂)a- and Z¹ is —S—S—; or
(c) b is 2, A¹ is

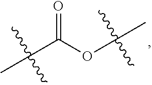 [SMILES: *OC(*)=O]

wherein the left hand side of the depicted structure is bound to the —(CH₂)a-, Z¹ is —S—S— and each c and d is independently selected from 3, 4, or 6.

In embodiments, the cationic lipid has a structure according to Formula (Ih):

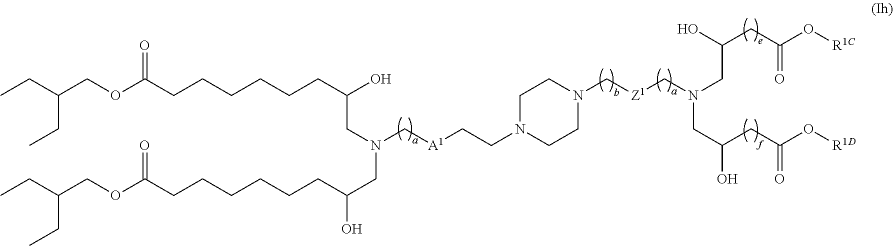

or a pharmaceutically acceptable salt thereof, optionally wherein:
(a) b is 2;
(b) b is 2, A¹ is

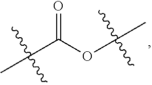 [SMILES: *OC(*)=O]

wherein the left hand side of the depicted structure is bound to the —(CH₂)a- and Z¹ is —S—S—; or
(c) b is 2, A¹ is

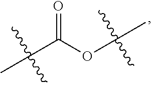 [SMILES: *OC(*)=O]

wherein the left hand side of the depicted structure is bound to the —(CH₂)a-, Z¹ is —S—S— and each e and f is independently selected from 3, 4, or 6.

In embodiments, the cationic lipid has a structure according to Formula (Ii):

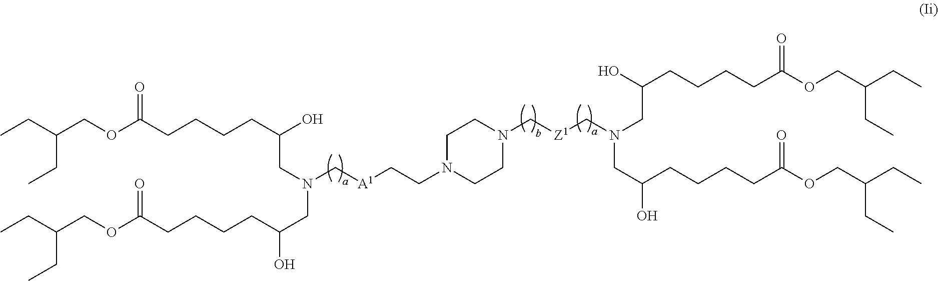

or a pharmaceutically acceptable salt thereof, optionally wherein:
(a) b is 2; or
(b) b is 2, A¹ is

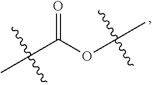 [SMILES: *OC(*)=O]

wherein the left hand side of the depicted structure is bound to the —(CH₂)a- and Z¹ is —S—S—.

In embodiments, the cationic lipid has a structure according to Formula (Ij):

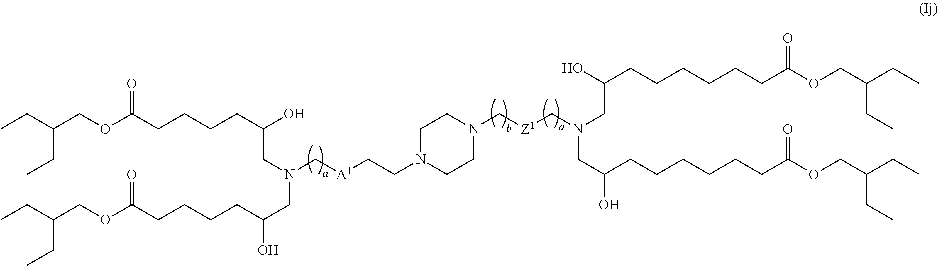

or a pharmaceutically acceptable salt thereof, optionally wherein:
(a) b is 2; or
(b) b is 2, A¹ is

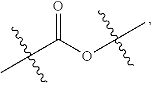 [SMILES: *OC(*)=O]

wherein the left hand side of the depicted structure is bound to the —(CH₂)a- and Z¹ is —S—S—.

In embodiments, the cationic lipid has a structure according to Formula (Ik):

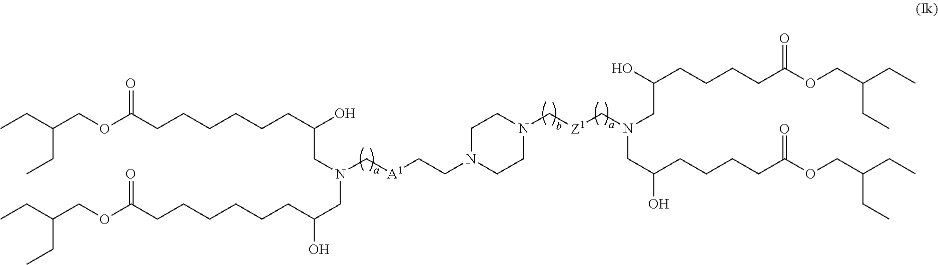

or a pharmaceutically acceptable salt thereof, optionally wherein:
(a) b is 2; or
(b) b is 2, A¹ is

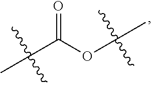 [SMILES: *OC(*)=O]

wherein the left hand side of the depicted structure is bound to the —(CH₂)a- and Z¹ is —S—S—.

In embodiments, the cationic lipid has a structure according to Formula (Im):

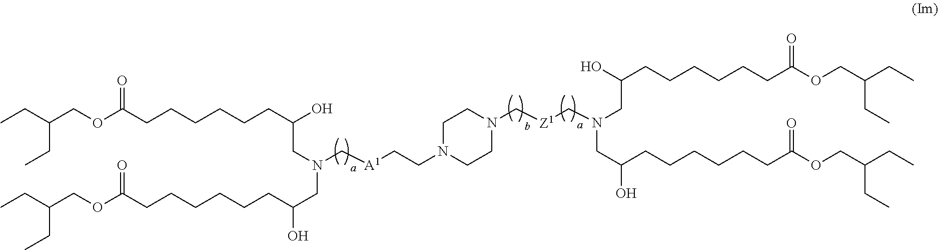

or a pharmaceutically acceptable salt thereof, optionally wherein:
(a) b is 2; or
(b) b is 2, A¹ is

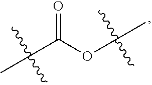 [SMILES: *OC(*)=O]

wherein the left hand side of the depicted structure is bound to the —(CH₂)a- and Z¹ is —S—S—.

In embodiments, the cationic lipid has a structure according to Formula (In):

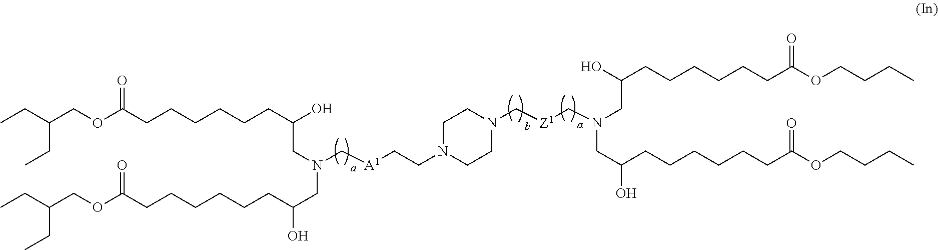

or a pharmaceutically acceptable salt thereof, optionally wherein:
(a) b is 2; or
(b) b is 2, A¹ is

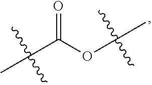 [SMILES: *OC(*)=O]

wherein the left hand side of the depicted structure is bound to the —(CH₂)a- and Z¹ is —S—S—.

In embodiments, the cationic lipid has a structure according to Formula (Io):

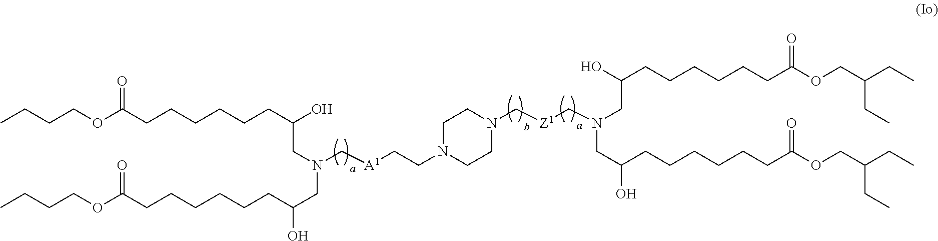

or a pharmaceutically acceptable salt thereof, optionally wherein:
(a) b is 2; or
(b) b is 2, A¹ is

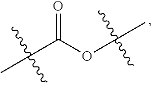 [SMILES: *OC(*)=O]

wherein the left hand side of the depicted structure is bound to the —(CH₂)a- and Z¹ is —S—S—.

In embodiments, the cationic lipid has a structure according to Formula (Ip):

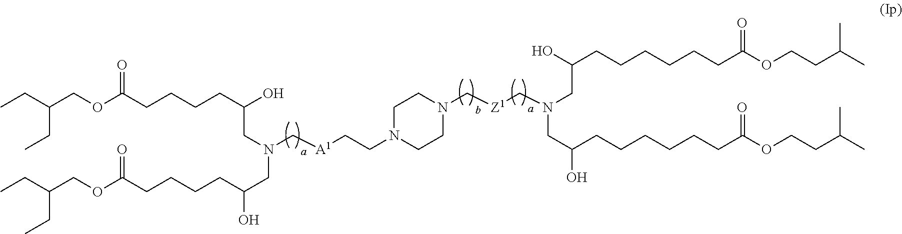

or a pharmaceutically acceptable salt thereof, optionally wherein:
(a) b is 2; or
(b) b is 2, A¹ is

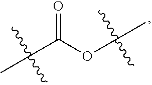 [SMILES: *OC(*)=O]

wherein the left hand side of the depicted structure is bound to the —(CH₂)a- and Z¹ is —S—S—.

In embodiments, the cationic lipid has a structure according to Formula (Iq):

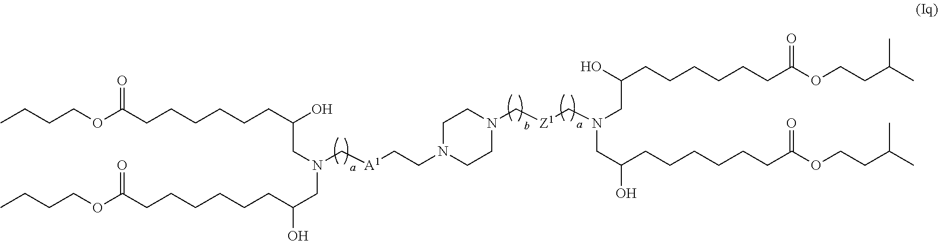

or a pharmaceutically acceptable salt thereof, optionally wherein:
(a) b is 2; or
(b) b is 2, A¹ is

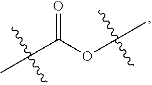 [SMILES: *OC(*)=O]

wherein the left hand side of the depicted structure is bound to the —(CH₂)a- and Z¹ is —S—S—.

In embodiments, A¹ and Z¹ are the same. In embodiments, A¹ and Z¹ are different.

In embodiments, A¹ is

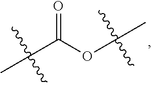 [SMILES: *OC(*)=O]

wherein the left hand side of the depicted structure is bound to the —(CH₂)a-. In embodiments, A¹ is

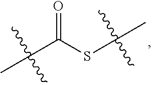 [SMILES: *SC(*)=O]

wherein the left hand side of the depicted structure is bound to the —(CH₂)a-. In embodiments, A¹ is-S—S—.

In embodiments, Z¹ is

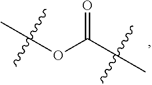 [SMILES: *OC(*)=O]

wherein the right hand side of the depicted structure is bound to the —(CH₂)a-. In embodiments, Z¹ is

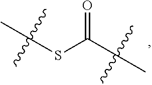 [SMILES: *SC(*)=O]

wherein the right hand side of the depicted structure is bound to the —(CH₂)a-. In embodiments, Z¹ is-S—S—.

In embodiments, b is 2, 3 or 4. In embodiments, b is 2 or 3. In embodiments, b is 1. In embodiments, b is 2. In embodiments, b is 3. In embodiments, b is 4. In embodiments, b is 5.

In embodiments, the cationic lipid has a structure according to Formula (Ir):

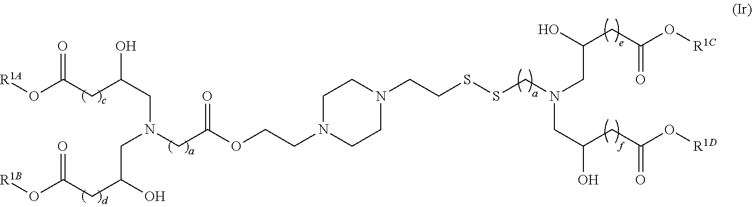

or a pharmaceutically acceptable salt thereof, optionally wherein each c, d, e and f is independently selected from 3, 4, or 6.

In embodiments, each a is 3. In embodiments, each a is 4. In embodiments, the value for the a on the left hand side of the depicted Formula is 3 and the value for the a on the right hand side of the depicted Formula is 4. In embodiments, the value for the a on the left hand side of the depicted Formula is 4 and the value for the a on the right hand side of the depicted Formula is 3.

In embodiments, c is 3, 4, or 6. In embodiments, c is 3. In embodiments, c is 4. In embodiments, c is 5. In embodiments, c is 6.

In embodiments, d is 3, 4, or 6. In embodiments, d is 3. In embodiments, d is 4. In embodiments, d is 5. In embodiments, d is 6.

In embodiments, e is 3, 4, or 6. In embodiments, e is 3. In embodiments, e is 4. In embodiments, e is 5. In embodiments, e is 6.

In embodiments, f is 3, 4, or 6. In embodiments, f is 3. In embodiments, f is 4. In embodiments, f is 5. In embodiments, f is 6.

In embodiments, each c, d, e and f is independently selected from 3, 4, or 6.

In embodiments, c, d, e and f are the same. In embodiments, c, d, e and f are 3. In embodiments, c, d, e and f are 4. In embodiments, c, d, e and f are 5. In embodiments, c, d, e and f are 6.

In embodiments, c and d are the same. In embodiments, c and d are 3. In embodiments, c and d are 4. In embodiments, c and d are 5. In embodiments, c and d are 6.

In embodiments, e and f are the same. In embodiments, e and f are 3. In embodiments, e and f are 4. In embodiments, e and f are 5. In embodiments, e and f are 6.

In embodiments, c and d are the same and e and f are the same, but wherein c and d are different to e and f. In embodiments, c and d are 3 and e and f are 4. In embodiments, c and d are 3 and e and f are 5. In embodiments, c and d are 3 and e and f are 6. In embodiments, c and d are 4 and e and f are 3. In embodiments, c and d are 4 and e and f are 5. In embodiments, c and d are 4 and e and f are 6. In embodiments, c and d are 5 and e and f are 3. In embodiments, c and d are 5 and e and f are 4. In embodiments, c and d are 5 and e and f are 6. In embodiments, c and d are 6 and e and f are 3. In embodiments, c and d are 6 and e and f are 4. In embodiments, c and d are 6 and e and f are 5.

In embodiments, each R^{1A}, R^{1B}, R^{1C} and R^{1D} is independently selected from optionally substituted (C₄-C₆)alkyl. In embodiments, each R^{1A}, R^{1B}, R^{1C} and R^{1D} is independently selected from optionally substituted (C₅-C₆)alkyl. In embodiments, each R^{1A}, R^{1B}, R^{1C} and R^{1D} is independently selected from optionally substituted (C₃-C₅)alkyl. In embodiments, each R^{1A}, R^{1B}, R^{1C} and R^{1D} is independently selected from optionally substituted (C₃-C₄)alkyl.

In embodiments, R^{1A} is optionally substituted C₃ alkyl. In embodiments, R^{1A} is optionally substituted C₄ alkyl. In embodiments, R^{1A} is optionally substituted C₅ alkyl. In embodiments, R^{1A} is optionally substituted C₆ alkyl.

In embodiments, R^{1B} is optionally substituted C₃ alkyl. In embodiments, R^{1B} is optionally substituted C₄ alkyl. In embodiments, R^{1B} is optionally substituted C₅ alkyl. In embodiments, R^{1B} is optionally substituted C₆ alkyl.

In embodiments, R^{1C} is optionally substituted C₃ alkyl. In embodiments, R^{1C} is optionally substituted C₄ alkyl. In embodiments, R^{1C} is optionally substituted C₅ alkyl. In embodiments, R^{1C} is optionally substituted C₆ alkyl.

In embodiments, R^{1D} is optionally substituted C₃ alkyl. In embodiments, R^{1D} is optionally substituted C₄ alkyl. In embodiments, R^{1D} is optionally substituted C₅ alkyl. In embodiments, R^{1D} is optionally substituted C₆ alkyl.

In embodiments, R^{1A}, R^{1B}, R^{1C} and R^{1D} are the same. In embodiments, R^{1A} and R^{1B} are the same. In embodiments, R^{1C} and R^{1D} are the same.

In embodiments, R^{1A} and R^{1B} are the same and R^{1C} and R^{1D} are the same, but wherein R^{1A} and R^{1B} are different to R^{1C} and R^{1D}.

In embodiments, each R^{1A}, R^{1B}, R^{1C} and R^{1D} where present is independently selected from:

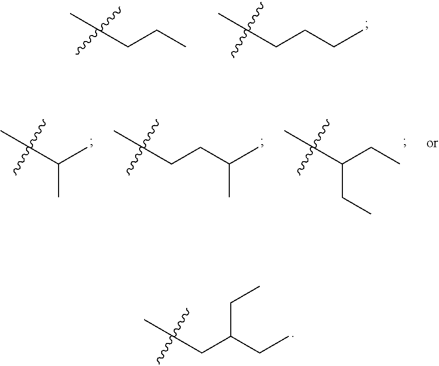

In embodiments, R^{1A} is

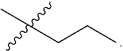 [SMILES: *CCC]

In embodiments, R^{1A} is

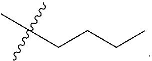 [SMILES: *CCCC]

In embodiments, R^{1A} is

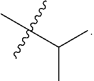 [SMILES: *C(C)C]

In embodiments, R^{1A} is

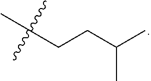 [SMILES: *CCC(C)C]

In embodiments, R^{1A} is

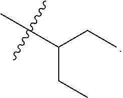 [SMILES: *C(CC)CC]

In embodiments, R^{1A} is

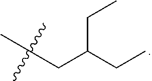 [SMILES: *CC(CC)CC]

In embodiments, R^{1B} is

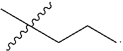 [SMILES: *CCC]

In embodiments, R^{1B} is

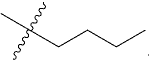 [SMILES: *CCCC]

In embodiments, R^{1B} is

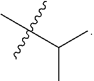 [SMILES: *C(C)C]

In embodiments, R^{1B} is

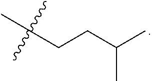 [SMILES: *CCC(C)C]

In embodiments, R^{1B} is

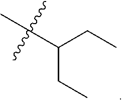 [SMILES: *C(CC)CC]

In embodiments, R^{1B} is

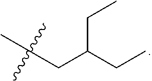 [SMILES: *CC(CC)CC]

In embodiments, R^{1C} is

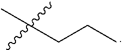 [SMILES: *CCC]

In embodiments, R^{1C} is

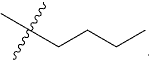 [SMILES: *CCCC]

In embodiments, R^{1C} is

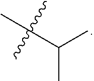 [SMILES: *C(C)C]

In embodiments, R^{1C} is

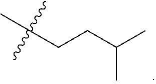 [SMILES: *CCC(C)C]

In embodiments, R^{1C} is

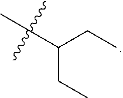 [SMILES: *C(CC)CC]

In embodiments, R^{1C} is

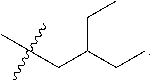 [SMILES: *CC(CC)CC]

In embodiments, R^{1D} is

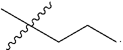 [SMILES: *CCC]

In embodiments, R^{1D} is

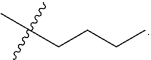 [SMILES: *CCCC]

In embodiments, R^{1D} is

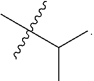 [SMILES: *C(C)C]

In embodiments, R^{1D} is

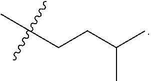 [SMILES: *CCC(C)C]

In embodiments, R^{1D} is

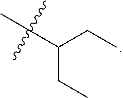 [SMILES: *C(CC)CC]

In embodiments, R^{1D} is

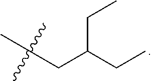 [SMILES: *CC(CC)CC]

In embodiments, c and d are 3 and R^{1A} and R^{1B} are

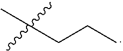 [SMILES: *CCC]

In embodiments, c and d are 4 and R^{1A} and R^{1B} are

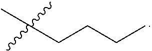 [SMILES: *CCCC]

In embodiments, c and d are 6 and R^{1A} and R^{1B} are

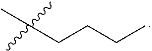 [SMILES: *CCCC]

In embodiments, c and d are 4 and R^{1A} and R^{1B} are

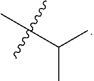 [SMILES: *C(C)C]

In embodiments, c and d are 6 and R^{1A} and R^{1B} are

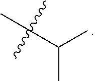 [SMILES: *C(C)C]

In embodiments, c and d are 4 and R^{1A} and R^{1B} are

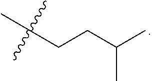 [SMILES: *CCC(C)C]

In embodiments, c and dare 6 and R^{1A} and R^{1B} are

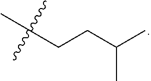 [SMILES: *CCC(C)C]

In embodiments, c and d are 3 and R^{1A} and R^{1B} are

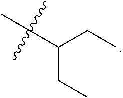 [SMILES: *C(CC)CC]

In embodiments, c and d are 4 and R^{1A} and R^{1B} are

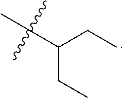 [SMILES: *C(CC)CC]

In embodiments, c and d are 6 and R^{1A} and R^{1B} are

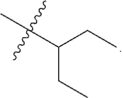 [SMILES: *C(CC)CC]

In embodiments, c and d are 3 and R^{1A} and R^{1B} are

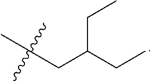 [SMILES: *CC(CC)CC]

In embodiments, c and d are 4 and R^{1A} and R^{1B} are

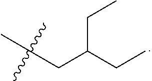 [SMILES: *CC(CC)CC]

In embodiments, c and d are 6 and R^{1A} and R^{1B} are

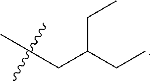 [SMILES: *CC(CC)CC]

In embodiments, e and f are 3 and R^{1C} and R^{1D} are

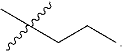 [SMILES: *CCC]

In embodiments, e and f are 4 and R^{1C} and R^{1D} are

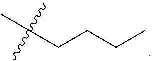 [SMILES: *CCCC]

In embodiments, e and f are 6 and R^{1C} and R^{1D} are

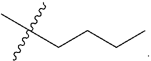 [SMILES: *CCCC]

In embodiments, e and f are 4 and R^{1C} and R^{1D} are

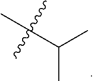 [SMILES: *C(C)C]

In embodiments, e and f are 6 and R^{1C} and R^{1D} are

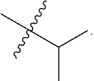 [SMILES: *C(C)C]

In embodiments, e and f are 4 and R^{1C} and R^{1D} are

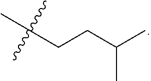 [SMILES: *CCC(C)C]

In embodiments, e and f are 6 and R^{1C} and R^{1D} are

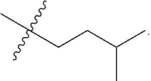 [SMILES: *CCC(C)C]

In embodiments, e and f are 3 and R^{1C} and R^{1D} are

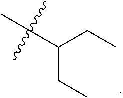 [SMILES: *C(CC)CC]

In embodiments, e and f are 4 and R^{1C} and R^{1D} are

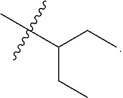 [SMILES: *C(CC)CC]

In embodiments, e and f are 6 and R^{1C} and R^{1D} are

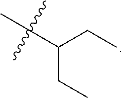 [SMILES: *C(CC)CC]

In embodiments, e and f are 3 and R^{1C} and R^{1D} are

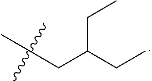 [SMILES: *CC(CC)CC]

In embodiments, e and f are 4 and R^{1C} and R^{1D} are

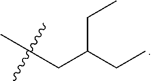 [SMILES: *CC(CC)CC]

In embodiments, e and f are 6 and R^{1C} and R^{1D} are

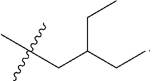 [SMILES: *CC(CC)CC]

In embodiments, each a is 4, c and d are 6, R^{1A} and R^{1B} are

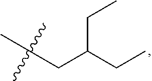 [SMILES: *CC(CC)CC]

e and f are 4 and R^{1C} and R^{1D} are

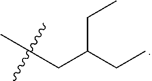 [SMILES: *CC(CC)CC]

In embodiments, the substituents are not optionally substituted.

In embodiments, the cationic lipids of the present invention have any one of the structures in Table A, Table B and/or Table C, or a pharmaceutically acceptable salt thereof.

In embodiments, the cationic lipids of the present invention have any one of the structures in the examples, or a pharmaceutically acceptable salt thereof.

In embodiments, provided herein is a composition comprising a cationic lipid of the present invention, and further comprising:
(i) one or more non-cationic lipids (e.g. a phospholipid, such as DOPE),
(ii) one or more cholesterol-based lipids (e.g. cholesterol) and
(iii) one or more PEG-modified lipids.

In embodiments, this composition is a lipid nanoparticle, optionally a liposome. In embodiments, the one or more cationic lipid(s) constitute(s) about 30 mol %-60 mol % of the lipid nanoparticle. In embodiments, the one or more cationic lipid(s) constitute(s) about 31 mol %-59 mol % of the lipid nanoparticle. In embodiments, the one or more cationic lipid(s) constitute(s) about 35 mol %-45 mol % of the lipid nanoparticle. In embodiments, the one or more cationic lipid(s) constitute(s) about 40 mol % of the lipid nanoparticle.

In embodiments, the one or more non-cationic lipid(s) constitute(s) about 10 mol %-50 mol % of the lipid nanoparticle. In embodiments, the one or more non-cationic lipid(s) constitute(s) about 11 mol %-49 mol % of the lipid nanoparticle. In embodiments, the one or more non-cationic lipid(s) constitute(s) about 20 mol %-40 mol % of the lipid nanoparticle. In embodiments, the one or more non-cationic lipid(s) constitute(s) about 25 mol %-35 mol % of the lipid nanoparticle. In embodiments, the one or more non-cationic lipid(s) constitute(s) about 30 mol % of the lipid nanoparticle.

In embodiments, the one or more PEG-modified lipid(s) constitute(s) about 1 mol %-10 mol % of the lipid nanoparticle. In embodiments, the one or more PEG-modified lipid(s) constitute(s) about 1.1 mol %-9 mol % of the lipid nanoparticle. In embodiments, the one or more PEG-modified lipid(s) constitute(s) about 1 mol %-5 mol % of the lipid nanoparticle. In embodiments, the one or more PEG-modified lipid(s) constitute(s) about 1.5 mol %-3 mol % of the lipid nanoparticle.

In embodiments, the cholesterol-based lipid constitutes about 10 mol %-50 mol % of the lipid nanoparticle. In embodiments, the cholesterol-based lipid constitutes about 11 mol %-49 mol % of the lipid nanoparticle. In embodiments, the cholesterol-based lipid constitutes about 20 mol %-40 mol % of the lipid nanoparticle. In embodiments, the cholesterol-based lipid constitutes about 25 mol %-35 mol % of the lipid nanoparticle. In embodiments, the cholesterol-based lipid constitutes about 27 mol %-28.5 mol % of the lipid nanoparticle.

In embodiments, the one or more cationic lipid(s) constitute(s) about 31 mol %-59 mol % of the lipid nanoparticle, the one or more non-cationic lipid(s) constitute(s) about 11 mol %-49 mol % of the lipid nanoparticle, the one or more PEG-modified lipid(s) constitute(s) about 1.1 mol %-9 mol % of the lipid nanoparticle, and the cholesterol-based lipid constitutes about 11 mol %-49 mol % of the lipid nanoparticle.

In embodiments, the one or more cationic lipid(s) constitute(s) about 35 mol %-45 mol % of the lipid nanoparticle, the one or more non-cationic lipid(s) constitute(s) about 25 mol %-35 mol % of the lipid nanoparticle, the one or more PEG-modified lipid(s) constitute(s) about 1 mol %-5 mol % of the lipid nanoparticle, and the cholesterol-based lipid constitutes about 25 mol %-35 mol % of the lipid nanoparticle.

In embodiments, the one or more cationic lipid(s) constitute(s) about 40 mol % of the lipid nanoparticle, the one or more non-cationic lipid(s) constitute(s) about 30 mol % of the lipid nanoparticle, the one or more PEG-modified lipid(s) constitute(s) about 1.5 mol %-3 mol % of the lipid nanoparticle, and the cholesterol-based lipid constitutes about 27 mol %-28.5 mol % of the lipid nanoparticle.

In embodiments, the lipid nanoparticle encapsulates a nucleic acid, optionally an mRNA encoding a peptide or protein. In embodiments, the lipid nanoparticle encapsulates an mRNA encoding a peptide or protein, optionally for use in a vaccine. In embodiments, the peptide is an antigen.

As used herein, the phrase “encapsulation percentage” refers to the fraction of therapeutic agent (e.g. mRNA) that is effectively encapsulated within a liposomal-based vehicle (e.g. a lipid nanoparticle) relative to the initial fraction of therapeutic agent present in the lipid phase. In embodiments, the lipid nanoparticles have an encapsulation percentage for mRNA of at least 50%. In embodiments, the lipid nanoparticles have an encapsulation percentage for mRNA of at least 55%. In embodiments, the lipid nanoparticles have an encapsulation percentage for mRNA of at least 60%. In embodiments, the lipid nanoparticles have an encapsulation percentage for mRNA of at least 65%. In embodiments, the lipid nanoparticles have an encapsulation percentage for mRNA of at least 70%. In embodiments, the lipid nanoparticles have an encapsulation percentage for mRNA of at least 75%. In embodiments, the lipid nanoparticles have an encapsulation percentage for mRNA of at least 80%. In embodiments, the lipid nanoparticles have an encapsulation percentage for mRNA of at least 85%. In embodiments, the lipid nanoparticles have an encapsulation percentage for mRNA of at least 90%. In embodiments, the lipid nanoparticles have an encapsulation percentage for mRNA of at least 95%. In embodiments, the encapsulation percentage is calculated by performing the Ribogreen assay (Invitrogen) with and without the presence of 0.1% Triton-X 100.

In embodiments, the composition of the present invention is for use in therapy.

In embodiments, the composition of the present invention is for use in a method of treating or preventing a disease amenable to treatment or prevention by the peptide or protein encoded by the mRNA, optionally wherein the mRNA encodes an antigen and/or wherein the disease is (a) a protein deficiency, optionally wherein the protein deficiency affects the liver, lung, brain or muscle, (b) an autoimmune disease, (c) an infectious disease, or (d) cancer.

In embodiments, a method for treating or preventing a disease is provided, wherein said method comprises administering to a subject in need thereof a composition of the present invention and wherein the disease is amenable to treatment or prevention by the peptide or protein encoded by the mRNA, optionally wherein the mRNA encodes an antigen and/or the disease is (a) a protein deficiency, optionally wherein the protein deficiency affects the liver, lung, brain or muscle, (b) an autoimmune disease, (c) an infectious disease, or (d) cancer.

In embodiments, the composition is administered intravenously, intrathecally or intramuscularly, or by pulmonary delivery, optionally through nebulization. In embodiments, the composition is administered intramuscularly. In embodiments, the composition is administered by intravenous administration.

### Exemplary Compounds

In embodiments, the cationic lipids of the present invention include compounds selected from those depicted in Table A, or a pharmaceutically acceptable salt thereof.

Exemplary compounds include those described in Table A, or a pharmaceutically acceptable salt thereof.

TABLE A

| Com- | |
|---|---|
| pound | |
| number | Compound structure |
| 1 | 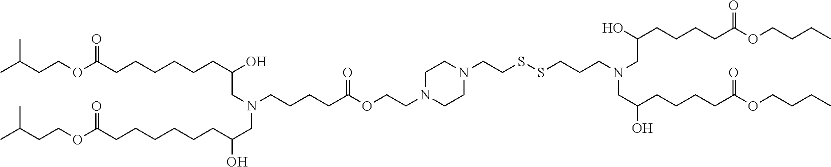 [SMILES: CCCCOC(=O)CCCCC(O)CN(CCCSSCCN1CCN(CCOC(=O)CCCCN(CC(O)CCCCCCC(=O)OCCC(C)C)CC(O)CCCCCCC(=O)OCCC(C)C)CC1)CC(O)CCCCC(=O)OCCCC] |
| 2 | 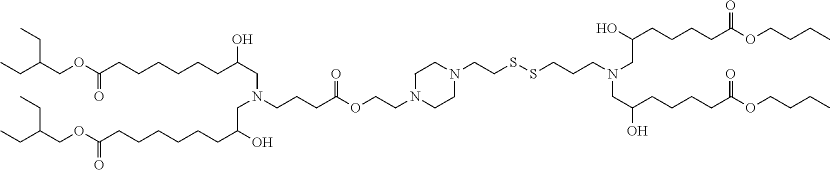 [SMILES: CCCCOC(=O)CCCCC(O)CN(CCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCC(=O)OCCCC] |
| 3 | 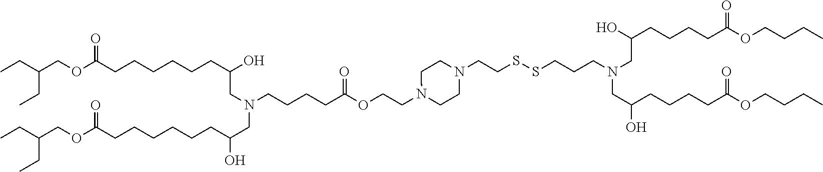 [SMILES: CCCCOC(=O)CCCCC(O)CN(CCCSSCCN1CCN(CCOC(=O)CCCCN(CC(O)CCCCCCC(=O)OCC(CC)CC)CC(O)CCCCCCC(=O)OCC(CC)CC)CC1)CC(O)CCCCC(=O)OCCCC] |
| 4 | 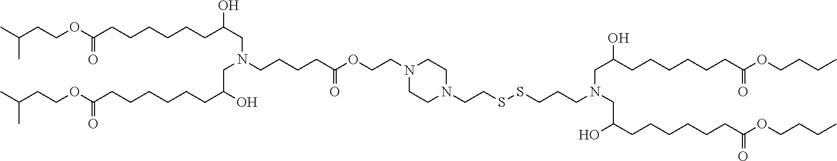 [SMILES: CCCCOC(=O)CCCCCCC(O)CN(CCCSSCCN1CCN(CCOC(=O)CCCCN(CC(O)CCCCCCC(=O)OCCC(C)C)CC(O)CCCCCCC(=O)OCCC(C)C)CC1)CC(O)CCCCCCC(=O)OCCCC] |
| 5 | 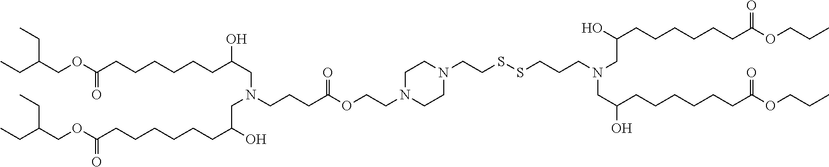 [SMILES: CCCOC(=O)CCCCCCC(O)CN(CCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCCCC(=O)OCCC] |
| 6 | 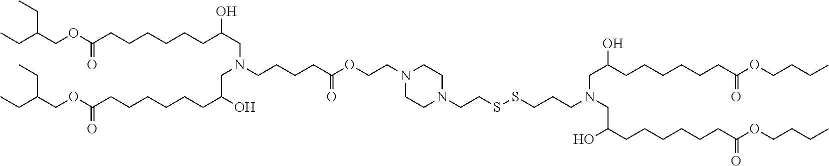 [SMILES: CCCCOC(=O)CCCCCCC(O)CN(CCCSSCCN1CCN(CCOC(=O)CCCCN(CC(O)CCCCCCC(=O)OCC(CC)CC)CC(O)CCCCCCC(=O)OCC(CC)CC)CC1)CC(O)CCCCCCC(=O)OCCCC] |
| 7 | 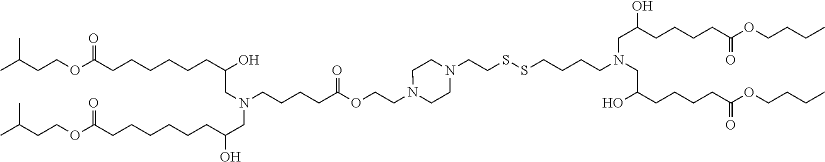 [SMILES: CCCCOC(=O)CCCCC(O)CN(CCCCSSCCN1CCN(CCOC(=O)CCCCN(CC(O)CCCCCCC(=O)OCCC(C)C)CC(O)CCCCCCC(=O)OCCC(C)C)CC1)CC(O)CCCCC(=O)OCCCC] |
| 8 | 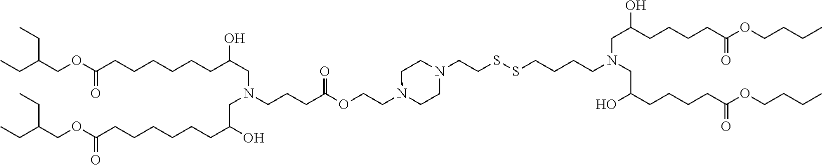 [SMILES: CCCCOC(=O)CCCCC(O)CN(CCCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCC(=O)OCCCC] |
| 9 | 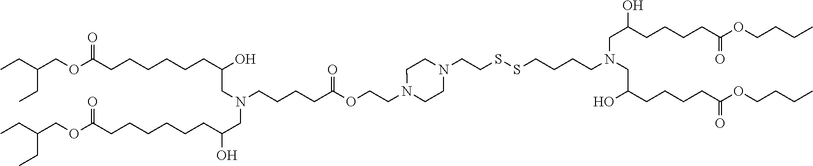 [SMILES: CCCCOC(=O)CCCCC(O)CN(CCCCSSCCN1CCN(CCOC(=O)CCCCN(CC(O)CCCCCCC(=O)OCC(CC)CC)CC(O)CCCCCCC(=O)OCC(CC)CC)CC1)CC(O)CCCCC(=O)OCCCC] |
| 10 | 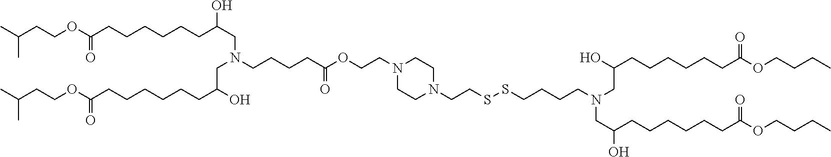 [SMILES: CCCCOC(=O)CCCCCCC(O)CN(CCCCSSCCN1CCN(CCOC(=O)CCCCN(CC(O)CCCCCCC(=O)OCCC(C)C)CC(O)CCCCCCC(=O)OCCC(C)C)CC1)CC(O)CCCCCCC(=O)OCCCC] |
| 11 | 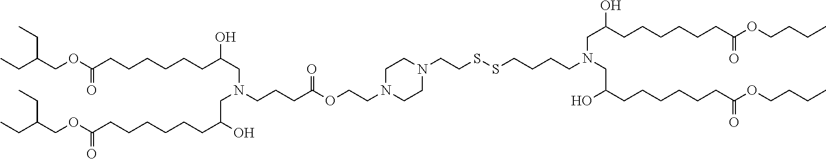 [SMILES: CCCCOC(=O)CCCCCCC(O)CN(CCCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCCCC(=O)OCCCC] |
| 12 | 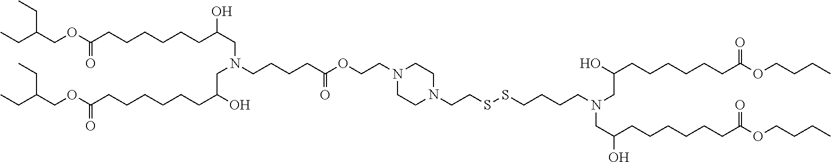 [SMILES: CCCCOC(=O)CCCCCCC(O)CN(CCCCSSCCN1CCN(CCOC(=O)CCCCN(CC(O)CCCCCCC(=O)OCC(CC)CC)CC(O)CCCCCCC(=O)OCC(CC)CC)CC1)CC(O)CCCCCCC(=O)OCCCC] |
| 13 | 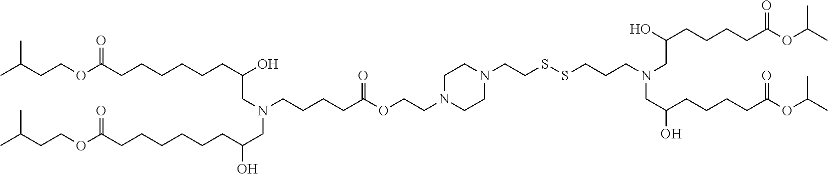 [SMILES: CC(C)CCOC(=O)CCCCCCC(O)CN(CCCCC(=O)OCCN1CCN(CCSSCCCN(CC(O)CCCCC(=O)OC(C)C)CC(O)CCCCC(=O)OC(C)C)CC1)CC(O)CCCCCCC(=O)OCCC(C)C] |
| 14 | 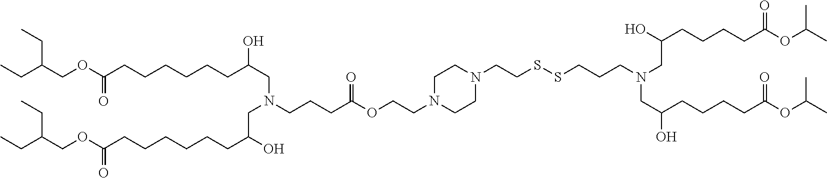 [SMILES: CCC(CC)COC(=O)CCCCCCC(O)CN(CCCC(=O)OCCN1CCN(CCSSCCCN(CC(O)CCCCC(=O)OC(C)C)CC(O)CCCCC(=O)OC(C)C)CC1)CC(O)CCCCCCC(=O)OCC(CC)CC] |
| 15 | 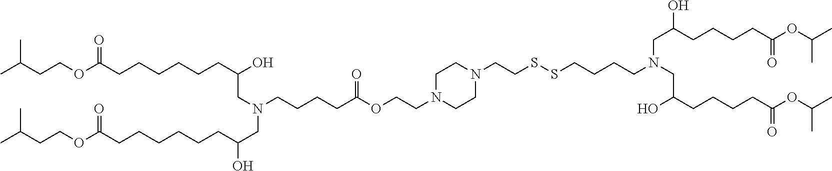 |
| 16 | 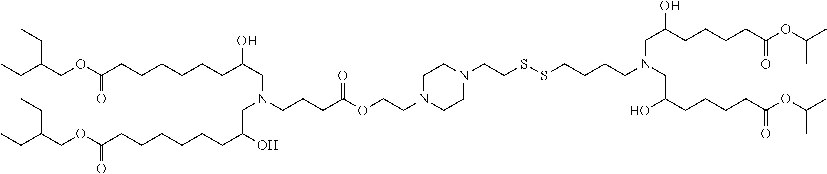 |
| 17 | 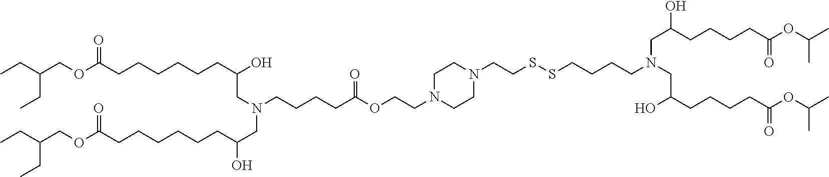 [SMILES: CCC(CC)COC(=O)CCCCCCC(O)CN(CCCCC(=O)OCCN1CCN(CCSSCCCCN(CC(O)CCCCC(=O)OC(C)C)CC(O)CCCCC(=O)OC(C)C)CC1)CC(O)CCCCCCC(=O)OCC(CC)CC] |
| 18 | 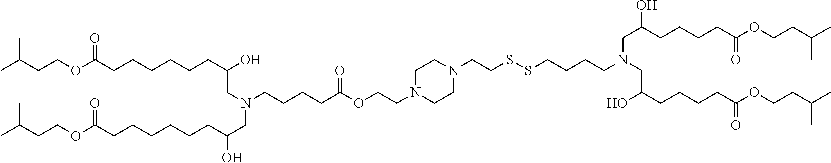 [SMILES: CC(C)CCOC(=O)CCCCCCC(O)CN(CCCCC(=O)OCCN1CCN(CCSSCCCCN(CC(O)CCCCC(=O)OCCC(C)C)CC(O)CCCCC(=O)OCCC(C)C)CC1)CC(O)CCCCCCC(=O)OCCC(C)C] |
| 19 | 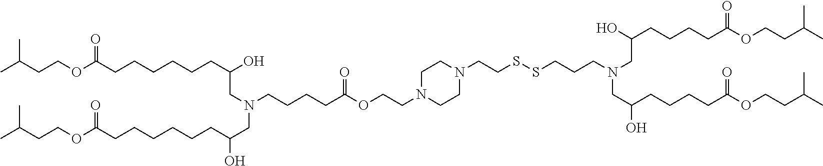 [SMILES: CC(C)CCOC(=O)CCCCCCC(O)CN(CCCCC(=O)OCCN1CCN(CCSSCCCN(CC(O)CCCCC(=O)OCCC(C)C)CC(O)CCCCC(=O)OCCC(C)C)CC1)CC(O)CCCCCCC(=O)OCCC(C)C] |
| 20 | 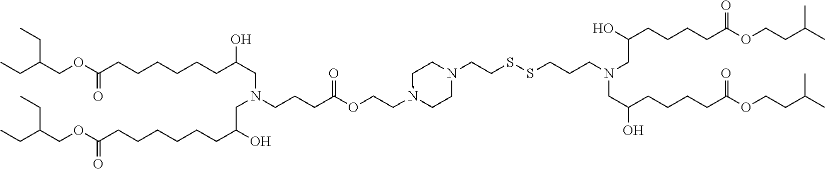 [SMILES: CCC(CC)COC(=O)CCCCCCC(O)CN(CCCC(=O)OCCN1CCN(CCSSCCCN(CC(O)CCCCC(=O)OCCC(C)C)CC(O)CCCCC(=O)OCCC(C)C)CC1)CC(O)CCCCCCC(=O)OCC(CC)CC] |
| 21 | 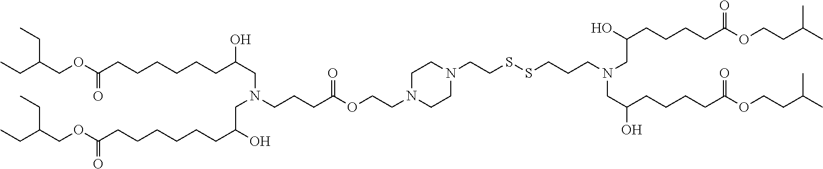 [SMILES: CCC(CC)COC(=O)CCCCCCC(O)CN(CCCC(=O)OCCN1CCN(CCSSCCCN(CC(O)CCCCC(=O)OCCC(C)C)CC(O)CCCCC(=O)OCCC(C)C)CC1)CC(O)CCCCCCC(=O)OCC(CC)CC] |
| 22 | 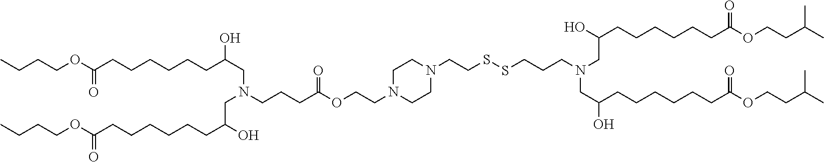 [SMILES: CCCCOC(=O)CCCCCCC(O)CN(CCCC(=O)OCCN1CCN(CCSSCCCN(CC(O)CCCCCCC(=O)OCCC(C)C)CC(O)CCCCCCC(=O)OCCC(C)C)CC1)CC(O)CCCCCCC(=O)OCCCC] |
| 23 | 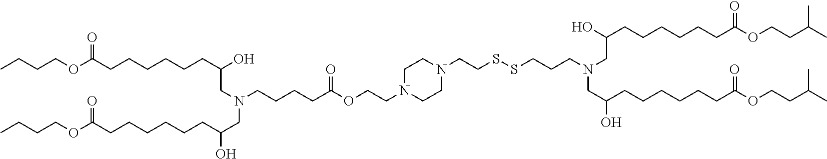 [SMILES: CCCCOC(=O)CCCCCCC(O)CN(CCCCC(=O)OCCN1CCN(CCSSCCCN(CC(O)CCCCCCC(=O)OCCC(C)C)CC(O)CCCCCCC(=O)OCCC(C)C)CC1)CC(O)CCCCCCC(=O)OCCCC] |
| 24 | 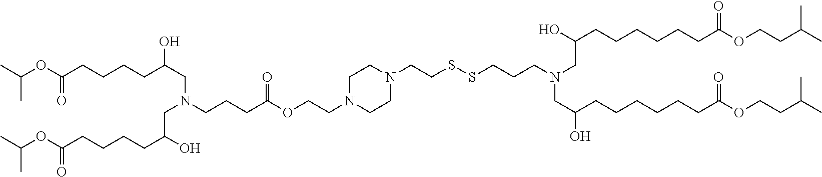 [SMILES: CC(C)CCOC(=O)CCCCCCC(O)CN(CCCSSCCN1CCN(CCOC(=O)CCCN(CC(O)CCCCC(=O)OC(C)C)CC(O)CCCCC(=O)OC(C)C)CC1)CC(O)CCCCCCC(=O)OCCC(C)C] |
| 25 | 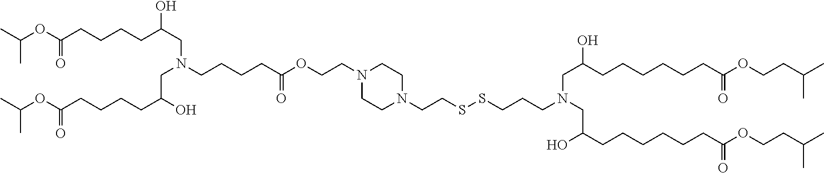 [SMILES: CC(C)CCOC(=O)CCCCCCC(O)CN(CCCSSCCN1CCN(CCOC(=O)CCCCN(CC(O)CCCCC(=O)OC(C)C)CC(O)CCCCC(=O)OC(C)C)CC1)CC(O)CCCCCCC(=O)OCCC(C)C] |
| 26 | 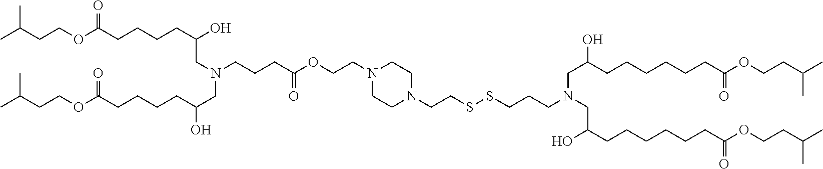 [SMILES: CC(C)CCOC(=O)CCCCCCC(O)CN(CCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCCC(C)C] |
| 27 | 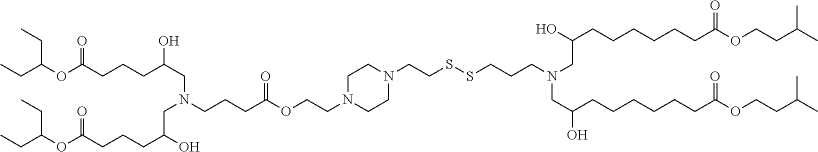 [SMILES: CCC(CC)OC(=O)CCCC(O)CN(CCCC(=O)OCCN1CCN(CCSSCCCN(CC(O)CCCCCCC(=O)OCCC(C)C)CC(O)CCCCCCC(=O)OCCC(C)C)CC1)CC(O)CCCC(=O)OC(CC)CC] |
| 28 | 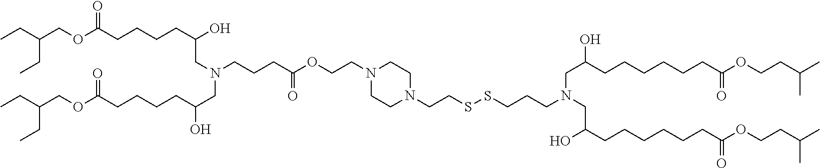 [SMILES: CCC(CC)COC(=O)CCCCC(O)CN(CCCC(=O)OCCN1CCN(CCSSCCCN(CC(O)CCCCCCC(=O)OCCC(C)C)CC(O)CCCCCCC(=O)OCCC(C)C)CC1)CC(O)CCCCC(=O)OCC(CC)CC] |
| 29 | 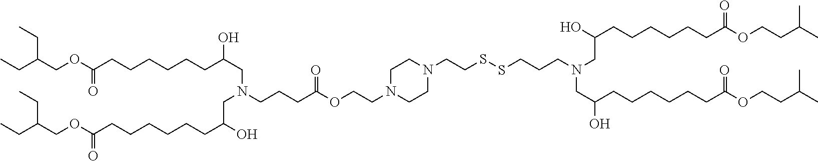 [SMILES: CCC(CC)COC(=O)CCCCCCC(O)CN(CCCC(=O)OCCN1CCN(CCSSCCCN(CC(O)CCCCCCC(=O)OCCC(C)C)CC(O)CCCCCCC(=O)OCCC(C)C)CC1)CC(O)CCCCCCC(=O)OCC(CC)CC] |
| 30 | 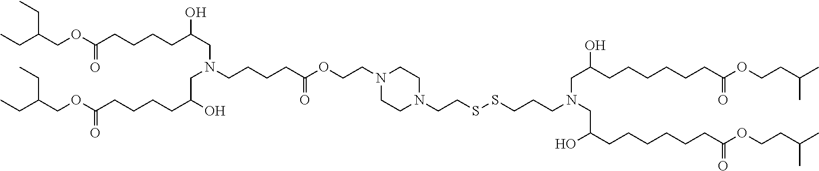 [SMILES: CCC(CC)COC(=O)CCCCC(O)CN(CCCCC(=O)OCCN1CCN(CCSSCCCN(CC(O)CCCCCCC(=O)OCCC(C)C)CC(O)CCCCCCC(=O)OCCC(C)C)CC1)CC(O)CCCCC(=O)OCC(CC)CC] |
| 31 | 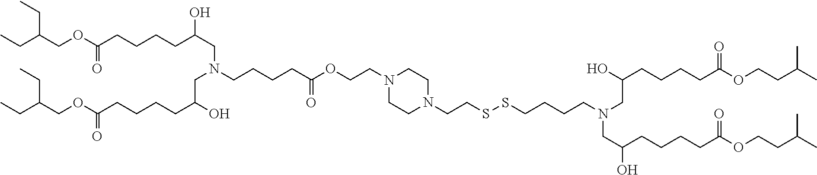 [SMILES: CCC(CC)COC(=O)CCCCC(O)CN(CCCCC(=O)OCCN1CCN(CCSSCCCCN(CC(O)CCCCC(=O)OCCC(C)C)CC(O)CCCCC(=O)OCCC(C)C)CC1)CC(O)CCCCC(=O)OCC(CC)CC] |
| 32 | 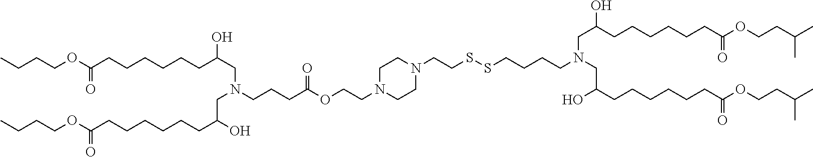 [SMILES: CCCCOC(=O)CCCCCCC(O)CN(CCCC(=O)OCCN1CCN(CCSSCCCCN(CC(O)CCCCCCC(=O)OCCC(C)C)CC(O)CCCCCCC(=O)OCCC(C)C)CC1)CC(O)CCCCCCC(=O)OCCCC] |
| 33 | 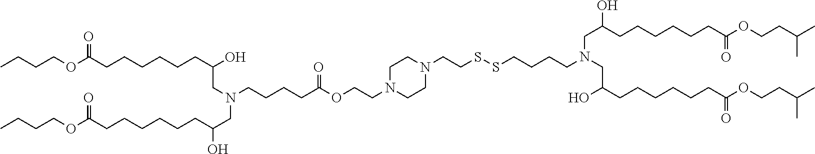 [SMILES: CCCCOC(=O)CCCCCCC(O)CN(CCCCC(=O)OCCN1CCN(CCSSCCCCN(CC(O)CCCCCCC(=O)OCCC(C)C)CC(O)CCCCCCC(=O)OCCC(C)C)CC1)CC(O)CCCCCCC(=O)OCCCC] |
| 34 | 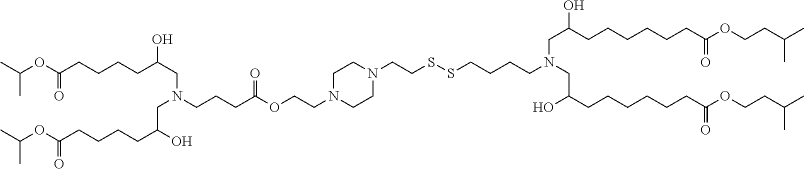 [SMILES: CC(C)CCOC(=O)CCCCCCC(O)CN(CCCCSSCCN1CCN(CCOC(=O)CCCN(CC(O)CCCCC(=O)OC(C)C)CC(O)CCCCC(=O)OC(C)C)CC1)CC(O)CCCCCCC(=O)OCCC(C)C] |
| 35 | 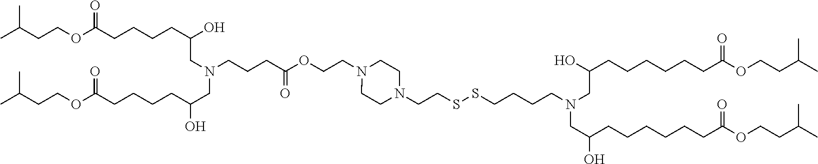 [SMILES: CC(C)CCOC(=O)CCCCCCC(O)CN(CCCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCCC(C)C] |
| 36 | 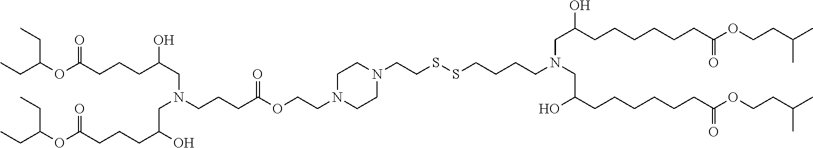 [SMILES: CCC(CC)OC(=O)CCCC(O)CN(CCCC(=O)OCCN1CCN(CCSSCCCCN(CC(O)CCCCCCC(=O)OCCC(C)C)CC(O)CCCCCCC(=O)OCCC(C)C)CC1)CC(O)CCCC(=O)OC(CC)CC] |
| 37 | 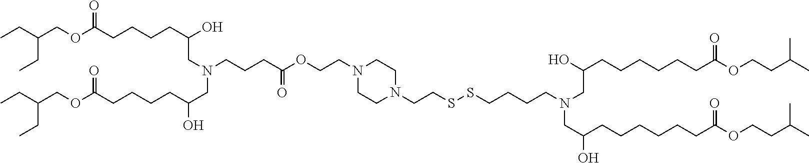 [SMILES: CCC(CC)COC(=O)CCCCC(O)CN(CCCC(=O)OCCN1CCN(CCSSCCCCN(CC(O)CCCCCCC(=O)OCCC(C)C)CC(O)CCCCCCC(=O)OCCC(C)C)CC1)CC(O)CCCCC(=O)OCC(CC)CC] |
| 38 | 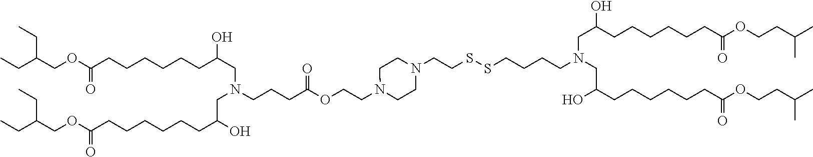 [SMILES: CCC(CC)COC(=O)CCCCCCC(O)CN(CCCC(=O)OCCN1CCN(CCSSCCCCN(CC(O)CCCCCCC(=O)OCCC(C)C)CC(O)CCCCCCC(=O)OCCC(C)C)CC1)CC(O)CCCCCCC(=O)OCC(CC)CC] |
| 39 | 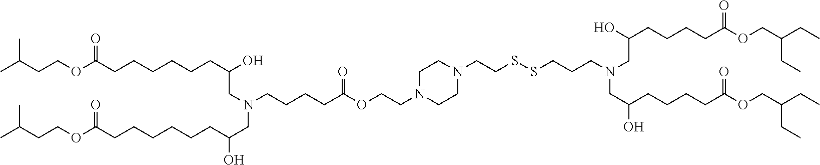 [SMILES: CCC(CC)COC(=O)CCCCC(O)CN(CCCSSCCN1CCN(CCOC(=O)CCCCN(CC(O)CCCCCCC(=O)OCCC(C)C)CC(O)CCCCCCC(=O)OCCC(C)C)CC1)CC(O)CCCCC(=O)OCC(CC)CC] |
| 40 | 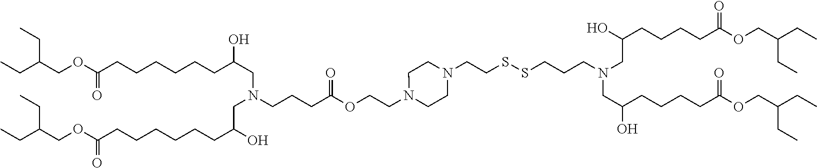 [SMILES: CCC(CC)COC(=O)CCCCCCC(O)CN(CCCC(=O)OCCN1CCN(CCSSCCCN(CC(O)CCCCC(=O)OCC(CC)CC)CC(O)CCCCC(=O)OCC(CC)CC)CC1)CC(O)CCCCCCC(=O)OCC(CC)CC] |
| 41 | 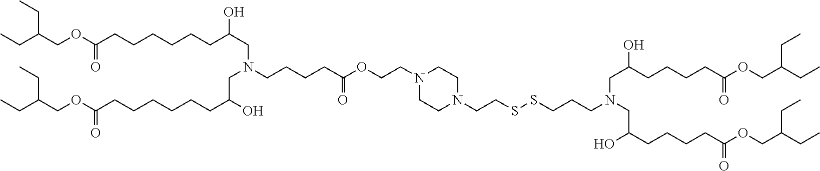 [SMILES: CCC(CC)COC(=O)CCCCCCC(O)CN(CCCCC(=O)OCCN1CCN(CCSSCCCN(CC(O)CCCCC(=O)OCC(CC)CC)CC(O)CCCCC(=O)OCC(CC)CC)CC1)CC(O)CCCCCCC(=O)OCC(CC)CC] |
| 42 | 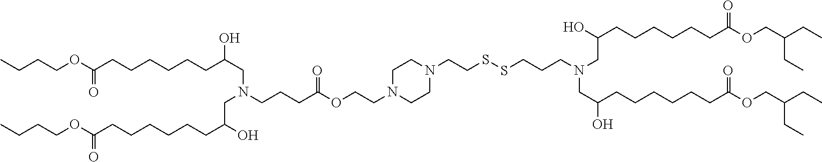 [SMILES: CCCCOC(=O)CCCCCCC(O)CN(CCCC(=O)OCCN1CCN(CCSSCCCN(CC(O)CCCCCCC(=O)OCC(CC)CC)CC(O)CCCCCCC(=O)OCC(CC)CC)CC1)CC(O)CCCCCCC(=O)OCCCC] |
| 43 | 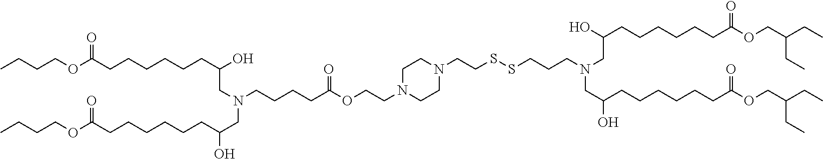 [SMILES: CCCCOC(=O)CCCCCCC(O)CN(CCCCC(=O)OCCN1CCN(CCSSCCCN(CC(O)CCCCCCC(=O)OCC(CC)CC)CC(O)CCCCCCC(=O)OCC(CC)CC)CC1)CC(O)CCCCCCC(=O)OCCCC] |
| 44 | 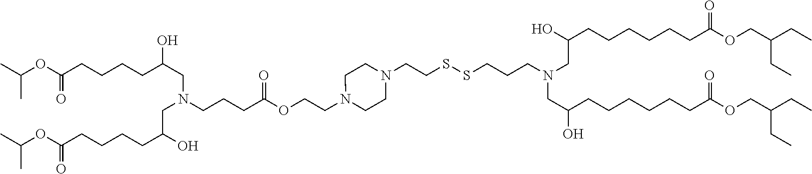 [SMILES: CCC(CC)COC(=O)CCCCCCC(O)CN(CCCSSCCN1CCN(CCOC(=O)CCCN(CC(O)CCCCC(=O)OC(C)C)CC(O)CCCCC(=O)OC(C)C)CC1)CC(O)CCCCCCC(=O)OCC(CC)CC] |
| 45 | 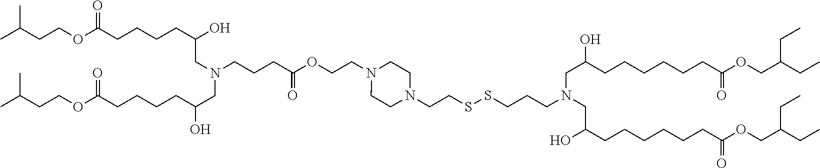 [SMILES: CCC(CC)COC(=O)CCCCCCC(O)CN(CCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCC(CC)CC] |
| 46 | 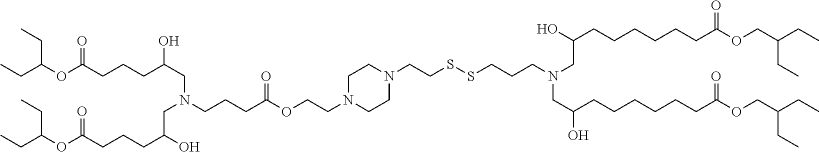 [SMILES: CCC(CC)COC(=O)CCCCCCC(O)CN(CCCSSCCN1CCN(CCOC(=O)CCCN(CC(O)CCCC(=O)OC(CC)CC)CC(O)CCCC(=O)OC(CC)CC)CC1)CC(O)CCCCCCC(=O)OCC(CC)CC] |
| 47 | 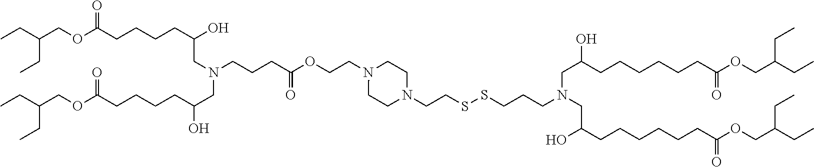 [SMILES: CCC(CC)COC(=O)CCCCCCC(O)CN(CCCSSCCN1CCN(CCOC(=O)CCCN(CC(O)CCCCC(=O)OCC(CC)CC)CC(O)CCCCC(=O)OCC(CC)CC)CC1)CC(O)CCCCCCC(=O)OCC(CC)CC] |
| 48 | 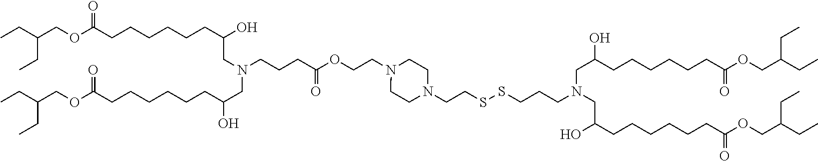 [SMILES: CCC(CC)COC(=O)CCCCCCC(O)CN(CCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCCCC(=O)OCC(CC)CC] |
| 49 | 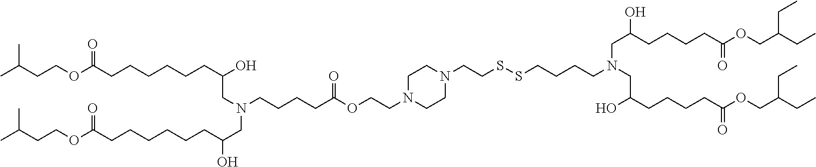 [SMILES: CCC(CC)COC(=O)CCCCC(O)CN(CCCCSSCCN1CCN(CCOC(=O)CCCCN(CC(O)CCCCCCC(=O)OCCC(C)C)CC(O)CCCCCCC(=O)OCCC(C)C)CC1)CC(O)CCCCC(=O)OCC(CC)CC] |
| 50 | 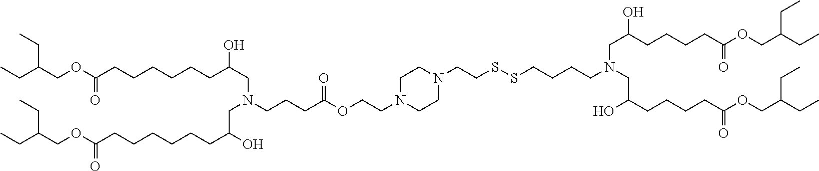 [SMILES: CCC(CC)COC(=O)CCCCCCC(O)CN(CCCC(=O)OCCN1CCN(CCSSCCCCN(CC(O)CCCCC(=O)OCC(CC)CC)CC(O)CCCCC(=O)OCC(CC)CC)CC1)CC(O)CCCCCCC(=O)OCC(CC)CC] |
| 51 | 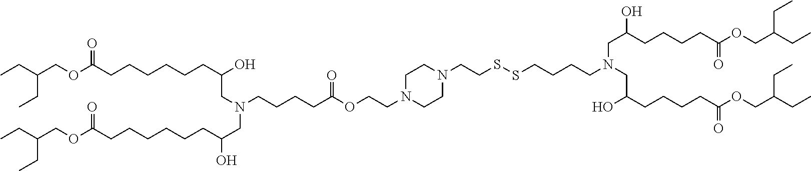 [SMILES: CCC(CC)COC(=O)CCCCCCC(O)CN(CCCCC(=O)OCCN1CCN(CCSSCCCCN(CC(O)CCCCC(=O)OCC(CC)CC)CC(O)CCCCC(=O)OCC(CC)CC)CC1)CC(O)CCCCCCC(=O)OCC(CC)CC] |
| 52 | 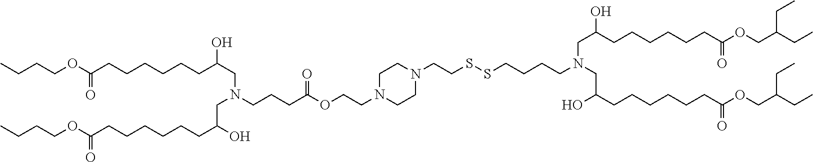 [SMILES: CCCCOC(=O)CCCCCCC(O)CN(CCCC(=O)OCCN1CCN(CCSSCCCCN(CC(O)CCCCCCC(=O)OCC(CC)CC)CC(O)CCCCCCC(=O)OCC(CC)CC)CC1)CC(O)CCCCCCC(=O)OCCCC] |
| 53 | 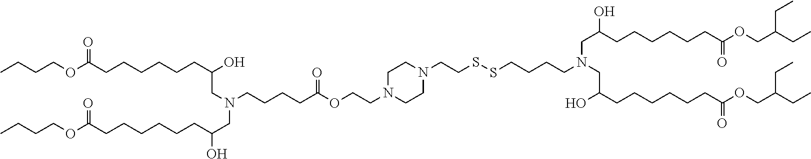 [SMILES: CCCCOC(=O)CCCCCCC(O)CN(CCCCC(=O)OCCN1CCN(CCSSCCCCN(CC(O)CCCCCCC(=O)OCC(CC)CC)CC(O)CCCCCCC(=O)OCC(CC)CC)CC1)CC(O)CCCCCCC(=O)OCCCC] |
| 54 | 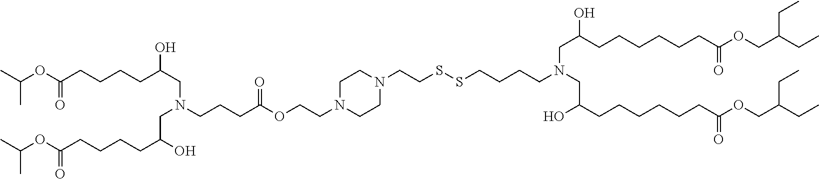 [SMILES: CCC(CC)COC(=O)CCCCCCC(O)CN(CCCCSSCCN1CCN(CCOC(=O)CCCN(CC(O)CCCCC(=O)OC(C)C)CC(O)CCCCC(=O)OC(C)C)CC1)CC(O)CCCCCCC(=O)OCC(CC)CC] |
| 55 | 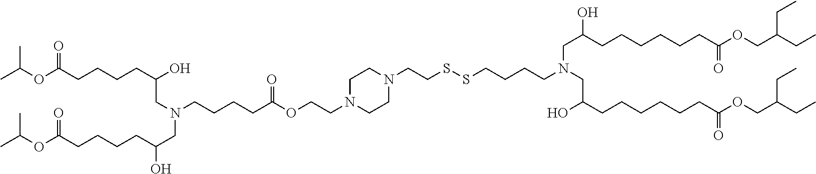 [SMILES: CCC(CC)COC(=O)CCCCCCC(O)CN(CCCCSSCCN1CCN(CCOC(=O)CCCCN(CC(O)CCCCC(=O)OC(C)C)CC(O)CCCCC(=O)OC(C)C)CC1)CC(O)CCCCCCC(=O)OCC(CC)CC] |
| 56 | 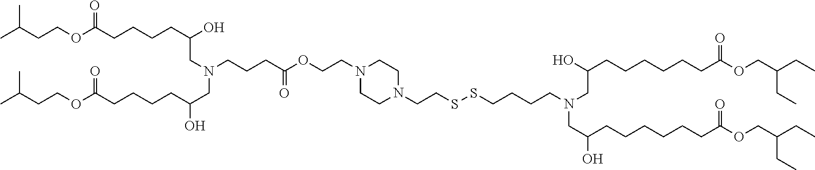 [SMILES: CCC(CC)COC(=O)CCCCCCC(O)CN(CCCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCC(CC)CC] |
| 57 | 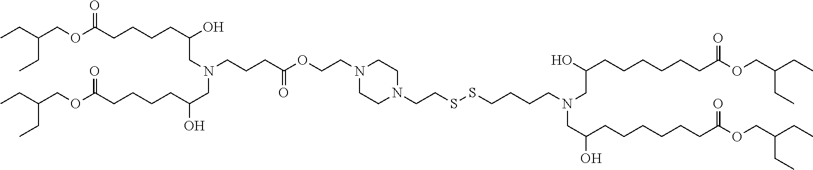 [SMILES: CCC(CC)COC(=O)CCCCCCC(O)CN(CCCCSSCCN1CCN(CCOC(=O)CCCN(CC(O)CCCCC(=O)OCC(CC)CC)CC(O)CCCCC(=O)OCC(CC)CC)CC1)CC(O)CCCCCCC(=O)OCC(CC)CC] |
| 58 | 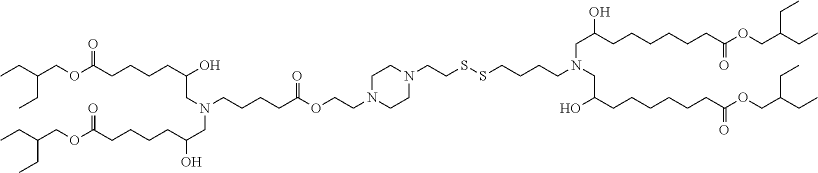 [SMILES: CCC(CC)COC(=O)CCCCCCC(O)CN(CCCCSSCCN1CCN(CCOC(=O)CCCCN(CC(O)CCCCC(=O)OCC(CC)CC)CC(O)CCCCC(=O)OCC(CC)CC)CC1)CC(O)CCCCCCC(=O)OCC(CC)CC] |
| 59 | 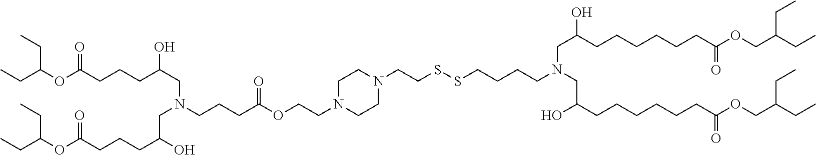 [SMILES: CCC(CC)COC(=O)CCCCCCC(O)CN(CCCCSSCCN1CCN(CCOC(=O)CCCN(CC(O)CCCC(=O)OC(CC)CC)CC(O)CCCC(=O)OC(CC)CC)CC1)CC(O)CCCCCCC(=O)OCC(CC)CC] |
| 60 | 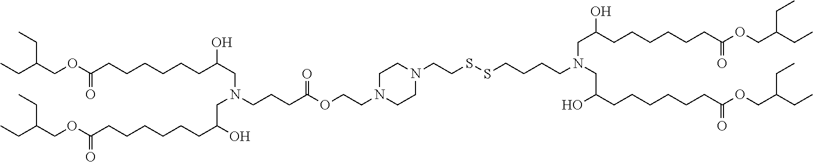 [SMILES: CCC(CC)COC(=O)CCCCCCC(O)CN(CCCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCCCC(=O)OCC(CC)CC] |

Any of the compounds 1-60 identified in Table A above may be provided in the form of a pharmaceutically acceptable salt and such salts are intended to be encompassed by the present invention.

Exemplary compounds include those described in Table B, or a pharmaceutically acceptable salt thereof.

TABLE B

| | |
|---|---|
| 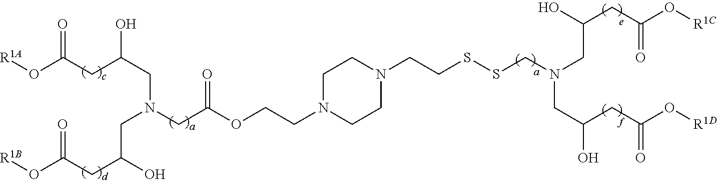 | Formula (Ir) |

| | | a on left hand side of Formula (Ir) = 3 | | | |
|---|---|---|---|---|---|
| | Compound number | 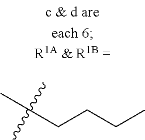 [SMILES: *CCCC] | 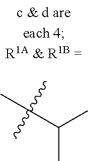 [SMILES: *C(C)C] | 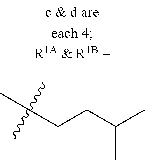 [SMILES: *CCC(C)C] | 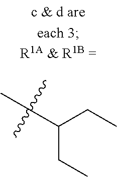 [SMILES: *C(CC)CC] |
| a on right | e & f are each | 61 | 62 | 63 | 64 |
| hand side of | 4; | | | | |
| Formula | R^{1C} & R^{1D} = | | | | |
| (Ir) = 3 | | | | | |
| | 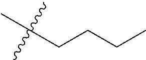 [SMILES: *CCCC] | | | | |
| | e & f are each 6; | 67 | 68 | 69 | 70 |
| | R^{1C} & R^{1D} = | | | | |
| | 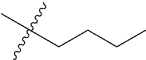 [SMILES: *CCCC] | | | | |
| | e & f are each 4; | 73 | 74 | 75 | 76 |
| | R^{1C} & R^{1D} = | | | | |
| | 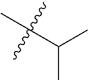 [SMILES: *C(C)C] | | | | |
| | e & f are each 4; | 80 | 81 | 82 | 83 |
| | R^{1C} & R^{1D} = | | | | |
| | 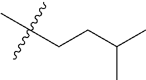 [SMILES: *CCC(C)C] | | | | |
| | e & f are each 6; | 22 | 24 | 26 | 27 |
| | R^{1C} & R^{1D} = | | | | |
| | 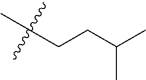 [SMILES: *CCC(C)C] | | | | |
| | e & f are each 4; | 88 | 89 | 90 | 91 |
| | R^{1C} & R^{1D} = | | | | |
| | 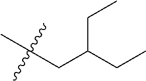 [SMILES: *CC(CC)CC] | | | | |
| | e & f are each 6; | 42 | 44 | 45 | 46 |
| | R^{1C} & R^{1D} = | | | | |
| | 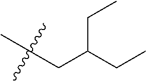 [SMILES: *CC(CC)CC] | | | | |
| a on right | e & f are each 4; | 96 | 97 | 98 | 99 |
| hand side of | 4; | | | | |
| Formula | R^{1C} & R^{1D} = | | | | |
| (Ir) = 4 | | | | | |
| | 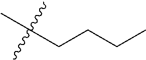 [SMILES: *CCCC] | | | | |
| | e & f are each 6; | 102 | 103 | 104 | 105 |
| | R^{1C} & R^{1D} = | | | | |
| | 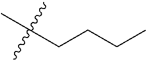 [SMILES: *CCCC] | | | | |
| | e & f are each 4; | 108 | 109 | 110 | 111 |
| | R^{1C} & R^{1D} = | | | | |
| | 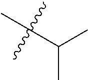 [SMILES: *C(C)C] | | | | |
| | e & f are each 4; | 114 | 115 | 116 | 117 |
| | R^{1C} & R^{1D} = | | | | |
| | 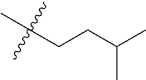 [SMILES: *CCC(C)C] | | | | |
| | e & f are each 6; | 32 | 34 | 35 | 36 |
| | R^{1C} & R^{1D} = | | | | |
| | 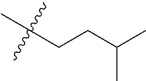 [SMILES: *CCC(C)C] | | | | |
| | e & f are each 4; | 123 | 124 | 125 | 126 |
| | R^{1C} & R^{1D} = | | | | |
| | 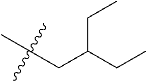 [SMILES: *CC(CC)CC] | | | | |
| | e & f are each 6; | 52 | 54 | 56 | 59 |
| | R^{1C} & R^{1D} = | | | | |
| | 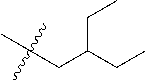 [SMILES: *CC(CC)CC] | | | | |

| | | | | a on left hand side of |
|---|---|---|---|---|
| | | a on left hand side of Formula (Ir) = 3 | | Formula (Ir) = 4 |
| | Compound number | 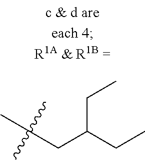 [SMILES: *CC(CC)CC] | 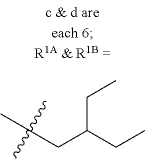 [SMILES: *CC(CC)CC] | 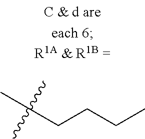 [SMILES: *CCCC] |
| a on the right hand side | e & f are each 4; | 65 | 2 | 66 |
| of Formula (Ir) = 3 | R^{1C} & R^{1D} = | | | |
| | 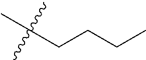 [SMILES: *CCCC] | | | |
| | e & f are each 6; | 71 | 155 | 72 |
| | R^{1C} & R^{1D} = | | | |
| | 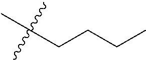 [SMILES: *CCCC] | | | |
| | e & f are each 4; | 77 | 14 | 78 |
| | R^{1C} & R^{1D} = | | | |
| | 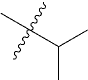 [SMILES: *C(C)C] | | | |
| | e & f are each 4; | 84 | 20 | 85 |
| | R^{1C} & R^{1D} = | | | |
| | 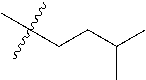 [SMILES: *CCC(C)C] | | | |
| | e & f are each 6; | 28 | 29 | 23 |
| | R^{1C} & R^{1D} = | | | |
| | 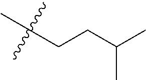 [SMILES: *CCC(C)C] | | | |
| | e & f are each 4; | 92 | 40 | 93 |
| | R^{1C} & R^{1D} = | | | |
| | 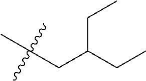 [SMILES: *CC(CC)CC] | | | |
| | e & f are each 6; | 47 | 48 | 43 |
| | R^{1C} & R^{1D} = | | | |
| | 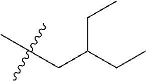 [SMILES: *CC(CC)CC] | | | |
| a on right hand side of | e & f are each 4; | 100 | 8 | 101 |
| Formula (Ir) = 4 | R^{1C} & R^{1D} = | | | |
| | 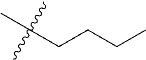 [SMILES: *CCCC] | | | |
| | e & f are each 6; | 106 | 11 | 107 |
| | R^{1C} & R^{1D} = | | | |
| | 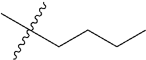 [SMILES: *CCCC] | | | |
| | e & f are each 4; | 112 | 16 | 113 |
| | R^{1C} & R^{1D} = | | | |
| | 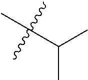 [SMILES: *C(C)C] | | | |
| | e & f are each 4; | 118 | 119 | 120 |
| | R^{1C} & R^{1D} = | | | |
| | 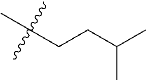 [SMILES: *CCC(C)C] | | | |
| | e & f are each 6; | 37 | 38 | 33 |
| | R^{1C} & R^{1D} = | | | |
| | 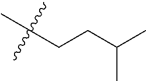 [SMILES: *CCC(C)C] | | | |
| | e & f are each 4; | 127 | 50 | 128 |
| | R^{1C} & R^{1D} = | | | |
| | 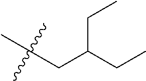 [SMILES: *CC(CC)CC] | | | |
| | e & f are each 6; | 57 | 60 | 53 |
| | R^{1C} & R^{1D} = | | | |
| | 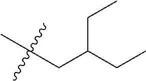 [SMILES: *CC(CC)CC] | | | |

| | | a on left hand side of Formula (Ir) = 4 | |
|---|---|---|---|
| | Compound number | 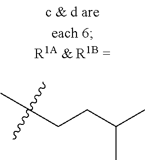 [SMILES: *CCC(C)C] | 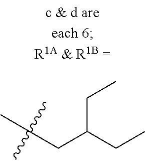 [SMILES: *CC(CC)CC] |
| a on the right hand side of | e & f are each 4; | 1 | 3 |
| Formula (Ir) = 3 | R^{1C} & R^{1D} = | | |
| | 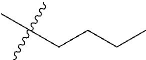 [SMILES: *CCCC] | | |
| | e & f are each 6; | 4 | 6 |
| | R^{1C} & R^{1D} = | | |
| | 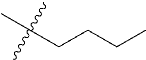 [SMILES: *CCCC] | | |
| | e & f are each 4; | 13 | 79 |
| | R^{1C} & R^{1D} = | | |
| | 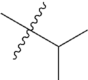 [SMILES: *C(C)C] | | |
| | e & f are each 4; | 19 | 21 |
| | R^{1C} & R^{1D} = | | |
| | 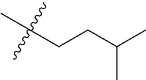 [SMILES: *CCC(C)C] | | |
| | e & f are each 6; | 86 | 87 |
| | R^{1C} & R^{1D} = | | |
| | 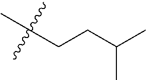 [SMILES: *CCC(C)C] | | |
| | e & f are each 6; | 39 | 41 |
| | R^{1C} & R^{1D} = | | |
| | 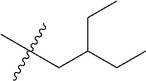 [SMILES: *CC(CC)CC] | | |
| | e & f are each 6; | 94 | 95 |
| | R^{1C} & R^{1D} = | | |
| | 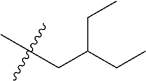 [SMILES: *CC(CC)CC] | | |
| a on right hand side of | e & f are each 4; | 7 | 9 |
| Formula (Ir) = 4 | R^{1C} & R^{1D} = | | |
| | 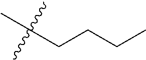 [SMILES: *CCCC] | | |
| | e & f are each 6; | 10 | 12 |
| | R^{1C} & R^{1D} = | | |
| | 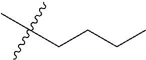 [SMILES: *CCCC] | | |
| | e & f are each 4; | 15 | 17 |
| | R^{1C} & R^{1D} = | | |
| | 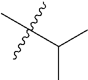 [SMILES: *C(C)C] | | |
| | e & f are each 4; | 18 | 31 |
| | R^{1C} & R^{1D} = | | |
| | 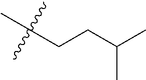 [SMILES: *CCC(C)C] | | |
| | e & f are each 6; | 121 | 122 |
| | R^{1C} & R^{1D} = | | |
| | 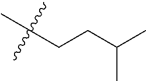 [SMILES: *CCC(C)C] | | |
| | e & f are each 4; | 49 | 51 |
| | R^{1C} & R^{1D} = | | |
| | 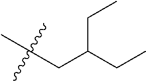 [SMILES: *CC(CC)CC] | | |
| | e & f are each 6; | 129 | 130 |
| | R^{1C} & R^{1D} = | | |
| | 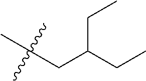 [SMILES: *CC(CC)CC] | | |

Any of the compounds 1-4, 6-24, 26-29, 31-54, 56-57, 59-130 and 155 identified in Table B above may be provided in the form of a pharmaceutically acceptable salt and such salts are intended to be encompassed by the present invention.

Exemplary compounds include those described in Table C, or a pharmaceutically acceptable salt thereof.

TABLE C

| | a on left hand | c & d | | a on right | e & f | |
|---|---|---|---|---|---|---|
| Compound | side of formula | are | | hand side of | are | |
| number | (Ir) = | each: | R^{1A} & R^{1B} = | Formula (Ir) = | each: | R^{1C} & R^{1D =} |
| 131 | 3 | 3 | 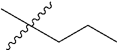 [SMILES: *CCC] | 3 | 4 | 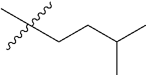 [SMILES: *CCC(C)C] |
| 132 | 3 | 3 | 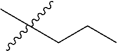 [SMILES: *CCC] | 3 | 6 | 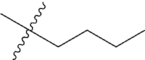 [SMILES: *CCCC] |
| 133 | 3 | 4 | 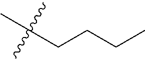 [SMILES: *CCCC] | 3 | 4 | 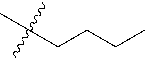 [SMILES: *CCCC] |
| 134 | 3 | 4 | 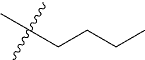 [SMILES: *CCCC] | 3 | 4 | 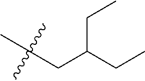 [SMILES: *CC(CC)CC] |
| 135 | 3 | 3 | 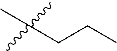 [SMILES: *CCC] | 4 | 4 | 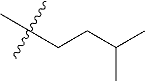 [SMILES: *CCC(C)C] |
| 136 | 3 | 3 | 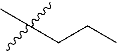 [SMILES: *CCC] | 4 | 4 | 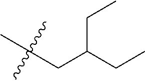 [SMILES: *CC(CC)CC] |
| 137 | 3 | 3 | 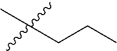 [SMILES: *CCC] | 4 | 6 | 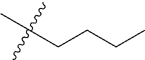 [SMILES: *CCCC] |
| 138 | 3 | 4 | 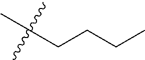 [SMILES: *CCCC] | 4 | 4 | 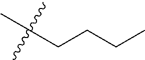 [SMILES: *CCCC] |
| 139 | 3 | 4 | 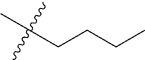 [SMILES: *CCCC] | 4 | 4 | 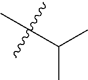 [SMILES: *C(C)C] |
| 140 | 4 | 4 | 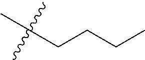 [SMILES: *CCCC] | 4 | 4 | 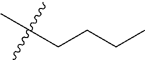 [SMILES: *CCCC] |
| 141 | 4 | 4 | 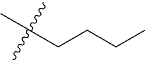 [SMILES: *CCCC] | 4 | 4 | 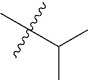 [SMILES: *C(C)C] |
| 142 | 3 | 4 | 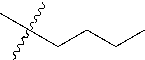 [SMILES: *CCCC] | 4 | 4 | 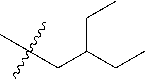 [SMILES: *CC(CC)CC] |
| 143 | 4 | 4 | 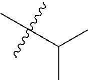 [SMILES: *C(C)C] | 3 | 4 | 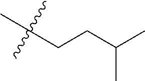 [SMILES: *CCC(C)C] |
| 144 | 4 | 4 | 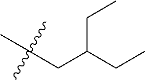 [SMILES: *CC(CC)CC] | 3 | 4 | 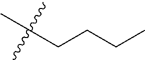 [SMILES: *CCCC] |
| 145 | 4 | 4 | 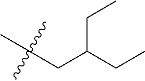 [SMILES: *CC(CC)CC] | 3 | 4 | 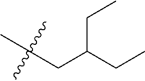 [SMILES: *CC(CC)CC] |
| 146 | 3 | 4 | 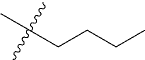 [SMILES: *CCCC] | 3 | 4 | 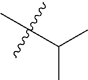 [SMILES: *C(C)C] |
| 147 | 3 | 4 | 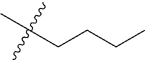 [SMILES: *CCCC] | 3 | 4 | 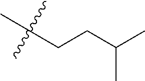 [SMILES: *CCC(C)C] |
| 148 | 4 | 4 | 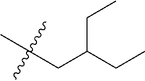 [SMILES: *CC(CC)CC] | 3 | 4 | 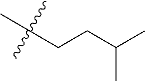 [SMILES: *CCC(C)C] |
| 149 | 3 | 4 | 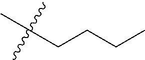 [SMILES: *CCCC] | 4 | 4 | 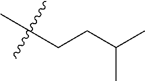 [SMILES: *CCC(C)C] |
| 150 | 4 | 4 | 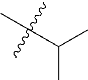 [SMILES: *C(C)C] | 3 | 4 | 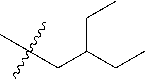 [SMILES: *CC(CC)CC] |
| 151 | 4 | 4 | 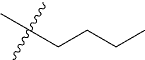 [SMILES: *CCCC] | 3 | 4 | 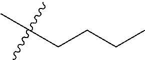 [SMILES: *CCCC] |
| 152 | 4 | 4 | 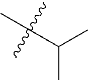 [SMILES: *C(C)C] | 3 | 4 | 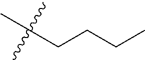 [SMILES: *CCCC] |
| 153 | 4 | 4 | 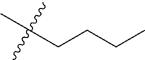 [SMILES: *CCCC] | 3 | 4 | 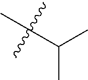 [SMILES: *C(C)C] |
| 154 | 3 | 3 | 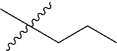 [SMILES: *CCC] | 3 | 4 | 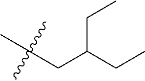 [SMILES: *CC(CC)CC] |

Any of the compounds 131-154 identified in Table C above may be provided in the form of a pharmaceutically acceptable salt and such salts are intended to be encompassed by the present invention.

The compounds of the invention as described herein can be prepared according to methods known in the art, including the exemplary syntheses of the Examples provided herein.

### Nucleic Acids

The compounds of the invention as described herein can be used to prepare compositions useful for the delivery of nucleic acids.

### Synthesis of Nucleic Acids

Nucleic acids according to the present invention may be synthesized according to any known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, mutated T7 or SP6 RNA polymerase), DNAse I, pyrophosphatase, and/or RNAse inhibitor. The exact conditions will vary according to the specific application.

In some embodiments, for the preparation of mRNA according to the invention, a DNA template is transcribed in vitro. A suitable DNA template typically has a promoter, for example a T3, T7, mutated T7 or SP6 promoter, for in vitro transcription, followed by desired nucleotide sequence for desired mRNA and a termination signal.

Desired mRNA sequence(s) according to the invention may be determined and incorporated into a DNA template using standard methods. For example, starting from a desired amino acid sequence (e.g., an enzyme sequence), a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

Modified mRNA

In some embodiments, mRNA according to the present invention may be synthesized as unmodified or modified mRNA. Modified mRNA comprises nucleotide modifications in the RNA. A modified mRNA according to the invention can thus include nucleotide modification that are, for example, backbone modifications, sugar modifications or base modifications. In some embodiments, mRNAs may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g., 1-methyl-adenine, 2-methyl-adenine, 2-methylthio-N-6-isopentenyl-adenine, N-6-methyl-adenine, N-6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5-methoxycarbonylmethyl-uracil, 5-methoxy-uracil, uracil-5-oxyacetic acid methyl ester, uracil-5-oxyacetic acid (v), 1-methyl-pseudouracil, queuosine, beta-D-mannosyl-queuosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

### Pharmaceutical Formulations of Cationic Lipids and Nucleic Acids

In certain embodiments, the compounds of the invention as described herein, as well as pharmaceutical and liposomal compositions comprising such lipids, can be used in formulations to facilitate the delivery of encapsulated materials (e.g., one or more polynucleotides such as mRNA) to, and subsequent transfection of one or more target cells. For example, in certain embodiments cationic lipids described herein (and compositions such as liposomal compositions comprising such lipids) are characterized as resulting in one or more of receptor-mediated endocytosis, clathrin-mediated and caveolae-mediated endocytosis, phagocytosis and macropinocytosis, fusogenicity, endosomal or lysosomal disruption and/or releasable properties that afford such compounds advantages relative other similarly classified lipids.

According to the present invention, a nucleic acid, e.g., mRNA encoding a protein (e.g., a full length, fragment or portion of a protein) as described herein may be delivered via a delivery vehicle comprising a compound of the invention as described herein.

As used herein, the terms “delivery vehicle,” “transfer vehicle,” “nanoparticle,” or grammatical equivalents thereof, are used interchangeably.

For example, the present invention provides a composition (e.g., a pharmaceutical composition) comprising a compound described herein and one or more polynucleotides. A composition (e.g., a pharmaceutical composition) may further comprise
(i) one or more cationic lipids,
(ii) one or more non-cationic lipids,
(iii) one or more cholesterol-based lipids and/or
(iv) one or more PEG-modified lipids.

In certain embodiments a composition exhibits an enhanced (e.g., increased) ability to transfect one or more target cells. Accordingly, also provided herein are methods of transfecting one or more target cells. Such methods generally comprise the step of contacting the one or more target cells with the cationic lipids and/or pharmaceutical compositions disclosed herein (e.g., a liposomal formulation comprising a compound described herein encapsulating one or more polynucleotides) such that the one or more target cells are transfected with the materials encapsulated therein (e.g., one or more polynucleotides). As used herein, the terms “transfect” or “transfection” refer to the intracellular introduction of one or more encapsulated materials (e.g., nucleic acids and/or polynucleotides) into a cell (e.g., into a target cell). The introduced polynucleotide may be stably or transiently maintained in the target cell. The term “transfection efficiency” refers to the relative amount of such encapsulated material (e.g., polynucleotides) up-taken by, introduced into, and/or expressed by the target cell which is subject to transfection. In practice, transfection efficiency may be estimated by the amount of a reporter polynucleotide product produced by the target cells following transfection. In certain embodiments, the compounds and pharmaceutical compositions described herein demonstrate high transfection efficiencies thereby improving the likelihood that appropriate dosages of the encapsulated materials (e.g., one or more polynucleotides) will be delivered to the site of pathology and subsequently expressed, while at the same time minimizing potential systemic adverse effects or toxicity associated with the compound or their encapsulated contents.

Following transfection of one or more target cells by, for example, the polynucleotides encapsulated in the one or more lipid nanoparticles comprising the pharmaceutical or liposomal compositions disclosed herein, the production of the product (e.g., a polypeptide or protein) encoded by such polynucleotide may be stimulated and the capability of such target cells to express the polynucleotide and produce, for example, a polypeptide or protein of interest is enhanced. For example, transfection of a target cell by one or more compounds or pharmaceutical compositions encapsulating mRNA will enhance (i.e., increase) the production of the protein or enzyme encoded by such mRNA.

Further, delivery vehicles described herein (e.g., liposomal delivery vehicles) may be prepared to preferentially distribute to other target tissues, cells or organs, such as the heart, lungs, kidneys, spleen or muscle. In embodiments, the delivery vehicles described herein (e.g., liposomal delivery vehicles) may be prepared to preferentially distribute to the lungs. In embodiments, the delivery vehicles described herein (e.g., liposomal delivery vehicles) may be prepared to preferentially distribute to muscle tissue. In embodiments, the lipid nanoparticles of the present invention may be prepared to achieve enhanced delivery to the target cells and tissues. For example, polynucleotides (e.g., mRNA) encapsulated in one or more of the compounds or pharmaceutical and liposomal compositions described herein can be delivered to and/or transfect targeted cells or tissues. In some embodiments, the encapsulated polynucleotides (e.g., mRNA) are capable of being expressed and functional polypeptide products produced (and in some instances excreted) by the target cell, thereby conferring a beneficial property to, for example the target cells or tissues. Such encapsulated polynucleotides (e.g., mRNA) may encode, for example, a hormone, enzyme, receptor, polypeptide, peptide or other protein of interest.

### Liposomal Delivery Vehicles

In some embodiments, a composition is a suitable delivery vehicle. In embodiments, a composition is a liposomal delivery vehicle, e.g., a lipid nanoparticle.

The terms “liposomal delivery vehicle” and “liposomal composition” are used interchangeably.

Enriching liposomal compositions with one or more of the cationic lipids disclosed herein may be used as a means of improving the safety profile or otherwise conferring one or more desired properties to such enriched liposomal composition (e.g., improved delivery of the encapsulated polynucleotides to one or more target cells and/or reduced in vivo toxicity of a liposomal composition). Accordingly, also contemplated are pharmaceutical compositions, and in particular liposomal compositions, that comprise one or more of the cationic lipids disclosed herein.

Thus, in certain embodiments, the compounds of the invention as described herein may be used as a component of a liposomal composition to facilitate or enhance the delivery and release of encapsulated materials (e.g., one or more therapeutic agents) to one or more target cells (e.g., by permeating or fusing with the lipid membranes of such target cells).

As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphophilic polymers and surfactants (e.g., polymerosomes, niosomes, etc.). In the context of the present invention, a liposomal delivery vehicle typically serves to transport a desired mRNA to a target cell or tissue.

In certain embodiments, such compositions (e.g., liposomal compositions) are loaded with or otherwise encapsulate materials, such as for example, one or more biologically-active polynucleotides (e.g., mRNA).

In embodiments, a composition (e.g., a pharmaceutical composition) comprises an mRNA encoding a peptide or protein, encapsulated within a liposome. In embodiments, a liposome comprises:
(i) one or more cationic lipids,
(ii) one or more non-cationic lipids,
(iii) one or more cholesterol-based lipids and
(iv) one or more PEG-modified lipids, wherein at least one cationic lipid is a compound of the invention as described herein.

In embodiments, a composition comprises an mRNA encoding for a peptide or protein (e.g., any peptide or protein described herein). In embodiments, a composition comprises an mRNA encoding for a peptide (e.g., any peptide described herein). In embodiments, a composition comprises an mRNA encoding for a protein (e.g., any protein described herein).

In embodiments, a composition (e.g., a pharmaceutical composition) comprises a nucleic acid encapsulated within a liposome, wherein the liposome comprises a compound described herein.

In embodiments, a nucleic acid is an mRNA encoding a peptide or protein. In embodiments, an mRNA encodes a peptide or protein for use in the delivery to or treatment of the lung of a subject or a lung cell. In embodiments, an mRNA encodes a peptide or protein for use in the delivery to or treatment of the liver of a subject or a liver cell. In embodiments, an mRNA encodes a peptide or protein for use in the delivery to or treatment of a muscle cell. In embodiments, an mRNA encodes a peptide or protein for use in the delivery to or treatment of an immune cell. Still other exemplary mRNAs are described herein.

In embodiments, a liposomal delivery vehicle (e.g., a lipid nanoparticle) can have a net positive charge.

In embodiments, a liposomal delivery vehicle (e.g., a lipid nanoparticle) can have a net negative charge.

In embodiments, a liposomal delivery vehicle (e.g., a lipid nanoparticle) can have a net neutral charge.

In embodiments, a lipid nanoparticle that encapsulates a nucleic acid (e.g., mRNA encoding a peptide or protein) comprises one or more compounds of the invention as described herein.

For example, the amount of a compound of the invention as described herein in a composition can be described as a percentage (“wt %”) of the combined dry weight of all lipids of a composition (e.g., the combined dry weight of all lipids present in a liposomal composition).

In embodiments of the pharmaceutical compositions described herein, a compound of the invention as described herein is present in an amount that is about 0.5 wt % to about 30 wt % (e.g., about 0.5 wt % to about 20 wt %) of the combined dry weight of all lipids present in a composition (e.g., a liposomal composition).

In embodiments, a compound of the invention as described herein is present in an amount that is about 1 wt % to about 30 wt %, about 1 wt % to about 20 wt %, about 1 wt % to about 15 wt %, about 1 wt % to about 10 wt %, or about 5 wt % to about 25 wt % of the combined dry weight of all lipids present in a composition (e.g., a liposomal composition). In embodiments, a compound of the invention as described herein is present in an amount that is about 0.5 wt % to about 5 wt %, about 1 wt % to about 10 wt %, about 5 wt % to about 20 wt %, or about 10 wt % to about 20 wt % of the combined dry weight of all lipids present in a composition such as a liposomal delivery vehicle.

In embodiments, the amount of a compound of the invention as described herein is present in an amount that is at least about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 96 wt %, about 97 wt %, about 98 wt %, or about 99 wt % of the combined dry weight of total lipids in a composition (e.g., a liposomal composition).

In embodiments, the amount of a compound of the invention as described herein is present in an amount that is no more than about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 96 wt %, about 97 wt %, about 98 wt %, or about 99 wt % of the combined dry weight of total lipids in a composition (e.g., a liposomal composition).

In embodiments, a composition (e.g., a liposomal delivery vehicle such as a lipid nanoparticle) comprises about 0.1 wt % to about 20 wt % (e.g., about 0.1 wt % to about 15 wt %) of a compound described herein. In embodiments, a delivery vehicle (e.g., a liposomal delivery vehicle such as a lipid nanoparticle) comprises about 0.5 wt %, about 1 wt %, about 3 wt %, about 5 wt %, or about 10 wt % of a compound described herein. In embodiments, a delivery vehicle (e.g., a liposomal delivery vehicle such as a lipid nanoparticle) comprises up to about 0.5 wt %, about 1 wt %, about 3 wt %, about 5 wt %, about 10 wt %, about 15 wt %, or about 20 wt % of a compound described herein. In embodiments, the percentage results in an improved beneficial effect (e.g., improved delivery to targeted tissues such as the liver, the lung or muscle).

The amount of a compound of the invention as described herein in a composition also can be described as a percentage (“mol %”) of the combined molar amounts of total lipids of a composition (e.g., the combined molar amounts of all lipids present in a liposomal delivery vehicle).

In embodiments of pharmaceutical compositions described herein, a compound of the invention as described herein is present in an amount that is about 0.5 mol % to about 50 mol % (e.g., about 0.5 mol % to about 20 mol %) of the combined molar amounts of all lipids present in a composition such as a liposomal delivery vehicle.

In embodiments, a compound of the invention as described herein is present in an amount that is about 0.5 mol % to about 5 mol %, about 1 mol % to about 10 mol %, about 5 mol % to about 20 mol %, about 10 mol % to about 20 mol %, about 15 mol % to about 30 mol %, about 20 mol % to about 35 mol %, about 25 mol % to about 40 mol %, about 30 mol % to about 45 mol %, about 35 mol % to about 50 mol %, about 40 mol % to about 55 mol %, or about 45 mol % to about 60 mol % of the combined molar amounts of all lipids present in a composition such as a liposomal delivery vehicle. In embodiments, a compound of the invention as described herein is present in an amount that is about 1 mol % to about 60 mol %, 1 mol % to about 50 mol %, 1 mol % to about 40 mol %, 1 mol % to about 30 mol %, about 1 mol % to about 20 mol %, about 1 mol % to about 15 mol %, about 1 mol % to about 10 mol %, about 5 mol % to about 55 mol %, about 5 mol % to about 45 mol %, about 5 mol % to about 35 mol % or about 5 mol % to about 25 mol % of the combined molar amounts of all lipids present in a composition such as a liposomal delivery vehicle.

In certain embodiments, a compound of the invention as described herein can comprise from about 0.1 mol % to about 50 mol %, or from 0.5 mol % to about 50 mol %, or from about 1 mol % to about 50 mol %, or from about 5 mol % to about 50 mol %, or from about 10 mol % to about 50 mol %, or from about 15 mol % to about 50 mol %, or from about 20 mol % to about 50 mol %, or from about 25 mol % to about 50 mol %, or from about 30 mol % to about 50 mol %, of the total amount of lipids in a composition (e.g., a liposomal delivery vehicle).

In certain embodiments, a compound of the invention as described herein can comprise greater than about 0.1 mol %, or greater than about 0.5 mol %, or greater than about 1 mol %, greater than about 5 mol %, greater than about 10 mol %, greater than about 20 mol %, greater than about 30 mol %, or greater than about 40 mol % of the total amount of lipids in the lipid nanoparticle.

In certain embodiments, a compound as described can comprise less than about 60 mol %, or less than about 55 mol %, or less than about 50 mol %, or less than about 45 mol %, or less than about 40 mol %, or less than about 35 mol %, less than about 30 mol %, or less than about 25 mol %, or less than about 10 mol %, or less than about 5 mol %, or less than about 1 mol % of the total amount of lipids in a composition (e.g., a liposomal delivery vehicle).

In embodiments, the amount of a compound of the invention as described herein is present in an amount that is at least about 5 mol %, about 10 mol %, about 15 mol %, about 20 mol %, about 25 mol %, about 30 mol %, about 35 mol %, about 40 mol %, about 45 mol %, about 50 mol %, about 55 mol %, about 60 mol %, about 65 mol %, about 70 mol %, about 75 mol %, about 80 mol %, about 85 mol %, about 90 mol %, about 95 mol %, about 96 mol %, about 97 mol %, about 98 mol %, or about 99 mol % of the combined molar amounts of total lipids in a composition (e.g., a liposomal composition).

In embodiments, the amount of a compound of the invention as described herein is present in an amount that is no more than about 5 mol %, about 10 mol %, about 15 mol %, about 20 mol %, about 25 mol %, about 30 mol %, about 35 mol %, about 40 mol %, about 45 mol %, about 50 mol %, about 55 mol %, about 60 mol %, about 65 mol %, about 70 mol %, about 75 mol %, about 80 mol %, about 85 mol %, about 90 mol %, about 95 mol %, about 96 mol %, about 97 mol %, about 98 mol %, or about 99 mol % of the combined molar amounts of total lipids in a composition (e.g., a liposomal composition).

In embodiments, the percentage results in an improved beneficial effect (e.g., improved delivery to targeted tissues such as the liver, the lung or muscle, optionally muscle).

In a typical embodiment, a composition of the invention (e.g., a liposomal composition) comprises:
(i) one or more cationic lipids,
(ii) one or more non-cationic lipids,
(iii) one or more cholesterol-based lipids, and
(iv) one or more PEG-modified lipids, wherein at least one cationic lipid is a compound of the invention as described herein.

For example, a composition suitable for practicing the invention has four lipid components comprising a compound of the invention as described herein as the cationic lipid component, and further comprising:
(i) a non-cationic lipid,
(ii) a cholesterol-based lipid and
(iii) a PEG-modified lipid.

The non-cationic lipid may be DOPE or DEPE. The cholesterol-based lipid may be cholesterol. The PEG-modified lipid may be DMG-PEG2K.

In further embodiments, pharmaceutical (e.g., liposomal) compositions comprise one or more of a PEG-modified lipid, a non-cationic lipid and a cholesterol lipid. In other embodiments, such pharmaceutical (e.g., liposomal) compositions comprise: one or more PEG-modified lipids; one or more non-cationic lipids; and one or more cholesterol lipids. In yet further embodiments, such pharmaceutical (e.g., liposomal) compositions comprise: one or more PEG-modified lipids and one or more cholesterol lipids.

In embodiments, a composition (e.g., lipid nanoparticle) that encapsulates a nucleic acid (e.g., mRNA encoding a peptide or protein) comprises one or more compounds of the invention as described herein, and one or more lipids selected from the group consisting of a cationic lipid, a non-cationic lipid, and a PEGylated lipid.

In embodiments, a composition (e.g., lipid nanoparticle) that encapsulates a nucleic acid (e.g., mRNA encoding a peptide or protein) comprises one or more compound of the invention as described herein; one or more lipids selected from the group consisting of a cationic lipid, a non-cationic lipid, and a PEGylated lipid; and further comprises a cholesterol-based lipid. Typically, such a composition has four lipid components comprising a compound of the invention as described herein as the cationic lipid component, and further comprising:
(i) a non-cationic lipid (e.g., DOPE),
(ii) a cholesterol-based lipid (e.g., cholesterol) and
(iii) a PEG-modified lipid (e.g., DMG-PEG2K).

In embodiments, a lipid nanoparticle that encapsulates a nucleic acid (e.g., mRNA encoding a peptide or protein) comprises one or more compounds of the invention as described herein, as well as one or more lipids selected from the group consisting of:
(i) a cationic lipid,
(ii) a non-cationic lipid,
(iii) a PEGylated lipid, and
(iv) a cholesterol-based lipid.

According to various embodiments, the selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the mRNA to be delivered. Additional considerations include, for example, the saturation of the alkyl chain, as well as the size, charge, pH, pKa, fusogenicity and toxicity of the selected lipid(s). Thus, the molar ratios may be adjusted accordingly.

In embodiments, a lipid nanoparticle of the present invention has a diameter of about 120 nm. In embodiments, a lipid nanoparticle of the present invention has a diameter of about 60-125 nm. In embodiments, a lipid nanoparticle of the present invention has a diameter of about 70-125 nm. In embodiments, a lipid nanoparticle of the present invention has a diameter of about 80-125 nm. In embodiments, a lipid nanoparticle of the present invention has a diameter of about 90-125 nm. In embodiments, a lipid nanoparticle of the present invention has a diameter of about 100-125 nm. In embodiments, a lipid nanoparticle of the present invention has a diameter of about 110-125 nm. In embodiments, a lipid nanoparticle of the present invention has a diameter of about 115-125 nm. In embodiments, a lipid nanoparticle of the present invention has a diameter of about 60-130 nm. In embodiments, a lipid nanoparticle of the present invention has a diameter of about 70-130 nm. In embodiments, a lipid nanoparticle of the present invention has a diameter of about 80-130 nm. In embodiments, a lipid nanoparticle of the present invention has a diameter of about 90-130 nm. In embodiments, a lipid nanoparticle of the present invention has a diameter of about 100-130 nm. In embodiments, a lipid nanoparticle of the present invention has a diameter of about 110-130 nm. In embodiments, the diameter of the lipid nanoparticle is determined using Dynamic light scattering (DLS). Dynamic Light Scattering (DLS) measurements, can be performed using a Malvern Instruments Zetasizer with a backscattering detector angle of 173° and a 4-mW, 633-nm He—Ne laser (Worcestershire, UK). The samples can be analyzed by diluting in 10% Trehalose and measuring the diameter in an optical grade polystyrene cuvette.

### Cationic Lipids

In addition to any of the compounds of the invention as described herein, a composition may comprise one or more additional cationic lipids.

In some embodiments, liposomes may comprise one or more additional cationic lipids. As used herein, the phrase “cationic lipid” refers to any of a number of lipid species that have a net positive charge at a selected pH, such as physiological pH. Several cationic lipids have been described in the literature, many of which are commercially available.

Suitable additional cationic lipids for use in the compositions include the cationic lipids as described in the literature.

### Helper Lipids

Compositions (e.g., liposomal compositions) may also comprise one or more helper lipids. Such helper lipids include non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), 1,2-Dierucoyl-sn-glycero-3-phosphoethanolamine (DEPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof. A non-cationic or helper lipid suitable for practicing the invention is dioleoylphosphatidylethanolamine (DOPE). Alternatively, 1,2-Dierucoyl-sn-glycero-3-phosphoethanolamine (DEPE) can be used as a non-cationic or helper lipid.

In some embodiments, a non-cationic lipid is a neutral lipid, i.e., a lipid that does not carry a net charge in the conditions under which the composition is formulated and/or administered.

In some embodiments, a non-cationic lipid may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 mol %, greater than about 10 mol %, greater than about 20 mol %, greater than about 30 mol %, or greater than about 40 mol %. In some embodiments, the percentage total non-cationic lipids in a liposome may be greater than about 5 mol %, greater than about 10 mol %, greater than about 20 mol %, greater than about 30 mol %, or greater than about 40 mol %. In some embodiments, the percentage of non-cationic lipid in a liposome is no more than about 5 mol %, no more than about 10 mol %, no more than about 20 mol %, no more than about 30 mol %, or no more than about 40 mol %. In some embodiments, the percentage total non-cationic lipids in a liposome may be no more than about 5 mol %, no more than about 10 mol %, no more than about 20 mol %, no more than about 30 mol %, or no more than about 40 mol %.

In some embodiments, a non-cationic lipid may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 wt %, greater than about 10 wt %, greater than about 20 wt %, greater than about 30 wt %, or greater than about 40 wt %. In some embodiments, the percentage total non-cationic lipids in a liposome may be greater than about 5 wt %, greater than about 10 wt %, greater than about 20 wt %, greater than about 30 wt %, or greater than about 40 wt %. In some embodiments, the percentage of non-cationic lipid in a liposome is no more than about 5 wt %, no more than about 10 wt %, no more than about 20 wt %, no more than about 30 wt %, or no more than about 40 wt %. In some embodiments, the percentage total non-cationic lipids in a liposome may be no more than about 5 wt %, no more than about 10 wt %, no more than about 20 wt %, no more than about 30 wt %, or no more than about 40 wt %.

### Cholesterol-Based Lipids

In some embodiments, a composition (e.g., a liposomal composition) comprising a cationic lipid of the present invention further comprises one or more cholesterol-based lipids. For example, a suitable cholesterol-based lipid for practicing the invention is cholesterol. Other suitable cholesterol-based lipids include, for example, DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), beta-sitosterol, or imidazole cholesterol ester (ICE), which has the following structure,

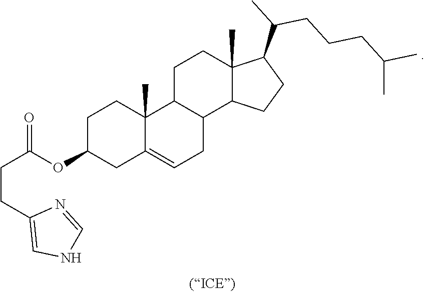 [SMILES: CC(C)CCCC(C)[C@H]1CCC2C3CC=C4C[C@@H](OC(=O)CCc5c[nH]cn5)CC[C@]4(C)C3CC[C@@]21C]

In some embodiments, a cholesterol-based lipid may be present in a molar ratio (mol %) of about 1% to about 30%, or about 5% to about 20% of the total lipids present in a liposome. In some embodiments, the percentage of cholesterol-based lipid in the lipid nanoparticle may be greater than about 5 mol %, greater than about 10 mol %, greater than about 20 mol %, greater than about 30 mol %, or greater than about 40 mol %. In some embodiments, the percentage of cholesterol-based lipid in the lipid nanoparticle may be no more than about 5 mol %, no more than about 10 mol %, no more than about 20 mol %, no more than about 30 mol %, or no more than about 40 mol %.

In some embodiments, a cholesterol-based lipid may be present in a weight ratio (wt %) of about 1% to about 30%, or about 5% to about 20% of the total lipids present in a liposome. In some embodiments, the percentage of cholesterol-based lipid in the lipid nanoparticle may be greater than about 5 wt %, greater than about 10 wt %, greater than about 20 wt %, greater than about 30 wt %, or greater than about 40 wt %. In some embodiments, the percentage of cholesterol-based lipid in the lipid nanoparticle may be no more than about 5 wt %, no more than about 10 wt %, no more than about 20 wt %, no more than about 30 wt %, or no more than about 40 wt %.

### PEGylated Lipids

In some embodiments, a composition (e.g., a liposomal composition) comprises one or more further PEGylated lipids. A suitable PEG-modified or PEGylated lipid for practicing the invention is 1,2-dimyristoyl-rac-glycero-3-methoxypolyethylene glycol-2000 (DMG-PEG2K).

For example, the use of polyethylene glycol (PEG)-modified phospholipids and derivatized lipids such as derivatized ceramides (PEG-CER), including N-octanoyl-sphingosine-1-[succinyl(methoxy polyethylene glycol)-2000](C8 PEG-2000 ceramide) is also contemplated by the present invention in combination with one or more of compounds of the invention as described herein and, in some embodiments, other lipids together which comprise the liposome. In some embodiments, particularly useful exchangeable lipids are PEG-ceramides having shorter acyl chains (e.g., (C₁₄) or (C₁₈)).

Contemplated further PEG-modified lipids (also referred to herein as a PEGylated lipid, which term is interchangeable with PEG-modified lipid) include, but are not limited to, a polyethylene glycol chain of up to 5 kDa in length covalently attached to a lipid with alkyl chain(s) of (C₆-C₂₀) length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid composition to the target cell, (Klibanov et al. (1990) FEBS Letters, 268 (1): 235-237), or they may be selected to rapidly exchange out of the formulation in vivo (see U.S. Pat. No. 5,885,613).

Further PEG-modified phospholipid and derivatized lipids of the present invention may be present in a molar ratio (mol %) from about 0% to about 10%, about 0.5% to about 10%, about 1% to about 10%, about 2% to about 10%, about 3% to about 5%, about 1% to about 5% or about 1.5% to about 3% of the total lipid present in the composition (e.g., a liposomal composition).

### Pharmaceutical Formulations and Therapeutic Uses

Compounds of the invention as described herein may be used in the preparation of compositions (e.g., to construct liposomal compositions) that facilitate or enhance the delivery and release of encapsulated materials (e.g., one or more therapeutic polynucleotides) to one or more target cells (e.g., by permeating or fusing with the lipid membranes of such target cells).

For example, when a liposomal composition (e.g., a lipid nanoparticle) comprises or is otherwise enriched with one or more of the compounds disclosed herein, the phase transition in the lipid bilayer of the one or more target cells may facilitate the delivery of the encapsulated materials (e.g., one or more therapeutic polynucleotides encapsulated in a lipid nanoparticle) into the one or more target cells.

Similarly, in certain embodiments compounds of the invention as described herein may be used to prepare liposomal vehicles that are characterized by their reduced toxicity in vivo. In certain embodiments, the reduced toxicity is a function of the high transfection efficiencies associated with the compositions disclosed herein, such that a reduced quantity of such composition may be administered to the subject to achieve a desired therapeutic response or outcome.

In certain embodiments, compounds of the invention as described herein may be used to prepare liposomal vehicles that are characterized by effective intramuscular delivery of mRNA. In certain embodiments, compounds of the invention as described herein may be used to prepare liposomal vehicles that are characterized by achieving high levels of peptide or protein expression when delivering mRNA encoding for said peptide or protein by intramuscular delivery.

Thus, pharmaceutical formulations comprising a compound described and nucleic acids provided by the present invention may be used for various therapeutic disease and/or disease prevention purposes. To facilitate delivery of nucleic acids in vivo, a compound described herein and nucleic acids can be formulated in combination with one or more additional pharmaceutical carriers, targeting ligands or stabilizing reagents. In some embodiments, a compound described herein can be formulated via pre-mixed lipid solution. In other embodiments, a composition comprising a compound described herein can be formulated using post-insertion techniques into the lipid membrane of the nanoparticles. Techniques for formulation and administration of drugs may be found in “Remington's Pharmaceutical Sciences,” Mack Publishing Co., Easton, Pa., latest edition.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal. In particular embodiments, the intramuscular administration is to a muscle selected from the group consisting of skeletal muscle, smooth muscle and cardiac muscle. In some embodiments the administration results in delivery of the nucleic acids to a muscle cell. In some embodiments the administration results in delivery of the nucleic acids to a hepatocyte (i.e., liver cell).

A common route for administering a liposomal composition of the invention may be intravenous delivery, in particular when treating metabolic disorders, especially those affecting the liver (e.g., ornithine transcarbamylase (OTC) deficiency). Alternatively, depending on the disease or disorder to be treated, the liposomal composition may be administered via pulmonary delivery (e.g., for the treatment of cystic fibrosis). For vaccination, a liposomal composition of the invention is typically administered intramuscularly. Diseases or disorders affecting the eye may be treated by administering a liposomal composition of the invention intravitreally.

Alternatively or additionally, pharmaceutical formulations of the invention may be administered in a local rather than systemic manner, for example, via injection of the pharmaceutical formulation directly into a targeted tissue (e.g., in a sustained release formulation). Local delivery can be affected in various ways, depending on the tissue to be targeted. Exemplary tissues in which mRNA may be delivered and/or expressed include, but are not limited to the liver, kidney, heart, spleen, serum, brain, skeletal muscle, lymph nodes, skin, and/or cerebrospinal fluid. In embodiments, the tissue to be targeted in the liver. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection.

Compositions described herein can comprise mRNA encoding peptides including those described herein (e.g., a polypeptide such as a protein).

In embodiments, a mRNA encodes a polypeptide.

In embodiments, a mRNA encodes a peptide. In embodiments, the peptide is an antigen.

In embodiments, a mRNA encodes a protein.

The present invention provides methods for delivering a composition having full-length mRNA molecules encoding a peptide or protein of interest for use in the treatment of a subject, e.g., a human subject or a cell of a human subject or a cell that is treated and delivered to a human subject.

### Delivery Methods

The route of delivery used in the methods of the invention allows for non-invasive, self-administration of the compounds of the invention. In some embodiments, the methods involve intranasal, intratracheal or pulmonary administration by aerosolization, nebulization, or instillation of a compositions comprising mRNA encoding a therapeutic peptide or protein in a suitable transfection or lipid carrier vehicles as described above. In some embodiments, the peptide or protein is encapsulated with a liposome. In some embodiments, the liposome comprises a lipid, which is a compound of the invention. As used herein below, administration of a compound of the invention includes administration of a composition comprising a compound of the invention.

Although the local cells and tissues of the lung represent a potential target capable of functioning as a biological depot or reservoir for production and secretion of the protein encoded by the mRNA, applicants have discovered that administration of the compounds of the invention to the lung via aerosolization, nebulization, or instillation results in the distribution of even non-secreted proteins outside the lung cells. Without wishing to be bound by any particular theory, it is contemplated that nanoparticle compositions of the invention pass, through the lung airway-blood barrier, resulting in translation of the intact nanoparticle to non-lung cells and tissues, such as, e.g., the heart, the liver, the spleen, the muscle, where it results in the production of the encoded peptide or protein in these non-lung tissues. Thus, the utility of the compounds of the invention and methods of the invention extend beyond production of therapeutic protein in lung cells and tissues of the lung and can be used to delivery to non-lung target cells and/or tissues. They are useful in the management and treatment of a large number of diseases. In certain embodiments, the compounds of the invention, used in the methods of the invention result in the distribution of the mRNA encapsulated nanoparticles and production of the encoded peptide or protein in the liver, spleen, heart, muscle and/or other non-lung cells. For example, administration of the compounds of the invention, by aerosolization, nebulization, or instillation to the lung will result in the composition itself and its peptide or protein product (e.g., an antigen or functional protein) will be detectable in both the local cells and tissues of the lung, as well as in peripheral target cells, tissues and organs as a result of translocation of the mRNA and delivery vehicle to non-lung cells.

In certain embodiments, the compounds of the invention may be employed in the methods of the invention to specifically target peripheral cells or tissues. Following the pulmonary delivery, it is contemplated the compounds of the invention cross the lung airway-blood barrier and distribute into cells other than the local lung cells. Accordingly, the compounds disclosed herein may be administered to a subject by way of the pulmonary route of administration, using a variety of approach known by those skilled in the art (e.g., by inhalation), and distribute to both the local target cells and tissues of the lung, as well as in peripheral non-lung cells and tissues (e.g., cells of the liver, spleen, kidneys, heart, skeletal muscle, lymph nodes, brain, cerebrospinal fluid, and plasma). As a result, both the local cells of the lung and the peripheral non-lung cells can serve as biological reservoirs or depots capable of producing and/or secreting a translation product encoded by one or more polynucleotides. Accordingly, the present invention is not limited to the treatment of lung diseases or conditions, but rather can be used as a non-invasive means of facilitating the delivery of polynucleotides, or the production of peptides or proteins encoded thereby, in peripheral organs, tissues and cells (e.g., hepatocytes) which would otherwise be achieved only by systemic administration. Exemplary peripheral non-lung cells include, but are not limited to, hepatocytes, epithelial cells, hematopoietic cells, epithelial cells, endothelial cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

Following administration of the composition to the subject, the peptide or protein product encoded by the mRNA (e.g., a functional protein or enzyme) is detectable in the peripheral target tissues for at least about one to seven days or longer following administration of the compound to the subject. The amount of peptide or protein product necessary to achieve a therapeutic effect will vary depending on the condition being treated, the peptide or protein encoded, and the condition of the patient. For example, the peptide or protein product may be detectable in the peripheral target tissues at a concentration (e.g., a therapeutic concentration) of at least 0.025-1.5 μg/ml (e.g., at least 0.050 μg/ml, at least 0.075 μg/ml, at least 0.1 μg/ml, at least 0.2 μg/ml, at least 0.3 μg/ml, at least 0.4 μg/ml, at least 0.5 μg/ml, at least 0.6 μg/ml, at least 0.7 μg/ml, at least 0.8 μg/ml, at least 0.9 μg/ml, at least 1.0 μg/ml, at least 1.1 μg/ml, at least 1.2 μg/ml, at least 1.3 μg/ml, at least 1.4 μg/ml, or at least 1.5 μg/ml), for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 days or longer following administration of the compound to the subject.

It has been demonstrated that nucleic acids can be delivered to the lungs by intratracheal administration of a liquid suspension of the compound and inhalation of an aerosol mist produced by a liquid nebulizer or the use of a dry powder apparatus such as that described in U.S. Pat. No. 5,780,014, incorporated herein by reference.

In certain embodiments, the compounds of the invention may be formulated such that they may be aerosolized or otherwise delivered as a particulate liquid or solid prior to or upon administration to the subject. Such compounds may be administered with the assistance of one or more suitable devices for administering such solid or liquid particulate compositions (such as, e.g., an aerosolized aqueous solution or suspension) to generate particles that are easily respirable or inhalable by the subject. In some embodiments, such devices (e.g., a metered dose inhaler, jet-nebulizer, ultrasonic nebulizer, dry-powder-inhalers, propellant-based inhaler or an insufflator) facilitate the administration of a predetermined mass, volume or dose of the compositions (e.g., about 0.5 mg/kg of mRNA per dose) to the subject. For example, in certain embodiments, the compounds of the invention are administered to a subject using a metered dose inhaler containing a suspension or solution comprising the compound and a suitable propellant. In certain embodiments, the compounds of the invention may be formulated as a particulate powder (e.g., respirable dry particles) intended for inhalation. In certain embodiments, compositions of the invention formulated as respirable particles are appropriately sized such that they may be respirable by the subject or delivered using a suitable device (e.g., a mean D50 or D90 particle size less than about 500 μm, 400 μm, 300 μm, 250 μm, 200 μm, 150 μm, 100 μm, 75 μm, 50 μm, 25 μm, 20 μm, 15 μm, 12.5 μm, 10 μm, 5 μm, 2.5 μm or smaller). In yet other embodiments, the compounds of the invention are formulated to include one or more pulmonary surfactants (e.g., lamellar bodies). In some embodiments, the compounds of the invention are administered to a subject such that a concentration of at least 0.05 mg/kg, at least 0.1 mg/kg, at least 0.5 mg/kg, at least 1.0 mg/kg, at least 2.0 mg/kg, at least 3.0 mg/kg, at least 4.0 mg/kg, at least 5.0 mg/kg, at least 6.0 mg/kg, at least 7.0 mg/kg, at least 8.0 mg/kg, at least 9.0 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg body weight is administered in a single dose. In some embodiments, the compounds of the invention are administered to a subject such that a total amount of at least 0.1 mg, at least 0.5 mg, at least 1.0 mg, at least 2.0 mg, at least 3.0 mg, at least 4.0 mg, at least 5.0 mg, at least 6.0 mg, at least 7.0 mg, at least 8.0 mg, at least 9.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg or at least 100 mg mRNA is administered in one or more doses.

### Synthesis of Compounds of the Invention

The cationic lipid MC3 is the current gold standard for in vivo delivery of e.g. siRNA (see WO2010/144740). However, the synthesis of this lipid involves a six-step process and requires handling of a Grignard reagent. In contrast, the present invention provides cationic lipids that can be prepared from readily available starting reagents, such as “Good's” buffers (see Table 1 below). These starting reagents can be coupled to cationic headgroups and lipid tails using coupling reactions, such as sulfonylation, acetylation and alkylation (see for example, Table 2 below).

TABLE 1
Examples of “Good” buffers

| “Good” | |
|---|---|
| Buffer | |
| name | Structure |
| HEPES | 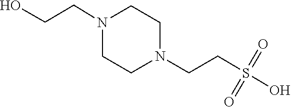 [SMILES: O=S(=O)(O)CCN1CCN(CCO)CC1] |
| HEPPS | 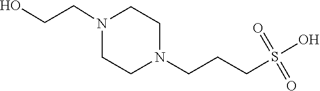 [SMILES: O=S(=O)(O)CCCN1CCN(CCO)CC1] |
| HEPBS | 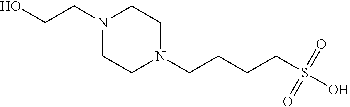 [SMILES: O=S(=O)(O)CCCCN1CCN(CCO)CC1] |
| PIPES | 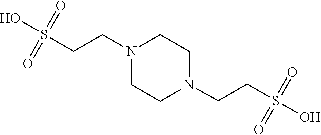 [SMILES: O=S(=O)(O)CCN1CCN(CCS(=O)(=O)O)CC1] |

TABLE 2
Examples of lipid chains that are suitable for the present invention

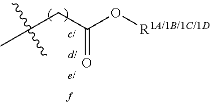

| Value of c, d, e or f | Structure of R^{1A}, R^{1B}, R^{1C} or R^{1D} |
|---|---|
| 3 | 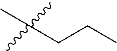 [SMILES: *CCC] |
| 3 | 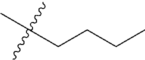 [SMILES: *CCCC] |
| 3 | 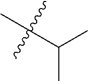 [SMILES: *C(C)C] |
| 3 | 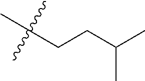 [SMILES: *CCC(C)C] |
| 3 | 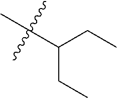 [SMILES: *C(CC)CC] |
| 3 | 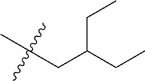 [SMILES: *CC(CC)CC] |
| 4 | 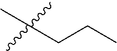 [SMILES: *CCC] |
| 4 | 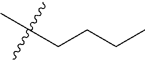 [SMILES: *CCCC] |
| 4 | 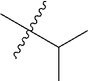 [SMILES: *C(C)C] |
| 4 | 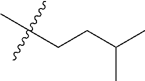 [SMILES: *CCC(C)C] |
| 4 | 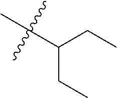 [SMILES: *C(CC)CC] |
| 4 | 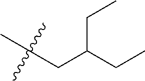 [SMILES: *CC(CC)CC] |
| 5 | 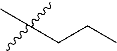 [SMILES: *CCC] |
| 5 | 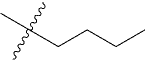 [SMILES: *CCCC] |
| 5 | 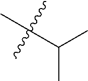 [SMILES: *C(C)C] |
| 5 | 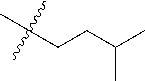 [SMILES: *CCC(C)C] |
| 5 | 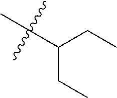 [SMILES: *C(CC)CC] |
| 5 | 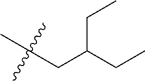 [SMILES: *CC(CC)CC] |
| 6 | 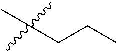 [SMILES: *CCC] |
| 6 | 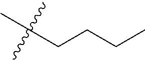 [SMILES: *CCCC] |
| 6 | 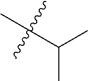 [SMILES: *C(C)C] |
| 6 | 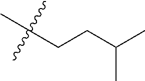 [SMILES: *CCC(C)C] |
| 6 | 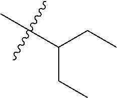 [SMILES: *C(CC)CC] |
| 6 | 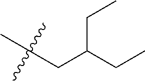 [SMILES: *CC(CC)CC] |

In embodiments, a cationic lipid described herein can be prepared by conjugating a “Good's” Buffer with a lipid, for example the carboxylic acid of a lipid, under suitable conditions. Exemplary “Good's” Buffers are described in Table 1, and exemplary lipid chains are described in Table 2. Accordingly, suitable cationic lipids include those resulting from any combination of the precursors described in Table 1 and Table 2.

In some embodiments, the sulfonic acid groups of compounds, such as “Good's” buffers can be derivatized by forming a sulfonyl chloride using reagents, such as oxalyl chloride. The resulting sulfonyl chloride can undergo a number of reactions, including but not limited to reduction with Zn/HCl to form the corresponding thiol and coupling to nucleophiles, such as amines and alcohols to form the corresponding sulfonamides and sulfonates (see for example, Scheme A below):

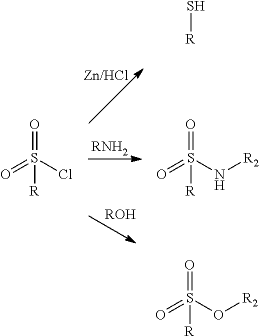

Using the chemistry outlined in scheme A it is possible to derivaties the sulfonic acid starting reagents with a range of suitable cationic lipid head groups and lipid chains.

Furthermore, compounds such as “Good's” buffers can be readily synthesized. For example, through nucleophilic ring opening of an episulfide with a piperazine (see for example, Scheme B below).

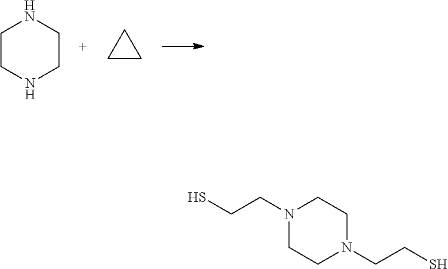

The compounds of the invention as described herein can be prepared according to methods known in the art, including the exemplary syntheses of the Examples provided herein.

### EXAMPLES

While certain compounds, compositions and methods of the present invention have been described with specificity in accordance with certain embodiments, the following examples serve only to illustrate the compounds of the invention and are not intended to limit the same.

Any of the compounds identified in the examples may be provided in the form of a pharmaceutically acceptable salt and such salts are intended to be encompassed by the present invention.

### LIST OF ABBREVIATIONS

APCI-MS: Atmospheric pressure chemical ionization mass spectrometry
EDCl: 1-Ethyl-3-(3-dimethylaminopropyl)carbodiimide
EtOAc: Ethyl acetate
MS: Mass spectrometry
Na₂SO₄: Sodium sulfate
SiO₂: Silicon dioxide
TLC: Thin layer chromatography

### Example 1: Synthesis of Compounds of the Present Invention

For example, the compounds of the invention may be prepared according to Schemes 1 and 2.

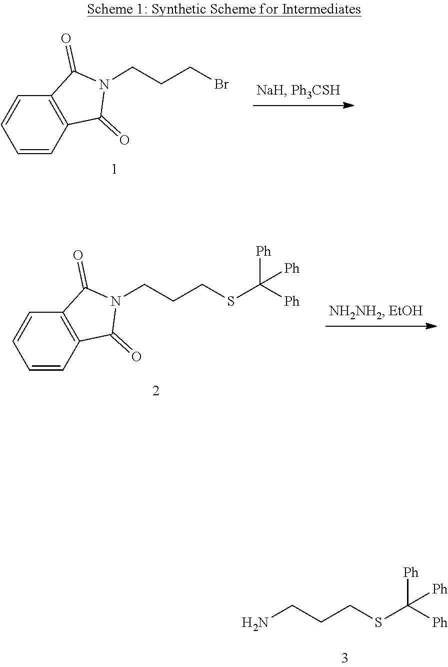 [SMILES: O=C1c2ccccc2C(=O)N1CCCBr ; O=C1c2ccccc2C(=O)N1CCCSC(c1ccccc1)(c1ccccc1)c1ccccc1 ; NCCCSC(c1ccccc1)(c1ccccc1)c1ccccc1]

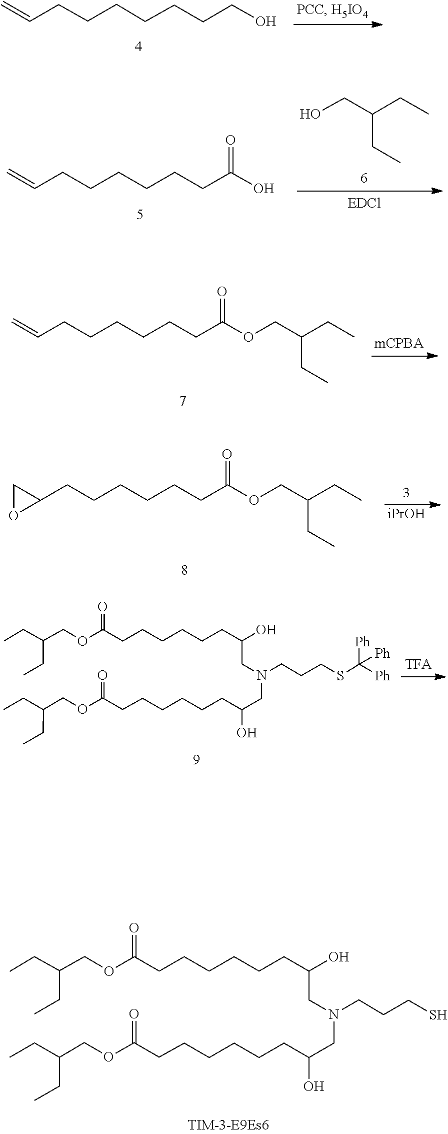

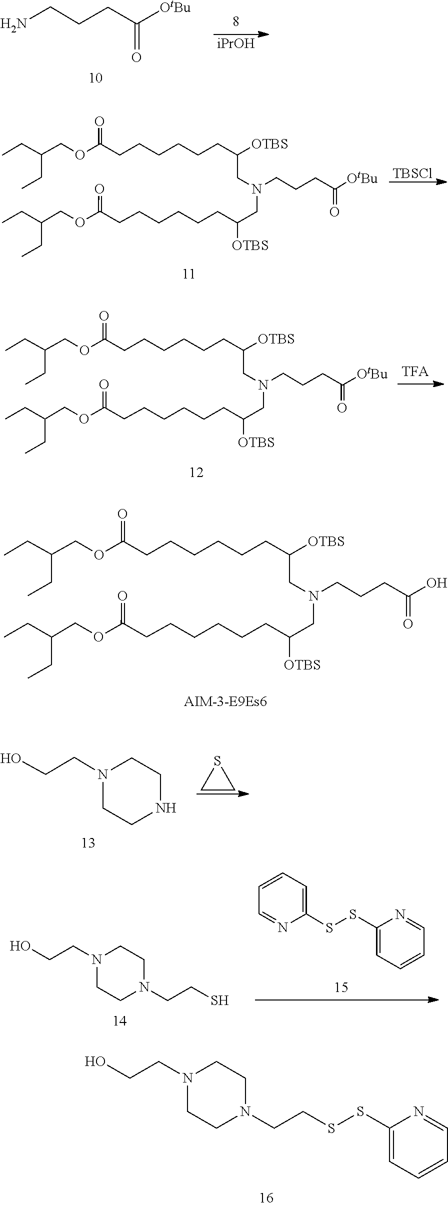

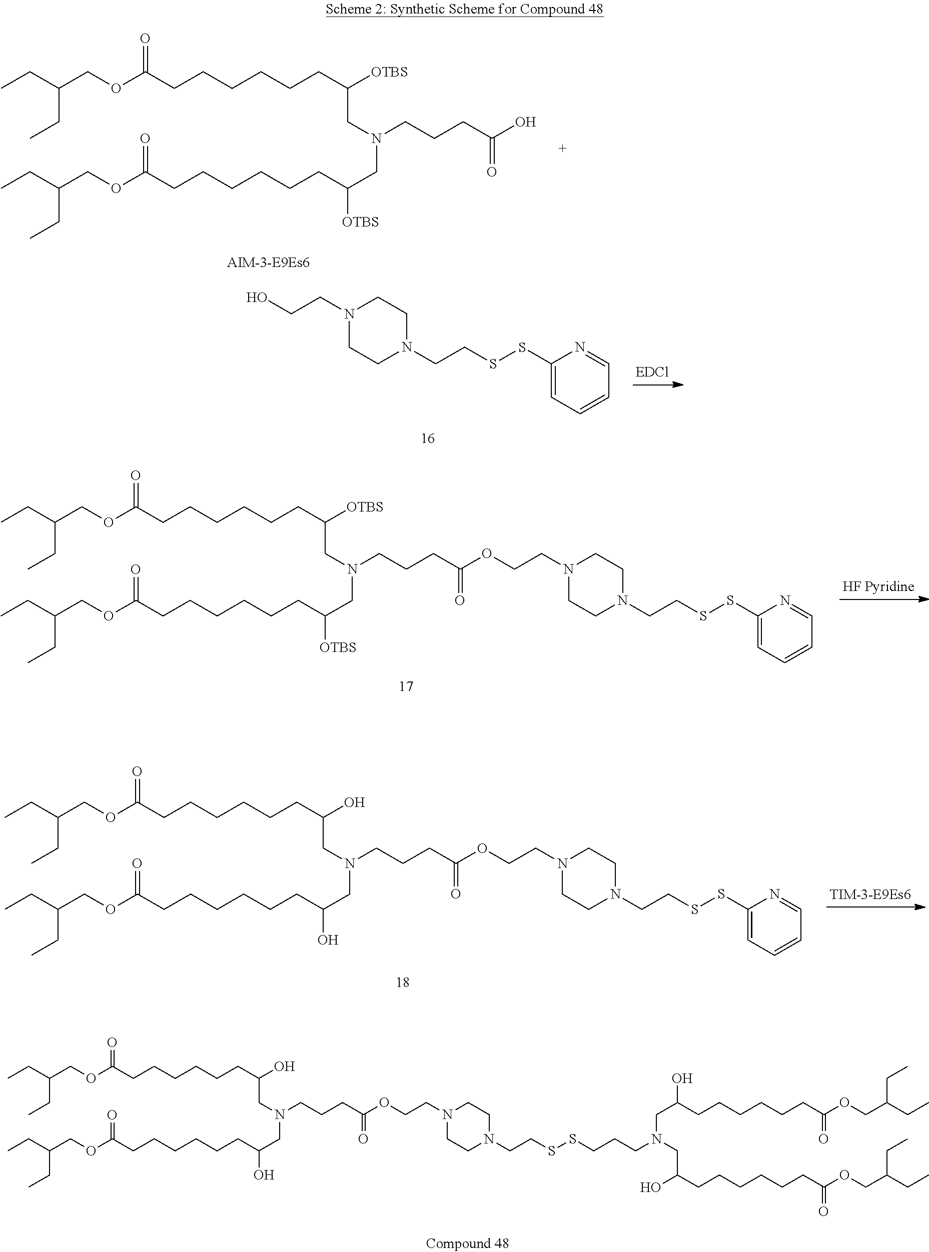 [SMILES: CCC(CC)COC(=O)CCCCCCC(CN(CCCC(=O)O)CC(CCCCCCC(=O)OCC(CC)CC)O[Si](C)(C)C(C)(C)C)O[Si](C)(C)C(C)(C)C ; OCCN1CCN(CCSSc2ccccn2)CC1 ; CCC(CC)COC(=O)CCCCCCC(CN(CCCC(=O)OCCN1CCN(CCSSc2ccccn2)CC1)CC(CCCCCCC(=O)OCC(CC)CC)O[Si](C)(C)C(C)(C)C)O[Si](C)(C)C(C)(C)C ; CCC(CC)COC(=O)CCCCCCC(O)CN(CCCC(=O)OCCN1CCN(CCSSc2ccccn2)CC1)CC(O)CCCCCCC(=O)OCC(CC)CC ; CCC(CC)COC(=O)CCCCCCC(O)CN(CCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCCCC(=O)OCC(CC)CC]

### Synthetic Procedure for Intermediate 3

### Step 1: Synthesis of 2-(3-(Tritylthio)propyl)isoindoline-1,3-dione (2)

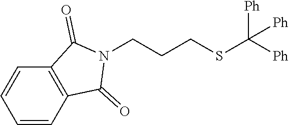 [SMILES: O=C1c2ccccc2C(=O)N1CCCSC(c1ccccc1)(c1ccccc1)c1ccccc1]

As depicted in Scheme 1: To a mixture of sodium hydride (30 g, 1.08 mole, 60% dispersion in mineral oil) in 600 mL N, N-dimethylformamide, was added triphenylmethanethiol (200 g, 0.724 mole) in portions at 0° C. After stirring for 1 h, a solution of N-(3-bromopropyl)phthalimide 1 (194.1 g, 0.724 mole) in 400 mL N, N-dimethylformamide was added slowly, and the resulting mixture was allowed to warm slowly to room temperature and stirred overnight. The reaction mixture was poured into 6 L ice-cold water, the solution was decanted, and the solid was dissolved in ethyl acetate and washed with brine. The organic layer was dried over Na₂SO₄ and concentrated to give 2-(3-(tritylthio)propyl)isoindoline-1,3-dione as white solid (252 g, 75%), which was used for the next step without further purification.

### Step 2: Synthesis of 3-(Tritylthio)propan-1-amine (3)

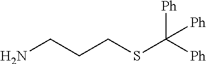 [SMILES: NCCCSC(c1ccccc1)(c1ccccc1)c1ccccc1]

As depicted in Scheme 1: A mixture of 2-(3-(tritylthio)propyl)isoindoline-1,3-dione 2 (252 g, 0.54 mole) and hydrazine hydrate (112 mL, 2.7 mole) in ethanol (3 L) was heated under nitrogen atmosphere to gentle reflux overnight. After cooled to room temperature, the reaction mixture was filtered through Celite, and then washed with ethanol. The combined filtrate was concentrated under reduced pressure, and the residue was dissolved in chloroform. After stirring for 15 min, the mixture was filtered and concentrated, and the crude was purified by flash column chromatography (SiO₂: 0 to 15% methanol in dichloromethane) to get 3-(tritylthio)propan-1-amine as oil (100 g, 55%).

### Synthetic Procedure for Epoxide 8

### Step 1: Synthesis of Non-8-enoic Acid (5)

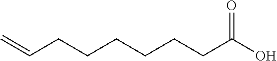

As depicted in Scheme 1: To a solution of periodic acid (353 g, 1.55 mole) in 2 L acetonitrile, a solution of non-8-en-1-ol 4 (100 g, 0.7 mole) was added at 0° C., and then a solution of pyridinium chlorochromate (3.23 g, 15 mmol) in 500 mL acetonitrile was added dropwise in 2 h. The resulting cloudy mixture was stirred at room temperature overnight. TLC showed complete reaction. The reaction mixture was diluted with 1 L EtOAc, and the solution was washed by water and brine. After dried over sodium sulfate, the organic layer was concentrated, and the crude was purified by column chromatography (SiO₂: 0 to 50% ethyl acetate in hexane) to get non-8-enoic acid as pale yellow oil (88 g, 80%).

### Step 2: Synthesis of 2-Ethylbutyl non-8-enoate (7)

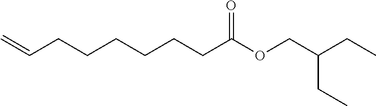 [SMILES: C=CCCCCCCC(=O)OCC(CC)CC]

As depicted in Scheme 1: To a mixture of non-8-enoic acid 5 (50 g, 0.32 mole) and 2-ethylbutanol 6 (39.2 g, 0.384 mole) in 250 mL dichloromethane, was added EDCl (73.6 g, 0.384 mole) and dimethylaminopyridine (7.8 g, 64 mmol), and then the reaction mixture was stirred overnight. MS and TLC analysis showed complete reaction. The reaction mixture was diluted with dichloromethane, and washed with saturated sodium bicarbonate, water and brine. After dried over sodium sulfate, the solvent was evaporated under vacuum, and the crude was purified via flash column chromatography (SiO₂: 0 to 20% ethyl acetate in hexane) to give 2-ethylbutyl non-8-enoate as colorless oil (71 g, 92%).

### Step 3: Synthesis of 2-Ethylbutyl 7-(oxiran-2-yl)heptanoate (8)

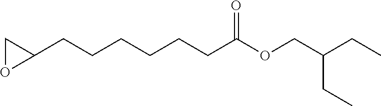 [SMILES: CCC(CC)COC(=O)CCCCCCC1CO1]

As depicted in Scheme 1: A solution of 2-ethylbutyl non-8-enoate 7 (71 g, 0.295 mole) in 500 mL dichloromethane was cooled to 0° C., and 3-chloroperbenzoic acid (99.3 g, 0.443 mole) was added. The reaction mixture was stirred at this temperature overnight. The suspension was filtered, and 1.2 M sodium bisulfite solution was added into the filtrate. After stirred for 1 h, the organic layer was separated, and then washed by sodium bicarbonate solution and brine. After dried over sodium sulfate, the solvent was evaporated to give 2-ethylbutyl 7-(oxiran-2-yl)heptanoate as colorless oil (70 g, 92%), which was used for the next step without purification.

### Synthetic Procedure for TIM-3-E9Es6

### Step 1: Synthesis of Bis(2-ethylbutyl) 9,9′-((3-(tritylthio)propyl)azanediyl)bis(8-hydroxynonanoate)

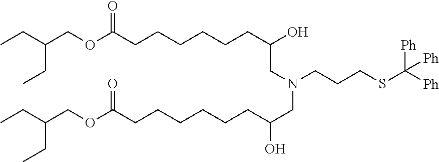 [SMILES: CCC(CC)COC(=O)CCCCCCC(O)CN(CCCSC(c1ccccc1)(c1ccccc1)c1ccccc1)CC(O)CCCCCCC(=O)OCC(CC)CC]

As depicted in Scheme 1: A mixture of 3-(tritylthio)propan-1-amine 3 (3.9 g, 11.7 mmol) and 2-ethylbutyl 7-(oxiran-2-yl)heptanoate 8 (8.0 g, 35.1 mmol) in 30 mL isopropanol was heated under nitrogen atmosphere to gentle reflux overnight. The reaction mixture was concentrated, and the crude was purified by flash column chromatography (SiO₂: 0 to 10% methanol in dichloromethane) to give bis(2-ethylbutyl) 9,9′-((3-(tritylthio)propyl)azanediyl)bis(8-hydroxynonanoate) as yellow oil (5.3 g, 53%).

### Step 2: Synthesis of Bis(2-ethylbutyl) 9,9′-((3-mercaptopropyl)azanediyl)bis(8-hydroxynonanoate) (TIM-3-E9Es6)

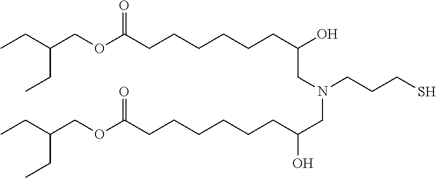 [SMILES: CCC(CC)COC(=O)CCCCCCC(O)CN(CCCS)CC(O)CCCCCCC(=O)OCC(CC)CC]

As depicted in Scheme 1: To a solution of bis(2-ethylbutyl) 9,9′-((3-(tritylthio)propyl)azanediyl)bis(8-hydroxynonanoate) 9 (169 mg, 0.20 mmol) and triethylsilane (0.1 mL, 0.6 mmol) in 10 mL dichloromethane, was added trifluoroacetic acid (0.1 mL, 1.0 mmol) slowly at 0° C. The resulting reaction mixture was warmed up to room temperature and stirred for 1 h. MS showed complete reaction. The volatile was evaporated, and the residue was evaporated with toluene three time under vacuum. The crude was used for the next step without further purification.

### Synthetic Procedure for AIM-3-E9Es6

### Step 1: Synthesis of Bis(2-ethylbutyl) 9,9′-((4-(tert-butoxy)-4-oxobutyl)azanediyl)bis(8-hydroxynonanoate) (11)

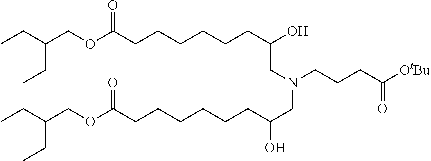 [SMILES: CCC(CC)COC(=O)CCCCCCC(O)CN(CCCC(=O)OC(C)(C)C)CC(O)CCCCCCC(=O)OCC(CC)CC]

As depicted in Scheme 1: A solution of tert-butyl 4-aminobutanoate 10 (3.3 g, 15.9 mmol), 2-ethylbutyl 7-(oxiran-2-yl)heptanoate 8 (9.0 g, 35.1 mmol) and diisopropylethylamine (5 mL, 28.7 mmol) in 5 mL isopropanol was heated to reflux for 3 days. MS showed complete reaction. After concentrated to dryness, the residue was purified by flash column chromatography (SiO₂: 0 to 100% ethyl acetate in hexane) to obtain bis(2-ethylbutyl) 9,9′-((4-(tert-butoxy)-4-oxobutyl)azanediyl)bis(8-hydroxynonanoate) as colorless oil (6.0 g, 56%).

### Step 2: Synthesis of Bis(2-ethylbutyl) 9,9′-((4-(tert-butoxy)-4-oxobutyl)azanediyl)bis(8-((tert-butyldimethylsilyl)oxy)nonanoate) (12)

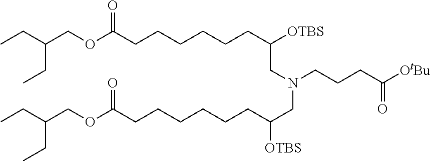 [SMILES: CCC(CC)COC(=O)CCCCCCC(CN(CCCC(=O)OC(C)(C)C)CC(CCCCCCC(=O)OCC(CC)CC)O[Si](C)(C)C(C)(C)C)O[Si](C)(C)C(C)(C)C]

As depicted in Scheme 1: To a solution of bis(2-ethylbutyl) 9,9′-((4-(tert-butoxy)-4-oxobutyl)azanediyl)bis(8-hydroxynonanoate) 11 (6.0 g, 8.7 mmol) in 50 mL dichloromethane, tert-butyldimethylsilyl chloride (5.3 g, 35 mmol), imidazole (0.6 g, 8.7 mmol) and dimethylaminopyridine (1.1 g, 8.7 mmol) were added, and the resulting mixture was heated to reflux 48 h. MS showed complete reaction. After cooled to room temperature, the reaction mixture was diluted with EtOAc, washed with water and brine. The combined organic layer was dried over sodium sulfate. After concentration, the residue was purified by flash column chromatography (SiO₂: 0 to 30% ethyl acetate in hexane) to obtain bis(2-ethylbutyl) 9,9′-((4-(tert-butoxy)-4-oxobutyl)azanediyl)bis(8-((tert-butyldimethylsilyl)oxy)nonanoate) as colorless oil (4.9 g, 61%).

### Step 7: Synthesis of 4-(Bis(2-((tert-butyldimethylsilyl)oxy)-9-(2-ethylbutoxy)-9-oxononyl)amino)butanoic acid (AIM-3-E9Es6)

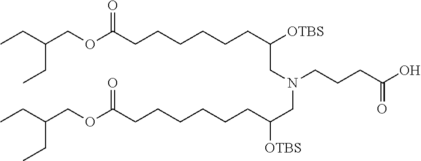 [SMILES: CCC(CC)COC(=O)CCCCCCC(CN(CCCC(=O)O)CC(CCCCCCC(=O)OCC(CC)CC)O[Si](C)(C)C(C)(C)C)O[Si](C)(C)C(C)(C)C]

As depicted in Scheme 1: A solution of bis(2-ethylbutyl) 9,9′-((4-(tert-butoxy)-4-oxobutyl)azanediyl)bis(8-((tert-butyldimethylsilyl)oxy)nonanoate) 12 (4.9 g, 5.36 mmol) in 15 mL dichloromethane was cooled to 0° C., and trifluoracetic acid (20 mL, 0.13 mole) was added dropwise, and the resulting mixture was stirred at room temperature overnight. MS showed complete reaction. Saturated sodium bicarbonate solution was added to adjust the solution to pH 7, and the mixture was extracted by dichloromethane. After dried over sodium sulfate, the solvent was removed under vacuum, and the residue was purified by flash column chromatography (SiO₂: 0 to 10% methanol in dichloromethane) to obtain 4-(bis(2-((tert-butyldimethylsilyl)oxy)-9-(2-ethylbutoxy)-9-oxononyl)amino)butanoic acid as colorless oil (4.1 g, 89%).

### Synthetic Procedure for Disulfide Intermediate 16

### Synthesis of 2-(4-(2-(Pyridin-2-yldisulfaneyl)ethyl)piperazin-1-yl)ethan-1-ol (16)

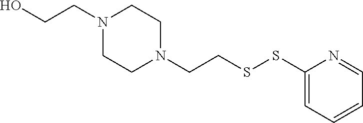 [SMILES: OCCN1CCN(CCSSc2ccccn2)CC1]

As depicted in Scheme 1: In a 2 L round-bottom flask, ethylene sulfide (18 g, 0.3 mole) was added into a solution of 2-(piperazin-1-yl)ethan-1-ol 13 (30.0 g, 0.23 mole) in 1500 mL dichloromethane, and the mixture was stirred at room temperature for 72 h. Pyridyl disulfide 15 (60.8 g, 0.276 mole) was added, and the reaction mixture was stirred at room temperature for 24 h. MS and TLC analysis indicated completion of the reaction. The reaction mixture was concentrated, and the residue was purified by flash column chromatography (SiO₂: 0 to 10% methanol in dichloromethane) to obtain 2-(4-(2-(pyridin-2-yldisulfaneyl)ethyl)piperazin-1-yl)ethan-1-ol as pale yellow oil (37 g, 53%).

### Synthetic Procedure for Compound 48

### Step 1: Synthesis of Bis(2-ethylbutyl) 9,9′-((4-oxo-4-(2-(4-(2-(pyridin-2-yldisulfaneyl)ethyl)piperazin-1-yl)ethoxy)butyl)azanediyl)bis(8-((tert-butyldimethylsilyl)oxy)nonanoate) (17)

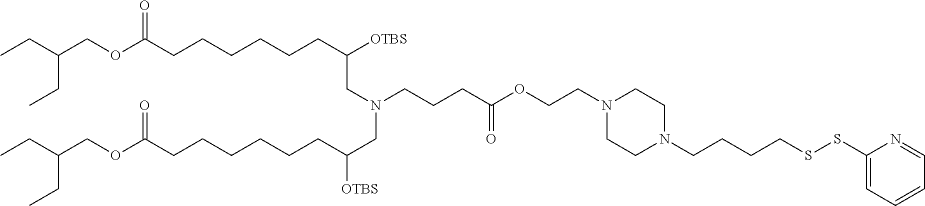 [SMILES: CCC(CC)COC(=O)CCCCCCC(CN(CCCC(=O)OCCN1CCN(CCCCSSc2ccccn2)CC1)CC(CCCCCCC(=O)OCC(CC)CC)O[Si](C)(C)C(C)(C)C)O[Si](C)(C)C(C)(C)C]

As depicted in Scheme 2: To a solution of 4-(bis(2-((tert-butyldimethylsilyl)oxy)-9-(2-ethylbutoxy)-9-oxononyl)amino)butanoic acid AIM-3-E9Es6 (2.2 g, 2.74 mmol) in 30 mL dichloromethane, was added EDCl (0.79 g, 4.11 mmol) and dimethylaminopyridine (67 mg, 0.54 mmol), and then a solution of 2-(4-(2-(pyridin-2-yldisulfaneyl)ethyl)piperazin-1-yl)ethan-1-ol 16 (0.98 g, 3.29 mmol) in 5 mL dichloromethane was added. The reaction mixture was stirred overnight. MS and TLC analysis showed complete reaction. The reaction mixture was diluted with dichloromethane, and washed with saturated sodium bicarbonate, water and brine. After dried over sodium sulfate, the solvent was evaporated under vacuum, and the crude was purified via flash column chromatography (SiO₂: 0 to 100% ethyl acetate with 1% triethylamine in hexane with 1% triethylamine, then 10% triethylamine in ethyl acetate, and then 25% triethylamine in ethyl acetate) to give bis(2-ethylbutyl) 9,9′-((4-oxo-4-(2-(4-(2-(pyridin-2-yldisulfaneyl)ethyl)piperazin-1-yl)ethoxy)butyl)azanediyl)bis(8-((tert-butyldimethylsilyl)oxy)nonanoate) as colorless oil (1.8 g, 60%).

### Step 2: Synthesis of Bis(2-ethylbutyl) 9,9′-((4-oxo-4-(2-(4-(2-(pyridin-2-yldisulfaneyl)ethyl)piperazin-1-yl)ethoxy)butyl)azanediyl)bis(8-hydroxynonanoate) (18)

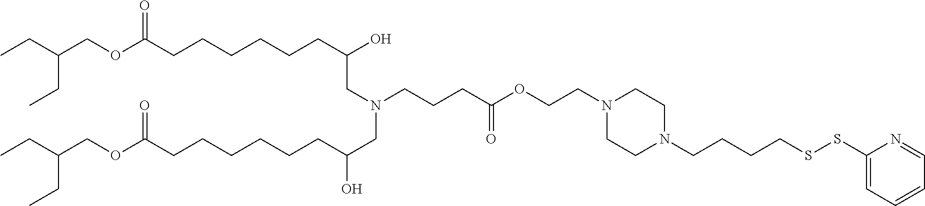 [SMILES: CCC(CC)COC(=O)CCCCCCC(O)CN(CCCC(=O)OCCN1CCN(CCCCSSc2ccccn2)CC1)CC(O)CCCCCCC(=O)OCC(CC)CC]

As depicted in Scheme 2: To a solution of bis(2-ethylbutyl) 9,9′-((4-oxo-4-(2-(4-(2-(pyridin-2-yldisulfaneyl)ethyl)piperazin-1-yl)ethoxy)butyl)azanediyl)bis(8-((tert-butyldimethylsilyl)oxy)nonanoate) 17 (1.8 g, 1.6 mmol) in 30 mL tetrahydrofuran/dichloromethane (1:1) at 0° C. was added hydrogen fluoride pyridine (70% HF, 1 mL, 34.5 mmol). The reaction mixture was warmed to room temperature and stirred for 16 h. MS and TLC analysis indicated complete reaction. The reaction was quenched by pouring slowly into saturated sodium bicarbonate, and then the resulting mixture was extracted with dichloromethane. Combined organic layer was washed with brine and dried over sodium sulfate. After concentration, the crude was purified by flash column chromatography (SiO₂: 0 to 100% ethyl acetate with 1% triethylamine in hexane with 1% triethylamine, then 10% triethylamine in ethyl acetate, and then 25% triethylamine in ethyl acetate) to give bis(2-ethylbutyl) 9,9′-((4-oxo-4-(2-(4-(2-(pyridin-2-yldisulfaneyl)ethyl)piperazin-1-yl)ethoxy)butyl)azanediyl)bis(8-hydroxynonanoate) was obtained as pale yellow oil (1.06 g, 73%).

### Step 3: Synthesis of Bis(2-ethylbutyl) 9,9′-((3-((2-(4-(2-((4-(bis(9-(2-ethylbutoxy)-2-hydroxy-9-oxononyl)amino)butanoyl)oxy)ethyl)piperazin-1-yl)ethyl)disulfaneyl)propyl)azanediyl)-bis(8-hydroxynonanoate) (Compound 48)

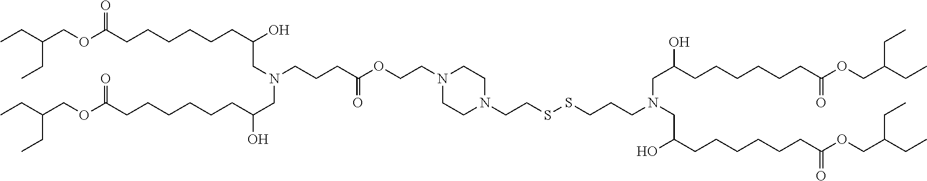 [SMILES: CCC(CC)COC(=O)CCCCCCC(O)CN(CCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCCCC(=O)OCC(CC)CC]

As depicted in Scheme 2: To a solution of bis(2-ethylbutyl) 9,9′-((4-oxo-4-(2-(4-(2-(pyridin-2-yldisulfaneyl)ethyl)piperazin-1-yl)ethoxy)butyl)azanediyl)bis(8-hydroxynonanoate) 18 (90 mg, 0.10 mmol) in 5 mL chloroform, was added a solution of crude bis(2-ethylbutyl) 9,9′-((3-mercaptopropyl)azanediyl)bis(8-hydroxynonanoate) TIM-3-E9Es6 (0.20 mmol). The reaction mixture was purged with nitrogen three times and then stirred at room temperature for 2 h. MS and TLC analysis indicated complete reaction. The reaction mixture was concentrated to dryness, and the crude was purified with flash column chromatography (SiO₂: 0 to 100% ethyl acetate with 1% triethylamine in hexane with 1% triethylamine, then 10% triethylamine in ethyl acetate) to give bis(2-ethylbutyl) 9,9′-((3-((2-(4-(2-((4-(bis(9-(2-ethylbutoxy)-2-hydroxy-9-oxononyl)amino)butanoyl)oxy)ethyl)piperazin-1-yl)ethyl)disulfaneyl)propyl)azanediyl)bis(8-hydroxynonanoate) as pale yellow oil (82 mg, 58%).

The other lipids of the present invention were prepared according to the representative procedures set out in Schemes 1 and 2 and described above.

### Diisopentyl 9,9′-((5-(2-(4-(2-((3-(bis(7-butoxy-2-hydroxy-7-oxoheptyl)amino)-propyl)disulfaneyl)ethyl)piperazin-1-yl)ethoxy)-5-oxopentyl)azanediyl)bis(8-hydroxynonanoate) (Compound 1)

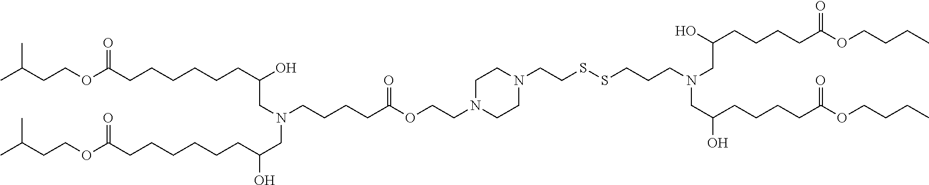 [SMILES: CCCCOC(=O)CCCCC(O)CN(CCCSSCCN1CCN(CCOC(=O)CCCCN(CC(O)CCCCCCC(=O)OCCC(C)C)CC(O)CCCCCCC(=O)OCCC(C)C)CC1)CC(O)CCCCC(=O)OCCCC]

1H NMR (300 MHz, CDCl3) δ 4.19 (t, 2H), 4.08 (t, 4H), 4.06 (t, 4H), 3.65 (m, 4H), 2.86-2.46 (m, 22H), 2.45-2.23 (m, 16H), 1.91-1.23 (m, 52H), 0.92 (t, 6H), 0.91 (d, 12H).

APCI-MS analysis: Calculated C66H126N4O14S2 [M+H]=1263.8, Observed=1263.9.

### Diisopentyl 9,9′-((5-(2-(4-(2-((4-(bis(2-hydroxy-7-(isopentyloxy)-7-oxoheptyl)amino)butyl)-disulfaneyl)ethyl)piperazin-1-yl)ethoxy)-5-oxopentyl)azanediyl)bis(8-hydroxynonanoate) (Compound 18)

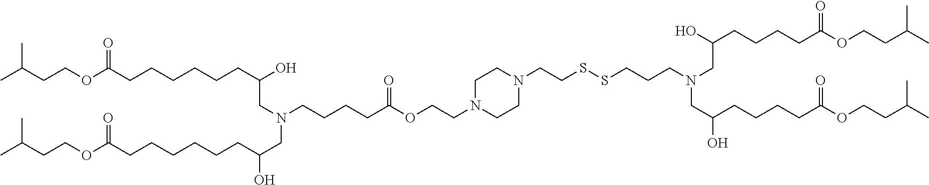 [SMILES: CC(C)CCOC(=O)CCCCCCC(O)CN(CCCCC(=O)OCCN1CCN(CCSSCCCN(CC(O)CCCCC(=O)OCCC(C)C)CC(O)CCCCC(=O)OCCC(C)C)CC1)CC(O)CCCCCCC(=O)OCCC(C)C]

¹H NMR (300 MHz, CDCl₃) δ 4.19 (t, 2H), 4.08 (t, 8H), 3.62 (m, 4H), 2.85-2.23 (m, 38H), 1.79-1.25 (m, 56H), 0.91 (d, 24H).

APCI-MS analysis: Calculated C69H132N4O14S2 [M+H]=1305.9, Observed=1305.9.

### Diisopentyl 9,9′-((5-(2-(4-(2-((4-(bis(7-(2-ethylbutoxy)-2-hydroxy-7-oxoheptyl)amino)-butyl)disulfaneyl)ethyl)piperazin-1-yl)ethoxy)-5-oxopentyl)azanediyl)bis(8-hydroxynonanoate) (Compound 49)

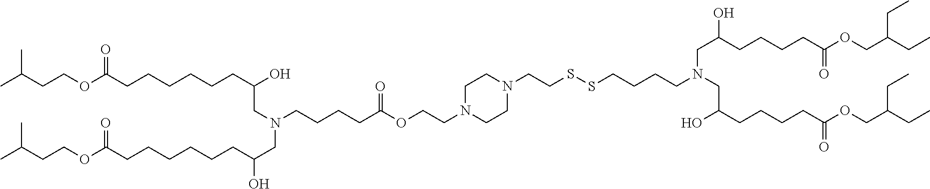 [SMILES: CCC(CC)COC(=O)CCCCC(O)CN(CCCCSSCCN1CCN(CCOC(=O)CCCCN(CC(O)CCCCCCC(=O)OCCC(C)C)CC(O)CCCCCCC(=O)OCCC(C)C)CC1)CC(O)CCCCC(=O)OCC(CC)CC]

¹H NMR (300 MHz, CDCl₃) δ 4.19 (t, 2H), 4.08 (t, 4H), 3.98 (d, 4H), 3.61 (m, 4H), 2.85-2.23 (m, 40H), 1.79-1.25 (m, 52H), 0.91 (d, 12H), 0.88 (t, 12H).

APCI-MS analysis: Calculated C71H136N4O14S2 [M+H]=1334.0, Observed=1334.0.

### Dibutyl 9,9′-((4-((2-(4-(2-((5-(bis(2-hydroxy-9-(isopentyloxy)-9-oxononyl)amino)-pentanoyl)oxy)ethyl)piperazin-1-yl)ethyl)disulfaneyl)butyl)azanediyl)bis(8-hydroxynonanoate) (Compound 10)

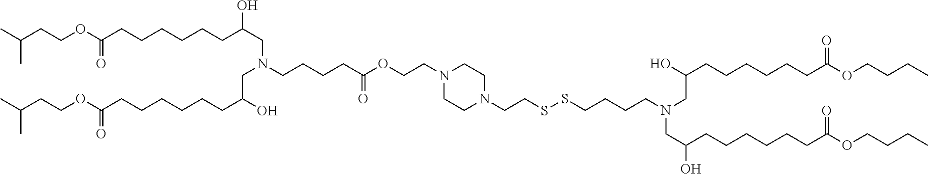 [SMILES: CCCCOC(=O)CCCCCCC(O)CN(CCCCSSCCN1CCN(CCOC(=O)CCCCN(CC(O)CCCCCCC(=O)OCCC(C)C)CC(O)CCCCCCC(=O)OCCC(C)C)CC1)CC(O)CCCCCCC(=O)OCCCC]

¹H NMR (300 MHz, CDCl₃) δ 4.19 (t, 2H), 4.08 (t, 4H), 4.05 (t, 4H), 3.64 (m, 4H), 2.86-2.23 (m, 40H), 1.75-1.23 (m, 64H), 0.92 (t, 6H), 0.91 (d, 12H).

APCI-MS analysis: Calculated C71H136N4O14S2 [M+H]=1334.0, Observed=1333.7.

### Diisopentyl 9,9′-((5-(2-(4-(2-((3-(bis(7-(2-ethylbutoxy)-2-hydroxy-7-oxoheptyl)amino)-propyl)disulfaneyl)ethyl)piperazin-1-yl)ethoxy)-5-oxopentyl)azanediyl)bis(8-hydroxynonanoate) (Compound 39)

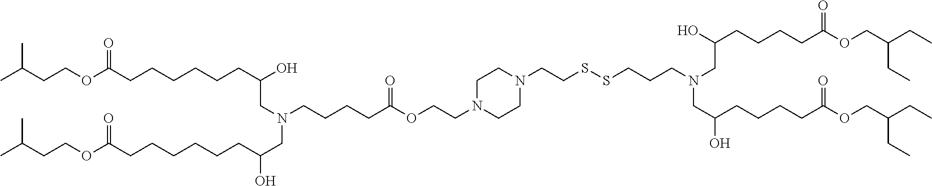 [SMILES: CCC(CC)COC(=O)CCCCC(O)CN(CCCSSCCN1CCN(CCOC(=O)CCCCN(CC(O)CCCCCCC(=O)OCCC(C)C)CC(O)CCCCCCC(=O)OCCC(C)C)CC1)CC(O)CCCCC(=O)OCC(CC)CC]

¹H NMR (300 MHz, CDCl₃) δ 4.19 (t, 2H), 4.08 (t, 4H), 3.98 (d, 4H), 3.62 (m, 4H), 2.85-2.23 (m, 38H), 1.79-1.25 (m, 58H), 0.91 (d, 12H), 0.88 (t, 12H).

APCI-MS analysis: Calculated C70H134N4O14S2 [M+H]=1319.9, Observed=1320.0.

### Diisopentyl 9,9′-((5-(2-(4-(2-((3-(bis(2-hydroxy-7-isopropoxy-7-oxoheptyl)amino)-propyl)disulfaneyl)ethyl)piperazin-1-yl)ethoxy)-5-oxopentyl)azanediyl)bis(8-hydroxynonanoate) (Compound 13)

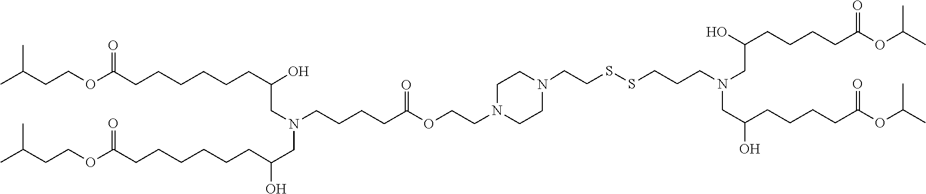 [SMILES: CC(C)CCOC(=O)CCCCCCC(O)CN(CCCCC(=O)OCCN1CCN(CCSSCCCN(CC(O)CCCCC(=O)OC(C)C)CC(O)CCCCC(=O)OC(C)C)CC1)CC(O)CCCCCCC(=O)OCCC(C)C]

¹H NMR (300 MHz, CDCl₃) δ 4.99 (hept, 2H), 4.19 (t, 2H), 4.08 (t, 4H), 3.62 (m, 4H), 2.85-2.22 (m, 38H), 1.85-1.24 (m, 44H), 1.22 (d, 12H), 0.91 (d, 12H).

APCI-MS analysis: Calculated C64H122N4O14S2 [M+H]=1235.8, Observed=1235.9.

### Diisopentyl 9,9′-((5-(2-(4-(2-((3-(bis(2-hydroxy-7-(isopentyloxy)-7-oxoheptyl)amino)-propyl)disulfaneyl)ethyl)piperazin-1-yl)ethoxy)-5-oxopentyl)azanediyl)bis(8-hydroxynonanoate) (Compound 19)

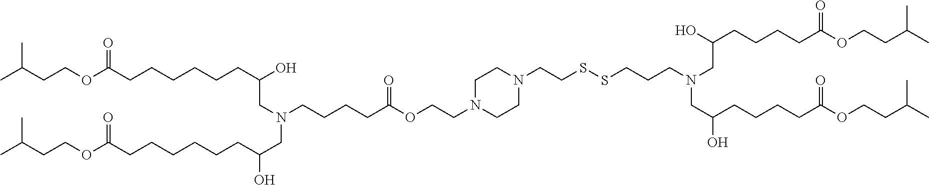 [SMILES: CC(C)CCOC(=O)CCCCCCC(O)CN(CCCCC(=O)OCCN1CCN(CCSSCCCN(CC(O)CCCCC(=O)OCCC(C)C)CC(O)CCCCC(=O)OCCC(C)C)CC1)CC(O)CCCCCCC(=O)OCCC(C)C]

¹H NMR (300 MHz, CDCl₃) δ 4.19 (t, 2H), 4.08 (t, 8H), 3.62 (m, 4H), 2.85-2.23 (m, 38H), 1.91-1.25 (m, 54H), 0.91 (d, 24H).

APCI-MS analysis: Calculated C68H130N4O14S2 [M+H]=1291.9, Observed=1292.0.

### Dibutyl 9,9′-((3-((2-(4-(2-((5-(bis(2-hydroxy-9-(isopentyloxy)-9-oxononyl)amino)-pentanoyl)oxy)ethyl)piperazin-1-yl)ethyl)disulfaneyl)propyl)azanediyl)bis(8-hydroxynonanoate) (Compound 4)

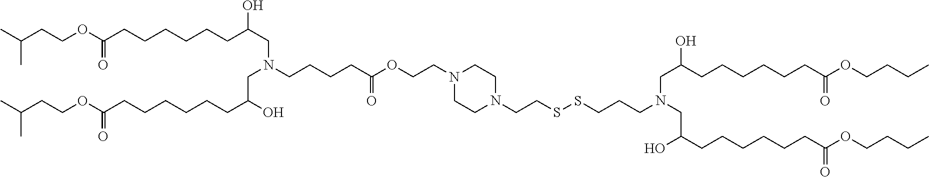 [SMILES: CCCCOC(=O)CCCCCCC(O)CN(CCCSSCCN1CCN(CCOC(=O)CCCCN(CC(O)CCCCCCC(=O)OCCC(C)C)CC(O)CCCCCCC(=O)OCCC(C)C)CC1)CC(O)CCCCCCC(=O)OCCCC]

¹H NMR (300 MHz, CDCl₃) δ 4.19 (t, 2H), 4.08 (t, 4H), 4.06 (t, 4H), 3.64 (m, 4H), 2.86-2.46 (m, 22H), 2.45-2.23 (m, 16H), 1.90-1.23 (m, 64H), 0.92 (t, 6H), 0.91 (d, 12H).

APCI-MS analysis: Calculated C70H134N4O14S2 [M+H]=1319.9, Observed=1319.0.

### Diisopentyl 9,9′-((5-(2-(4-(2-((4-(bis(7-butoxy-2-hydroxy-7-oxoheptyl)amino)butyl)-disulfaneyl)ethyl)piperazin-1-yl)ethoxy)-5-oxopentyl)azanediyl)bis(8-hydroxynonanoate) (Compound 7)

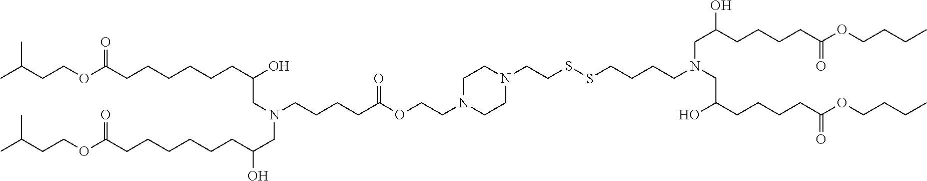 [SMILES: CCCCOC(=O)CCCCC(O)CN(CCCCSSCCN1CCN(CCOC(=O)CCCCN(CC(O)CCCCCCC(=O)OCCC(C)C)CC(O)CCCCCCC(=O)OCCC(C)C)CC1)CC(O)CCCCC(=O)OCCCC]

¹H NMR (300 MHz, CDCl₃) δ 4.19 (t, 2H), 4.08 (t, 4H), 4.06 (t, 4H), 3.61 (m, 4H), 2.84-2.46 (m, 22H), 2.45-2.23 (m, 16H), 1.80-1.25 (m, 54H), 0.92 (t, 6H), 0.91 (d, 12H).

APCI-MS analysis: Calculated C67H128N4O14S2 [M+H]=1277.9, Observed=1278.0.

### Diisopentyl 9,9′-((5-(2-(4-(2-((4-(bis(2-hydroxy-7-isopropoxy-7-oxoheptyl)amino)-butyl)disulfaneyl)ethyl)piperazin-1-yl)ethoxy)-5-oxopentyl)azanediyl)bis(8-hydroxynonanoate) (Compound 15)

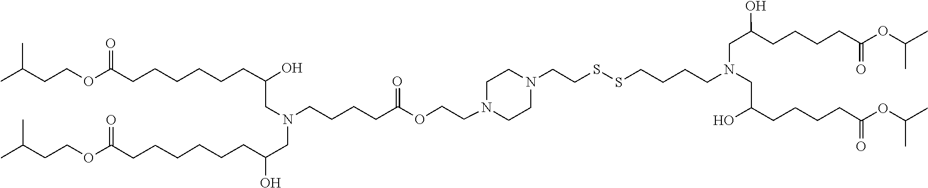 [SMILES: CC(C)CCOC(=O)CCCCCCC(O)CN(CCCCC(=O)OCCN1CCN(CCSSCCCCN(CC(O)CCCCC(=O)OC(C)C)CC(O)CCCCC(=O)OC(C)C)CC1)CC(O)CCCCCCC(=O)OCCC(C)C]

¹H NMR (300 MHz, CDCl₃) δ 4.99 (hept, 2H), 4.19 (t, 2H), 4.08 (t, 4H), 3.61 (m, 4H), 2.85-2.22 (m, 38H), 1.78-1.24 (m, 46H), 1.22 (d, 12H), 0.91 (d, 12H).

APCI-MS analysis: Calculated C65H124N4O14S2 [M+H]=1249.8, Observed=1249.9.

### Bis(2-ethylbutyl) 9,9′-((4-(2-(4-(2-((3-(bis(2-hydroxy-7-isopropoxy-7-oxoheptyl)amino)propyl)disulfaneyl)ethyl)piperazin-1-yl)ethoxy)-4-oxobutyl)azanediyl)-bis(8-hydroxynonanoate) (Compound 14)

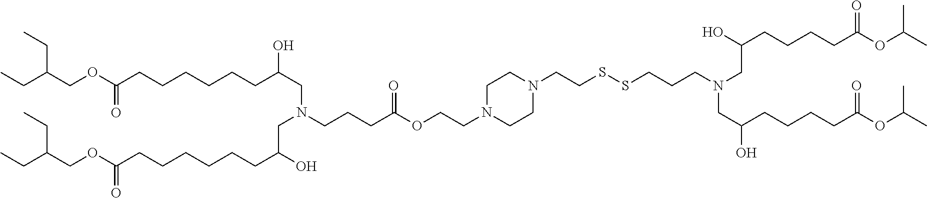 [SMILES: CCC(CC)COC(=O)CCCCCCC(O)CN(CCCC(=O)OCCN1CCN(CCSSCCCN(CC(O)CCCCC(=O)OC(C)C)CC(O)CCCCC(=O)OC(C)C)CC1)CC(O)CCCCCCC(=O)OCC(CC)CC]

¹H NMR (300 MHz, CDCl₃) δ 4.99 (hept, 2H), 4.19 (t, 2H), 3.98 (d, 4H), 3.63 (m, 4H), 2.84-2.35 (m, 30H), 2.28 (q, 8H), 1.92-1.74 (m, 5H), 1.68-1.56 (m, 9H), 1.54-1.26 (m, 32H), 1.22 (d, 12H), 0.88 (t, 12H).

APCI-MS analysis: Calculated C65H124N4O14S2 [M+H]=1249.8, Observed=1249.7.

### Bis(2-ethylbutyl) 9,9′-((4-(2-(4-(2-((3-(bis(2-hydroxy-7-(isopentyloxy)-7-oxoheptyl)amino)propyl)disulfaneyl)ethyl)piperazin-1-yl)ethoxy)-4-oxobutyl)azanediyl)-bis(8-hydroxynonanoate) (Compound 20)

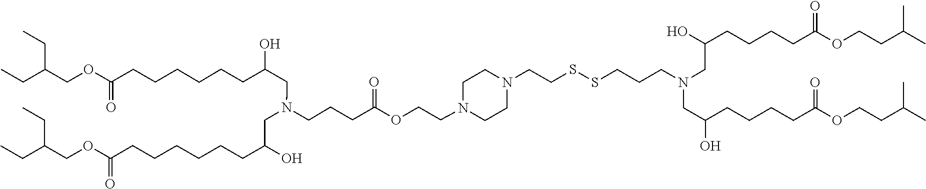 [SMILES: CCC(CC)COC(=O)CCCCCCC(O)CN(CCCC(=O)OCCN1CCN(CCSSCCCN(CC(O)CCCCC(=O)OCCC(C)C)CC(O)CCCCC(=O)OCCC(C)C)CC1)CC(O)CCCCCCC(=O)OCC(CC)CC]

¹H NMR (300 MHz, CDCl₃) δ 4.20 (t, 2H), 4.08 (t, 4H), 3.98 (d, 4H), 3.65 (m, 4H), 2.84-2.32 (m, 32H), 2.29 (dt, 8H), 1.92-1.74 (m, 5H), 1.72-1.56 (m, 9H), 1.54-1.26 (m, 36H), 0.91 (d, 12H), 0.88 (t, 12H).

APCI-MS analysis: Calculated C69H132N4O14S2 [M+H]=1305.9, Observed=1305.8.

### Bis(2-ethylbutyl) 9,9′-((4-(2-(4-(2-((3-(bis(7-(2-ethylbutoxy)-2-hydroxy-7-oxoheptyl)amino)propyl)disulfaneyl)ethyl)piperazin-1-yl)ethoxy)-4-oxobutyl)azanediyl)-bis(8-hydroxynonanoate) (Compound 40)

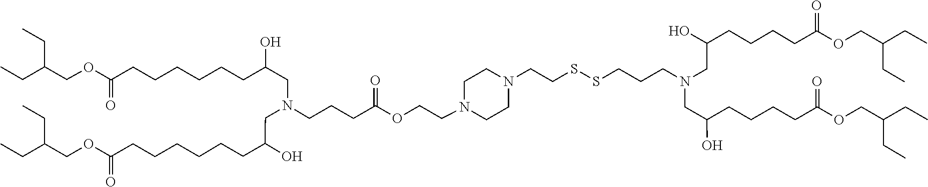 [SMILES: CCC(CC)COC(=O)CCCCCCC(O)CN(CCCC(=O)OCCN1CCN(CCSSCCCN(CC(O)CCCCC(=O)OCC(CC)CC)CC(O)CCCCC(=O)OCC(CC)CC)CC1)CC(O)CCCCCCC(=O)OCC(CC)CC]

¹H NMR (300 MHz, CDCl₃) δ 4.21 (t, 2H), 3.97 (d, 8H), 3.78 (m, 6H), 2.94-2.39 (m, 28H), 2.29 (dt, 8H), 1.92-1.74 (m, 4H), 1.72-1.26 (m, 52H), 0.88 (t, 24H).

APCI-MS analysis: Calculated C71H136N4O14S2 [M+H]=1334.0, Observed=1333.8.

### Bis(2-ethylbutyl) 9,9′-((4-(2-(4-(2-((3-(bis(2-hydroxy-9-oxo-9-propoxynonyl)amino)propyl)disulfaneyl)ethyl)piperazin-1-yl)ethoxy)-4-oxobutyl)azanediyl)bis(8-hydroxynonanoate) (Compound 5)

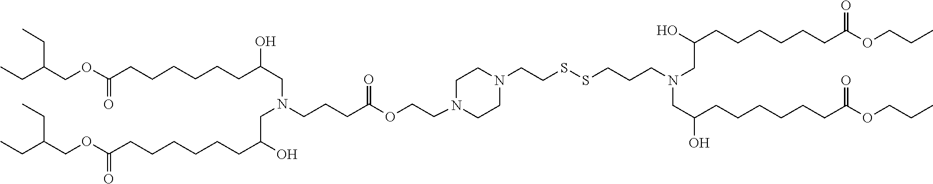 [SMILES: CCCOC(=O)CCCCCCC(O)CN(CCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCCCC(=O)OCCC]

¹H NMR (300 MHz, CDCl₃) δ 4.19 (t, 2H), 4.05 (t, 4H), 3.99 (d, 4H), 3.65 (bs, 4H), 2.84-2.39 (m, 28H), 2.29 (t, 4H), 2.28 (t, 4H), 1.92-1.74 (m, 6H), 1.68-1.55 (m, 14H), 1.52-1.24 (m, 44H), 0.92 (t, 6H), 0.88 (t, 12H).

APCI-MS analysis: Calculated C71H136N4O14S2 [M+H]=1334.0, Observed=1334.0.

### Bis(2-ethylbutyl) 9,9′-((4-(2-(4-(2-((3-(bis(2-hydroxy-9-(isopentyloxy)-9-oxononyl)-amino)propyl)disulfaneyl)ethyl)piperazin-1-yl)ethoxy)-4-oxobutyl)azanediyl)bis(8-hydroxynonanoate) (Compound 29)

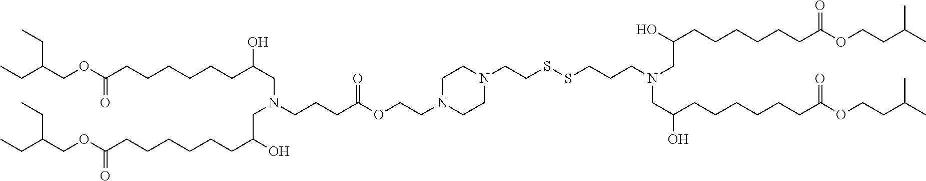 [SMILES: CCC(CC)COC(=O)CCCCCCC(O)CN(CCCC(=O)OCCN1CCN(CCSSCCCN(CC(O)CCCCCCC(=O)OCCC(C)C)CC(O)CCCCCCC(=O)OCCC(C)C)CC1)CC(O)CCCCCCC(=O)OCC(CC)CC]

¹H NMR (300 MHz, CDCl₃) δ 4.19 (t, 2H), 4.08 (t, 4H), 3.98 (d, 4H), 3.67 (m, 4H), 2.85-2.25 (m, 38H), 1.92-1.78 (m, 4H), 1.74-1.26 (m, 56H), 0.91 (d, 12H), 0.88 (t, 12H).

APCI-MS analysis: Calculated C73H140N4O14S2 [M+H]=1362.0, Observed=1362.0.

### Bis(2-ethylbutyl) 9,9′-((3-((2-(4-(2-((4-(bis(9-(2-ethylbutoxy)-2-hydroxy-9-oxononyl)-amino)butanoyl)oxy)ethyl)piperazin-1-yl)ethyl)disulfaneyl)propyl)azanediyl)bis(8-hydroxynonanoate) (Compound 48)

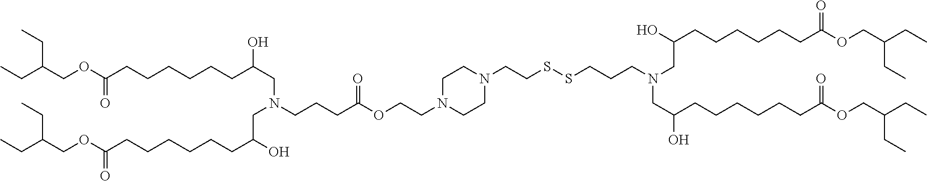 [SMILES: CCC(CC)COC(=O)CCCCCCC(O)CN(CCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCCCC(=O)OCC(CC)CC]

¹H NMR (300 MHz, CDCl₃) δ 4.20 (t, 2H), 3.98 (d, 8H), 3.67 (m, 4H), 2.88-2.35 (m, 30H), 2.29 (t, 8H), 1.96-1.78 (m, 4H), 1.70-1.28 (m, 60H), 0.88 (t, 24H).

APCI-MS analysis: Calculated C75H144N4O14S2 [M+H]=1390.1, Observed=1390.1.

### Bis(2-ethylbutyl) 9,9′-((5-(2-(4-(2-((3-(bis(7-butoxy-2-hydroxy-7-oxoheptyl)amino)-propyl)disulfaneyl)ethyl)piperazin-1-yl)ethoxy)-5-oxopentyl)azanediyl)bis(8-hydroxynonanoate) (Compound 3)

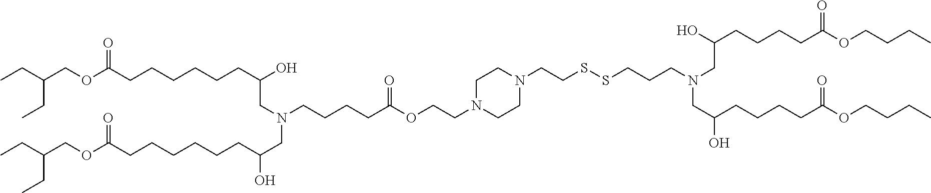 [SMILES: CCCCOC(=O)CCCCC(O)CN(CCCSSCCN1CCN(CCOC(=O)CCCCN(CC(O)CCCCCCC(=O)OCC(CC)CC)CC(O)CCCCCCC(=O)OCC(CC)CC)CC1)CC(O)CCCCC(=O)OCCCC]

¹H NMR (300 MHz, CDCl₃) δ 4.19 (t, 2H), 4.06 (t, 4H), 3.98 (d, 4H), 3.63 (m, 4H), 2.86-2.46 (m, 22H), 2.45-2.23 (m, 16H), 1.91-1.23 (m, 62H), 0.93 (t, 6H), 0.88 (t, 12H).

APCI-MS analysis: Calculated C68H130N4O14S2 [M+H]=1291.9, Observed=1291.9.

### Bis(2-ethylbutyl) 9,9′-((5-(2-(4-(2-((3-(bis(2-hydroxy-7-(isopentyloxy)-7-oxoheptyl)amino)-propyl)disulfaneyl)ethyl)piperazin-1-yl)ethoxy)-5-oxopentyl)azanediyl)bis(8-hydroxynonanoate) (Compound 21)

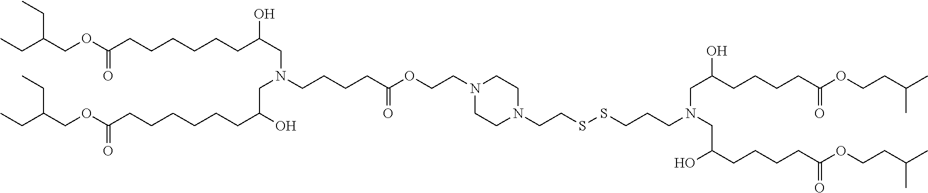 [SMILES: CCC(CC)COC(=O)CCCCCCC(O)CN(CCCCC(=O)OCCN1CCN(CCSSCCCN(CC(O)CCCCC(=O)OCCC(C)C)CC(O)CCCCC(=O)OCCC(C)C)CC1)CC(O)CCCCCCC(=O)OCC(CC)CC]

¹H NMR (300 MHz, CDCl₃) δ 4.19 (t, 2H), 4.08 (t, 4H), 3.98 (d, 4H), 3.61 (m, 4H), 2.86-2.46 (m, 22H), 2.45-2.23 (m, 16H), 1.90-1.24 (m, 58H), 0.91 (d, 12H), 0.88 (t, 12H).

APCI-MS analysis: Calculated C70H134N4O14S2 [M+H]=1319.9, Observed=1320.0.

### Bis(2-ethylbutyl) 9,9′-((5-(2-(4-(2-((3-(bis(7-(2-ethylbutoxy)-2-hydroxy-7-oxoheptyl)amino)-propyl)disulfaneyl)ethyl)piperazin-1-yl)ethoxy)-5-oxopentyl)azanediyl)bis(8-hydroxynonanoate) (Compound 41)

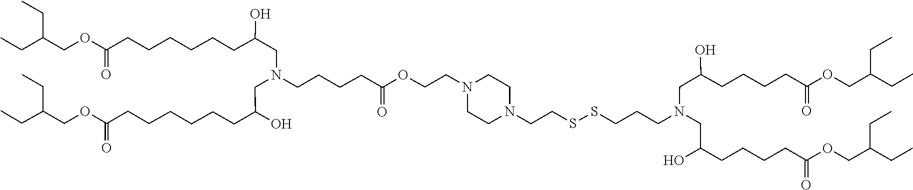 [SMILES: CCC(CC)COC(=O)CCCCCCC(O)CN(CCCCC(=O)OCCN1CCN(CCSSCCCN(CC(O)CCCCC(=O)OCC(CC)CC)CC(O)CCCCC(=O)OCC(CC)CC)CC1)CC(O)CCCCCCC(=O)OCC(CC)CC]

¹H NMR (300 MHz, CDCl₃) δ 4.19 (t, 2H), 3.98 (d, 8H), 3.63 (m, 4H), 2.86-2.46 (m, 22H), 2.45-2.23 (m, 16H), 1.88-1.24 (m, 62H), 0.88 (t, 24H).

APCI-MS analysis: Calculated C72H138N4O14S2 [M+H]=1348.0, Observed=1348.0.

### Bis(2-ethylbutyl) 9,9′-((4-(2-(4-(2-((4-(bis(2-hydroxy-7-isopropoxy-7-oxoheptyl)amino)-butyl)disulfaneyl)ethyl)piperazin-1-yl)ethoxy)-4-oxobutyl)azanediyl)bis(8-hydroxynonanoate) (Compound 16)

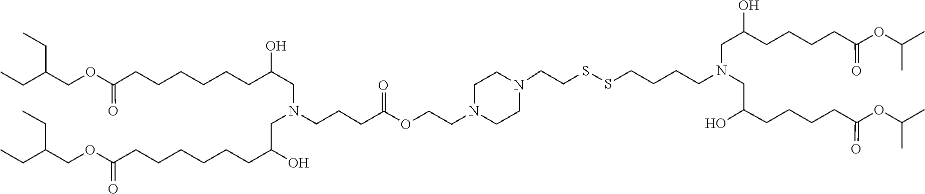 [SMILES: CCC(CC)COC(=O)CCCCCCC(O)CN(CCCC(=O)OCCN1CCN(CCSSCCCCN(CC(O)CCCCC(=O)OC(C)C)CC(O)CCCCC(=O)OC(C)C)CC1)CC(O)CCCCCCC(=O)OCC(CC)CC]

¹H NMR (300 MHz, CDCl₃) δ 4.99 (hept, 2H), 4.19 (t, 2H), 3.98 (d, 4H), 3.66 (m, 4H), 2.85-2.24 (m, 40H), 1.86-1.75 (m, 4H), 1.70-1.26 (m, 46H), 1.22 (d, 12H), 0.88 (t, 12H).

APCI-MS analysis: Calculated C66H126N4O14S2 [M+H]=1263.8, Observed=1263.9.

### Bis(2-ethylbutyl) 9,9′-((4-(2-(4-(2-((4-(bis(7-(2-ethylbutoxy)-2-hydroxy-7-oxoheptyl)amino)-butyl)disulfaneyl)ethyl)piperazin-1-yl)ethoxy)-4-oxobutyl)azanediyl)bis(8-hydroxynonanoate) (Compound 50)

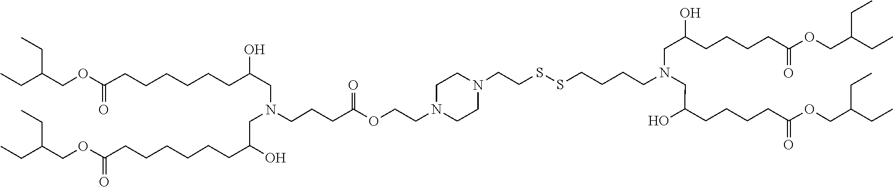 [SMILES: CCC(CC)COC(=O)CCCCCCC(O)CN(CCCC(=O)OCCN1CCN(CCSSCCCCN(CC(O)CCCCC(=O)OCC(CC)CC)CC(O)CCCCC(=O)OCC(CC)CC)CC1)CC(O)CCCCCCC(=O)OCC(CC)CC]

¹H NMR (300 MHz, CDCl₃) δ 4.19 (t, 2H), 3.98 (d, 8H), 3.61 (m, 4H), 2.84-2.26 (m, 36H), 1.83-1.28 (m, 64H), 0.88 (t, 24H).

APCI-MS analysis: Calculated C72H138N4O14S2 [M+H]=1348.0, Observed=1348.0.

### Dibutyl 9,9′-((4-((2-(4-(2-((4-(bis(9-(2-ethylbutoxy)-2-hydroxy-9-oxononyl)amino)-butanoyl)oxy)ethyl)piperazin-1-yl)ethyl)disulfaneyl)butyl)azanediyl)bis(8-hydroxynonanoate) (Compound 11)

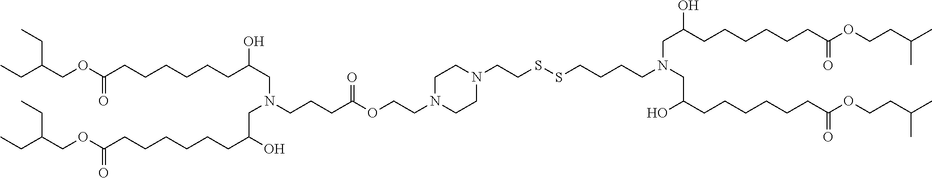 [SMILES: CCC(CC)COC(=O)CCCCCCC(O)CN(CCCC(=O)OCCN1CCN(CCSSCCCCN(CC(O)CCCCCCC(=O)OCCC(C)C)CC(O)CCCCCCC(=O)OCCC(C)C)CC1)CC(O)CCCCCCC(=O)OCC(CC)CC]

¹H NMR (300 MHz, CDCl₃) δ 4.19 (t, 2H), 4.06 (t, 4H), 3.98 (d, 4H), 3.62 (m, 4H), 2.84-2.25 (m, 36H), 1.83-1.28 (m, 70H), 0.92 (t, 6H), 0.88 (t, 12H).

APCI-MS analysis: Calculated C72H138N4O14S2 [M+H]=1348.0, Observed=1348.0.

### Bis(2-ethylbutyl) 9,9′-((4-(2-(4-(2-((4-(bis(2-hydroxy-9-(isopentyloxy)-9-oxononyl)amino)butyl)disulfaneyl)ethyl)piperazin-1-yl)ethoxy)-4-oxobutyl)azanediyl)bis(8-hydroxynonanoate) (Compound 38)

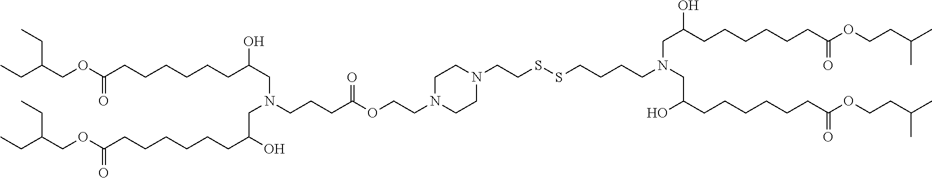 [SMILES: CCC(CC)COC(=O)CCCCCCC(O)CN(CCCC(=O)OCCN1CCN(CCSSCCCCN(CC(O)CCCCCCC(=O)OCCC(C)C)CC(O)CCCCCCC(=O)OCCC(C)C)CC1)CC(O)CCCCCCC(=O)OCC(CC)CC]

¹H NMR (300 MHz, CDCl₃) δ 4.19 (t, 2H), 4.08 (t, 4H), 3.98 (d, 4H), 3.61 (m, 4H), 2.85-2.25 (m, 38H), 1.85-1.24 (m, 62H), 0.91 (d, 12H), 0.88 (t, 12H).

APCI-MS analysis: Calculated C74H142N4O14S2 [M+H]=1376.0, Observed=1376.1.

### Bis(2-ethylbutyl) 9,9′-((4-((2-(4-(2-((4-(bis(9-(2-ethylbutoxy)-2-hydroxy-9-oxononyl)amino)butanoyl)oxy)ethyl)piperazin-1-yl)ethyl)disulfaneyl)butyl)azanediyl)bis(8-hydroxynonanoate) (Compound 60)

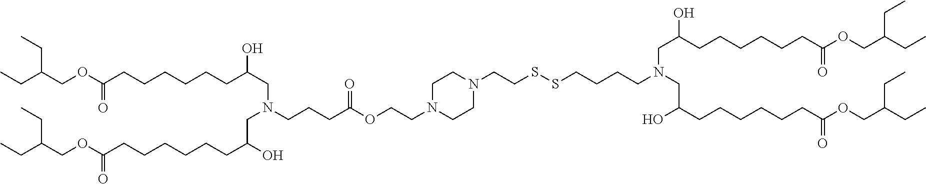 [SMILES: CCC(CC)COC(=O)CCCCCCC(O)CN(CCCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCCCC(=O)OCC(CC)CC]

¹H NMR (300 MHz, CDCl₃) δ 4.19 (t, 2H), 3.98 (d, 8H), 3.62 (m, 4H), 2.85-2.25 (m, 36H), 1.85-1.24 (m, 72H), 0.88 (t, 24H).

APCI-MS analysis: Calculated C76H146N4O14S2 [M+H]=1404.1, Observed=1404.0.

### Bis(2-ethylbutyl) 9,9′-((5-(2-(4-(2-((4-(bis(7-butoxy-2-hydroxy-7-oxoheptyl)amino)butyl)-disulfaneyl)ethyl)piperazin-1-yl)ethoxy)-5-oxopentyl)azanediyl)bis(8-hydroxynonanoate) (Compound 9)

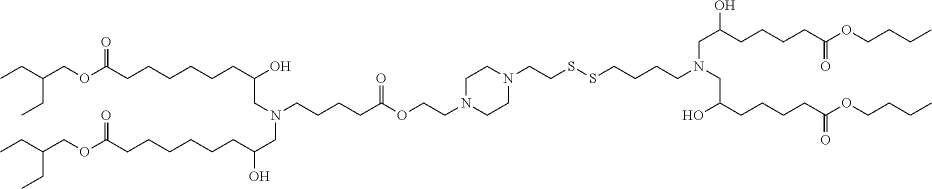 [SMILES: CCCCOC(=O)CCCCC(O)CN(CCCCSSCCN1CCN(CCOC(=O)CCCCN(CC(O)CCCCCCC(=O)OCC(CC)CC)CC(O)CCCCCCC(=O)OCC(CC)CC)CC1)CC(O)CCCCC(=O)OCCCC]

¹H NMR (300 MHz, CDCl₃) δ 4.19 (t, 2H), 4.06 (t, 4H), 3.98 (d, 4H), 3.61 (m, 4H), 2.86-2.46 (m, 22H), 2.45-2.23 (m, 16H), 1.80-1.23 (m, 58H), 0.92 (t, 6H), 0.88 (t, 12H).

APCI-MS analysis: Calculated C69H132N4O14S2 [M+H]=1305.9, Observed=1306.0.

### Bis(2-ethylbutyl) 9,9′-((5-(2-(4-(2-((4-(bis(7-(2-ethylbutoxy)-2-hydroxy-7-oxoheptyl)amino)-butyl)disulfaneyl)ethyl)piperazin-1-yl)ethoxy)-5-oxopentyl)azanediyl)bis(8-hydroxynonanoate) (Compound 51)

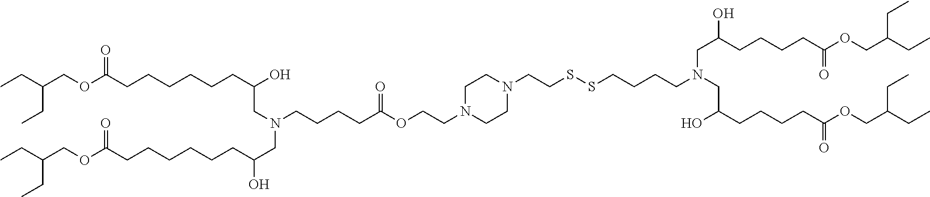 [SMILES: CCC(CC)COC(=O)CCCCCCC(O)CN(CCCCC(=O)OCCN1CCN(CCSSCCCCN(CC(O)CCCCC(=O)OCC(CC)CC)CC(O)CCCCC(=O)OCC(CC)CC)CC1)CC(O)CCCCCCC(=O)OCC(CC)CC]

¹H NMR (300 MHz, CDCl₃) δ 4.19 (t, 2H), 3.98 (d, 8H), 3.61 (m, 4H), 2.86-2.46 (m, 22H), 2.45-2.23 (m, 16H), 1.75-1.24 (m, 60H), 0.88 (t, 24H).

APCI-MS analysis: Calculated C73H140N4O14S2 [M+H]=1362.0, Observed=1362.0.

### Dibutyl 9,9′-((3-((2-(4-(2-((5-(bis(9-(2-ethylbutoxy)-2-hydroxy-9-oxononyl)amino)-pentanoyl)oxy)ethyl)piperazin-1-yl)ethyl)disulfaneyl)propyl)azanediyl)bis(8-hydroxynonanoate) (Compound 12)

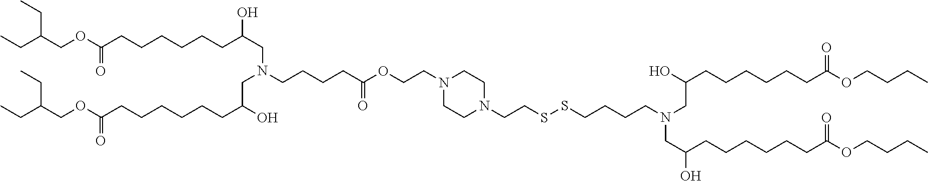 [SMILES: CCCCOC(=O)CCCCCCC(O)CN(CCCCSSCCN1CCN(CCOC(=O)CCCCN(CC(O)CCCCCCC(=O)OCC(CC)CC)CC(O)CCCCCCC(=O)OCC(CC)CC)CC1)CC(O)CCCCCCC(=O)OCCCC]

¹H NMR (300 MHz, CDCl₃) δ 4.19 (t, 2H), 4.06 (t, 4H), 3.98 (d, 4H), 3.63 (m, 4H), 2.86-2.23 (m, 38H), 1.70-1.23 (m, 62H), 0.92 (t, 6H), 0.88 (t, 12H).

APCI-MS analysis: Calculated C71H136N4O14S2 [M+H]=1362.0, Observed=1361.5.

### Bis(2-ethylbutyl) 9,9′-((5-(2-(4-(2-((4-(bis(2-hydroxy-7-(isopentyloxy)-7-oxoheptyl)amino)-butyl)disulfaneyl)ethyl)piperazin-1-yl)ethoxy)-5-oxopentyl)azanediyl)bis(8-hydroxynonanoate) (Compound 31)

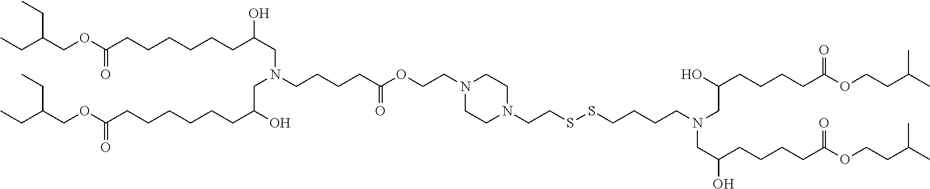 [SMILES: CCC(CC)COC(=O)CCCCCCC(O)CN(CCCCC(=O)OCCN1CCN(CCSSCCCCN(CC(O)CCCCC(=O)OCCC(C)C)CC(O)CCCCC(=O)OCCC(C)C)CC1)CC(O)CCCCCCC(=O)OCC(CC)CC]

¹H NMR (300 MHz, CDCl₃) δ 4.19 (t, 2H), 4.08 (t, 4H), 3.98 (d, 4H), 3.61 (m, 4H), 2.86-2.46 (m, 22H), 2.45-2.23 (m, 16H), 1.80-1.24 (m, 60H), 0.91 (d, 12H), 0.88 (t, 12H).

APCI-MS analysis: Calculated C71H136N4O14S2 [M+H]=1334.0, Observed=1333.9.

### Dibutyl 9,9′-((3-((2-(4-(2-((5-(bis(9-(2-ethylbutoxy)-2-hydroxy-9-oxononyl)amino)-pentanoyl)oxy)ethyl)piperazin-1-yl)ethyl)disulfaneyl)propyl)azanediyl)bis(8-hydroxynonanoate) (Compound 6)

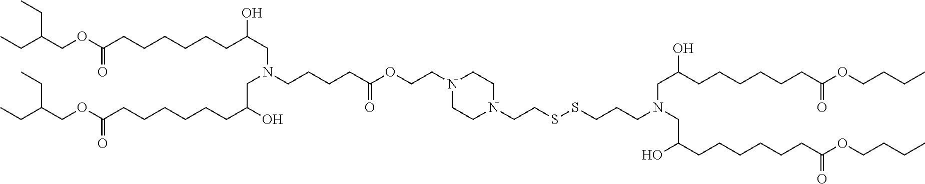 [SMILES: CCCCOC(=O)CCCCCCC(O)CN(CCCSSCCN1CCN(CCOC(=O)CCCCN(CC(O)CCCCCCC(=O)OCC(CC)CC)CC(O)CCCCCCC(=O)OCC(CC)CC)CC1)CC(O)CCCCCCC(=O)OCCCC]

¹H NMR (300 MHz, CDCl₃) δ 4.19 (t, 2H), 4.06 (t, 4H), 3.98 (d, 4H), 3.61 (m, 4H), 2.86-2.46 (m, 22H), 2.45-2.23 (m, 16H), 1.90-1.23 (m, 68H), 0.92 (t, 6H), 0.88 (d, 12H).

APCI-MS analysis: Calculated C72H138N4O14S2 [M+H]=1348.0, Observed=1346.9.

### Bis(2-ethylbutyl) 9,9′-((4-(2-(4-(2-((3-(bis(7-butoxy-2-hydroxy-7-oxoheptyl)amino)-propyl)disulfaneyl)ethyl)piperazin-1-yl)ethoxy)-4-oxobutyl)azanediyl)bis(8-hydroxynonanoate) (Compound 2)

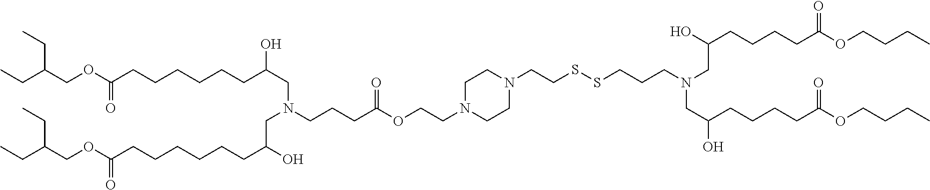 [SMILES: CCCCOC(=O)CCCCC(O)CN(CCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCC(=O)OCCCC]

¹H NMR (300 MHz, CDCl₃) δ 4.20 (t, 2H), 4.06 (t, 4H), 3.98 (d, 4H), 3.64 (m, 4H), 2.84-2.24 (m, 40H), 1.92-1.26 (m, 56H), 0.92 (d, 6H), 0.88 (t, 12H).

APCI-MS analysis: Calculated C67H128N4O14S2 [M+H]=1277.8, Observed=1277.9.

### Bis(2-ethylbutyl) 9,9′-((4-(2-(4-(2-((4-(bis(7-butoxy-2-hydroxy-7-oxoheptyl)amino)-butyl)disulfaneyl)ethyl)piperazin-1-yl)ethoxy)-4-oxobutyl)azanediyl)bis(8-hydroxynonanoate) (Compound 8)

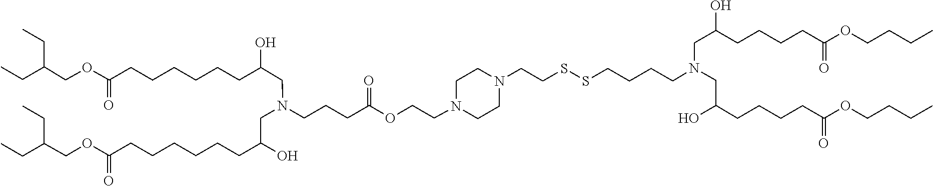 [SMILES: CCCCOC(=O)CCCCC(O)CN(CCCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCC(=O)OCCCC]

¹H NMR (300 MHz, CDCl₃) δ 4.19 (t, 2H), 4.06 (t, 4H), 3.98 (d, 4H), 3.61 (m, 4H), 2.84-2.26 (m, 34H), 1.85-1.28 (m, 60H), 0.92 (t, 6H), 0.88 (t, 12H).

APCI-MS analysis: Calculated C68H130N4O14S2 [M+H]=1291.9, Observed=1291.9.

### Bis(2-ethylbutyl) 9,9′-((5-(2-(4-(2-((4-(bis(2-hydroxy-7-isopropoxy-7-oxoheptyl)amino)-butyl)disulfaneyl)ethyl)piperazin-1-yl)ethoxy)-5-oxopentyl)azanediyl)bis(8-hydroxynonanoate) (Compound 17)

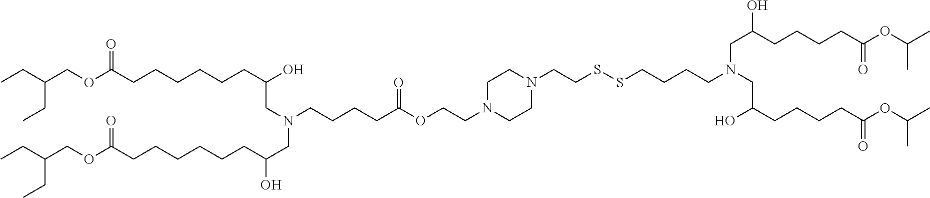 [SMILES: CCC(CC)COC(=O)CCCCCCC(O)CN(CCCCC(=O)OCCN1CCN(CCSSCCCCN(CC(O)CCCCC(=O)OC(C)C)CC(O)CCCCC(=O)OC(C)C)CC1)CC(O)CCCCCCC(=O)OCC(CC)CC]

¹H NMR (300 MHz, CDCl₃) δ 4.99 (hept, 2H), 4.19 (t, 2H), 3.98 (d, 4H), 3.62 (m, 4H), 2.85-2.23 (m, 38H), 1.83-1.25 (m, 50H), 1.22 (d, 12H), 0.88 (t, 12H).

APCI-MS analysis: Calculated C67H128N4O14S2 [M+H]=1277.9, Observed=1277.9.

### Diisopentyl 9,9′-((3-((2-(4-(2-((4-(bis(2-hydroxy-6-oxo-6-(pentan-3-yloxy)hexyl)amino)-butanoyl)oxy)ethyl)piperazin-1-yl)ethyl)disulfaneyl)propyl)azanediyl)bis(8-hydroxynonanoate) (Compound 27)

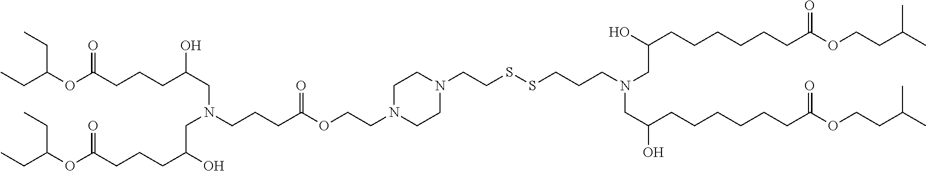 [SMILES: CCC(CC)OC(=O)CCCC(O)CN(CCCC(=O)OCCN1CCN(CCSSCCCN(CC(O)CCCCCCC(=O)OCCC(C)C)CC(O)CCCCCCC(=O)OCCC(C)C)CC1)CC(O)CCCC(=O)OC(CC)CC]

¹H NMR (300 MHz, CDCl₃) δ 4.77 (pent, 2H), 4.19 (t, 2H), 4.08 (t, 4H), 3.65 (m, 4H), 2.85-2.25 (m, 38H), 1.90-1.24 (m, 46H), 0.91 (d, 12H), 0.86 (t, 12H).

APCI-MS analysis: Calculated C65H124N4O14S2 [M+H]=1249.8, Observed=1249.8.

### Diisopentyl 9,9′-((4-((2-(4-(2-((4-(bis(2-hydroxy-6-oxo-6-(pentan-3-yloxy)hexyl)amino)-butanoyl)oxy)ethyl)piperazin-1-yl)ethyl)disulfaneyl)butyl)azanediyl)bis(8-hydroxynonanoate) (Compound 36)

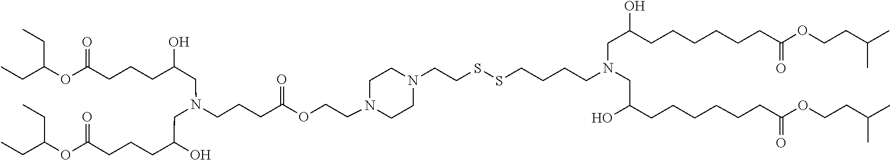 [SMILES: CCC(CC)OC(=O)CCCC(O)CN(CCCC(=O)OCCN1CCN(CCSSCCCCN(CC(O)CCCCCCC(=O)OCCC(C)C)CC(O)CCCCCCC(=O)OCCC(C)C)CC1)CC(O)CCCC(=O)OC(CC)CC]

¹H NMR (300 MHz, CDCl₃) δ 4.77 (pent, 2H), 4.19 (t, 2H), 4.08 (t, 4H), 3.62 (m, 4H), 2.85-2.25 (m, 36H), 1.86-1.24 (m, 54H), 0.91 (d, 12H), 0.86 (t, 12H).

APCI-MS analysis: Calculated C66H126N4O14S2 [M+H]=1263.8, Observed=1263.9.

### Diisopentyl 9,9′-((3-((2-(4-(2-((4-(bis(2-hydroxy-7-isopropoxy-7-oxoheptyl)amino)-butanoyl)oxy)ethyl)piperazin-1-yl)ethyl)disulfaneyl)propyl)azanediyl)bis(8-hydroxynonanoate) (Compound 24)

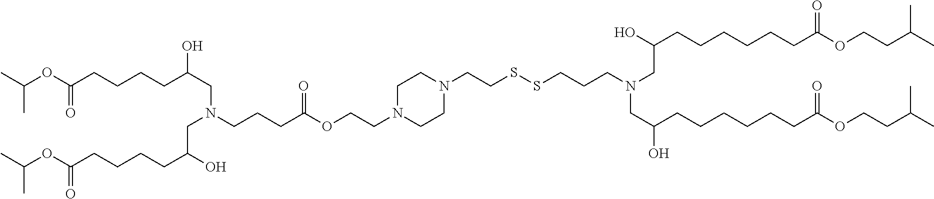 [SMILES: CC(C)CCOC(=O)CCCCCCC(O)CN(CCCSSCCN1CCN(CCOC(=O)CCCN(CC(O)CCCCC(=O)OC(C)C)CC(O)CCCCC(=O)OC(C)C)CC1)CC(O)CCCCCCC(=O)OCCC(C)C]

¹H NMR (300 MHz, CDCl₃) δ 4.99 (hept, 2H), 4.19 (t, 2H), 4.08 (t, 4H), 3.65 (m, 4H), 2.85-2.24 (m, 40H), 1.92-1.78 (m, 4H), 1.72-1.26 (m, 36H), 1.23 (d, 12H), 0.91 (t, 12H).

APCI-MS analysis: Calculated C63H120N4O14S2 [M+H]=1221.7, Observed=1221.8.

### Diisopentyl 9,9′-((4-((2-(4-(2-((4-(bis(2-hydroxy-7-isopropoxy-7-oxoheptyl)amino)-butanoyl)oxy)ethyl)piperazin-1-yl)ethyl)disulfaneyl)butyl)azanediyl)bis(8-hydroxynonanoate) (Compound 34)

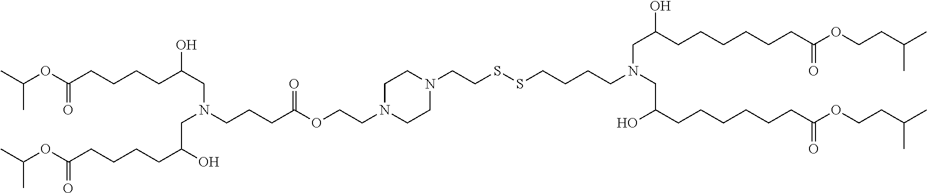 [SMILES: CC(C)CCOC(=O)CCCCCCC(O)CN(CCCCSSCCN1CCN(CCOC(=O)CCCN(CC(O)CCCCC(=O)OC(C)C)CC(O)CCCCC(=O)OC(C)C)CC1)CC(O)CCCCCCC(=O)OCCC(C)C]

¹H NMR (300 MHz, CDCl₃) δ 4.99 (hept, 2H), 4.19 (t, 2H), 4.08 (t, 4H), 3.61 (m, 4H), 2.85-2.24 (m, 36H), 1.86-1.27 (m, 46H), 1.22 (d, 12H), 0.91 (t, 12H).

APCI-MS analysis: Calculated C64H122N4O14S2 [M+H]=1235.8, Observed=1235.9.

### Diisopentyl 9,9′-((3-((2-(4-(2-((5-(bis(2-hydroxy-7-isopropoxy-7-oxoheptyl)amino)-pentanoyl)oxy)ethyl)piperazin-1-yl)ethyl)disulfaneyl)propyl)azanediyl)bis(8-hydroxynonanoate) (Compound 25)

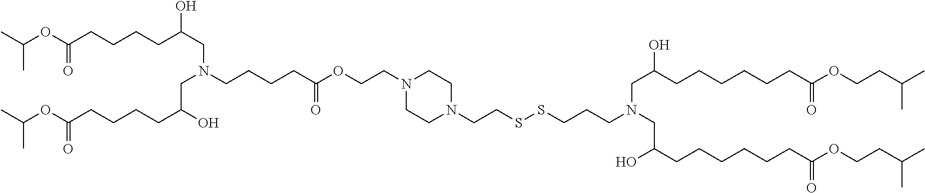 [SMILES: CC(C)CCOC(=O)CCCCCCC(O)CN(CCCSSCCN1CCN(CCOC(=O)CCCCN(CC(O)CCCCC(=O)OC(C)C)CC(O)CCCCC(=O)OC(C)C)CC1)CC(O)CCCCCCC(=O)OCCC(C)C]

¹H NMR (300 MHz, CDCl₃) δ 4.99 (hept, 2H), 4.19 (t, 2H), 4.08 (t, 8H), 3.63 (m, 4H), 2.85-2.24 (m, 40H), 1.95-1.27 (m, 40H), 1.22 (d, 12H), 0.91 (t, 12H).

APCI-MS analysis: Calculated C64H122N4O14S2 [M+H]=1235.8, Observed=1235.9.

### Diisopentyl 9,9′-((3-((2-(4-(2-((4-(bis(2-hydroxy-7-(isopentyloxy)-7-oxoheptyl)amino)-butanoyl)oxy)ethyl)piperazin-1-yl)ethyl)disulfaneyl)propyl)azanediyl)bis(8-hydroxynonanoate) (Compound 26)

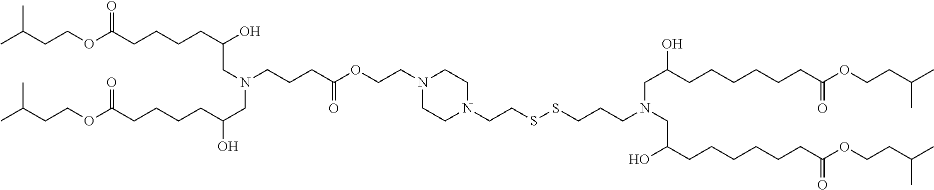 [SMILES: CC(C)CCOC(=O)CCCCCCC(O)CN(CCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCCC(C)C]

¹H NMR (300 MHz, CDCl₃) δ 4.19 (t, 2H), 4.08 (t, 8H), 3.63 (m, 4H), 2.84-2.46 (m, 22H), 2.43-2.23 (m, 16H), 1.91-1.29 (m, 48H), 0.91 (d, 24H).

APCI-MS analysis: Calculated C67H128N4O14S2 [M+H]=1277.9, Observed=1277.9.

### Diisopentyl 9,9′-((4-((2-(4-(2-((4-(bis(2-hydroxy-7-(isopentyloxy)-7-oxoheptyl)amino)-butanoyl)oxy)ethyl)piperazin-1-yl)ethyl)disulfaneyl)butyl)azanediyl)bis(8-hydroxynonanoate) (Compound 35)

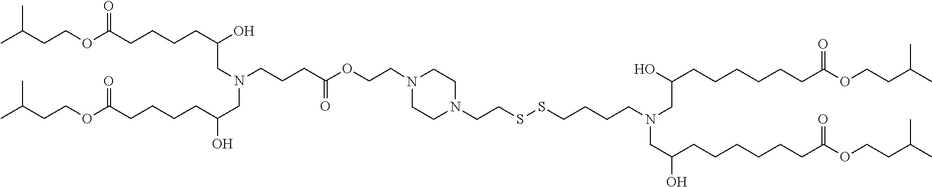 [SMILES: CC(C)CCOC(=O)CCCCCCC(O)CN(CCCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCCC(C)C]

¹HNMR (300 MHz, CDCl₃) δ 4.19 (t, 2H), 4.08 (t, 8H), 3.61 (m, 4H), 2.85-2.21 (m, 38H), 1.85-1.25 (m, 50H), 0.91 (d, 24H).

APCI-MS analysis: Calculated C68H130N4O14S2 [M+H]=1291.9, Observed=1291.8.

### Diisopentyl 9,9′-((3-((2-(4-(2-((4-(bis(7-(2-ethylbutoxy)-2-hydroxy-7-oxoheptyl)amino)butanoyl)oxy)ethyl)piperazin-1-yl)ethyl)disulfaneyl)propyl)-azanediyl)bis(8-hydroxynonanoate) (Compound 28)

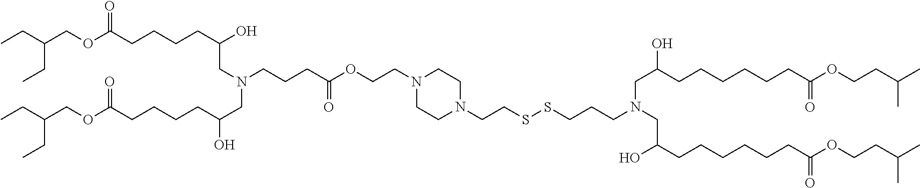 [SMILES: CCC(CC)COC(=O)CCCCC(O)CN(CCCC(=O)OCCN1CCN(CCSSCCCN(CC(O)CCCCCCC(=O)OCCC(C)C)CC(O)CCCCCCC(=O)OCCC(C)C)CC1)CC(O)CCCCC(=O)OCC(CC)CC]

¹H NMR (300 MHz, CDCl₃) δ 4.20 (t, 2H), 4.08 (t, 4H), 3.98 (d, 4H), 3.68 (m, 4H), 2.88-2.45 (m, 22H), 2.42-2.24 (m, 16H), 1.95-1.26 (m, 52H), 0.95-0.86 (m, 24H).

APCI-MS analysis: Calculated C69H132N4O14S2 [M+H]=1305.9, Observed=1305.8.

### Diisopentyl 9,9′-((3-((2-(4-(2-((5-(bis(7-(2-ethylbutoxy)-2-hydroxy-7-oxoheptyl)amino)-pentanoyl)oxy)ethyl)piperazin-1-yl)ethyl)disulfaneyl)propyl)azanediyl)bis(8-hydroxynonanoate)) (Compound 30)

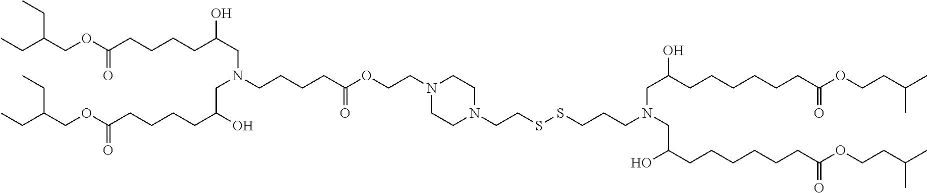 [SMILES: CCC(CC)COC(=O)CCCCC(O)CN(CCCCC(=O)OCCN1CCN(CCSSCCCN(CC(O)CCCCCCC(=O)OCCC(C)C)CC(O)CCCCCCC(=O)OCCC(C)C)CC1)CC(O)CCCCC(=O)OCC(CC)CC]

¹H NMR (300 MHz, CDCl₃) δ 4.19 (t, 2H), 4.08 (t, 4H), 3.98 (d, 4H), 3.63 (m, 4H), 2.85-2.25 (m, 40H), 1.95-1.24 (m, 52H), 0.91 (t, 12H), 0.88 (t, 12H).

APCI-MS analysis: Calculated C70H134N4O14S2 [M+H]=1319.9, Observed=1320.0.

### Diisopentyl 9,9′-((4-((2-(4-(2-((4-(bis(7-(2-ethylbutoxy)-2-hydroxy-7-oxoheptyl)amino)butanoyl)oxy)ethyl)piperazin-1-yl)ethyl)disulfaneyl)butyl)-azanediyl)bis(8-hydroxynonanoate) (Compound 37)

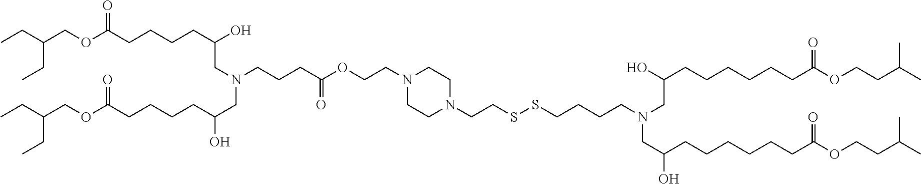 [SMILES: CCC(CC)COC(=O)CCCCC(O)CN(CCCC(=O)OCCN1CCN(CCSSCCCCN(CC(O)CCCCCCC(=O)OCCC(C)C)CC(O)CCCCCCC(=O)OCCC(C)C)CC1)CC(O)CCCCC(=O)OCC(CC)CC]

¹H NMR (300 MHz, CDCl₃) δ 4.19 (t, 2H), 4.06 (t, 4H), 3.98 (d, 4H), 3.62 (m, 4H), 2.84-2.28 (m, 38H), 1.95-1.22 (m, 54H), 0.95-0.86 (m, 24H).

APCI-MS analysis: Calculated C70H134N4O14S2 [M+H]=1319.9, Observed=1319.8.

### Dibutyl 9,9′-((4-(2-(4-(2-((3-(bis(2-hydroxy-9-(isopentyloxy)-9-oxononyl)amino)propyl)disulfaneyl)ethyl)piperazin-1-yl)ethoxy)-4-oxobutyl)azanediyl)bis(8-hydroxynonanoate) (Compound 22)

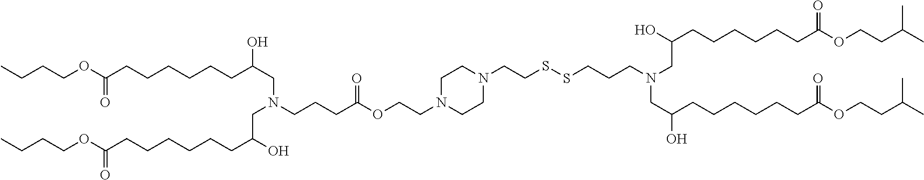 [SMILES: CCCCOC(=O)CCCCCCC(O)CN(CCCC(=O)OCCN1CCN(CCSSCCCN(CC(O)CCCCCCC(=O)OCCC(C)C)CC(O)CCCCCCC(=O)OCCC(C)C)CC1)CC(O)CCCCCCC(=O)OCCCC]

¹H NMR (300 MHz, CDCl₃) δ 4.20 (t, 2H), 4.06 (m, 8H), 3.64 (m, 4H), 2.88-2.45 (m, 22H), 2.42-2.24 (m, 16H), 1.95-1.26 (m, 58H), 0.95-0.88 (m, 18H).

APCI-MS analysis: Calculated C69H132N4O14S2 [M+H]=1305.9, Observed=1305.8.

### Dibutyl 9,9′-((4-(2-(4-(2-((4-(bis(2-hydroxy-9-(isopentyloxy)-9-oxononyl)amino)butyl)disulfaneyl)ethyl)piperazin-1-yl)ethoxy)-4-oxobutyl)azanediyl)bis(8-hydroxynonanoate) (Compound 32)

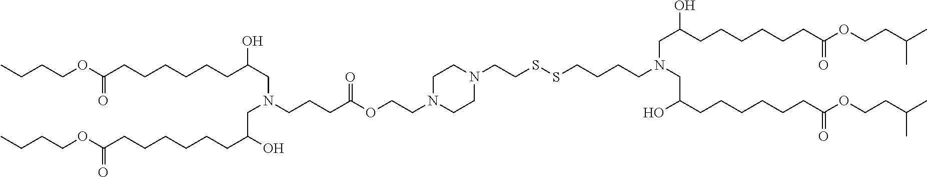 [SMILES: CCCCOC(=O)CCCCCCC(O)CN(CCCC(=O)OCCN1CCN(CCSSCCCCN(CC(O)CCCCCCC(=O)OCCC(C)C)CC(O)CCCCCCC(=O)OCCC(C)C)CC1)CC(O)CCCCCCC(=O)OCCCC]

¹H NMR (300 MHz, CDCl₃) δ 4.20 (t, 2H), 4.06 (m, 8H), 3.61 (m, 4H), 2.82-2.46 (m, 22H), 2.43-2.25 (m, 16H), 1.90-1.22 (m, 60H), 0.93-0.85 (m, 18H).

APCI-MS analysis: Calculated C70H134N4O14S2 [M+H]=1319.9, Observed=1319.8.

### Dibutyl 9,9′-((5-(2-(4-(2-((4-(bis(2-hydroxy-9-(isopentyloxy)-9-oxononyl)-amino)butyl)disulfaneyl)ethyl)piperazin-1-yl)ethoxy)-5-oxopentyl)azanediyl)-bis(8-hydroxynonanoate) (Compound 33)

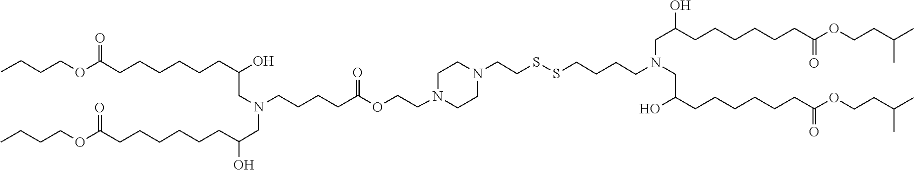 [SMILES: CCCCOC(=O)CCCCCCC(O)CN(CCCCC(=O)OCCN1CCN(CCSSCCCCN(CC(O)CCCCCCC(=O)OCCC(C)C)CC(O)CCCCCCC(=O)OCCC(C)C)CC1)CC(O)CCCCCCC(=O)OCCCC]

¹H NMR (300 MHz, CDCl₃) δ 4.19 (t, 2H), 4.08 (t, 4H), 4.06 (t, 4H), 3.61 (m, 4H), 2.85-2.25 (m, 38H), 1.80-1.25 (m, 62H), 0.92 (t, 6H), 0.91 (d, 12H).

APCI-MS analysis: Calculated C71H136N4O14S2 [M+H]=1334.0, Observed=1334.0.

### Dibutyl 9,9′-((5-(2-(4-(2-((3-(bis(2-hydroxy-9-(isopentyloxy)-9-oxononyl)amino)-propyl)disulfaneyl)ethyl)piperazin-1-yl)ethoxy)-5-oxopentyl)azanediyl)bis(8-hydroxynonanoate) (Compound 23)

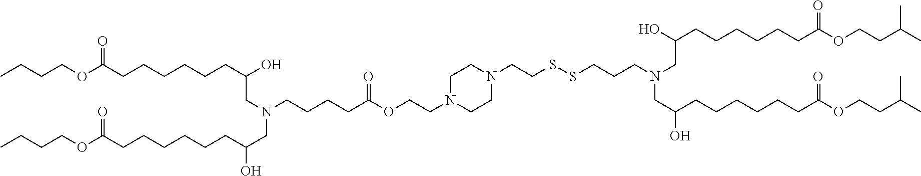 [SMILES: CCCCOC(=O)CCCCCCC(O)CN(CCCCC(=O)OCCN1CCN(CCSSCCCN(CC(O)CCCCCCC(=O)OCCC(C)C)CC(O)CCCCCCC(=O)OCCC(C)C)CC1)CC(O)CCCCCCC(=O)OCCCC]

¹H NMR (300 MHz, CDCl₃) δ 4.19 (t, 2H), 4.08 (t, 4H), 4.06 (t, 4H), 3.61 (m, 4H), 2.86-2.46 (m, 22H), 2.45-2.25 (m, 16H), 1.91-1.25 (m, 60H), 0.92 (t, 6H), 0.91 (d, 12H).

APCI-MS analysis: Calculated C70H134N4O14S2 [M+H]=1319.9, Observed=1319.9.

### Bis(2-ethylbutyl) 9,9′-((3-((2-(4-(2-((4-(bis(2-hydroxy-6-oxo-6-(pentan-3-yloxy)hexyl)-amino)butanoyl)oxy)ethyl)piperazin-1-yl)ethyl)disulfaneyl)propyl)azanediyl)bis(8-hydroxynonanoate) (Compound 46)

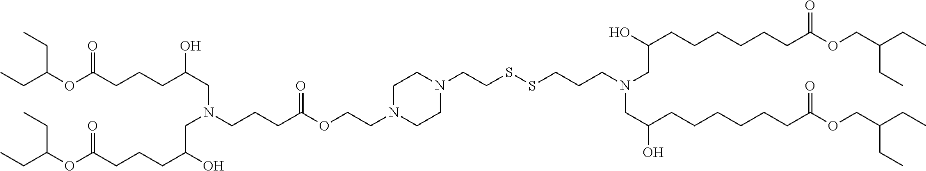 [SMILES: CCC(CC)COC(=O)CCCCCCC(O)CN(CCCSSCCN1CCN(CCOC(=O)CCCN(CC(O)CCCC(=O)OC(CC)CC)CC(O)CCCC(=O)OC(CC)CC)CC1)CC(O)CCCCCCC(=O)OCC(CC)CC]

¹H NMR (300 MHz, CDCl₃) δ 4.75 (pent, 2H), 4.19 (t, 2H), 3.98 (d, 4H), 3.64 (m, 4H), 2.85-2.25 (m, 40H), 1.90-1.24 (m, 52H), 0.88 (d, 12H), 0.86 (t, 12H).

APCI-MS analysis: Calculated C67H128N4O14S2 [M+H]=1277.9, Observed=1277.9.

### Bis(2-ethylbutyl) 9,9′-((4-((2-(4-(2-((4-(bis(2-hydroxy-6-oxo-6-(pentan-3-yloxy)hexyl)-amino)butanoyl)oxy)ethyl)piperazin-1-yl)ethyl)disulfaneyl)butyl)azanediyl)bis(8-hydroxynonanoate) (Compound 59)

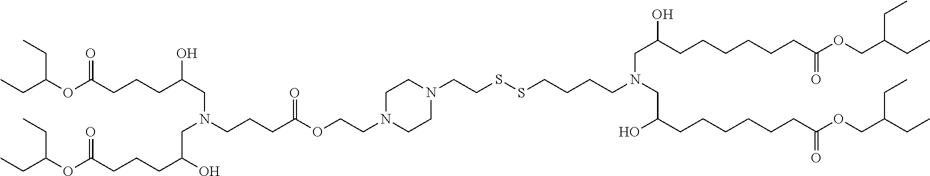 [SMILES: CCC(CC)COC(=O)CCCCCCC(O)CN(CCCCSSCCN1CCN(CCOC(=O)CCCN(CC(O)CCCC(=O)OC(CC)CC)CC(O)CCCC(=O)OC(CC)CC)CC1)CC(O)CCCCCCC(=O)OCC(CC)CC]

¹H NMR (300 MHz, CDCl₃) δ 4.75 (pent, 2H), 4.19 (t, 2H), 3.98 (d, 4H), 3.61 (m, 4H), 2.85-2.25 (m, 38H), 1.86-1.24 (m, 52H), 0.88 (t, 12H), 0.86 (t, 12H).

APCI-MS analysis: Calculated C68H130N4O14S2 [M+H]=1291.9, Observed=1291.9.

### Bis(2-ethylbutyl) 9,9′-((3-((2-(4-(2-((4-(bis(2-hydroxy-7-isopropoxy-7-oxoheptyl)-amino)butanoyl)oxy)ethyl)piperazin-1-yl)ethyl)disulfaneyl)propyl)azanediyl)bis(8-hydroxynonanoate) (Compound 44)

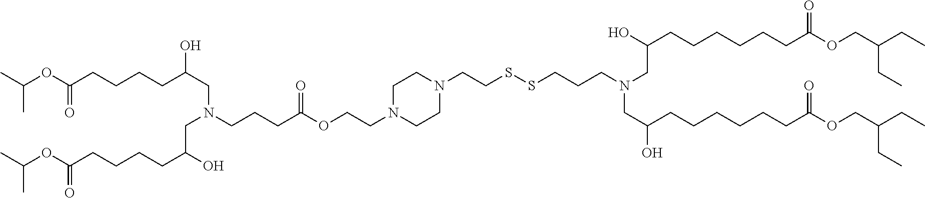 [SMILES: CCC(CC)COC(=O)CCCCCCC(O)CN(CCCSSCCN1CCN(CCOC(=O)CCCN(CC(O)CCCCC(=O)OC(C)C)CC(O)CCCCC(=O)OC(C)C)CC1)CC(O)CCCCCCC(=O)OCC(CC)CC]

¹H NMR (300 MHz, CDCl₃) δ 4.99 (hept, 2H), 4.19 (t, 2H), 3.98 (t, 4H), 3.64 (m, 4H), 2.85-2.24 (m, 36H), 1.90-1.78 (m, 4H), 1.68-1.26 (m, 44H), 1.22 (d, 12H), 0.88 (t, 12H).

APCI-MS analysis: Calculated C65H124N4O14S2 [M+H]=1249.8, Observed=1249.9.

### Bis(2-ethylbutyl) 9,9′-((4-((2-(4-(2-((5-(bis(2-hydroxy-7-isopropoxy-7-oxoheptyl)amino)-pentanoyl)oxy)ethyl)piperazin-1-yl)ethyl)disulfaneyl)butyl)azanediyl)bis(8-hydroxynonanoate) (Compound 55)

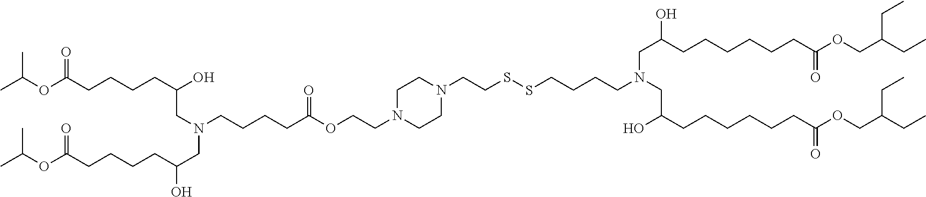 [SMILES: CCC(CC)COC(=O)CCCCCCC(O)CN(CCCCSSCCN1CCN(CCOC(=O)CCCCN(CC(O)CCCCC(=O)OC(C)C)CC(O)CCCCC(=O)OC(C)C)CC1)CC(O)CCCCCCC(=O)OCC(CC)CC]

¹H NMR (300 MHz, CDCl₃) δ 4.99 (hept, 2H), 4.19 (t, 2H), 3.98 (d, 4H), 3.64 (m, 4H), 2.85-2.24 (m, 40H), 1.78-1.29 (m, 48H), 1.21 (d, 12H), 0.88 (t, 12H).

APCI-MS analysis: Calculated C67H128N4O14S2 [M+H]=1277.9, Observed=1277.0.

### Bis(2-ethylbutyl) 9,9′-((4-((2-(4-(2-((4-(bis(2-hydroxy-7-isopropoxy-7-oxoheptyl)amino)-butanoyl)oxy)ethyl)piperazin-1-yl)ethyl)disulfaneyl)butyl)azanediyl)bis(8-hydroxynonanoate) (Compound 54)

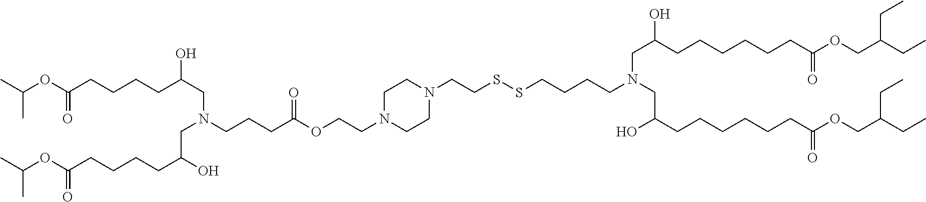 [SMILES: CCC(CC)COC(=O)CCCCCCC(O)CN(CCCCSSCCN1CCN(CCOC(=O)CCCN(CC(O)CCCCC(=O)OC(C)C)CC(O)CCCCC(=O)OC(C)C)CC1)CC(O)CCCCCCC(=O)OCC(CC)CC]

¹H NMR (300 MHz, CDCl₃) δ 4.99 (hept, 2H), 4.19 (t, 2H), 3.98 (d, 4H), 3.61 (m, 4H), 2.85-2.24 (m, 38H), 1.86-1.27 (m, 48H), 1.22 (d, 12H), 0.88 (t, 12H).

APCI-MS analysis: Calculated C66H126N4O14S2 [M+H]=1263.8, Observed=1263.9.

### Bis(2-ethylbutyl) 9,9′-((4-((2-(4-(2-((4-(bis(2-hydroxy-7-(isopentyloxy)-7-oxoheptyl)amino)-butanoyl)oxy)ethyl)piperazin-1-yl)ethyl)disulfaneyl)butyl)azanediyl)bis(8-hydroxynonanoate) (Compound 56)

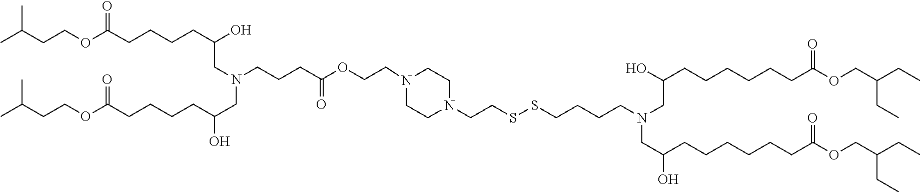 [SMILES: CCC(CC)COC(=O)CCCCCCC(O)CN(CCCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCC(CC)CC]

¹H NMR (300 MHz, CDCl₃) δ 4.19 (t, 2H), 4.08 (t, 4H), 3.98 (d, 4H), 3.61 (m, 4H), 2.84-2.45 (m, 22H), 2.44-2.25 (m, 16H), 1.83-1.28 (m, 54H), 0.91 (d, 12H), 0.88 (t, 12H).

APCI-MS analysis: Calculated C70H134N4O14S2 [M+H]=1318.9, Observed=1319.0.

### Bis(2-ethylbutyl) 9,9′-((3-((2-(4-(2-((4-(bis(2-hydroxy-7-(isopentyloxy)-7-oxoheptyl)amino)-butanoyl)oxy)ethyl)piperazin-1-yl)ethyl)disulfaneyl)propyl)azanediyl)bis(8-hydroxynonanoate) (Compound 45)

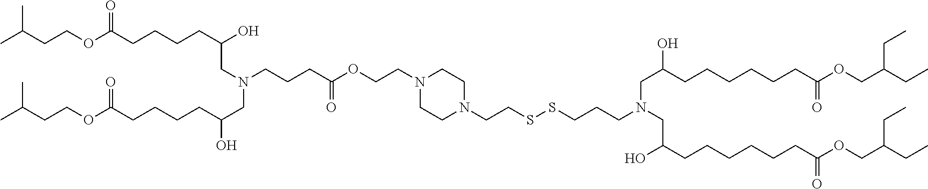 [SMILES: CCC(CC)COC(=O)CCCCCCC(O)CN(CCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCC(CC)CC]

¹H NMR (300 MHz, CDCl₃) δ 4.19 (t, 2H), 4.08 (t, 4H), 3.98 (d, 4H), 3.61 (m, 4H), 2.84-2.46 (m, 22H), 2.45-2.23 (m, 16H), 1.91-1.29 (m, 52H), 0.91 (d, 12H), 0.88 (t, 12H).

APCI-MS analysis: Calculated C69H132N4O14S2 [M+H]=1305.9, Observed=1305.9.

### Bis(2-ethylbutyl) 9,9′-((3-((2-(4-(2-((4-(bis(7-(2-ethylbutoxy)-7-hydroxy-7-oxoheptyl)amino)butanoyl)oxy)ethyl)piperazin-1-yl)ethyl)disulfaneyl)propyl)-azanediyl)bis(8-hydroxynonanoate) (Compound 47)

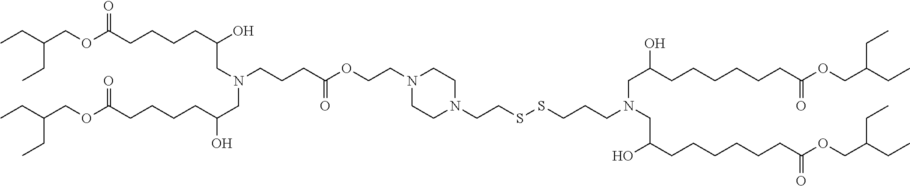 [SMILES: CCC(CC)COC(=O)CCCCCCC(O)CN(CCCSSCCN1CCN(CCOC(=O)CCCN(CC(O)CCCCC(=O)OCC(CC)CC)CC(O)CCCCC(=O)OCC(CC)CC)CC1)CC(O)CCCCCCC(=O)OCC(CC)CC]

¹H NMR (300 MHz, CDCl₃) δ 4.21 (t, 2H), 3.98 (d, 8H), 3.74 (m, 4H), 2.82-2.46 (m, 22H), 2.43-2.25 (m, 16H), 1.99-1.25 (m, 56H), 0.93-0.85 (m, 24H).

APCI-MS analysis: Calculated C71H136N4O14S2 [M+H]=1334.0, Observed=1133.8.

### Bis(2-ethylbutyl) 9,9′-((4-((2-(4-(2-((4-(bis(7-(2-ethylbutoxy)-2-hydroxy-7-oxoheptyl)amino)butanoyl)oxy)ethyl)piperazin-1-yl)ethyl)disulfaneyl)butyl)azanediyl)bis(8-hydroxynonanoate) (Compound 57)

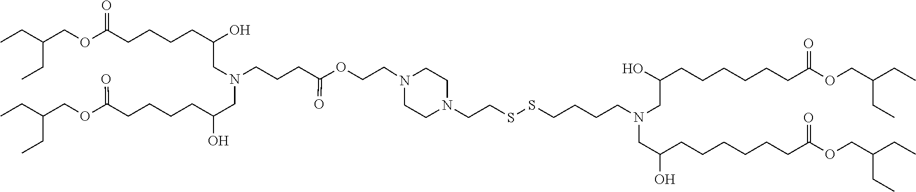 [SMILES: CCC(CC)COC(=O)CCCCCCC(O)CN(CCCCSSCCN1CCN(CCOC(=O)CCCN(CC(O)CCCCC(=O)OCC(CC)CC)CC(O)CCCCC(=O)OCC(CC)CC)CC1)CC(O)CCCCCCC(=O)OCC(CC)CC]

¹H NMR (300 MHz, CDCl₃) δ 4.19 (t, 2H), 3.98 (d, 8H), 3.74 (m, 4H), 2.83-2.25 (m, 38H), 1.90-1.22 (m, 58H), 0.88 (t, 24H).

APCI-MS analysis: Calculated C72H138N4O14S2 [M+H]=1348.0, Observed=1347.9.

### Bis(2-ethylbutyl) 9,9′-((4-((2-(4-(2-((5-(bis(7-(2-ethylbutoxy)-2-hydroxy-7-oxoheptyl)-amino)pentanoyl)oxy)ethyl)piperazin-1-yl)ethyl)disulfaneyl)butyl)azanediyl)bis(8-hydroxynonanoate) (Compound 58)

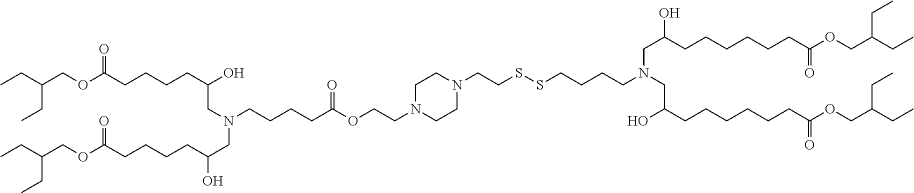 [SMILES: CCC(CC)COC(=O)CCCCCCC(O)CN(CCCCSSCCN1CCN(CCOC(=O)CCCCN(CC(O)CCCCC(=O)OCC(CC)CC)CC(O)CCCCC(=O)OCC(CC)CC)CC1)CC(O)CCCCCCC(=O)OCC(CC)CC]

¹H NMR (300 MHz, CDCl₃) δ 4.19 (t, 2H), 3.98 (d, 8H), 3.63 (m, 4H), 2.85-2.25 (m, 40H), 1.90-1.24 (m, 62H), 0.88 (t, 24H).

APCI-MS analysis: Calculated C73H140N4O14S2 [M+H]=1362.0, Observed=1361.2.

### Dibutyl 9,9′-((5-(2-(4-(2-((4-(bis(9-(2-ethyl butoxy)-2-hydroxy-9-oxononyl)amino)propyl)disulfaneyl)ethyl)piperazin-1-yl)ethoxy)-4-oxobutyl)azanediyl)bis(8-hydroxynonanoate) (Compound 42)

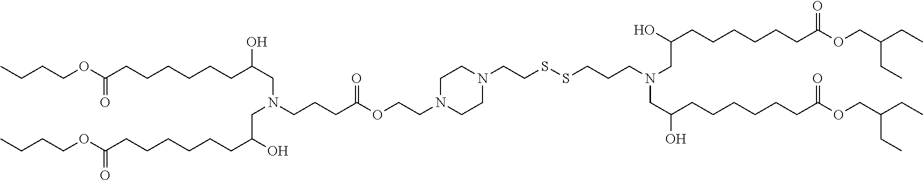 [SMILES: CCCCOC(=O)CCCCCCC(O)CN(CCCC(=O)OCCN1CCN(CCSSCCCN(CC(O)CCCCCCC(=O)OCC(CC)CC)CC(O)CCCCCCC(=O)OCC(CC)CC)CC1)CC(O)CCCCCCC(=O)OCCCC]

¹H NMR (300 MHz, CDCl₃) δ 4.19 (t, 2H), 4.04 (t, 4H), 3.98 (d, 4H), 3.72 (m, 4H), 2.84-2.28 (m, 38H), 1.95-1.22 (m, 62H), 0.95-0.85 (m, 18H).

APCI-MS analysis: Calculated C71H136N4O14S2 [M+H]=1334.0, Observed=1333.8.

### Dibutyl 9,9′-((4-((2-(4-(2-((4-(bis(9-butoxy-2-hydroxy-9-oxononyl)amino)butanoyl)oxy)ethyl)piperazin-1-yl)ethyl)disulfaneyl)butyl)azanediyl)bis(8-hydroxynonanoate) (Compound 52)

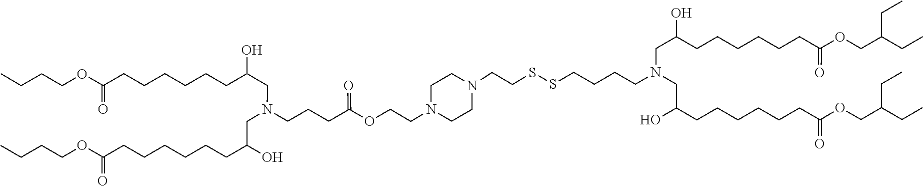 [SMILES: CCCCOC(=O)CCCCCCC(O)CN(CCCC(=O)OCCN1CCN(CCSSCCCCN(CC(O)CCCCCCC(=O)OCC(CC)CC)CC(O)CCCCCCC(=O)OCC(CC)CC)CC1)CC(O)CCCCCCC(=O)OCCCC]

¹H NMR (300 MHz, CDCl₃) δ 4.19 (t, 2H), 4.06 (t, 4H), 3.98 (d, 4H), 3.79 (m, 4H), 2.84-2.28 (m, 38H), 1.95-1.22 (m, 64H), 0.95-0.85 (m, 18H).

APCI-MS analysis: Calculated C72H138N4O14S2 [M+H]=1348.0, Observed=1347.9.

### Dibutyl 9,9′-((5-(2-(4-(2-((4-(bis(9-(2-ethyl butoxy)-2-hydroxy-9-oxononyl)amino)-butyl)disulfaneyl)ethyl)piperazin-1-yl)ethoxy)-5-oxopentyl)azanediyl)bis(8-hydroxynonanoate) (Compound 53)

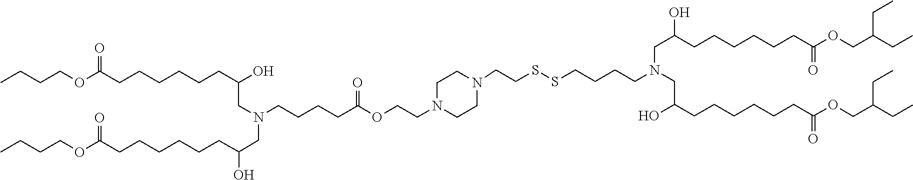 [SMILES: CCCCOC(=O)CCCCCCC(O)CN(CCCCC(=O)OCCN1CCN(CCSSCCCCN(CC(O)CCCCCCC(=O)OCC(CC)CC)CC(O)CCCCCCC(=O)OCC(CC)CC)CC1)CC(O)CCCCCCC(=O)OCCCC]

¹H NMR (300 MHz, CDCl₃) δ 4.19 (t, 2H), 4.05 (t, 4H), 3.98 (d, 4H), 3.61 (m, 4H), 2.84-2.45 (m, 22H), 2.44-2.25 (m, 16H), 1.77-1.26 (m, 66H), 0.92 (t, 6H), 0.88 (t, 12H).

APCI-MS analysis: Calculated C73H140N4O14S2 [M+H]=1362.0, Observed=1362.0.

### Dibutyl 9,9′-((5-(2-(4-(2-((3-(bis(9-(2-ethyl butoxy)-2-hydroxy-9-oxononyl)amino)-propyl)disulfaneyl)ethyl)piperazin-1-yl)ethoxy)-5-oxopentyl)azanediyl)bis(8-hydroxynonanoate) (Compound 43)

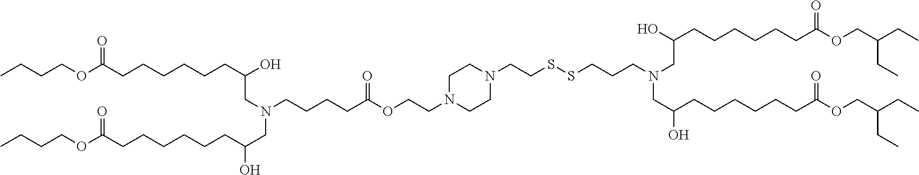 [SMILES: CCCCOC(=O)CCCCCCC(O)CN(CCCCC(=O)OCCN1CCN(CCSSCCCN(CC(O)CCCCCCC(=O)OCC(CC)CC)CC(O)CCCCCCC(=O)OCC(CC)CC)CC1)CC(O)CCCCCCC(=O)OCCCC]

¹H NMR (300 MHz, CDCl₃) δ 4.19 (t, 2H), 4.06 (t, 4H), 3.98 (d, 4H), 3.62 (m, 4H), 2.86-2.46 (m, 22H), 2.45-2.23 (m, 16H), 1.91-1.23 (m, 64H), 0.92 (t, 6H), 0.88 (t, 12H).

APCI-MS analysis: Calculated C72H138N4O14S2 [M+H]=1348.0, Observed=1348.0.

### Bis(2-ethylbutyl) 7,7′-((3-((2-(4-(2-((4-(bis(7-(2-ethylbutoxy)-2-hydroxy-7-oxoheptyl)amino)butanoyl)oxy)ethyl)piperazin-1-yl)ethyl)disulfaneyl)propyl)azanediyl)bis(6-hydroxyheptanoate) (Compound 92)

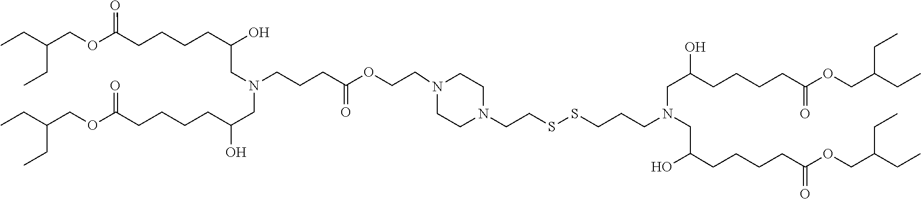 [SMILES: CCC(CC)COC(=O)CCCCC(O)CN(CCCSSCCN1CCN(CCOC(=O)CCCN(CC(O)CCCCC(=O)OCC(CC)CC)CC(O)CCCCC(=O)OCC(CC)CC)CC1)CC(O)CCCCC(=O)OCC(CC)CC]

¹H NMR (300 MHz, Methanol-d₄) δ 4.21 (t, 2H), 4.01 (d, 8H), 3.62 (m, 4H), 2.88-2.50 (m, 22H), 2.45-2.28 (m, 16H), 1.89-1.73 (m, 4H), 1.64 (m, 8H), 1.56-1.45 (m, 12H), 1.37 (m, 24H), 0.91 (t, 24H).

APCI-MS analysis: Calculated C67H128N4O14S2 [M+H]=1277.9, Observed=1277.8.

### Dibutyl 9,9′-((4-(2-(4-(2-((3-(bis(2-hydroxy-7-isopropoxy-7-oxoheptyl)amino)propyl)disulfaneyl)ethyl)piperazin-1-yl)ethoxy)-4-pentyl)azanediyl)bis(8-hydroxynonanoate) (Compound 78)

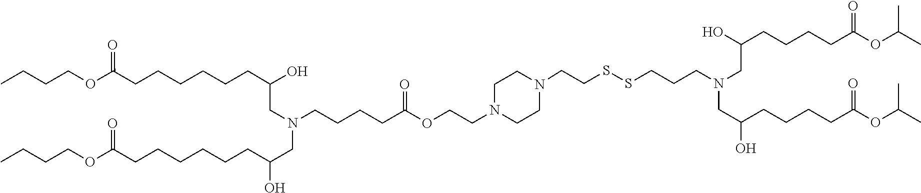 [SMILES: CCCCOC(=O)CCCCCCC(O)CN(CCCCC(=O)OCCN1CCN(CCSSCCCN(CC(O)CCCCC(=O)OC(C)C)CC(O)CCCCC(=O)OC(C)C)CC1)CC(O)CCCCCCC(=O)OCCCC]

¹H NMR (300 MHz, CDCl₃) δ 4.99 (hept, 2H), 4.18 (t, 2H), 4.05 (t, 4H), 3.64 (m, 4H), 2.86-2.21 (m, 38H), 1.90-1.28 (m, 46H), 1.22 (d, 12H), 0.90 (t, 6H).

APCI-MS analysis: Calculated C62H118N4O14S2 [M+H]=1207.8, Observed=1207.8.

### Di butyl 7,7′-((3-((2-(4-(2-((4-(bis(7-(2-ethylbutoxy)-2-hydroxy-7-oxoheptyl)amino)butanoyl)oxy)ethyl)piperazin-1-yl)ethyl)disulfaneyl)propyl)-azanediyl)bis(6-hydroxyheptanoate) (GL-HEPES-E3-E7-Es6-DS-3-E7-E4)

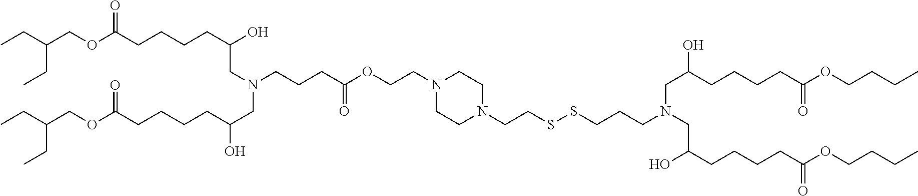 [SMILES: CCCCOC(=O)CCCCC(O)CN(CCCSSCCN1CCN(CCOC(=O)CCCN(CC(O)CCCCC(=O)OCC(CC)CC)CC(O)CCCCC(=O)OCC(CC)CC)CC1)CC(O)CCCCC(=O)OCCCC]

¹H NMR (300 MHz, CDCl₃) δ 4.19 (t, 2H), 4.06 (t, 4H), 3.98 (d, 4H), 3.64 (m, 4H), 2.84-2.45 (m, 22H), 2.42-2.24 (m, 16H), 1.85-1.73 (m, 4H), 1.72-1.46 (m, 20H), 1.45-1.29 (m, 22H), 0.93 (t, 6H), 0.88 (t, 12H).

APCI-MS analysis: Calculated C63H120N4O14S2 [M+H]=1221.8, Observed=1221.7.

### Bis(2-ethylbutyl) 7,7′-((4-(2-(4-(2-((4-(bis(2-hydroxy-7-isopropoxy-7-oxoheptyl)amino)butyl)disulfaneyl)ethyl)piperazin-1-yl)ethoxy)-4-oxobutyl)azanediyl)bis(6-hydroxyheptanoate) (GL-HEPES-E3-E7-Es6-DS-4-E7-Ei3)

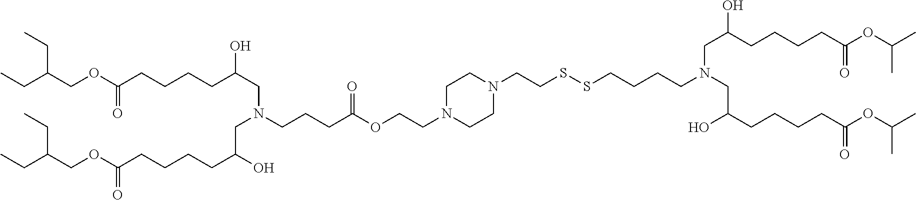 [SMILES: CCC(CC)COC(=O)CCCCC(O)CN(CCCC(=O)OCCN1CCN(CCSSCCCCN(CC(O)CCCCC(=O)OC(C)C)CC(O)CCCCC(=O)OC(C)C)CC1)CC(O)CCCCC(=O)OCC(CC)CC]

¹H NMR (300 MHz, CDCl₃) δ 4.99 (hept, 2H), 4.19 (t, 2H), 3.98 (d, 4H), 3.62 (m, 4H), 2.83-2.21 (m, 38H), 1.85-1.24 (m, 40H), 1.22 (d, 12H), 0.86 (t, 12H).

APCI-MS analysis: Calculated C62H118N4O14S2 [M+H]=1207.8, Observed=1207.7.

### Dibutyl 9,9′-((4-(2-(4-(2-((3-(bis(2-hydroxy-7-(isopentyloxy)-7-oxoheptyl)amino)propyl)disulfaneyl)ethyl)piperazin-1-yl)ethoxy)-4-oxobutyl)azanediyl)bis(8-hydroxynonanoate) (GL-HEPES-E3-E9-E4-DS-3-E7-Ei5)

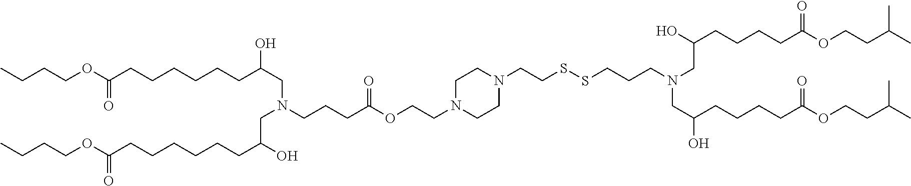 [SMILES: CCCCOC(=O)CCCCCCC(O)CN(CCCC(=O)OCCN1CCN(CCSSCCCN(CC(O)CCCCC(=O)OCCC(C)C)CC(O)CCCCC(=O)OCCC(C)C)CC1)CC(O)CCCCCCC(=O)OCCCC]

¹H NMR (300 MHz, CDCl₃) δ 4.19 (t, 2H), 4.06 (t, 8H), 3.70 (m, 4H), 2.84-2.46 (m, 22H), 2.43-2.26 (m, 16H), 1.96-1.25 (m, 50H), 0.95-0.88 (m, 18H).

APCI-MS analysis: Calculated C65H124N4O14S2 [M+H]=1249.8, Observed=1249.8.

### Dibutyl 9,9′-((4-(2-(4-(2-((4-(bis(2-hydroxy-7-(isopentyloxy)-7-oxoheptyl)amino)butyl)disulfaneyl)ethyl)piperazin-1-yl)ethoxy)-4-oxobutyl)azanediyl)bis(8-hydroxynonanoate) (GL-HEPES-E3-E9-E4-DS-4-E7-Ei5)

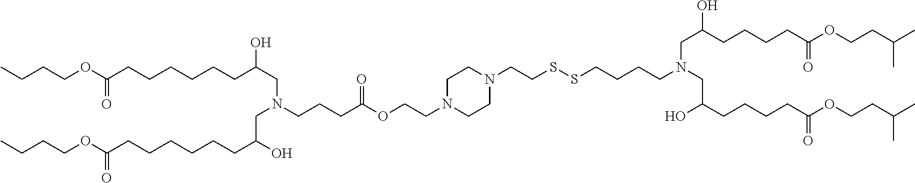 [SMILES: CCCCOC(=O)CCCCCCC(O)CN(CCCC(=O)OCCN1CCN(CCSSCCCCN(CC(O)CCCCC(=O)OCCC(C)C)CC(O)CCCCC(=O)OCCC(C)C)CC1)CC(O)CCCCCCC(=O)OCCCC]

¹H NMR (300 MHz, CDCl₃) δ 4.19 (t, 2H), 4.06 (m, 8H), 3.75 (m, 4H), 2.84-2.46 (m, 22H), 2.43-2.25 (m, 16H), 1.92-1.25 (m, 52H), 0.95-0.88 (m, 18H).

APCI-MS analysis: Calculated C66H126N4O14S2 [M+H]=1263.9, Observed=1263.7.

### Bis(2-ethylbutyl) 7,7′-((4-(2-(4-(2-((3-(bis(2-hydroxy-7-isopropoxy-7-oxoheptyl)amino)propyl)disulfaneyl)ethyl)piperazin-1-yl)ethoxy)-4-oxobutyl)azanediyl)bis(6-hydroxyheptanoate) (GL-HEPES-E3-E7-Es6-DS-3-E7-Ei3)

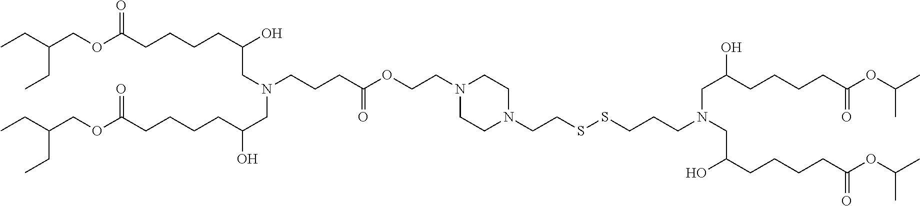 [SMILES: CCC(CC)COC(=O)CCCCC(O)CN(CCCC(=O)OCCN1CCN(CCSSCCCN(CC(O)CCCCC(=O)OC(C)C)CC(O)CCCCC(=O)OC(C)C)CC1)CC(O)CCCCC(=O)OCC(CC)CC]

¹H NMR (300 MHz, CDCl₃) δ 4.99 (hept, 2H), 4.19 (t, 2H), 3.98 (d, 4H), 3.65 (m, 4H), 2.86-2.21 (m, 38H), 1.90-1.28 (m, 38H), 1.22 (d, 12H), 0.89 (t, 12H).

APCI-MS analysis: Calculated C61H116N4O14S2 [M+H]=1193.7, Observed=1193.6.

### Dibutyl 9,9′-((4-(2-(4-(2-((3-(bis(2-hydroxy-7-isopropoxy-7-oxoheptyl)amino)propyl)disulfaneyl)ethyl)piperazin-1-yl)ethoxy)-4-oxobutyl)azanediyl)bis(8-hydroxynonanoate) (GL-HEPES-E3-E9-E4-DS-3-E7-Ei3)

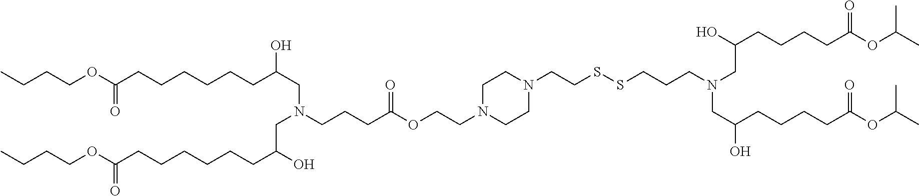 [SMILES: CCCCOC(=O)CCCCCCC(O)CN(CCCC(=O)OCCN1CCN(CCSSCCCN(CC(O)CCCCC(=O)OC(C)C)CC(O)CCCCC(=O)OC(C)C)CC1)CC(O)CCCCCCC(=O)OCCCC]

¹H NMR (300 MHz, CDCl₃) δ 4.99 (hept, 2H), 4.19 (t, 2H), 4.04 (d, 4H), 3.62 (m, 4H), 2.86-2.21 (m, 38H), 1.90-1.28 (m, 44H), 1.22 (d, 12H), 0.89 (t, 6H).

APCI-MS analysis: Calculated C61H116N4O14S2 [M+H]=1193.7, Observed=1193.7.

### Dibutyl 9,9′-((4-((2-(4-(2-((4-(bis(7-(2-ethylbutoxy)-2-hydroxy-7-oxoheptyl)amino)butanoyl)oxy)ethyl)piperazin-1-yl)ethyl)disulfaneyl)butyl)azanediyl)bis(8-hydroxynonanoate) (GL-HEPES-E3-E7-Es6-DS-4-E9-E4)

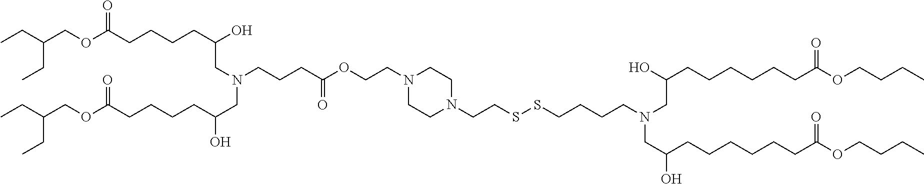 [SMILES: CCCCOC(=O)CCCCCCC(O)CN(CCCCSSCCN1CCN(CCOC(=O)CCCN(CC(O)CCCCC(=O)OCC(CC)CC)CC(O)CCCCC(=O)OCC(CC)CC)CC1)CC(O)CCCCCCC(=O)OCCCC]

¹H NMR (300 MHz, CDCl₃) δ 4.19 (t, 2H), 4.06 (t, 4H), 3.98 (d, 4H), 3.64 (m, 4H), 2.84-2.45 (m, 22H), 2.42-2.24 (m, 16H), 1.90-1.28 (m, 56H), 0.93 (t, 6H), 0.88 (t, 12H).

APCI-MS analysis: Calculated C68H130N4O14S2 [M+H]=1291.9, Observed=1291.8.

### Diisopentyl 9,9′-((3-((2-(4-(2-((4-(bis(7-(2-ethylbutoxy)-2-hydroxy-7-oxoheptyl)amino)butanoyl)oxy)ethyl)piperazin-1-yl)ethyl)disulfaneyl)propyl)azanediyl)bis(8-hydroxynonanoate) (GL-HEPES-E3-E7-Es6-DS-3-E9-Ei5)

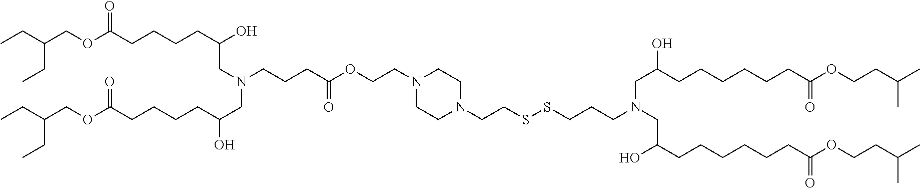 [SMILES: CCC(CC)COC(=O)CCCCC(O)CN(CCCC(=O)OCCN1CCN(CCSSCCCN(CC(O)CCCCCCC(=O)OCCC(C)C)CC(O)CCCCCCC(=O)OCCC(C)C)CC1)CC(O)CCCCC(=O)OCC(CC)CC]

¹H NMR (300 MHz, CDCl₃) δ 4.20 (t, 2H), 4.08 (t, 4H), 3.98 (d, 4H), 3.68 (m, 4H), 2.88-2.45 (m, 22H), 2.42-2.24 (m, 16H), 1.95-1.26 (m, 52H), 0.95-0.86 (m, 24H).

APCI-MS analysis: Calculated C69H132N4O14S2 [M+H]=1305.9, Observed=1305.8.

### dibutyl 9,9′-((4-(2-(4-(2-((3-(bis(2-hydroxy-9-(isopentyloxy)-9-oxononyl)amino)propyl)disulfaneyl)ethyl)piperazin-1-yl)ethoxy)-4-oxobutyl)azanediyl)bis(8-hydroxynonanoate) (GL-HEPES-E3-E9-E4-DS-3-E9-Ei5)

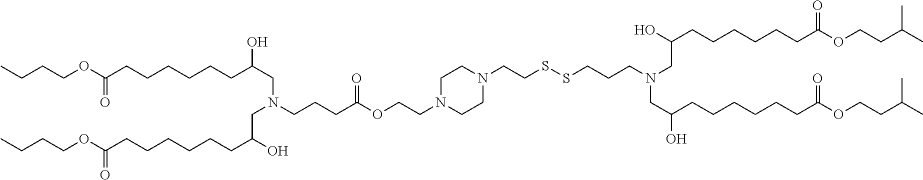 [SMILES: CCCCOC(=O)CCCCCCC(O)CN(CCCC(=O)OCCN1CCN(CCSSCCCN(CC(O)CCCCCCC(=O)OCCC(C)C)CC(O)CCCCCCC(=O)OCCC(C)C)CC1)CC(O)CCCCCCC(=O)OCCCC]

¹H NMR (300 MHz, CDCl₃) δ 4.20 (t, 2H), 4.06 (m, 8H), 3.64 (m, 4H), 2.88-2.45 (m, 22H), 2.42-2.24 (m, 16H), 1.95-1.26 (m, 58H), 0.95-0.88 (m, 18H).

APCI-MS analysis: Calculated C69H132N4O14S2 [M+H]=1305.9, Observed=1305.8.

### Bis(2-ethylbutyl) 9,9′-((3-((2-(4-(2-((4-(bis(7-(2-ethylbutoxy)-2-hydroxy-7-oxoheptyl)amino)butanoyl)oxy)ethyl)piperazin-1-yl)ethyl)disulfaneyl)propyl)azanediyl)bis(8-hydroxynonanoate) (GL-HEPES-E3-E7-Es6-DS-3-E9-Es6)

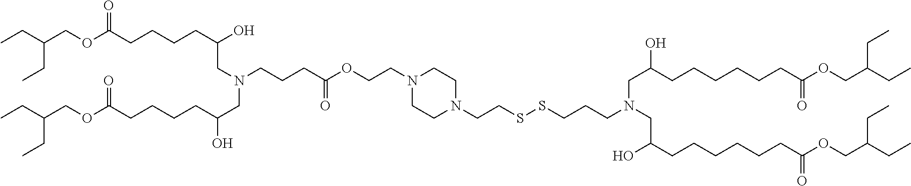 [SMILES: CCC(CC)COC(=O)CCCCCCC(O)CN(CCCSSCCN1CCN(CCOC(=O)CCCN(CC(O)CCCCC(=O)OCC(CC)CC)CC(O)CCCCC(=O)OCC(CC)CC)CC1)CC(O)CCCCCCC(=O)OCC(CC)CC]

¹H NMR (300 MHz, CDCl₃) δ 4.21 (t, 2H), 3.98 (d, 8H), 3.74 (m, 4H), 2.82-2.46 (m, 22H), 2.43-2.25 (m, 16H), 1.99-1.25 (m, 56H), 0.93-0.85 (m, 24H).

APCI-MS analysis: Calculated C71H136N4O14S2 [M+H]=1334.0, Observed=1133.8.

### Diisopropyl 7,7′-((3-((2-(4-(2-((4-(bis(2-hydroxy-6-oxo-6-(pentan-3-yloxy)hexyl)amino)butanoyl)oxy)ethyl)piperazin-1-yl)ethyl)disulfaneyl)propyl)azanediyl)bis(6-hydroxyheptanoate) (GL-HEPES-E3-E6-Es5-DS-3-E7-Ei3)

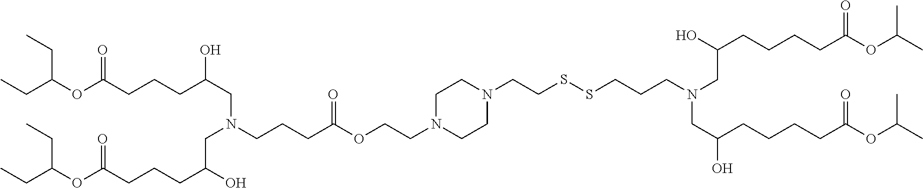 [SMILES: CCC(CC)OC(=O)CCCC(O)CN(CCCC(=O)OCCN1CCN(CCSSCCCN(CC(O)CCCCC(=O)OC(C)C)CC(O)CCCCC(=O)OC(C)C)CC1)CC(O)CCCC(=O)OC(CC)CC]

¹H NMR (300 MHz, CDCl₃) δ 4.99 (hept, 2H), 4.74 (pent, 2H), 4.19 (t, 2H), 3.65 (m, 4H), 3.32-3.00 (bs, 4H), 2.83-2.24 (m, 38H), 1.91-1.74 (m, 2H), 1.70-1.36 (m, 30H), 1.22 (d, 12H), 0.86 (t, 12H).

APCI-MS analysis: Calculated C57H108N4O14S2 [M+H]=1137.6, Observed=1137.6.

### Diisopropyl 7,7′-((3-((2-(4-(2-((4-(bis(2-hydroxy-7-isopropoxy-7-oxoheptyl)amino)butanoyl)oxy)ethyl)piperazin-1-yl)ethyl)disulfaneyl)propyl)azanediyl)bis(6-hydroxyheptanoate) (GL-HEPES-E3-E7-Ei3-DS-3-E7-Ei3)

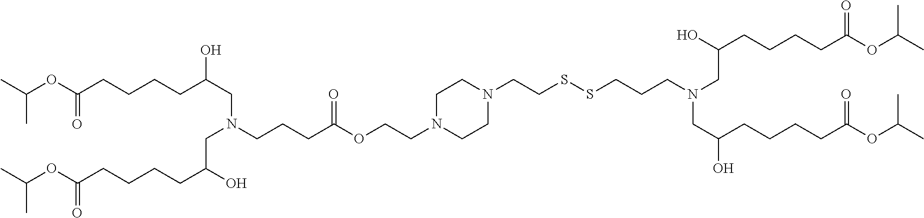 [SMILES: CC(C)OC(=O)CCCCC(O)CN(CCCSSCCN1CCN(CCOC(=O)CCCN(CC(O)CCCCC(=O)OC(C)C)CC(O)CCCCC(=O)OC(C)C)CC1)CC(O)CCCCC(=O)OC(C)C]

¹H NMR (300 MHz, CDCl₃) δ 4.99 (hept, 4H), 4.19 (t, 2H), 3.68 (m, 4H), 3.32-3.00 (bs, 4H), 2.87-2.35 (m, 30H), 2.26 (t, 8H), 1.93-1.74 (m, 4H), 1.70-1.56 (m, 8H), 1.54-1.33 (m, 16H), 1.22 (d, 24H).

APCI-MS analysis: Calculated C55H104N4O14S2 [M+H]=1109.5, Observed=1109.6.

### Bis(2-ethylbutyl) 9,9′-((4-(2-(4-(2-((3-(bis(2-hydroxy-7-isopropoxy-7-oxoheptyl)amino)propyl)disulfaneyl)ethyl)piperazin-1-yl)ethoxy)-4-oxobutyl)azanediyl)bis(8-hydroxynonanoate) (GL-HEPES-E3-E9-Es6-DS-3-E7-Ei3)

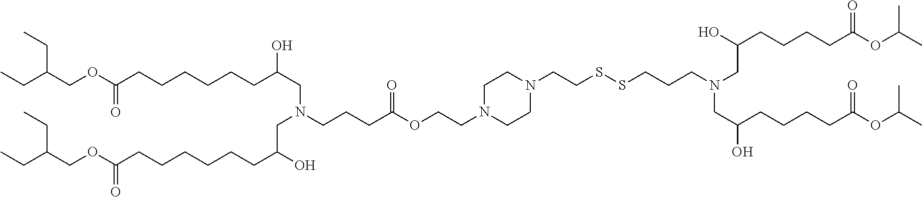 [SMILES: CCC(CC)COC(=O)CCCCCCC(O)CN(CCCC(=O)OCCN1CCN(CCSSCCCN(CC(O)CCCCC(=O)OC(C)C)CC(O)CCCCC(=O)OC(C)C)CC1)CC(O)CCCCCCC(=O)OCC(CC)CC]

¹H NMR (300 MHz, CDCl₃) δ 4.99 (hept, 2H), 4.19 (t, 2H), 3.98 (d, 4H), 3.63 (m, 4H), 2.84-2.35 (m, 30H), 2.28 (q, 8H), 1.92-1.74 (m, 5H), 1.68-1.56 (m, 9H), 1.54-1.26 (m, 32H), 1.22 (d, 12H), 0.88 (t, 12H).

APCI-MS analysis: Calculated C65H124N4O14S2 [M+H]=1249.8, Observed=1249.7.

### Dibutyl 9,9′-((4-(2-(4-(2-((3-(bis(9-(2-ethyl butoxy)-2-hydroxy-9-oxononyl)amino)propyl)disulfaneyl)ethyl)piperazin-1-yl)ethoxy)-4-oxobutyl)azanediyl)bis(8-hydroxynonanoate) (GL-HEPES-E3-E9-E4-DS-3-E9-Es6)

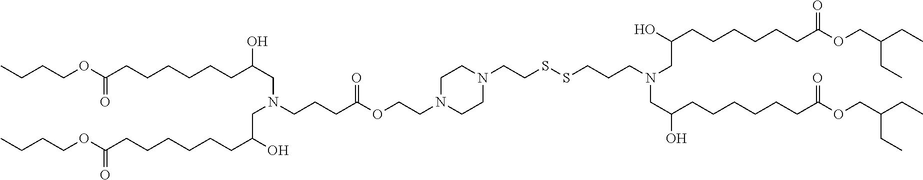 [SMILES: CCCCOC(=O)CCCCCCC(O)CN(CCCC(=O)OCCN1CCN(CCSSCCCN(CC(O)CCCCCCC(=O)OCC(CC)CC)CC(O)CCCCCCC(=O)OCC(CC)CC)CC1)CC(O)CCCCCCC(=O)OCCCC]

¹H NMR (300 MHz, CDCl₃) δ 4.19 (t, 2H), 4.04 (t, 4H), 3.98 (d, 4H), 3.72 (m, 4H), 2.84-2.28 (m, 38H), 1.95-1.22 (m, 62H), 0.95-0.85 (m, 18H).

APCI-MS analysis: Calculated C71H136N4O14S2 [M+H]=1334.0, Observed=1333.8.

### Diisopentyl 9,9′-((4-((2-(4-(2-((4-(bis(7-(2-ethylbutoxy)-2-hydroxy-7-oxoheptyl)amino)butanoyl)oxy)ethyl)piperazin-1-yl)ethyl)disulfaneyl)butyl)azanediyl)bis(8-hydroxynonanoate) (GL-HEPES-E3-E7-Es6-DS-4-E9-Ei5)

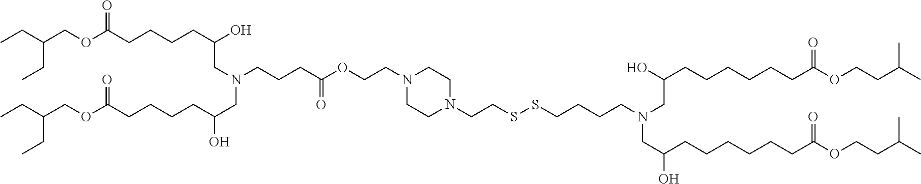 [SMILES: CCC(CC)COC(=O)CCCCC(O)CN(CCCC(=O)OCCN1CCN(CCSSCCCCN(CC(O)CCCCCCC(=O)OCCC(C)C)CC(O)CCCCCCC(=O)OCCC(C)C)CC1)CC(O)CCCCC(=O)OCC(CC)CC]

¹H NMR (300 MHz, CDCl₃) δ 4.19 (t, 2H), 4.06 (t, 4H), 3.98 (d, 4H), 3.62 (m, 4H), 2.84-2.28 (m, 38H), 1.95-1.22 (m, 54H), 0.95-0.86 (m, 24H).

APCI-MS analysis: Calculated C70H134N4O14S2 [M+H]=1319.9, Observed=1319.8.

### Dibutyl 9,9′-((4-(2-(4-(2-((4-(bis(2-hydroxy-9-(isopentyloxy)-9-oxononyl)amino)butyl)disulfaneyl)ethyl)piperazin-1-yl)ethoxy)-4-oxobutyl)azanediyl)bis(8-hydroxynonanoate) (GL-HEPES-E3-E9-E4-DS-4-E9-Ei5)

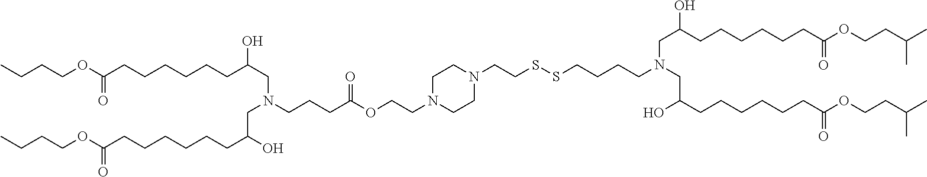 [SMILES: CCCCOC(=O)CCCCCCC(O)CN(CCCC(=O)OCCN1CCN(CCSSCCCCN(CC(O)CCCCCCC(=O)OCCC(C)C)CC(O)CCCCCCC(=O)OCCC(C)C)CC1)CC(O)CCCCCCC(=O)OCCCC]

¹H NMR (300 MHz, CDCl₃) δ 4.20 (t, 2H), 4.06 (m, 8H), 3.61 (m, 4H), 2.82-2.46 (m, 22H), 2.43-2.25 (m, 16H), 1.90-1.22 (m, 60H), 0.93-0.85 (m, 18H).

APCI-MS analysis: Calculated C70H134N4O14S2 [M+H]=1319.9, Observed=1319.8.

### Diisopentyl 7,7′-((3-((2-(4-(2-((4-(bis(2-hydroxy-7-isopropoxy-7-oxoheptyl)amino)butanoyl)oxy)ethyl)piperazin-1-yl)ethyl)disulfaneyl)propyl)-azanediyl)bis(6-hydroxyheptanoate) (GL-HEPES-E3-E7-Ei3-DS-3-E7-Ei5)

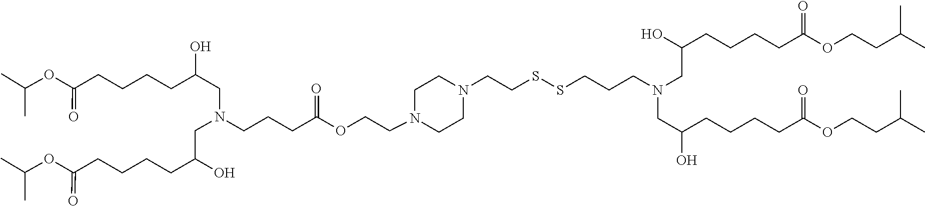 [SMILES: CC(C)CCOC(=O)CCCCC(O)CN(CCCSSCCN1CCN(CCOC(=O)CCCN(CC(O)CCCCC(=O)OC(C)C)CC(O)CCCCC(=O)OC(C)C)CC1)CC(O)CCCCC(=O)OCCC(C)C]

¹H NMR (300 MHz, CDCl₃) δ 4.99 (hept, 2H), 4.19 (t, 2H), 4.08 (t, 4H), 3.64 (m, 4H), 2.82-2.35 (m, 24H), 2.28 (t, 8H), 1.92-1.74 (m, 6H), 1.72-1.56 (m, 12H), 1.50 (q, 8H), 1.44-1.32 (m, 14H), 1.22 (d, 12H), 0.91 (d, 12H).

APCI-MS analysis: Calculated C59H112N4O14S2 [M+H]=1165.6, Observed=1165.7.

### Bis(2-ethylbutyl) 9,9′-((4-(2-(4-(2-((3-(bis(2-hydroxy-7-(isopentyloxy)-7-oxoheptyl)amino)propyl)disulfaneyl)ethyl)piperazin-1-yl)ethoxy)-4-oxobutyl)azanediyl)-bis(8-hydroxynonanoate) (GL-HEPES-E3-E9-Es6-DS-3-E7-Ei5)

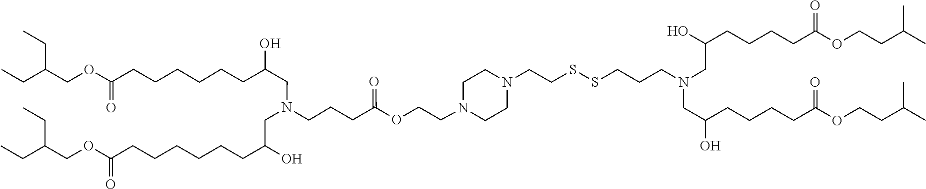 [SMILES: CCC(CC)COC(=O)CCCCCCC(O)CN(CCCC(=O)OCCN1CCN(CCSSCCCN(CC(O)CCCCC(=O)OCCC(C)C)CC(O)CCCCC(=O)OCCC(C)C)CC1)CC(O)CCCCCCC(=O)OCC(CC)CC]

¹H NMR (300 MHz, CDCl₃) δ 4.20 (t, 2H), 4.08 (t, 4H), 3.98 (d, 4H), 3.65 (m, 4H), 2.84-2.32 (m, 32H), 2.29 (dt, 8H), 1.92-1.74 (m, 5H), 1.72-1.56 (m, 9H), 1.54-1.26 (m, 36H), 0.91 (d, 12H), 0.88 (t, 12H).

APCI-MS analysis: Calculated C69H132N4O14S2 [M+H]=1305.9, Observed=1305.8.

### Bis(2-ethylbutyl) 9,9′-((4-((2-(4-(2-((4-(bis(7-(2-ethylbutoxy)-2-hydroxy-7-oxoheptyl)amino)butanoyl)oxy)ethyl)piperazin-1-yl)ethyl)disulfaneyl)butyl)azanediyl)bis(8-hydroxynonanoate) (GL-HEPES-E3-E7-Es6-DS-4-E9-Es6)

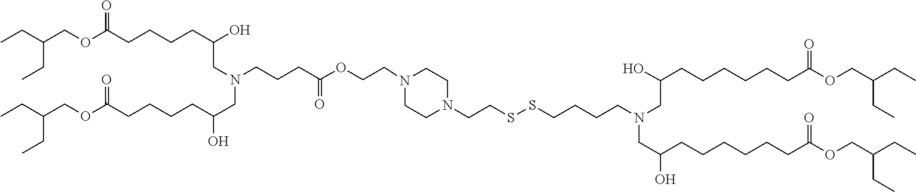 [SMILES: CCC(CC)COC(=O)CCCCCCC(O)CN(CCCCSSCCN1CCN(CCOC(=O)CCCN(CC(O)CCCCC(=O)OCC(CC)CC)CC(O)CCCCC(=O)OCC(CC)CC)CC1)CC(O)CCCCCCC(=O)OCC(CC)CC]

¹H NMR (300 MHz, CDCl₃) δ 4.19 (t, 2H), 3.98 (d, 8H), 3.74 (m, 4H), 2.83-2.25 (m, 38H), 1.90-1.22 (m, 58H), 0.88 (t, 24H).

APCI-MS analysis: Calculated C72H138N4O14S2 [M+H]=1348.0, Observed=1347.9.

### Dibutyl 9,9′-((4-((2-(4-(2-((4-(bis(9-butoxy-2-hydroxy-9-oxononyl)amino)butanoyl)oxy)ethyl)piperazin-1-yl)ethyl)disulfaneyl)butyl)azanediyl)bis(8-hydroxynonanoate) (GL-HEPES-E3-E9-E4-DS-4-E9-Es6)

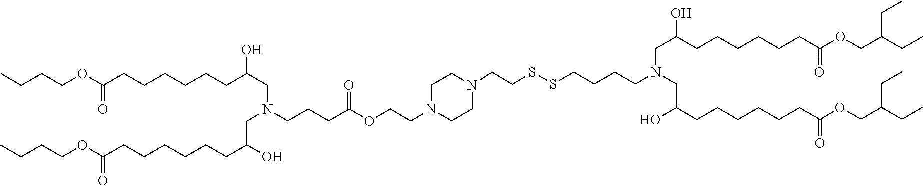 [SMILES: CCCCOC(=O)CCCCCCC(O)CN(CCCC(=O)OCCN1CCN(CCSSCCCCN(CC(O)CCCCCCC(=O)OCC(CC)CC)CC(O)CCCCCCC(=O)OCC(CC)CC)CC1)CC(O)CCCCCCC(=O)OCCCC]

¹H NMR (300 MHz, CDCl₃) δ 4.19 (t, 2H), 4.06 (t, 4H), 3.98 (d, 4H), 3.79 (m, 4H), 2.84-2.28 (m, 38H), 1.95-1.22 (m, 64H), 0.95-0.85 (m, 18H).

APCI-MS analysis: Calculated C72H138N4O14S2 [M+H]=1348.0, Observed=1347.9.

### Diisopentyl 7,7′-((4-(2-(4-(2-((3-(bis(2-hydroxy-7-isopropoxy-7-oxoheptyl)amino)propyl)disulfaneyl)ethyl)piperazin-1-yl)ethoxy)-4-oxobutyl)azanediyl)-bis(6-hydroxyheptanoate) (GL-HEPES-E3-E7-Ei5-DS-3-E7-Ei3)

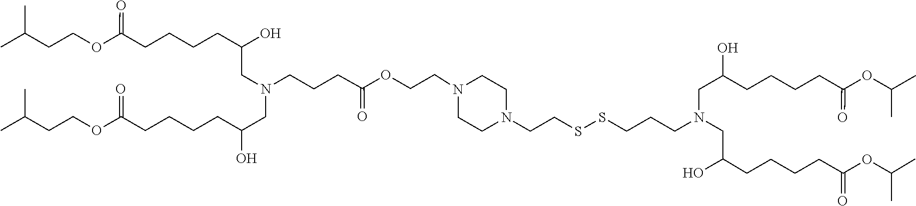 [SMILES: CC(C)CCOC(=O)CCCCC(O)CN(CCCC(=O)OCCN1CCN(CCSSCCCN(CC(O)CCCCC(=O)OC(C)C)CC(O)CCCCC(=O)OC(C)C)CC1)CC(O)CCCCC(=O)OCCC(C)C]

¹H NMR (300 MHz, CDCl₃) δ 4.99 (hept, 2H), 4.19 (t, 2H), 4.08 (t, 4H), 3.62 (m, 4H), 2.86-2.21 (m, 38H), 1.90-1.26 (m, 34H), 1.22 (d, 12H), 0.92 (d, 12H).

APCI-MS analysis: Calculated C59H112N4O14S2 [M+H]=1165.7, Observed=1165.8.

### Dibutyl 9,9′-((4-(2-(4-(2-((3-(bis(2-hydroxy-7-isopropoxy-7-oxoheptyl)amino)propyl)disulfaneyl)ethyl)piperazin-1-yl)ethoxy)-4-pentyl)azanediyl)bis(8-hydroxynonanoate) (GL-HEPES-E4-E9-E4-DS-3-E7-Ei3)

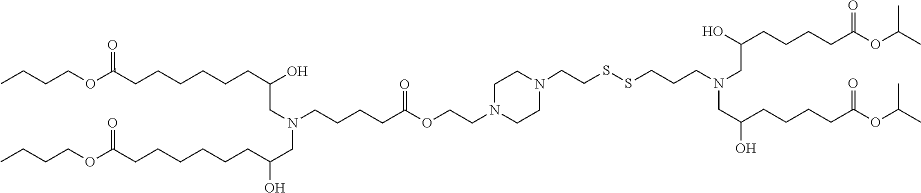 [SMILES: CCCCOC(=O)CCCCCCC(O)CN(CCCCC(=O)OCCN1CCN(CCSSCCCN(CC(O)CCCCC(=O)OC(C)C)CC(O)CCCCC(=O)OC(C)C)CC1)CC(O)CCCCCCC(=O)OCCCC]

¹H NMR (300 MHz, CDCl₃) δ 4.99 (hept, 2H), 4.18 (t, 2H), 4.05 (t, 4H), 3.64 (m, 4H), 2.86-2.21 (m, 38H), 1.90-1.28 (m, 46H), 1.22 (d, 12H), 0.90 (t, 6H).

APCI-MS analysis: Calculated C62H118N4O14S2 [M+H]=1207.8, Observed=1207.8.

### Diisopentyl 7,7′-((4-(2-(4-(2-((3-(bis(2-hydroxy-7-isopropoxy-7-oxoheptyl)amino)butyl)disulfaneyl)ethyl)piperazin-1-yl)ethoxy)-4-oxobutyl)azanediyl)bis(6-hydroxyheptanoate) (GL-HEPES-E3-E7-Ei5-DS-4-E7-Ei3)

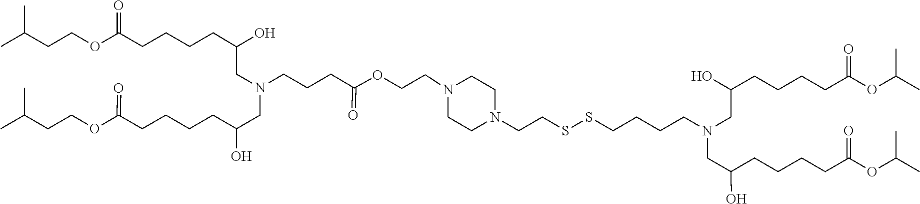 [SMILES: CC(C)CCOC(=O)CCCCC(O)CN(CCCC(=O)OCCN1CCN(CCSSCCCCN(CC(O)CCCCC(=O)OC(C)C)CC(O)CCCCC(=O)OC(C)C)CC1)CC(O)CCCCC(=O)OCCC(C)C]

¹H NMR (300 MHz, CDCl₃) δ 4.99 (hept, 2H), 4.19 (t, 2H), 4.08 (t, 4H), 3.61 (m, 4H), 2.83-2.23 (m, 38H), 1.84-1.30 (m, 36H), 0.92 (d, 12H), 0.86 (d, 12H).

APCI-MS analysis: Calculated C60H114N4O14S2 [M+H]=1179.7, Observed=1179.8.

### Dibutyl 9,9′-((4-(2-(4-(2-((3-(bis(2-hydroxy-7-isopropoxy-7-oxoheptyl)amino)butyl)disulfaneyl)ethyl)piperazin-1-yl)ethoxy)-4-pentyl)azanediyl)bis(8-hydroxynonanoate) (GL (GL-HEPES-E4-E9-E4-DS-4-E7-Ei3)

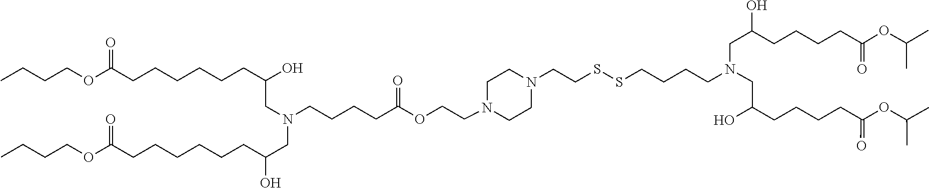 [SMILES: CCCCOC(=O)CCCCCCC(O)CN(CCCCC(=O)OCCN1CCN(CCSSCCCCN(CC(O)CCCCC(=O)OC(C)C)CC(O)CCCCC(=O)OC(C)C)CC1)CC(O)CCCCCCC(=O)OCCCC]

¹H NMR (300 MHz, CDCl₃) δ 4.99 (hept, 2H), 4.19 (t, 2H), 4.05 (t, 4H), 3.60 (m, 4H), 2.86-2.21 (m, 38H), 1.86-1.25 (m, 48H), 1.21 (d, 12H), 0.92 (t, 6H).

APCI-MS analysis: Calculated C63H120N4O14S2 [M+H]=1221.8, Observed=1221.8.

### Bis(2-ethylbutyl) 7,7′-((3-((2-(4-(2-((4-(bis(2-hydroxy-7-isopropoxy-7-oxoheptyl)amino)butanoyl)oxy)ethyl)piperazin-1-yl)ethyl)disulfaneyl)propyl)-azanediyl)bis(6-hydroxyheptanoate) (GL-HEPES-E3-E7-Ei3-DS-3-E7-Es6)

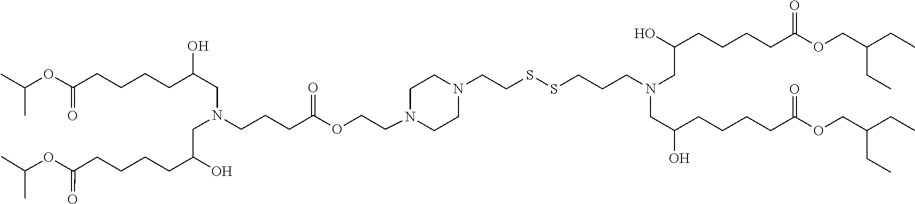 [SMILES: CCC(CC)COC(=O)CCCCC(O)CN(CCCSSCCN1CCN(CCOC(=O)CCCN(CC(O)CCCCC(=O)OC(C)C)CC(O)CCCCC(=O)OC(C)C)CC1)CC(O)CCCCC(=O)OCC(CC)CC]

¹H NMR (300 MHz, CDCl₃) δ 4.98 (hept, 2H), 4.21 (t, 2H), 3.97 (d, 4H), 3.74 (m, 6H), 2.92-2.38 (m, 28H), 2.29 (dt, 8H), 1.98-1.78 (m, 4H), 1.72-1.29 (m, 34H), 1.21 (d, 12H), 0.88 (t, 12H).

APCI-MS analysis: Calculated C61H116N4O14S2 [M+H]=1193.7, Observed=1193.7.

### Bis(2-ethylbutyl) 9,9′-((4-(2-(4-(2-((3-(bis(7-(2-ethylbutoxy)-2-hydroxy-7-oxoheptyl)amino)propyl)disulfaneyl)ethyl)piperazin-1-yl)ethoxy)-4-oxobutyl)azanediyl)-bis(8-hydroxynonanoate) (GL-HEPES-E3-E9-Es6-DS-3-E7-Es6)

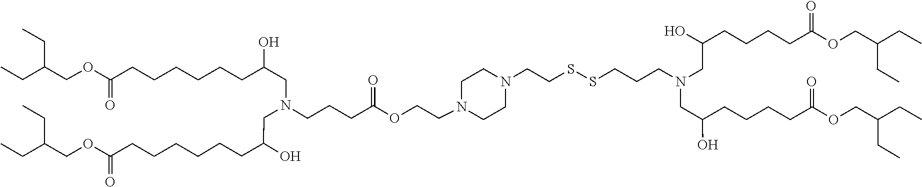 [SMILES: CCC(CC)COC(=O)CCCCCCC(O)CN(CCCC(=O)OCCN1CCN(CCSSCCCN(CC(O)CCCCC(=O)OCC(CC)CC)CC(O)CCCCC(=O)OCC(CC)CC)CC1)CC(O)CCCCCCC(=O)OCC(CC)CC]

¹H NMR (300 MHz, CDCl₃) δ 4.21 (t, 2H), 3.97 (d, 8H), 3.78 (m, 6H), 2.94-2.39 (m, 28H), 2.29 (dt, 8H), 1.92-1.74 (m, 4H), 1.72-1.26 (m, 52H), 0.88 (t, 24H).

APCI-MS analysis: Calculated C71H136N4O14S2 [M+H]=1334.0, Observed=1333.8.

### Dibutyl 9,9′-((3-((2-(4-(2-((4-(bis(2-hydroxy-7-isopropoxy-7-oxoheptyl)amino)butanoyl)oxy)ethyl)piperazin-1-yl)ethyl)disulfaneyl)propyl)-azanediyl)bis(8-hydroxynonanoate) (GL-HEPES-E3-E7-Ei3-DS-3-E9-E4)

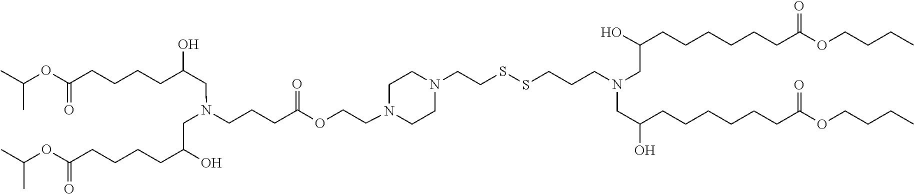 [SMILES: CCCCOC(=O)CCCCCCC(O)CN(CCCSSCCN1CCN(CCOC(=O)CCCN(CC(O)CCCCC(=O)OC(C)C)CC(O)CCCCC(=O)OC(C)C)CC1)CC(O)CCCCCCC(=O)OCCCC]

¹H NMR (300 MHz, CDCl₃) δ 4.98 (hept, 2H), 4.20 (t, 2H), 4.05 (t, 4H), 3.63 (bs, 4H), 2.82-2.24 (m, 36H), 1.92-1.74 (m, 6H), 1.68-1.55 (m, 12H), 1.50-1.27 (m, 28H), 1.22 (d, 12H), 0.92 (t, 6H).

APCI-MS analysis: Calculated C61H116N4O14S2 [M+H]=1193.7, Observed=1193.8.

### Bis(2-ethylbutyl) 9,9′-((4-(2-(4-(2-((3-(bis(2-hydroxy-9-oxo-9-propoxynonyl)amino)propyl)disulfaneyl)ethyl)piperazin-1-yl)ethoxy)-4-oxobutyl)azanediyl)bis(8-hydroxynonanoate) (GL-HEPES-E3-E9-Es6-DS-3-E9-E4)

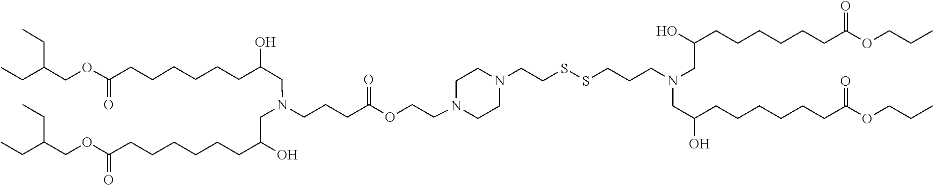 [SMILES: CCCOC(=O)CCCCCCC(O)CN(CCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCCCC(=O)OCCC]

¹H NMR (300 MHz, CDCl₃) δ 4.19 (t, 2H), 4.05 (t, 4H), 3.99 (d, 4H), 3.65 (bs, 4H), 2.84-2.39 (m, 28H), 2.29 (t, 4H), 2.28 (t, 4H), 1.92-1.74 (m, 6H), 1.68-1.55 (m, 14H), 1.52-1.24 (m, 44H), 0.92 (t, 6H), 0.88 (t, 12H).

APCI-MS analysis: Calculated C71H136N4O14S2 [M+H]=1334.0, Observed=1334.0.

### Dibutyl 9,9′-((4-(2-(4-(2-((3-(bis(2-hydroxy-7-isopentyloxy-7-oxoheptyl)amino)-propyl)disulfaneyl)ethyl)piperazin-1-yl)ethoxy)-4-pentyl)azanediyl)bis(8-hydroxynonanoate) (GL-HEPES-E4-E9-E4-DS-3-E7-Ei5)

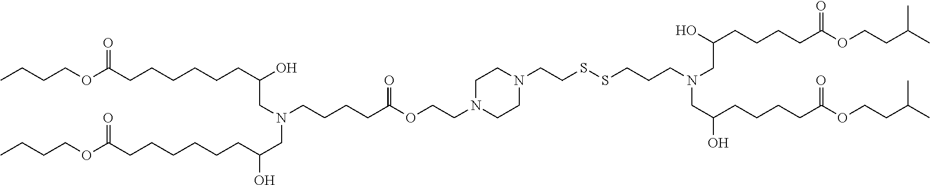 [SMILES: CCCCOC(=O)CCCCCCC(O)CN(CCCCC(=O)OCCN1CCN(CCSSCCCN(CC(O)CCCCC(=O)OCCC(C)C)CC(O)CCCCC(=O)OCCC(C)C)CC1)CC(O)CCCCCCC(=O)OCCCC]

¹H NMR (300 MHz, CDCl₃) δ 4.19 (t, 2H), 4.06 (t, 8H), 3.62 (m, 4H), 2.83-2.23 (m, 38H), 1.91-1.22 (m, 52H), 0.95-0.86 (m, 18H).

APCI-MS analysis: Calculated C66H126N4O14S2 [M+H]=1263.9, Observed=1263.9.

### Dibutyl 9,9′-((4-((2-(4-(2-((4-(bis(2-hydroxy-7-(isopentyloxy)-7-oxoheptyl)amino)-butanoyl)oxy)ethyl)piperazin-1-yl)ethyl)disulfaneyl)butyl)azanediyl)bis(8-hydroxynonanoate) (GL-HEPES-E3-E7-Ei5-DS-4-E9-E4)

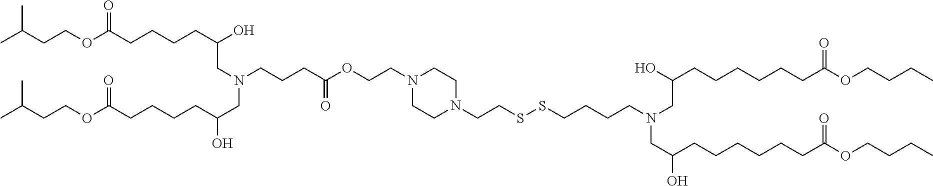 [SMILES: CCCCOC(=O)CCCCCCC(O)CN(CCCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCCCC]

¹H NMR (300 MHz, CDCl₃) δ 4.19 (t, 2H), 4.06 (t, 8H), 3.61 (m, 4H), 2.85-2.23 (m, 38H), 1.89-1.25 (m, 52H), 0.95-0.86 (m, 18H).

APCI-MS analysis: Calculated C66H126N4O14S2 [M+H]=1263.9, Observed=1263.9.

### Dibutyl 9,9′-((4-((2-(4-(2-((4-(bis(2-hydroxy-7-(isopentyloxy)-7-oxoheptyl)amino)-butanoyl)oxy)ethyl)piperazin-1-yl)ethyl)disulfaneyl)propyl)azanediyl)bis(8-hydroxynonanoate) (GL-HEPES-E3-E7Ei5-DS-3-E9E4)

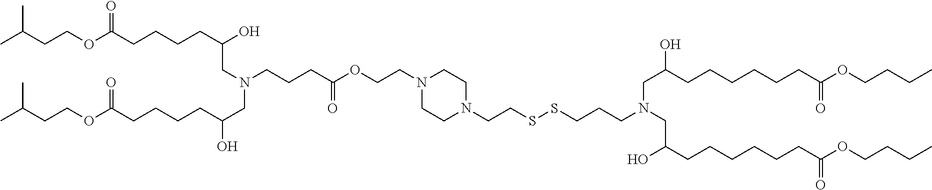 [SMILES: CCCCOC(=O)CCCCCCC(O)CN(CCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCCCC]

¹H NMR (300 MHz, CDCl₃) δ 4.19 (t, 2H), 4.08 (t, 8H), 3.62 (m, 4H), 2.85-2.23 (m, 38H), 1.90-1.25 (m, 50H), 0.95-0.86 (m, 18H).

APCI-MS analysis: Calculated C65H124N4O14S2 [M+H]=1249.8, Observed=1249.9.

### Diisopentyl 9,9′-((3-((2-(4-(2-((4-(bis(2-hydroxy-6-oxo-6-(pentan-3-yloxy)hexyl)amino)-butanoyl)oxy)ethyl)piperazin-1-yl)ethyl)disulfaneyl)propyl)azanediyl)bis(8-hydroxynonanoate) (GL-HEPES-E3-E6Es5-DS-3-E9Ei5)

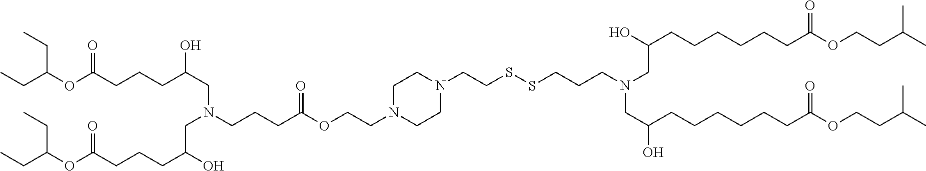 [SMILES: CCC(CC)OC(=O)CCCC(O)CN(CCCC(=O)OCCN1CCN(CCSSCCCN(CC(O)CCCCCCC(=O)OCCC(C)C)CC(O)CCCCCCC(=O)OCCC(C)C)CC1)CC(O)CCCC(=O)OC(CC)CC]

¹H NMR (300 MHz, CDCl₃) δ 4.77 (pent, 2H), 4.19 (t, 2H), 4.08 (t, 4H), 3.65 (m, 4H), 2.85-2.25 (m, 38H), 1.90-1.24 (m, 46H), 0.91 (d, 12H), 0.86 (t, 12H).

APCI-MS analysis: Calculated C65H124N4O14S2 [M+H]=1249.8, Observed=1249.8.

### Diisopentyl 9,9′-((3-((2-(4-(2-((4-(bis(2-hydroxy-7-isopropoxy-7-oxoheptyl)amino)-butanoyl)oxy)ethyl)piperazin-1-yl)ethyl)disulfaneyl)propyl)azanediyl)bis(8-hydroxynonanoate) (GL-HEPES-E3-E7Ei3-DS-3-E9Ei5)

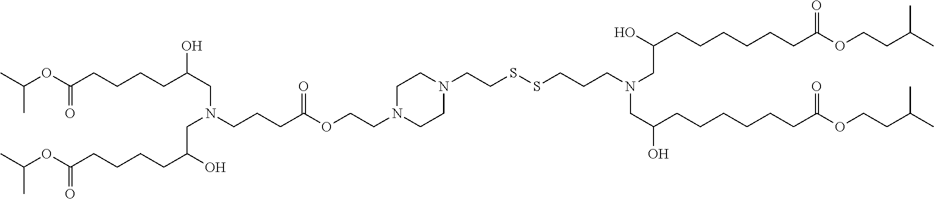 [SMILES: CC(C)CCOC(=O)CCCCCCC(O)CN(CCCSSCCN1CCN(CCOC(=O)CCCN(CC(O)CCCCC(=O)OC(C)C)CC(O)CCCCC(=O)OC(C)C)CC1)CC(O)CCCCCCC(=O)OCCC(C)C]

¹H NMR (300 MHz, CDCl₃) δ 4.99 (hept, 2H), 4.19 (t, 2H), 4.08 (t, 4H), 3.65 (m, 4H), 2.85-2.24 (m, 40H), 1.92-1.78 (m, 4H), 1.72-1.26 (m, 36H), 1.23 (d, 12H), 0.91 (t, 12H).

APCI-MS analysis: Calculated C63H120N4O14S2 [M+H]=1221.7, Observed=1221.8.

### Bis(2-ethylbutyl) 9,9′-((4-(2-(4-(2-((3-(bis(2-hydroxy-9-(isopentyloxy)-9-oxononyl)-amino)propyl)disulfaneyl)ethyl)piperazin-1-yl)ethoxy)-4-oxobutyl)azanediyl)bis(8-hydroxynonanoate) (GL-HEPES-E3-E9Es6-DS-3-E9Ei5)

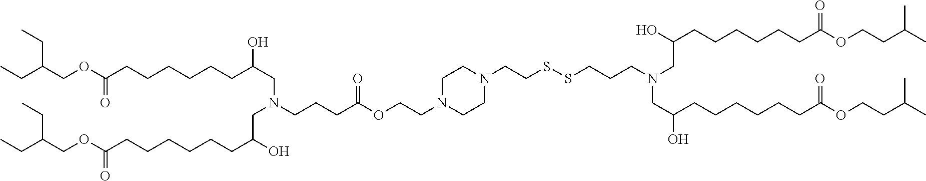 [SMILES: CCC(CC)COC(=O)CCCCCCC(O)CN(CCCC(=O)OCCN1CCN(CCSSCCCN(CC(O)CCCCCCC(=O)OCCC(C)C)CC(O)CCCCCCC(=O)OCCC(C)C)CC1)CC(O)CCCCCCC(=O)OCC(CC)CC]

¹H NMR (300 MHz, CDCl₃) δ 4.19 (t, 2H), 4.08 (t, 4H), 3.98 (d, 4H), 3.67 (m, 4H), 2.85-2.25 (m, 38H), 1.92-1.78 (m, 4H), 1.74-1.26 (m, 56H), 0.91 (d, 12H), 0.88 (t, 12H).

APCI-MS analysis: Calculated C73H140N4O14S2 [M+H]=1362.0, Observed=1362.0.

### Diisopentyl 7,7′-((4-(2-(4-(2-((3-(bis(2-hydroxy-7-isopentyloxy-7-oxoheptyl)amino)butyl)-disulfaneyl)ethyl)piperazin-1-yl)ethoxy)-4-oxobutyl)azanediyl)bis(6-hydroxyheptanoate) (GL-HEPES-E3-E7Ei5-DS-4-E7Ei5)

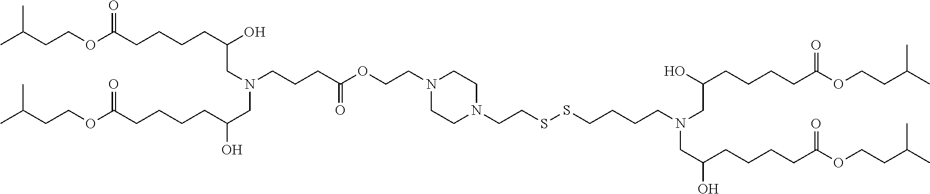 [SMILES: CC(C)CCOC(=O)CCCCC(O)CN(CCCCSSCCN1CCN(CCOC(=O)CCCN(CC(O)CCCCC(=O)OCCC(C)C)CC(O)CCCCC(=O)OCCC(C)C)CC1)CC(O)CCCCC(=O)OCCC(C)C]

¹H NMR (300 MHz, CDCl₃) δ 4.19 (t, 2H), 4.08 (t, 8H), 3.63 (m, 4H), 2.84-2.46 (m, 22H), 2.43-2.26 (m, 16H), 1.84-1.33 (m, 42H), 0.91 (d, 24H).

APCI-MS analysis: Calculated C64H122N4O14S2 [M+H]=1235.8, Observed=1235.9.

### Dibutyl 9,9′-((4-(2-(4-(2-((4-(bis(2-hydroxy-7-(isopentyloxy)-7-oxoheptyl)amino)butyl)-disulfaneyl)ethyl)piperazin-1-yl)ethoxy)-5-oxopentyl)azanediyl)bis(8-hydroxynonanoate) (GL-HEPES-E4-E9E4-DS-4-E7Ei5)

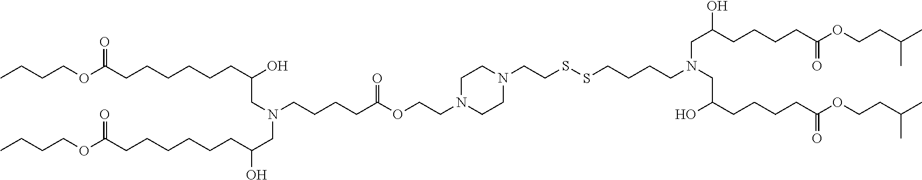 [SMILES: CCCCOC(=O)CCCCCCC(O)CN(CCCCC(=O)OCCN1CCN(CCSSCCCCN(CC(O)CCCCC(=O)OCCC(C)C)CC(O)CCCCC(=O)OCCC(C)C)CC1)CC(O)CCCCCCC(=O)OCCCC]

¹H NMR (300 MHz, CDCl₃) δ 4.19 (t, 2H), 4.08 (t, 4H), 4.05 (t, 4H), 3.62 (m, 4H), 2.86-2.46 (m, 22H), 2.45-2.25 (m, 16H), 1.79-1.25 (m, 54H), 0.92 (t, 6H), 0.90 (d, 12H).

APCI-MS analysis: Calculated C67H128N4O14S2 [M+H]=1277.9, Observed=1277.9.

### Bis(2-ethylbutyl) 7,7′-((3-((2-(4-(2-((4-(bis(2-hydroxy-7-(isopentyloxy)-7-oxoheptyl)amino)-butanoyl)oxy)ethyl)piperazin-1-yl)ethyl)disulfaneyl)propyl)azanediyl)bis(6-hydroxyheptanoate) (GL-HEPES-E3-E7Ei5-DS-3-E7Es6)

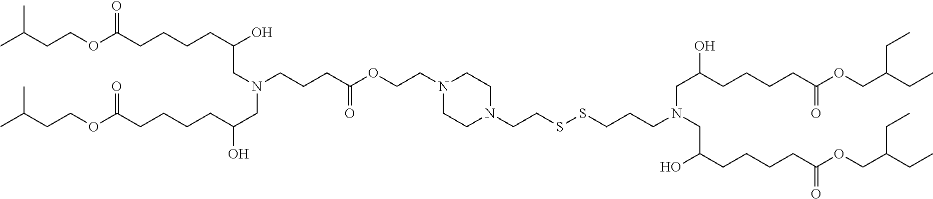 [SMILES: CCC(CC)COC(=O)CCCCC(O)CN(CCCSSCCN1CCN(CCOC(=O)CCCN(CC(O)CCCCC(=O)OCCC(C)C)CC(O)CCCCC(=O)OCCC(C)C)CC1)CC(O)CCCCC(=O)OCC(CC)CC]

¹H NMR (300 MHz, CDCl₃) δ 4.19 (t, 2H), 4.08 (t, 4H), 3.98 (d, 4H), 3.64 (m, 4H), 2.84-2.45 (m, 22H), 2.44-2.25 (m, 16H), 1.85-1.28 (m, 44H), 0.91 (d, 12H), 0.88 (t, 12H).

APCI-MS analysis: Calculated C65H124N4O14S2 [M+H]=1249.8, Observed=1249.9.

### Bis(2-ethylbutyl) 9,9′-((3-((2-(4-(2-((4-(bis(2-hydroxy-6-oxo-6-(pentan-3-yloxy)hexyl)amino)butanoyl)oxy)ethyl)piperazin-1-yl)ethyl)disulfaneyl)propyl)azanediyl)bis(8-hydroxynonanoate) (GL-HEPES-E3-E6Es5-DS-3-E9Es6)

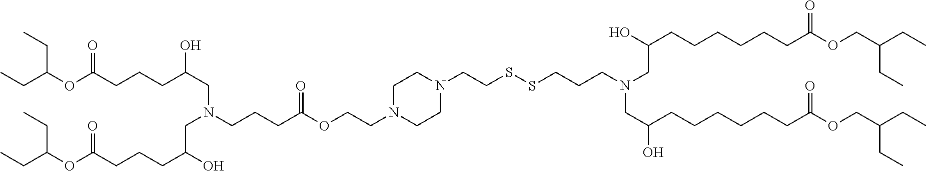 [SMILES: CCC(CC)COC(=O)CCCCCCC(O)CN(CCCSSCCN1CCN(CCOC(=O)CCCN(CC(O)CCCC(=O)OC(CC)CC)CC(O)CCCC(=O)OC(CC)CC)CC1)CC(O)CCCCCCC(=O)OCC(CC)CC]

¹H NMR (300 MHz, CDCl₃) δ 4.75 (pent, 2H), 4.19 (t, 2H), 3.98 (d, 4H), 3.64 (m, 4H), 2.85-2.25 (m, 40H), 1.90-1.24 (m, 52H), 0.88 (d, 12H), 0.86 (t, 12H).

APCI-MS analysis: Calculated C67H128N4O14S2 [M+H]=1277.9, Observed=1277.9.

### Bis(2-ethylbutyl) 9,9′-((3-((2-(4-(2-((4-(bis(2-hydroxy-7-isopropoxy-7-oxoheptyl)-amino)butanoyl)oxy)ethyl)piperazin-1-yl)ethyl)disulfaneyl)propyl)azanediyl)bis(8-hydroxynonanoate) (GL-HEPES-E3-E7Ei3-DS-3-E9Es6)

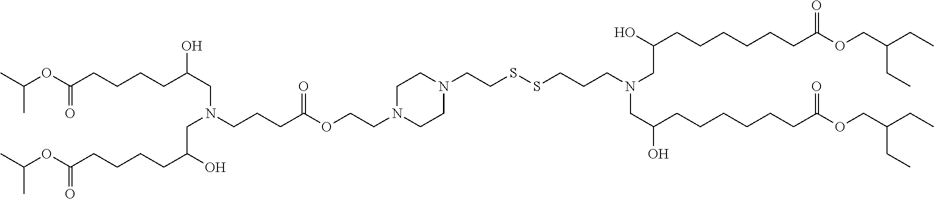 [SMILES: CCC(CC)COC(=O)CCCCCCC(O)CN(CCCSSCCN1CCN(CCOC(=O)CCCN(CC(O)CCCCC(=O)OC(C)C)CC(O)CCCCC(=O)OC(C)C)CC1)CC(O)CCCCCCC(=O)OCC(CC)CC]

¹H NMR (300 MHz, CDCl₃) δ 4.99 (hept, 2H), 4.19 (t, 2H), 3.98 (t, 4H), 3.64 (m, 4H), 2.85-2.24 (m, 36H), 1.90-1.78 (m, 4H), 1.68-1.26 (m, 44H), 1.22 (d, 12H), 0.88 (t, 12H).

APCI-MS analysis: Calculated C65H124N4O14S2 [M+H]=1249.8, Observed=1249.9.

### 7-Oxoheptyl)amino)propyl)disulfaneyl)ethyl)piperazin-1-yl)ethoxy)-5-oxopentyl)-azanediyl)bis(8-hydroxynonanoate) (GL-HEPES-E4-E9E4-DS-3-E7Es6)

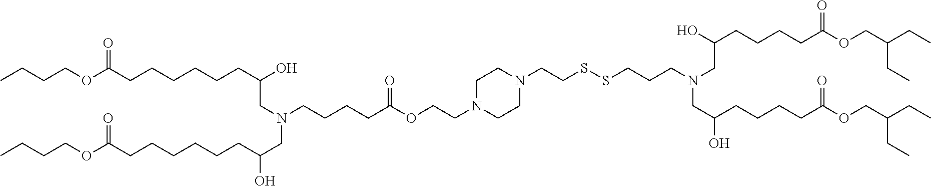 [SMILES: CCCCOC(=O)CCCCCCC(O)CN(CCCCC(=O)OCCN1CCN(CCSSCCCN(CC(O)CCCCC(=O)OCC(CC)CC)CC(O)CCCCC(=O)OCC(CC)CC)CC1)CC(O)CCCCCCC(=O)OCCCC]

¹H NMR (300 MHz, CDCl₃) δ 4.19 (t, 2H), 4.06 (t, 4H), 3.98 (d, 4H), 3.62 (m, 4H), 2.85-2.45 (m, 22H), 2.44-2.24 (m, 16H), 1.92-1.25 (m, 58H), 0.92 (t, 6H), 0.88 (t, 12H).

APCI-MS analysis: Calculated C68H130N4O14S2 [M+H]=1291.9, Observed=1291.9.

### Bis(2-ethylbutyl) 7,7′-((4-((2-(4-(2-((4-(bis(2-hydroxy-7-(isopentyloxy)-7-oxoheptyl)amino)-butanoyl)oxy)ethyl)piperazin-1-yl)ethyl)disulfaneyl)butyl)azanediyl)bis(6-hydroxyheptanoate) (GL-HEPES-E3-E7Ei5-DS-4-E7Es6)

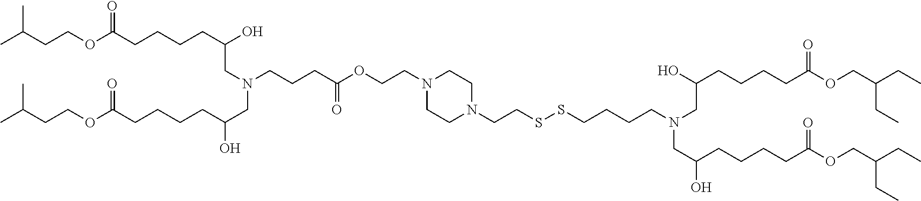 [SMILES: CCC(CC)COC(=O)CCCCC(O)CN(CCCCSSCCN1CCN(CCOC(=O)CCCN(CC(O)CCCCC(=O)OCCC(C)C)CC(O)CCCCC(=O)OCCC(C)C)CC1)CC(O)CCCCC(=O)OCC(CC)CC]

¹H NMR (300 MHz, CDCl₃) δ 4.19 (t, 2H), 4.08 (t, 4H), 3.98 (d, 4H), 3.61 (m, 4H), 2.84-2.45 (m, 22H), 2.44-2.25 (m, 16H), 1.86-1.28 (m, 46H), 0.91 (d, 12H), 0.88 (t, 12H).

APCI-MS analysis: Calculated C66H126N4O14S2 [M+H]=1263.8, Observed=1263.9.

### Dibutyl 9,9′-((5-(2-(4-(2-((4-(bis(7-(2-ethyl butoxy)-2-hydroxy-7-oxoheptyl)amino)butyl)disulfaneyl)ethyl)piperazin-1-yl)ethoxy)-5-oxopentyl)azanediyl)bis(8-hydroxynonanoate) (GL-HEPES-E4-E9E4-DS-4-E7Es6)

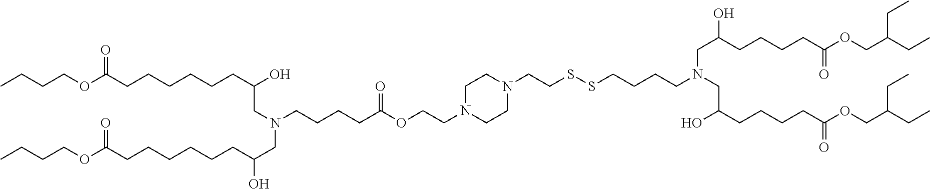 [SMILES: CCCCOC(=O)CCCCCCC(O)CN(CCCCC(=O)OCCN1CCN(CCSSCCCCN(CC(O)CCCCC(=O)OCC(CC)CC)CC(O)CCCCC(=O)OCC(CC)CC)CC1)CC(O)CCCCCCC(=O)OCCCC]

¹H NMR (300 MHz, CDCl₃) δ 4.19 (t, 2H), 4.06 (t, 4H), 3.98 (d, 4H), 3.61 (m, 4H), 2.85-2.45 (m, 22H), 2.44-2.24 (m, 16H), 1.78-1.26 (m, 58H), 0.92 (t, 6H), 0.88 (m, 12H).

APCI-MS analysis: Calculated C69H132N4O14S2 [M+H]=1305.9, Observed=1306.0.

### Diisopentyl 9,9′-((3-((2-(4-(2-((4-(bis(2-hydroxy-7-(isopentyloxy)-7-oxoheptyl)amino)butanoyl)oxy)ethyl)piperazin-1-yl)ethyl)disulfaneyl)propyl)azanediyl)bis(8-hydroxynonanoate) (GL-HEPES-E3-E7Ei5-DS-3-E9Ei5)

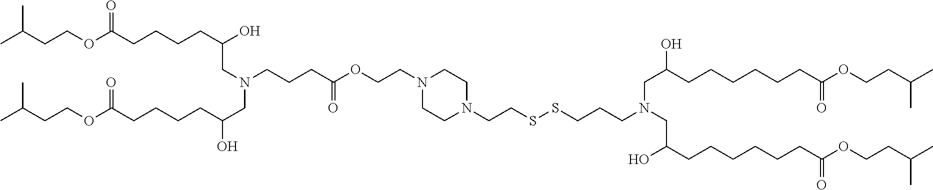 [SMILES: CC(C)CCOC(=O)CCCCCCC(O)CN(CCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCCC(C)C]

¹H NMR (300 MHz, CDCl₃) δ 4.19 (t, 2H), 4.08 (t, 8H), 3.63 (m, 4H), 2.84-2.46 (m, 22H), 2.43-2.23 (m, 16H), 1.91-1.29 (m, 48H), 0.91 (d, 24H).

APCI-MS analysis: Calculated C67H128N4O14S2 [M+H]=1277.9, Observed=1277.9.

### Dibutyl 9,9′-((5-(2-(4-(2-((3-(bis(2-hydroxy-9-(isopentyloxy)-9-oxononyl)amino)propyl)disulfaneyl)ethyl)piperazin-1-yl)ethoxy)-5-oxopentyl)azanediyl)bis(8-hydroxynonanoate) (GL-HEPES-E4-E9E4-DS-3-E9Ei5)

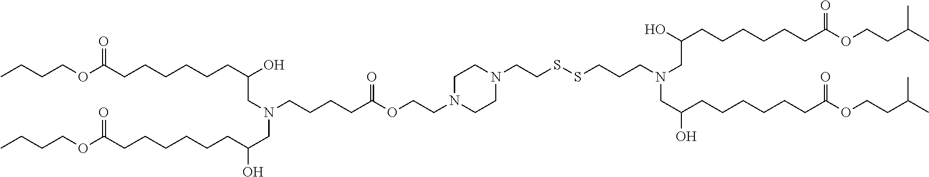 [SMILES: CCCCOC(=O)CCCCCCC(O)CN(CCCCC(=O)OCCN1CCN(CCSSCCCN(CC(O)CCCCCCC(=O)OCCC(C)C)CC(O)CCCCCCC(=O)OCCC(C)C)CC1)CC(O)CCCCCCC(=O)OCCCC]

1H NMR (300 MHz, CDCl₃) δ 4.19 (t, 2H), 4.08 (t, 4H), 4.06 (t, 4H), 3.61 (m, 4H), 2.86-2.46 (m, 22H), 2.45-2.25 (m, 16H), 1.91-1.25 (m, 60H), 0.92 (t, 6H), 0.91 (d, 12H).

APCI-MS analysis: Calculated C70H134N4O14S2 [M+H]=1319.9, Observed=1319.9.

### Bis(2-ethylbutyl) 9,9′-((3-((2-(4-(2-((4-(bis(9-(2-ethylbutoxy)-2-hydroxy-9-oxononyl)amino)butanoyl)oxy)ethyl)piperazin-1-yl)ethyl)disulfaneyl)propyl)azanediyl)bis(8-hydroxynonanoate) (GL-HEPES-E3-E9Es6-DS-3-E9Es6)

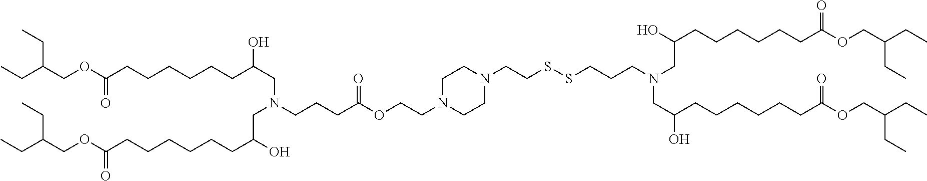 [SMILES: CCC(CC)COC(=O)CCCCCCC(O)CN(CCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCCCC(=O)OCC(CC)CC]

¹H NMR (300 MHz, CDCl₃) δ 4.20 (t, 2H), 3.98 (d, 8H), 3.67 (m, 4H), 2.88-2.35 (m, 30H), 2.29 (t, 8H), 1.96-1.78 (m, 4H), 1.70-1.28 (m, 60H), 0.88 (t, 24H).

APCI-MS analysis: Calculated C75H144N4O14S2 [M+H]=1390.1, Observed=1390.1.

### Diisopropyl 7,7′-((4-((2-(4-(2-((4-(bis(2-hydroxy-7-isopropoxy-7-oxoheptyl)amino)butanoyl)oxy)ethyl)piperazin-1-yl)ethyl)disulfaneyl)butyl)azanediyl)bis(6-hydroxyheptanoate) (GL-HEPES-E3-E7Ei3-DS-4-E7Ei3)

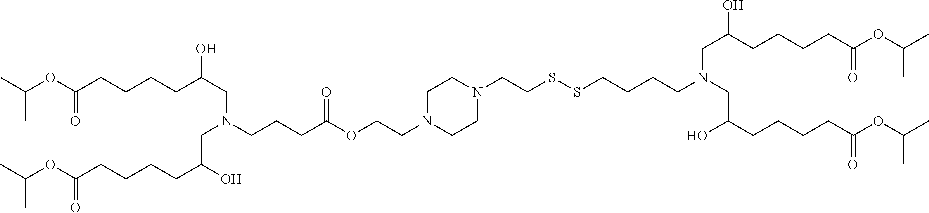 [SMILES: CC(C)OC(=O)CCCCC(O)CN(CCCCSSCCN1CCN(CCOC(=O)CCCN(CC(O)CCCCC(=O)OC(C)C)CC(O)CCCCC(=O)OC(C)C)CC1)CC(O)CCCCC(=O)OC(C)C]

¹H NMR (300 MHz, CDCl₃) δ 4.99 (hept, 4H), 4.20 (t, 2H), 3.65 (m, 4H), 2.85-2.34 (m, 28H), 2.27 (t, 8H), 1.92-1.32 (m, 36H), 1.22 (d, 24H).

APCI-MS analysis: Calculated C56H106N4O14S2 [M+H]=1123.6, Observed=1123.7.

### Bis(2-ethylbutyl) 9,9′-((4-(2-(4-(2-((4-(bis(2-hydroxy-7-isopropoxy-7-oxoheptyl)amino)-butyl)disulfaneyl)ethyl)piperazin-1-yl)ethoxy)-4-oxobutyl)azanediyl)bis(8-hydroxynonanoate) (GL-HEPES-E3-E9Es6-DS-4-E7Ei3)

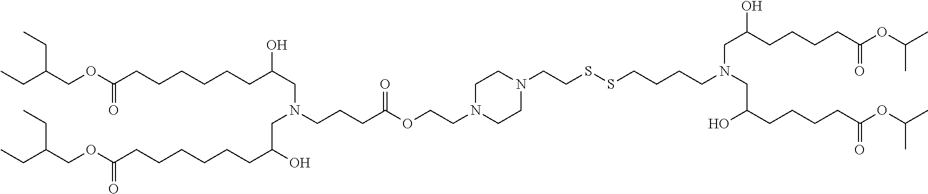 [SMILES: CCC(CC)COC(=O)CCCCCCC(O)CN(CCCC(=O)OCCN1CCN(CCSSCCCCN(CC(O)CCCCC(=O)OC(C)C)CC(O)CCCCC(=O)OC(C)C)CC1)CC(O)CCCCCCC(=O)OCC(CC)CC]

¹H NMR (300 MHz, CDCl₃) δ 4.99 (hept, 2H), 4.19 (t, 2H), 3.98 (d, 4H), 3.66 (m, 4H), 2.85-2.24 (m, 40H), 1.86-1.75 (m, 4H), 1.70-1.26 (m, 46H), 1.22 (d, 12H), 0.88 (t, 12H).

APCI-MS analysis: Calculated C66H126N4O14S2 [M+H]=1263.8, Observed=1263.9.

### Dibutyl 7,7′-((3-((2-(4-(2-((4-(bis(2-hydroxy-7-(isopentyloxy)-7-oxoheptyl)amino)-butanoyl)oxy)ethyl)piperazin-1-yl)ethyl)disulfaneyl)propyl)azanediyl)bis(6-hydroxyheptanoate) (GL-HEPES-E3-E7Ei5-DS-3-E7E4)

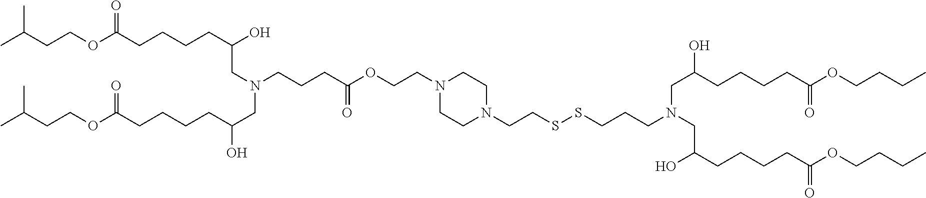 [SMILES: CCCCOC(=O)CCCCC(O)CN(CCCSSCCN1CCN(CCOC(=O)CCCN(CC(O)CCCCC(=O)OCCC(C)C)CC(O)CCCCC(=O)OCCC(C)C)CC1)CC(O)CCCCC(=O)OCCCC]

¹H NMR (300 MHz, CDCl₃) δ 4.19 (t, 2H), 4.08 (t, 4H), 4.06 (t, 4H), 3.64 (m, 4H), 2.86-2.46 (m, 22H), 2.45-2.25 (m, 16H), 1.91-1.28 (m, 42H), 0.92 (t, 6H), 0.91 (d, 12H).

APCI-MS analysis: Calculated C61H116N4O14S2 [M+H]=1193.7, Observed=1193.9.

### Dibutyl 9,9′-((5-(2-(4-(2-((3-(bis(7-butoxy-2-hydroxy-7-oxoheptyl)amino)-propyl)disulfaneyl)ethyl)piperazin-1-yl)ethoxy)-5-oxopentyl)azanediyl)bis(8-hydroxynonanoate) (GL-HEPES-E4-E9E4-DS-3-E7E4)

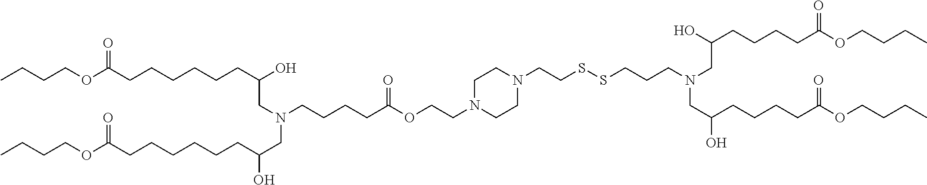 [SMILES: CCCCOC(=O)CCCCCCC(O)CN(CCCCC(=O)OCCN1CCN(CCSSCCCN(CC(O)CCCCC(=O)OCCCC)CC(O)CCCCC(=O)OCCCC)CC1)CC(O)CCCCCCC(=O)OCCCC]

¹H NMR (300 MHz, CDCl₃) δ 4.19 (t, 2H), 4.06 (t, 8H), 3.66 (m, 4H), 2.85-2.46 (m, 22H), 2.43-2.23 (m, 16H), 1.90-1.70 (m, 4H), 1.69-1.25 (m, 54H), 0.92 (t, 12H).

APCI-MS analysis: Calculated C64H122N4O14S2 [M+H]=1235.8, Observed=1235.9.

### Dibutyl 7,7′-((4-((2-(4-(2-((4-(bis(2-hydroxy-7-(isopentyloxy)-7-oxoheptyl)amino)-butanoyl)oxy)ethyl)piperazin-1-yl)ethyl)disulfaneyl)butyl)azanediyl)bis(6-hydroxyheptanoate) (GL-HEPES-E3-E7Ei5-DS-4-E7E4)

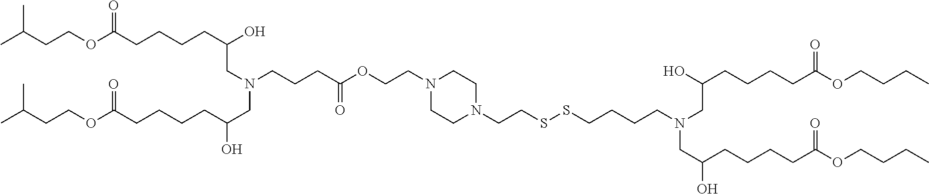 [SMILES: CCCCOC(=O)CCCCC(O)CN(CCCCSSCCN1CCN(CCOC(=O)CCCN(CC(O)CCCCC(=O)OCCC(C)C)CC(O)CCCCC(=O)OCCC(C)C)CC1)CC(O)CCCCC(=O)OCCCC]

¹H NMR (300 MHz, CDCl₃) δ 4.19 (t, 2H), 4.08 (t, 4H), 4.06 (t, 4H), 3.62 (m, 4H), 2.82-2.46 (m, 22H), 2.43-2.25 (m, 16H), 1.86-1.22 (m, 44H), 0.92 (t, 6H), 0.91 (d, 12H).

APCI-MS analysis: Calculated C62H118N4O14S2 [M+H]=1207.7, Observed=1207.8.

### Dibutyl 9,9′-((5-(2-(4-(2-((4-(bis(7-butoxy-2-hydroxy-7-oxoheptyl)amino)-butyl)disulfaneyl)ethyl)piperazin-1-yl)ethoxy)-5-oxopentyl)azanediyl)bis(8-hydroxynonanoate) (GL-HEPES-E4-E9E4-DS-4-E7E4)

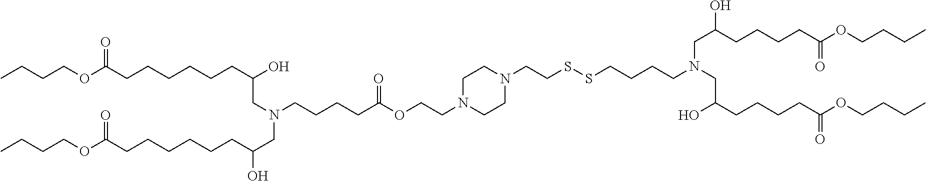 [SMILES: CCCCOC(=O)CCCCCCC(O)CN(CCCCC(=O)OCCN1CCN(CCSSCCCCN(CC(O)CCCCC(=O)OCCCC)CC(O)CCCCC(=O)OCCCC)CC1)CC(O)CCCCCCC(=O)OCCCC]

¹H NMR (300 MHz, CDCl₃) δ 4.19 (t, 2H), 4.06 (t, 8H), 3.61 (m, 4H), 2.83-2.25 (m, 38H), 1.85-1.25 (m, 56H), 0.92 (t, 12H).

APCI-MS analysis: Calculated C65H124N4O14S2 [M+H]=1249.8, Observed=1249.9.

### Dibutyl 7,7′-((4-((2-(4-(2-((4-(bis(7-(2-ethylbutoxy)-2-hydroxy-7-oxoheptyl)amino)-butanoyl)oxy)ethyl)piperazin-1-yl)ethyl)disulfaneyl)butyl)azanediyl)bis(6-hydroxyheptanoate) (GL-HEPES-E3-E7Es6-DS-4-E7E4)

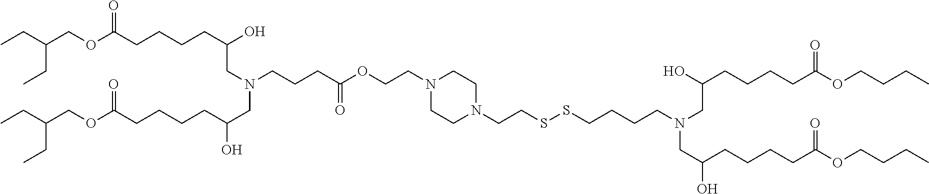 [SMILES: CCCCOC(=O)CCCCC(O)CN(CCCCSSCCN1CCN(CCOC(=O)CCCN(CC(O)CCCCC(=O)OCC(CC)CC)CC(O)CCCCC(=O)OCC(CC)CC)CC1)CC(O)CCCCC(=O)OCCCC]

¹H NMR (300 MHz, CDCl₃) δ 4.19 (t, 2H), 4.06 (t, 4H), 3.98 (d, 4H), 3.61 (m, 4H), 2.84-2.25 (m, 38H), 1.90-1.22 (m, 48H), 0.92 (t, 6H), 0.88 (t, 12H).

APCI-MS analysis: Calculated C64H122N4O14S2 [M+H]=1235.8, Observed=1235.9.

### Dibutyl 9,9′-((4-(2-(4-(2-((4-(bis(2-hydroxy-7-(isopentyloxy)-7-oxoheptyl)amino)-butyl)disulfaneyl)ethyl)piperazin-1-yl)ethoxy)-4-oxobutyl)azanediyl)bis(8-hydroxynonanoate) (GL-HEPES-E3-E9E4-DS-4-E7E4)

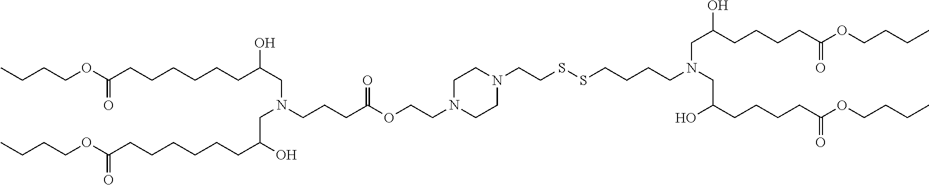 [SMILES: CCCCOC(=O)CCCCCCC(O)CN(CCCC(=O)OCCN1CCN(CCSSCCCCN(CC(O)CCCCC(=O)OCCCC)CC(O)CCCCC(=O)OCCCC)CC1)CC(O)CCCCCCC(=O)OCCCC]

¹H NMR (300 MHz, CDCl₃) δ 4.19 (t, 2H), 4.06 (t, 8H), 3.61 (m, 4H), 2.83-2.25 (m, 38H), 1.85-1.25 (m, 54H), 0.92 (t, 12H).

APCI-MS analysis: Calculated C64H122N4O14S2 [M+H]=1235.8, Observed=1235.9.

### Diisopentyl 7,7′-((4-((2-(4-(2-((4-(bis(2-hydroxy-7-isopropoxy-7-oxoheptyl)amino)-butanoyl)oxy)ethyl)piperazin-1-yl)ethyl)disulfaneyl)butyl)azanediyl)bis(6-hydroxyheptanoate) (GL-HEPES-E3-E7Ei3-DS-4-E7Ei5)

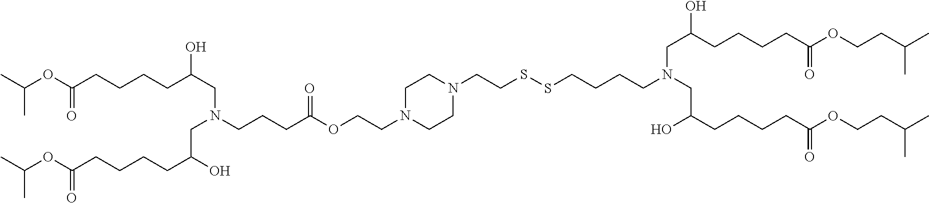 [SMILES: CC(C)CCOC(=O)CCCCC(O)CN(CCCCSSCCN1CCN(CCOC(=O)CCCN(CC(O)CCCCC(=O)OC(C)C)CC(O)CCCCC(=O)OC(C)C)CC1)CC(O)CCCCC(=O)OCCC(C)C]

¹H NMR (300 MHz, CDCl₃) δ 4.99 (hept, 4H), 4.19 (t, 2H), 4.08 (t, 4H), 3.63 (m, 4H), 2.84-2.24 (m, 36H), 1.84-1.32 (m, 40H), 1.22 (d, 12H), 0.91 (d, 12H).

APCI-MS analysis: Calculated C60H114N4O14S2 [M+H]=1179.7, Observed=1179.8.

### Dibutyl 7,7′-((3-((2-(4-(2-((4-(bis(2-hydroxy-7-isopropoxy-7-oxoheptyl)amino)-butanoyl)oxy)ethyl)piperazin-1-yl)ethyl)disulfaneyl)propyl)azanediyl)bis(6-hydroxyheptanoate) (GL-HEPES-E3-E7Ei3-DS-3-E7E4)

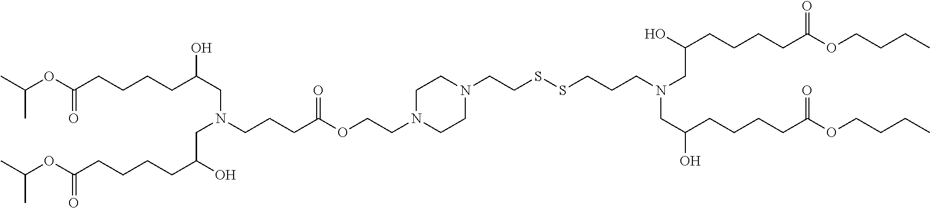 [SMILES: CCCCOC(=O)CCCCC(O)CN(CCCSSCCN1CCN(CCOC(=O)CCCN(CC(O)CCCCC(=O)OC(C)C)CC(O)CCCCC(=O)OC(C)C)CC1)CC(O)CCCCC(=O)OCCCC]

¹H NMR (300 MHz, CDCl₃) δ 4.99 (hept, 4H), 4.20 (t, 2H), 4.06 (t, 4H), 3.64 (m, 4H), 2.87-2.24 (m, 36H), 1.90-1.32 (m, 40H), 1.22 (d, 12H), 0.92 (t, 6H).

APCI-MS analysis: Calculated C57H108N4O14S2 [M+H]=1136.7, Observed=1137.8.

### Bis(2-ethylbutyl) 9,9′-((4-(2-(4-(2-((3-(bis(7-butoxy-2-hydroxy-7-oxoheptyl)amino)-propyl)disulfaneyl)ethyl)piperazin-1-yl)ethoxy)-4-oxobutyl)azanediyl)bis(8-hydroxynonanoate) (GL-HEPES-E3-E9Es6-DS-3-E7E4)

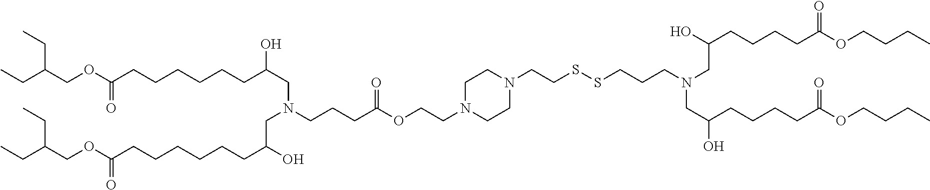 [SMILES: CCCCOC(=O)CCCCC(O)CN(CCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCC(=O)OCCCC]

¹H NMR (300 MHz, CDCl₃) δ 4.20 (t, 2H), 4.06 (t, 4H), 3.98 (d, 4H), 3.64 (m, 4H), 2.84-2.24 (m, 40H), 1.92-1.26 (m, 56H), 0.92 (d, 6H), 0.88 (t, 12H).

APCI-MS analysis: Calculated C67H128N4O14S2 [M+H]=1277.8, Observed=1277.9.

### Bis(2-ethylbutyl) 9,9′-((3-((2-(4-(2-((4-(bis(2-hydroxy-7-(isopentyloxy)-7-oxoheptyl)amino)-butanoyl)oxy)ethyl)piperazin-1-yl)ethyl)disulfaneyl)propyl)azanediyl)bis(8-hydroxynonanoate) (GL-HEPES-E3-E7Ei5-DS-3-E9Es6)

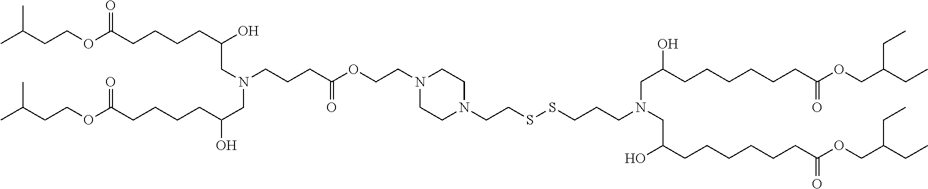 [SMILES: CCC(CC)COC(=O)CCCCCCC(O)CN(CCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCC(CC)CC]

¹H NMR (300 MHz, CDCl₃) δ 4.19 (t, 2H), 4.08 (t, 4H), 3.98 (d, 4H), 3.61 (m, 4H), 2.84-2.46 (m, 22H), 2.45-2.23 (m, 16H), 1.91-1.29 (m, 52H), 0.91 (d, 12H), 0.88 (t, 12H).

APCI-MS analysis: Calculated C69H132N4O14S2 [M+H]=1305.9, Observed=1305.9.

### Dibutyl 9,9′-((5-(2-(4-(2-((3-(bis(9-(2-ethyl butoxy)-2-hydroxy-9-oxononyl)amino)-propyl)disulfaneyl)ethyl)piperazin-1-yl)ethoxy)-5-oxopentyl)azanediyl)bis(8-hydroxynonanoate) (GL-HEPES-E4-E9E4-DS-3-E9Es6)

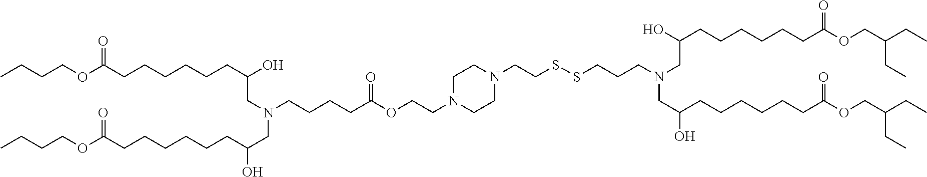 [SMILES: CCCCOC(=O)CCCCCCC(O)CN(CCCCC(=O)OCCN1CCN(CCSSCCCN(CC(O)CCCCCCC(=O)OCC(CC)CC)CC(O)CCCCCCC(=O)OCC(CC)CC)CC1)CC(O)CCCCCCC(=O)OCCCC]

¹H NMR (300 MHz, CDCl₃) δ 4.19 (t, 2H), 4.06 (t, 4H), 3.98 (d, 4H), 3.62 (m, 4H), 2.86-2.46 (m, 22H), 2.45-2.23 (m, 16H), 1.91-1.23 (m, 64H), 0.92 (t, 6H), 0.88 (t, 12H).

APCI-MS analysis: Calculated C72H138N4O14S2 [M+H]=1348.0, Observed=1348.0.

### Diisopentyl 9,9′-((4-((2-(4-(2-((4-(bis(2-hydroxy-7-(isopentyloxy)-7-oxoheptyl)amino)butanoyl)oxy)ethyl)piperazin-1-yl)ethyl)disulfaneyl)butyl)azanediyl)bis(8-hydroxynonanoate) (GL-HEPES-E3-E7Ei5-DS-4-E9Ei5)

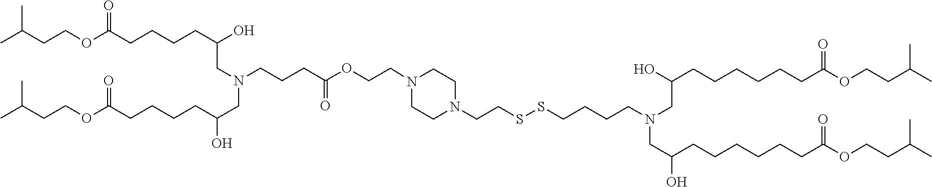 [SMILES: CC(C)CCOC(=O)CCCCCCC(O)CN(CCCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCCC(C)C]

¹HNMR (300 MHz, CDCl₃) δ 4.19 (t, 2H), 4.08 (t, 8H), 3.61 (m, 4H), 2.85-2.21 (m, 38H), 1.85-1.25 (m, 50H), 0.91 (d, 24H).

APCI-MS analysis: Calculated C68H130N4O14S2 [M+H]=1291.9, Observed=1291.8.

### Dibutyl 7,7′-((4-((2-(4-(2-((4-(bis(2-hydroxy-6-oxo-6-(pentan-3-yloxy)hexyl)amino)-butanoyl)oxy)ethyl)piperazin-1-yl)ethyl)disulfaneyl)butyl)azanediyl)bis(6-hydroxyheptanoate) (GL-HEPES-E3-E6Es5-DS-4-E7E4)

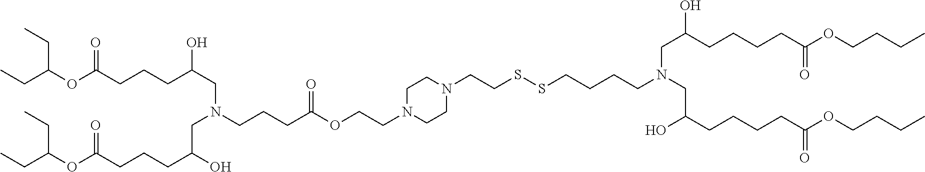 [SMILES: CCCCOC(=O)CCCCC(O)CN(CCCCSSCCN1CCN(CCOC(=O)CCCN(CC(O)CCCC(=O)OC(CC)CC)CC(O)CCCC(=O)OC(CC)CC)CC1)CC(O)CCCCC(=O)OCCCC]

¹H NMR (300 MHz, CDCl₃) δ 4.75 (pent, 2H), 4.20 (t, 2H), 4.06 (t, 4H), 3.63 (m, 4H), 2.85-2.28 (m, 30H), 1.84-1.33 (m, 54H), 0.92 (d, 6H), 0.86 (t, 12H).

APCI-MS analysis: Calculated C60H114N4O14S2 [M+H]=1179.7, Observed=1179.8.

### Dibutyl 7,7′-((4-((2-(4-(2-((4-(bis(2-hydroxy-7-isopropoxy-7-oxoheptyl)amino)-butanoyl)oxy)ethyl)piperazin-1-yl)ethyl)disulfaneyl)butyl)azanediyl)bis(6-hydroxyheptanoate) (GL-HEPES-E3-E7Ei3-DS-4-E7E4)

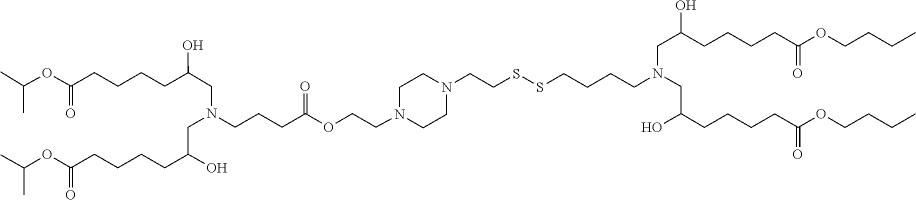 [SMILES: CCCCOC(=O)CCCCC(O)CN(CCCCSSCCN1CCN(CCOC(=O)CCCN(CC(O)CCCCC(=O)OC(C)C)CC(O)CCCCC(=O)OC(C)C)CC1)CC(O)CCCCC(=O)OCCCC]

¹H NMR (300 MHz, CDCl₃) δ 4.99 (hept, 2H), 4.19 (t, 2H), 4.06 (t, 4H), 3.62 (m, 4H), 2.84-2.24 (m, 34H), 1.85-1.30 (m, 40H), 1.22 (d, 12H), 0.92 (t, 12H).

APCI-MS analysis: Calculated C58H110N4O14S2 [M+H]=1151.6, Observed=1151.7.

### Bis(2-ethylbutyl) 9,9′-((4-(2-(4-(2-((4-(bis(7-butoxy-2-hydroxy-7-oxoheptyl)amino)-butyl)disulfaneyl)ethyl)piperazin-1-yl)ethoxy)-4-oxobutyl)azanediyl)bis(8-hydroxynonanoate) (GL-HEPES-E3-E9Es6-DS-4-E7E4)

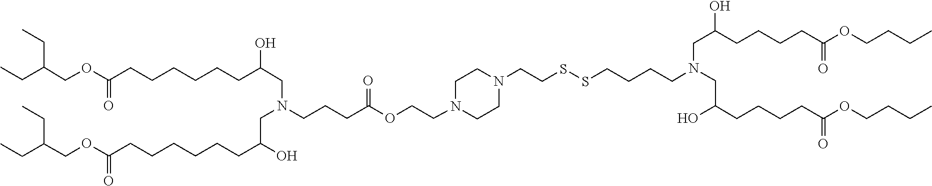 [SMILES: CCCCOC(=O)CCCCC(O)CN(CCCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCC(=O)OCCCC]

¹H NMR (300 MHz, CDCl₃) δ 4.19 (t, 2H), 4.06 (t, 4H), 3.98 (d, 4H), 3.61 (m, 4H), 2.84-2.26 (m, 34H), 1.85-1.28 (m, 60H), 0.92 (t, 6H), 0.88 (t, 12H).

APCI-MS analysis: Calculated C68H130N4O14S2 [M+H]=1291.9, Observed=1291.9.

### Dibutyl 9,9′-((5-(2-(4-(2-((4-(bis(2-hydroxy-9-(isopentyloxy)-9-oxononyl)-amino)butyl)disulfaneyl)ethyl)piperazin-1-yl)ethoxy)-5-oxopentyl)azanediyl)-bis(8-hydroxynonanoate) (GL-HEPES-E4-E9E4-DS-4-E9Ei5)

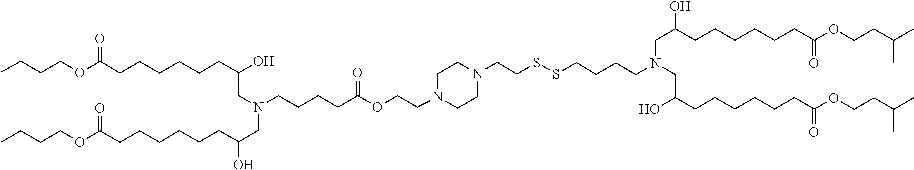 [SMILES: CCCCOC(=O)CCCCCCC(O)CN(CCCCC(=O)OCCN1CCN(CCSSCCCCN(CC(O)CCCCCCC(=O)OCCC(C)C)CC(O)CCCCCCC(=O)OCCC(C)C)CC1)CC(O)CCCCCCC(=O)OCCCC]

NMR (300 MHz, CDCl₃) δ 4.19 (t, 2H), 4.08 (t, 4H), 4.06 (t, 4H), 3.61 (m, 4H), 2.85-2.25 (m, 38H), 1.80-1.25 (m, 62H), 0.92 (t, 6H), 0.91 (d, 12H).

APCI-MS analysis: Calculated C71H136N4O14S2 [M+H]=1334.0, Observed=1334.0.

### Bis(2-ethylbutyl) 9,9′-((4-((2-(4-(2-((4-(bis(2-hydroxy-7-(isopentyloxy)-7-oxoheptyl)amino)-butanoyl)oxy)ethyl)piperazin-1-yl)ethyl)disulfaneyl)butyl)azanediyl)bis(8-hydroxynonanoate) (GL-HEPES-E3-E7Ei5-DS-4-E9Es6)

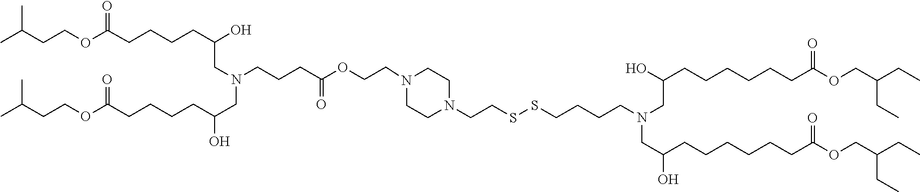 [SMILES: CCC(CC)COC(=O)CCCCCCC(O)CN(CCCCSSCCN1CCN(CCOC(=O)CCCN(CC(O)CCCCC(=O)OCCC(C)C)CC(O)CCCCC(=O)OCCC(C)C)CC1)CC(O)CCCCCCC(=O)OCC(CC)CC]

¹HNMR (300 MHz, CDCl₃) δ 4.19 (t, 2H), 4.08 (t, 4H), 3.98 (d, 4H), 3.61 (m, 4H), 2.84-2.45 (m, 22H), 2.44-2.25 (m, 16H), 1.83-1.28 (m, 54H), 0.91 (d, 12H), 0.88 (t, 12H).

APCI-MS analysis: Calculated C70H134N4O14S2 [M+H]=1318.9, Observed=1319.0.

### Dibutyl 9,9′-((5-(2-(4-(2-((4-(bis(9-(2-ethyl butoxy)-2-hydroxy-9-oxononyl)amino)-butyl)disulfaneyl)ethyl)piperazin-1-yl)ethoxy)-5-oxopentyl)azanediyl)bis(8-hydroxynonanoate) (GL-HEPES-E4-E9E4-DS-4-E9Es6)

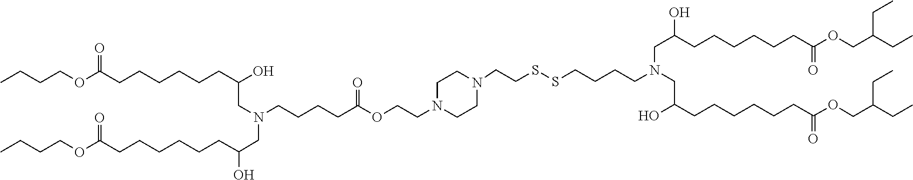 [SMILES: CCCCOC(=O)CCCCCCC(O)CN(CCCCC(=O)OCCN1CCN(CCSSCCCCN(CC(O)CCCCCCC(=O)OCC(CC)CC)CC(O)CCCCCCC(=O)OCC(CC)CC)CC1)CC(O)CCCCCCC(=O)OCCCC]

¹HNMR (300 MHz, CDCl₃) δ 4.19 (t, 2H), 4.05 (t, 4H), 3.98 (d, 4H), 3.61 (m, 4H), 2.84-2.45 (m, 22H), 2.44-2.25 (m, 16H), 1.77-1.26 (m, 66H), 0.92 (t, 6H), 0.88 (t, 12H).

APCI-MS analysis: Calculated C73H140N4O14S2 [M+H]=1362.0, Observed=1362.0.

### Bis(2-ethylbutyl) 7,7′-((4-((2-(4-(2-((4-(bis(2-hydroxy-7-isopropoxy-7-oxoheptyl)amino)-butanoyl)oxy)ethyl)piperazin-1-yl)ethyl)disulfaneyl)butyl)azanediyl)bis(6-hydroxyheptanoate) (GL-HEPES-E3-E7Ei3-DS-4-E7Es6)

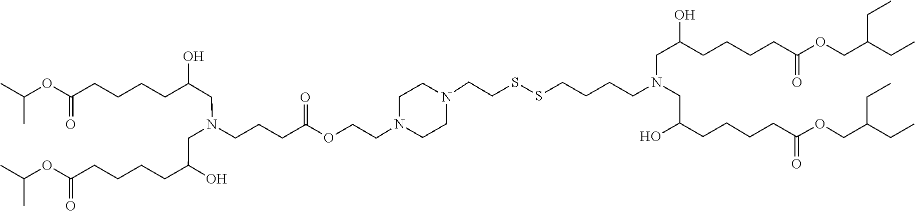 [SMILES: CCC(CC)COC(=O)CCCCC(O)CN(CCCCSSCCN1CCN(CCOC(=O)CCCN(CC(O)CCCCC(=O)OC(C)C)CC(O)CCCCC(=O)OC(C)C)CC1)CC(O)CCCCC(=O)OCC(CC)CC]

¹H NMR (300 MHz, CDCl₃) δ 5.01 (hept, 2H), 4.19 (t, 2H), 3.98 (d, 4H), 3.63 (m, 4H), 2.83-2.24 (m, 34H), 1.82-1.29 (m, 44H), 1.22 (d, 12H), 0.88 (t, 12H).

APCI-MS analysis: Calculated C62H118N4O14S2 [M+H]=1207.7, Observed=1207.8.

### Bis(2-ethylbutyl) 9,9′-((4-(2-(4-(2-((4-(bis(7-(2-ethylbutoxy)-2-hydroxy-7-oxoheptyl)amino)-butyl)disulfaneyl)ethyl)piperazin-1-yl)ethoxy)-4-oxobutyl)azanediyl)bis(8-hydroxynonanoate) (GL-HEPES-E3-E9Es6-DS-4-E7Es6)

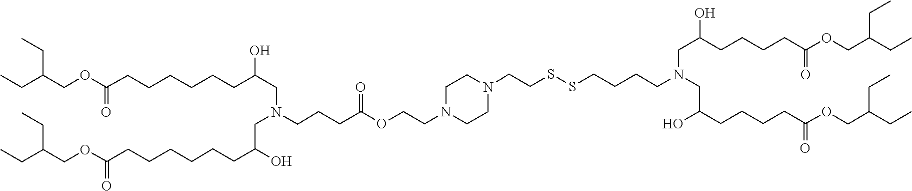 [SMILES: CCC(CC)COC(=O)CCCCCCC(O)CN(CCCC(=O)OCCN1CCN(CCSSCCCCN(CC(O)CCCCC(=O)OCC(CC)CC)CC(O)CCCCC(=O)OCC(CC)CC)CC1)CC(O)CCCCCCC(=O)OCC(CC)CC]

¹H NMR (300 MHz, CDCl₃) δ 4.19 (t, 2H), 3.98 (d, 8H), 3.61 (m, 4H), 2.84-2.26 (m, 36H), 1.83-1.28 (m, 64H), 0.88 (t, 24H).

APCI-MS analysis: Calculated C72H138N4O14S2 [M+H]=1348.0, Observed=1348.0.

### Dibutyl 9,9′-((4-((2-(4-(2-((4-(bis(2-hydroxy-7-isopropoxy-7-oxoheptyl)amino)-butanoyl)oxy)ethyl)piperazin-1-yl)ethyl)disulfaneyl)butyl)azanediyl)bis(8-hydroxynonanoate (GL-HEPES-E3-E7Ei3-DS-4-E9E4)

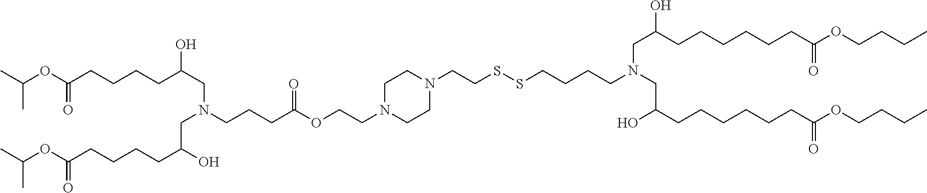 [SMILES: CCCCOC(=O)CCCCCCC(O)CN(CCCCSSCCN1CCN(CCOC(=O)CCCN(CC(O)CCCCC(=O)OC(C)C)CC(O)CCCCC(=O)OC(C)C)CC1)CC(O)CCCCCCC(=O)OCCCC]

¹H NMR (300 MHz, CDCl₃) δ 4.99 (hept, 2H), 4.19 (t, 2H), 4.06 (t, 4H), 3.61 (m, 4H), 2.83-2.24 (m, 36H), 1.82-1.27 (m, 52H), 1.22 (d, 12H), 0.93 (t, 6H).

APCI-MS analysis: Calculated C62H118N4O14S2 [M+H]=1207.7, Observed=1207.8.

### Dibutyl 9,9′-((4-((2-(4-(2-((4-(bis(9-(2-ethylbutoxy)-2-hydroxy-9-oxononyl)amino)-butanoyl)oxy)ethyl)piperazin-1-yl)ethyl)disulfaneyl)butyl)azanediyl)bis(8-hydroxynonanoate) (GL-HEPES-E3-E9Es6-DS-4-E9E4)

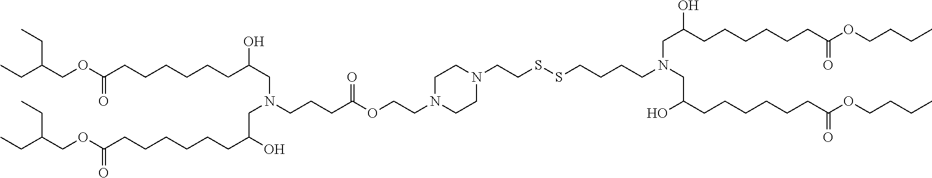 [SMILES: CCCCOC(=O)CCCCCCC(O)CN(CCCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCCCC(=O)OCCCC]

¹H NMR (300 MHz, CDCl₃) δ 4.19 (t, 2H), 4.06 (t, 4H), 3.98 (d, 4H), 3.62 (m, 4H), 2.84-2.25 (m, 36H), 1.83-1.28 (m, 70H), 0.92 (t, 6H), 0.88 (t, 12H).

APCI-MS analysis: Calculated C72H138N4O14S2 [M+H]=1348.0, Observed=1348.0.

### Diisopentyl 9,9′-((5-(2-(4-(2-((3-(bis(2-hydroxy-7-isopropoxy-7-oxoheptyl)amino)-propyl)disulfaneyl)ethyl)piperazin-1-yl)ethoxy)-5-oxopentyl)azanediyl)bis(8-hydroxynonanoate) (GL-HEPES-E4-E9Ei5-DS-3-E7Ei3)

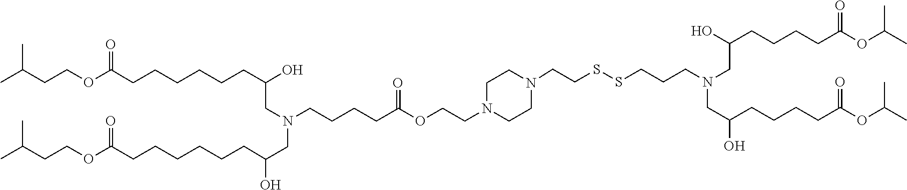 [SMILES: CC(C)CCOC(=O)CCCCCCC(O)CN(CCCCC(=O)OCCN1CCN(CCSSCCCN(CC(O)CCCCC(=O)OC(C)C)CC(O)CCCCC(=O)OC(C)C)CC1)CC(O)CCCCCCC(=O)OCCC(C)C]

¹H NMR (300 MHz, CDCl₃) δ 4.99 (hept, 2H), 4.19 (t, 2H), 4.08 (t, 4H), 3.62 (m, 4H), 2.85-2.22 (m, 38H), 1.85-1.24 (m, 44H), 1.22 (d, 12H), 0.91 (d, 12H).

APCI-MS analysis: Calculated C64H122N4O14S2 [M+H]=1235.8, Observed=1235.9.

### Dibutyl 9,9′-((4-(2-(4-(2-((3-(bis(2-hydroxy-7-isopropoxy-7-oxoheptyl)amino)propyl)disulfaneyl)ethyl)piperazin-1-yl)ethoxy)-4-pentyl)azanediyl)bis(8-hydroxynonanoate) (GL-HEPES-E4-E9E4-DS-3-E7Ei3)

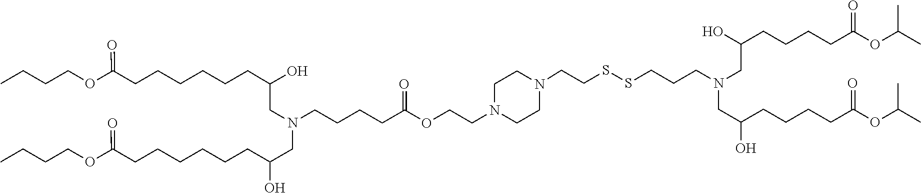 [SMILES: CCCCOC(=O)CCCCCCC(O)CN(CCCCC(=O)OCCN1CCN(CCSSCCCN(CC(O)CCCCC(=O)OC(C)C)CC(O)CCCCC(=O)OC(C)C)CC1)CC(O)CCCCCCC(=O)OCCCC]

¹H NMR (300 MHz, CDCl₃) δ 4.99 (hept, 2H), 4.18 (t, 2H), 4.05 (t, 4H), 3.64 (m, 4H), 2.86-2.21 (m, 38H), 1.90-1.28 (m, 46H), 1.22 (d, 12H), 0.90 (t, 6H).

APCI-MS analysis: Calculated C62H118N4O14S2 [M+H]=1207.8, Observed=1207.8.

### Diisopentyl 9,9′-((5-(2-(4-(2-((4-(bis(2-hydroxy-7-isopropoxy-7-oxoheptyl)amino)-butyl)disulfaneyl)ethyl)piperazin-1-yl)ethoxy)-5-oxopentyl)azanediyl)bis(8-hydroxynonanoate) (GL-HEPES-E4-E9Ei5-DS-4-E7Ei3)

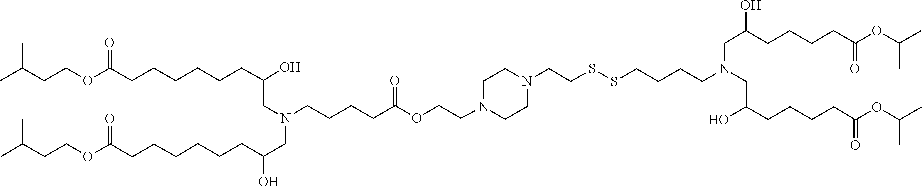 [SMILES: CC(C)CCOC(=O)CCCCCCC(O)CN(CCCCC(=O)OCCN1CCN(CCSSCCCCN(CC(O)CCCCC(=O)OC(C)C)CC(O)CCCCC(=O)OC(C)C)CC1)CC(O)CCCCCCC(=O)OCCC(C)C]

¹H NMR (300 MHz, CDCl₃) δ 4.99 (hept, 2H), 4.19 (t, 2H), 4.08 (t, 4H), 3.61 (m, 4H), 2.85-2.22 (m, 38H), 1.78-1.24 (m, 46H), 1.22 (d, 12H), 0.91 (d, 12H).

APCI-MS analysis: Calculated C65H124N4O14S2 [M+H]=1249.8, Observed=1249.9.

### Diisopentyl 9,9′-((4-((2-(4-(2-((4-(bis(2-hydroxy-6-oxo-6-(pentan-3-yloxy)hexyl)amino)-butanoyl)oxy)ethyl)piperazin-1-yl)ethyl)disulfaneyl)butyl)azanediyl)bis(8-hydroxynonanoate) (GL-HEPES-E3-E6Es5-DS-4-E9Ei5)

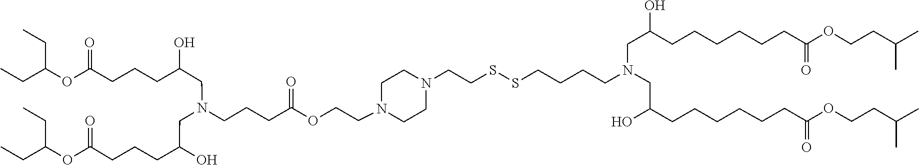 [SMILES: CCC(CC)OC(=O)CCCC(O)CN(CCCC(=O)OCCN1CCN(CCSSCCCCN(CC(O)CCCCCCC(=O)OCCC(C)C)CC(O)CCCCCCC(=O)OCCC(C)C)CC1)CC(O)CCCC(=O)OC(CC)CC]

¹H NMR (300 MHz, CDCl₃) δ 4.77 (pent, 2H), 4.19 (t, 2H), 4.08 (t, 4H), 3.62 (m, 4H), 2.85-2.25 (m, 36H), 1.86-1.24 (m, 54H), 0.91 (d, 12H), 0.86 (t, 12H).

APCI-MS analysis: Calculated C66H126N4O14S2 [M+H]=1263.8, Observed=1263.9.

### Diisopentyl 9,9′-((4-((2-(4-(2-((4-(bis(2-hydroxy-7-isopropoxy-7-oxoheptyl)amino)-butanoyl)oxy)ethyl)piperazin-1-yl)ethyl)disulfaneyl)butyl)azanediyl)bis(8-hydroxynonanoate) (GL-HEPES-E3-E7Ei3-DS-4-E9Ei5)

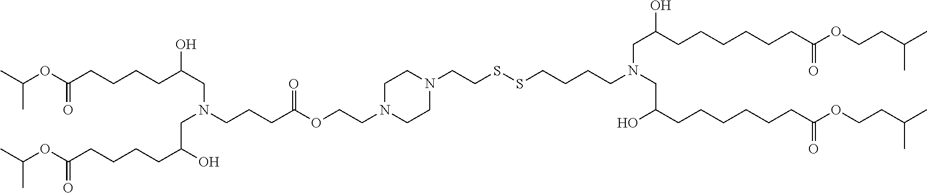 [SMILES: CC(C)CCOC(=O)CCCCCCC(O)CN(CCCCSSCCN1CCN(CCOC(=O)CCCN(CC(O)CCCCC(=O)OC(C)C)CC(O)CCCCC(=O)OC(C)C)CC1)CC(O)CCCCCCC(=O)OCCC(C)C]

¹H NMR (300 MHz, CDCl₃) δ 4.99 (hept, 2H), 4.19 (t, 2H), 4.08 (t, 4H), 3.61 (m, 4H), 2.85-2.24 (m, 36H), 1.86-1.27 (m, 46H), 1.22 (d, 12H), 0.91 (t, 12H).

APCI-MS analysis: Calculated C64H122N4O14S2 [M+H]=1235.8, Observed=1235.9.

### Bis(2-ethylbutyl) 9,9′-((4-(2-(4-(2-((4-(bis(2-hydroxy-9-(isopentyloxy)-9-oxononyl)amino)butyl)disulfaneyl)ethyl)piperazin-1-yl)ethoxy)-4-oxobutyl)azanediyl)bis(8-hydroxynonanoate) (GL-HEPES-E3-E9Es6-DS-4-E9Ei5)

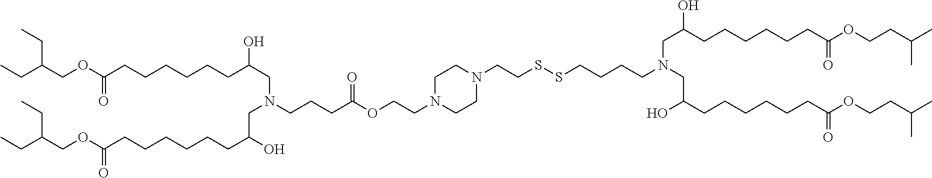 [SMILES: CCC(CC)COC(=O)CCCCCCC(O)CN(CCCC(=O)OCCN1CCN(CCSSCCCCN(CC(O)CCCCCCC(=O)OCCC(C)C)CC(O)CCCCCCC(=O)OCCC(C)C)CC1)CC(O)CCCCCCC(=O)OCC(CC)CC]

¹H NMR (300 MHz, CDCl₃) δ 4.19 (t, 2H), 4.08 (t, 4H), 3.98 (d, 4H), 3.61 (m, 4H), 2.85-2.25 (m, 38H), 1.85-1.24 (m, 62H), 0.91 (d, 12H), 0.88 (t, 12H).

APCI-MS analysis: Calculated C74H142N4O14S2 [M+H]=1376.0, Observed=1376.1.

### Bis(2-ethylbutyl) 9,9′-((4-((2-(4-(2-((4-(bis(2-hydroxy-6-oxo-6-(pentan-3-yloxy)hexyl)-amino)butanoyl)oxy)ethyl)piperazin-1-yl)ethyl)disulfaneyl)butyl)azanediyl)bis(8-hydroxynonanoate) (GL-HEPES-E3-E6Es5-DS-4-E9Es6)

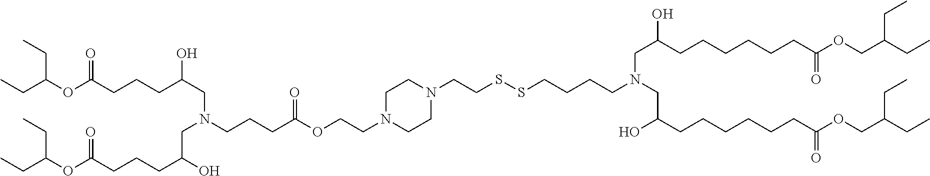 [SMILES: CCC(CC)COC(=O)CCCCCCC(O)CN(CCCCSSCCN1CCN(CCOC(=O)CCCN(CC(O)CCCC(=O)OC(CC)CC)CC(O)CCCC(=O)OC(CC)CC)CC1)CC(O)CCCCCCC(=O)OCC(CC)CC]

¹H NMR (300 MHz, CDCl₃) δ 4.75 (pent, 2H), 4.19 (t, 2H), 3.98 (d, 4H), 3.61 (m, 4H), 2.85-2.25 (m, 38H), 1.86-1.24 (m, 52H), 0.88 (t, 12H), 0.86 (t, 12H).

APCI-MS analysis: Calculated C68H130N4O14S2 [M+H]=1291.9, Observed=1291.9.

### Bis(2-ethylbutyl) 9,9′-((5-(2-(4-(2-((4-(bis(2-hydroxy-7-isopropoxy-7-oxoheptyl)amino)-butyl)disulfaneyl)ethyl)piperazin-1-yl)ethoxy)-5-oxopentyl)azanediyl)bis(8-hydroxynonanoate) (GL-HEPES-E4-E9Es6-DS-4-E7Ei3)

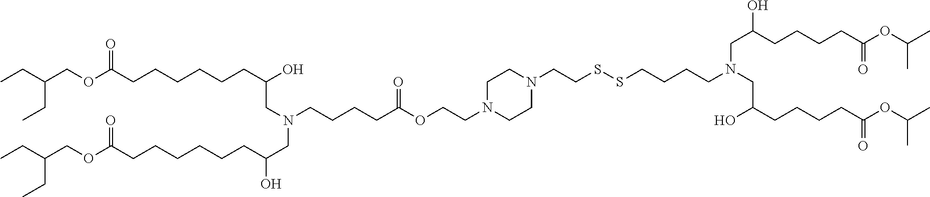 [SMILES: CCC(CC)COC(=O)CCCCCCC(O)CN(CCCCC(=O)OCCN1CCN(CCSSCCCCN(CC(O)CCCCC(=O)OC(C)C)CC(O)CCCCC(=O)OC(C)C)CC1)CC(O)CCCCCCC(=O)OCC(CC)CC]

¹H NMR (300 MHz, CDCl₃) δ 4.99 (hept, 2H), 4.19 (t, 2H), 3.98 (d, 4H), 3.62 (m, 4H), 2.85-2.23 (m, 38H), 1.83-1.25 (m, 50H), 1.22 (d, 12H), 0.88 (t, 12H).

APCI-MS analysis: Calculated C67H128N4O14S2 [M+H]=1277.9, Observed=1277.9.

### Diisopentyl 9,9′-((5-(2-(4-(2-((3-(bis(7-butoxy-2-hydroxy-7-oxoheptyl)amino)-propyl)disulfaneyl)ethyl)piperazin-1-yl)ethoxy)-5-oxopentyl)azanediyl)bis(8-hydroxynonanoate) (GL-HEPES-E4-E9Ei5-DS-3-E7E4)

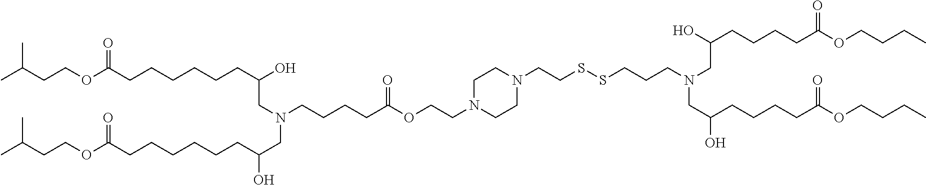 [SMILES: CCCCOC(=O)CCCCC(O)CN(CCCSSCCN1CCN(CCOC(=O)CCCCN(CC(O)CCCCCCC(=O)OCCC(C)C)CC(O)CCCCCCC(=O)OCCC(C)C)CC1)CC(O)CCCCC(=O)OCCCC]

¹H NMR (300 MHz, CDCl₃) δ 4.19 (t, 2H), 4.08 (t, 4H), 4.06 (t, 4H), 3.65 (m, 4H), 2.86-2.46 (m, 22H), 2.45-2.23 (m, 16H), 1.91-1.23 (m, 52H), 0.92 (t, 6H), 0.91 (d, 12H).

APCI-MS analysis: Calculated C66H126N4O14S2 [M+H]=1263.8, Observed=1263.9.

### Bis(2-ethylbutyl) 9,9′-((5-(2-(4-(2-((3-(bis(7-butoxy-2-hydroxy-7-oxoheptyl)amino)-propyl)disulfaneyl)ethyl)piperazin-1-yl)ethoxy)-5-oxopentyl)azanediyl)bis(8-hydroxynonanoate) (GL-HEPES-E4-E9Es6-DS-3-E7E4)

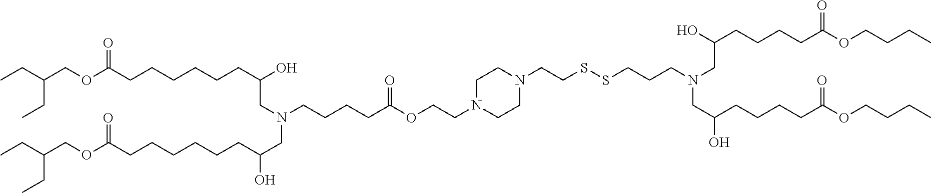 [SMILES: CCCCOC(=O)CCCCC(O)CN(CCCSSCCN1CCN(CCOC(=O)CCCCN(CC(O)CCCCCCC(=O)OCC(CC)CC)CC(O)CCCCCCC(=O)OCC(CC)CC)CC1)CC(O)CCCCC(=O)OCCCC]

¹H NMR (300 MHz, CDCl₃) δ 4.19 (t, 2H), 4.06 (t, 4H), 3.98 (d, 4H), 3.63 (m, 4H), 2.86-2.46 (m, 22H), 2.45-2.23 (m, 16H), 1.91-1.23 (m, 62H), 0.93 (t, 6H), 0.88 (t, 12H).

APCI-MS analysis: Calculated C68H130N4O14S2 [M+H]=1291.9, Observed=1291.9.

### Bis(2-ethylbutyl) 9,9′-((4-((2-(4-(2-((4-(bis(9-(2-ethylbutoxy)-2-hydroxy-9-oxononyl)amino)butanoyl)oxy)ethyl)piperazin-1-yl)ethyl)disulfaneyl)butyl)azanediyl)bis(8-hydroxynonanoate) (GL-HEPES-E3-E9Es6-DS-4-E9Es6)

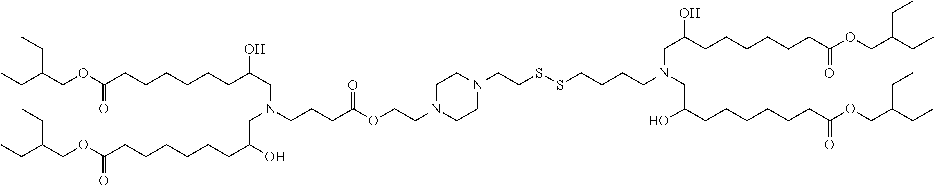 [SMILES: CCC(CC)COC(=O)CCCCCCC(O)CN(CCCCSSCCN1CCN(CCOC(=O)CCCN(CC(O)CCCCCCC(=O)OCC(CC)CC)CC(O)CCCCCCC(=O)OCC(CC)CC)CC1)CC(O)CCCCCCC(=O)OCC(CC)CC]

¹H NMR (300 MHz, CDCl₃) δ 4.19 (t, 2H), 3.98 (d, 8H), 3.62 (m, 4H), 2.85-2.25 (m, 36H), 1.85-1.24 (m, 72H), 0.88 (t, 24H).

APCI-MS analysis: Calculated C76H146N4O14S2 [M+H]=1404.1, Observed=1404.0.

### Diisopentyl 9,9′-((5-(2-(4-(2-((4-(bis(7-butoxy-2-hydroxy-7-oxoheptyl)amino)butyl)-disulfaneyl)ethyl)piperazin-1-yl)ethoxy)-5-oxopentyl)azanediyl)bis(8-hydroxynonanoate) (GL-HEPES-E4-E9Ei5-DS-4-E7E4)

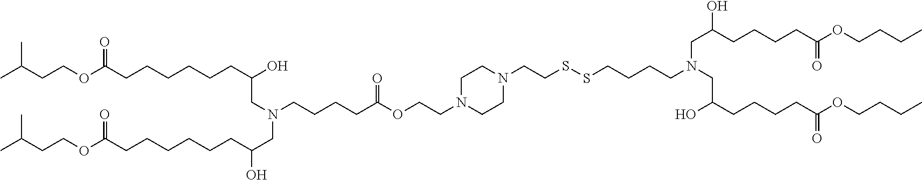 [SMILES: CCCCOC(=O)CCCCC(O)CN(CCCCSSCCN1CCN(CCOC(=O)CCCCN(CC(O)CCCCCCC(=O)OCCC(C)C)CC(O)CCCCCCC(=O)OCCC(C)C)CC1)CC(O)CCCCC(=O)OCCCC]

¹H NMR (300 MHz, CDCl₃) δ 4.19 (t, 2H), 4.08 (t, 4H), 4.06 (t, 4H), 3.61 (m, 4H), 2.84-2.46 (m, 22H), 2.45-2.23 (m, 16H), 1.80-1.25 (m, 54H), 0.92 (t, 6H), 0.91 (d, 12H).

APCI-MS analysis: Calculated C67H128N4O14S2 [M+H]=1277.9, Observed=1278.0.

### Bis(2-ethylbutyl) 9,9′-((5-(2-(4-(2-((4-(bis(7-butoxy-2-hydroxy-7-oxoheptyl)amino)butyl)-disulfaneyl)ethyl)piperazin-1-yl)ethoxy)-5-oxopentyl)azanediyl)bis(8-hydroxynonanoate) (GL-HEPES-E4-E9Es6-DS-4-E7E4)

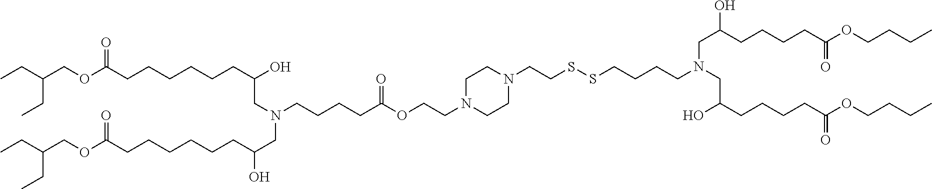 [SMILES: CCCCOC(=O)CCCCC(O)CN(CCCCSSCCN1CCN(CCOC(=O)CCCCN(CC(O)CCCCCCC(=O)OCC(CC)CC)CC(O)CCCCCCC(=O)OCC(CC)CC)CC1)CC(O)CCCCC(=O)OCCCC]

¹H NMR (300 MHz, CDCl₃) δ 4.19 (t, 2H), 4.06 (t, 4H), 3.98 (d, 4H), 3.61 (m, 4H), 2.86-2.46 (m, 22H), 2.45-2.23 (m, 16H), 1.80-1.23 (m, 58H), 0.92 (t, 6H), 0.88 (t, 12H).

APCI-MS analysis: Calculated C69H132N4O14S2 [M+H]=1305.9, Observed=1306.0.

### Diisopentyl 9,9′-((5-(2-(4-(2-((3-(bis(2-hydroxy-7-(isopentyloxy)-7-oxoheptyl)amino)-propyl)disulfaneyl)ethyl)piperazin-1-yl)ethoxy)-5-oxopentyl)azanediyl)bis(8-hydroxynonanoate (GL-HEPES-E4-E9Ei5-DS-3-E7Ei5)

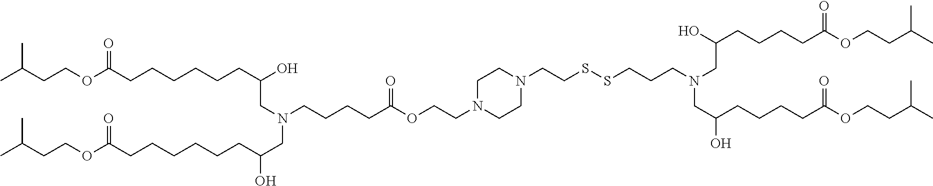 [SMILES: CC(C)CCOC(=O)CCCCCCC(O)CN(CCCCC(=O)OCCN1CCN(CCSSCCCN(CC(O)CCCCC(=O)OCCC(C)C)CC(O)CCCCC(=O)OCCC(C)C)CC1)CC(O)CCCCCCC(=O)OCCC(C)C]

¹H NMR (300 MHz, CDCl₃) δ 4.19 (t, 2H), 4.08 (t, 8H), 3.62 (m, 4H), 2.85-2.23 (m, 38H), 1.91-1.25 (m, 54H), 0.91 (d, 24H).

APCI-MS analysis: Calculated C68H130N4O14S2 [M+H]=1291.9, Observed=1292.0.

### Diisopropyl 7,7′-((3-((2-(4-(2-((5-(bis(2-hydroxy-7-isopropoxy-7-oxoheptyl)amino)-pentanoyl)oxy)ethyl)piperazin-1-yl)ethyl)disulfaneyl)propyl)azanediyl)bis(6-hydroxyheptanoate) (GL-HEPES-E4-E7Ei3-DS-3-E7Ei3)

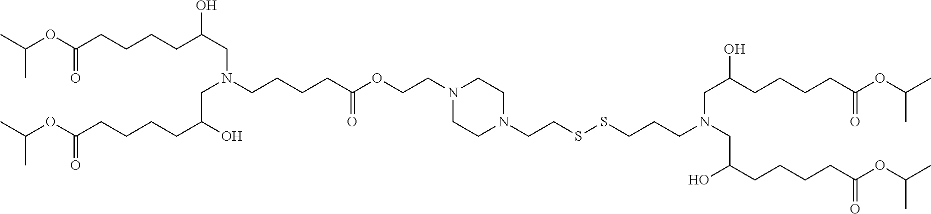 [SMILES: CC(C)OC(=O)CCCCC(O)CN(CCCCC(=O)OCCN1CCN(CCSSCCCN(CC(O)CCCCC(=O)OC(C)C)CC(O)CCCCC(=O)OC(C)C)CC1)CC(O)CCCCC(=O)OC(C)C]

¹H NMR (300 MHz, CDCl₃) δ 4.99 (hept, 4H), 4.19 (t, 2H), 3.62 (m, 4H), 2.85-2.24 (m, 34H), 1.95-1.32 (m, 38H), 1.22 (d, 24H).

APCI-MS analysis: Calculated C56H106N4O14S2 [M+H]=1123.6, Observed=1123.7.

### Bis(2-ethylbutyl) 7,7′-((5-(2-(4-(2-((3-(bis(2-hydroxy-7-isopropoxy-7-oxoheptyl)amino)-propyl)disulfaneyl)ethyl)piperazin-1-yl)ethoxy)-5-oxopentyl)azanediyl)bis(6-hydroxyheptanoate) (GL-HEPES-E4-E7Es6-DS-3-E7Ei3)

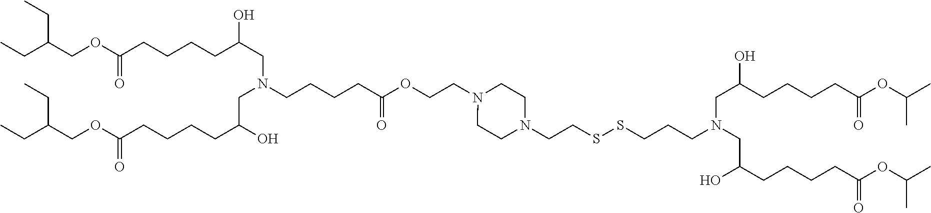 [SMILES: CCC(CC)COC(=O)CCCCC(O)CN(CCCCC(=O)OCCN1CCN(CCSSCCCN(CC(O)CCCCC(=O)OC(C)C)CC(O)CCCCC(=O)OC(C)C)CC1)CC(O)CCCCC(=O)OCC(CC)CC]

¹H NMR (300 MHz, CDCl₃) δ 4.99 (hept, 2H), 4.19 (t, 2H), 3.98 (d, 4H), 3.65 (m, 4H), 2.85-2.23 (m, 40H), 1.88-1.29 (m, 42H), 1.22 (d, 12H), 0.88 (t, 12H).

APCI-MS analysis: Calculated C62H118N4O14S2 [M+H]=1207.7, Observed=1207.9.

### Dibutyl 7,7′-((3-((2-(4-(2-((5-(bis(2-hydroxy-7-isopropoxy-7-oxoheptyl)amino)-pentanoyl)oxy)ethyl)piperazin-1-yl)ethyl)disulfaneyl)propyl)azanediyl)bis(6-hydroxyheptanoate) (GL-HEPES-E4-E7Ei3-DS-3-E7E4)

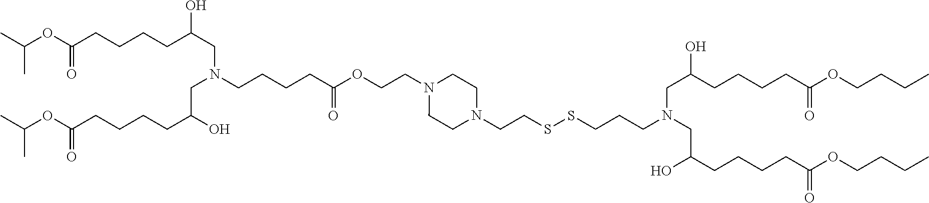 [SMILES: CCCCOC(=O)CCCCC(O)CN(CCCSSCCN1CCN(CCOC(=O)CCCCN(CC(O)CCCCC(=O)OC(C)C)CC(O)CCCCC(=O)OC(C)C)CC1)CC(O)CCCCC(=O)OCCCC]

¹H NMR (300 MHz, CDCl₃) δ 4.99 (hept, 2H), 4.19 (t, 2H), 4.06 (t, 4H), 3.62 (m, 4H), 2.85-2.24 (m, 40H), 1.95-1.30 (m, 40H), 1.22 (d, 12H), 0.92 (t, 6H).

APCI-MS analysis: Calculated C58H110N4O14S2 [M+H]=1151.6, Observed=1151.8.

### Dibutyl 7,7′-((3-((2-(4-(2-((5-(bis(7-(2-ethylbutoxy)-2-hydroxy-7-oxoheptyl)amino)-pentanoyl)oxy)ethyl)piperazin-1-yl)ethyl)disulfaneyl)propyl)azanediyl)bis(6-hydroxyheptanoate) (GL-HEPES-E4-E7Es6-DS-3-E7E4)

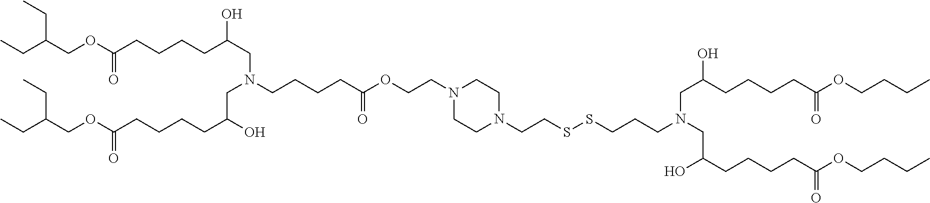 [SMILES: CCCCOC(=O)CCCCC(O)CN(CCCSSCCN1CCN(CCOC(=O)CCCCN(CC(O)CCCCC(=O)OCC(CC)CC)CC(O)CCCCC(=O)OCC(CC)CC)CC1)CC(O)CCCCC(=O)OCCCC]

¹H NMR (300 MHz, CDCl₃) δ 4.19 (t, 2H), 4.06 (t, 4H), 3.98 (d, 4H), 3.65 (m, 4H), 2.85-2.28 (m, 38H), 1.95-1.24 (m, 52H), 0.92 (t, 6H), 0.88 (t, 12H).

APCI-MS analysis: Calculated C64H122N4O14S2 [M+H]=1235.8, Observed=1235.8.

### Bis(2-ethylbutyl) 9,9′-((5-(2-(4-(2-((3-(bis(2-hydroxy-7-(isopentyloxy)-7-oxoheptyl)amino)-propyl)disulfaneyl)ethyl)piperazin-1-yl)ethoxy)-5-oxopentyl)azanediyl)bis(8-hydroxynonanoate) (GL-HEPES-E4-E9Es6-DS-3-E7Ei5)

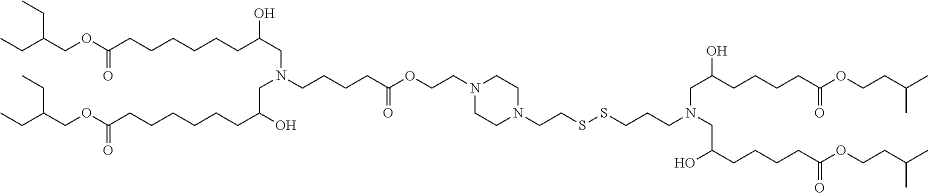 [SMILES: CCC(CC)COC(=O)CCCCCCC(O)CN(CCCCC(=O)OCCN1CCN(CCSSCCCN(CC(O)CCCCC(=O)OCCC(C)C)CC(O)CCCCC(=O)OCCC(C)C)CC1)CC(O)CCCCCCC(=O)OCC(CC)CC]

¹H NMR (300 MHz, CDCl₃) δ 4.19 (t, 2H), 4.08 (t, 4H), 3.98 (d, 4H), 3.61 (m, 4H), 2.86-2.46 (m, 22H), 2.45-2.23 (m, 16H), 1.90-1.24 (m, 58H), 0.91 (d, 12H), 0.88 (t, 12H).

APCI-MS analysis: Calculated C70H134N4O14S2 [M+H]=1319.9, Observed=1320.0.

### Diisopentyl 9,9′-((5-(2-(4-(2-((4-(bis(2-hydroxy-7-(isopentyloxy)-7-oxoheptyl)amino)butyl)-disulfaneyl)ethyl)piperazin-1-yl)ethoxy)-5-oxopentyl)azanediyl)bis(8-hydroxynonanoate) (GL-HEPES-E4-E9Ei5-DS-4-E7Ei5)

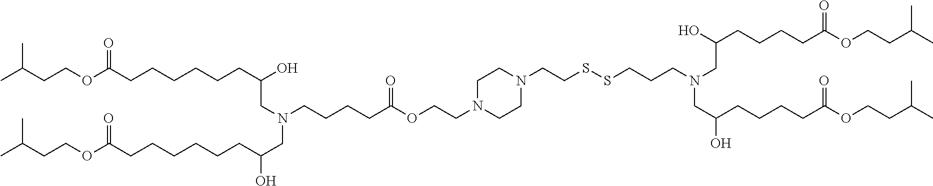 [SMILES: CC(C)CCOC(=O)CCCCCCC(O)CN(CCCCC(=O)OCCN1CCN(CCSSCCCN(CC(O)CCCCC(=O)OCCC(C)C)CC(O)CCCCC(=O)OCCC(C)C)CC1)CC(O)CCCCCCC(=O)OCCC(C)C]

¹H NMR (300 MHz, CDCl₃) δ 4.19 (t, 2H), 4.08 (t, 8H), 3.62 (m, 4H), 2.85-2.23 (m, 38H), 1.79-1.25 (m, 56H), 0.91 (d, 24H).

APCI-MS analysis: Calculated C69H132N4O14S2 [M+H]=1305.9, Observed=1305.9.

### Bis(2-ethylbutyl) 9,9′-((5-(2-(4-(2-((4-(bis(2-hydroxy-7-(isopentyloxy)-7-oxoheptyl)amino)-butyl)disulfaneyl)ethyl)piperazin-1-yl)ethoxy)-5-oxopentyl)azanediyl)bis(8-hydroxynonanoate) (GL-HEPES-E4-E9Es6-DS-4-E7Ei5)

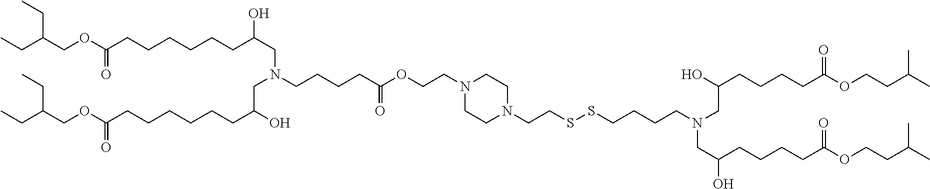 [SMILES: CCC(CC)COC(=O)CCCCCCC(O)CN(CCCCC(=O)OCCN1CCN(CCSSCCCCN(CC(O)CCCCC(=O)OCCC(C)C)CC(O)CCCCC(=O)OCCC(C)C)CC1)CC(O)CCCCCCC(=O)OCC(CC)CC]

¹H NMR (300 MHz, CDCl₃) δ 4.19 (t, 2H), 4.08 (t, 4H), 3.98 (d, 4H), 3.61 (m, 4H), 2.86-2.46 (m, 22H), 2.45-2.23 (m, 16H), 1.80-1.24 (m, 60H), 0.91 (d, 12H), 0.88 (t, 12H).

APCI-MS analysis: Calculated C71H136N4O14S2 [M+H]=1334.0, Observed=1333.9.

### Diisopentyl 9,9′-((3-((2-(4-(2-((5-(bis(2-hydroxy-7-isopropoxy-7-oxoheptyl)amino)-pentanoyl)oxy)ethyl)piperazin-1-yl)ethyl)disulfaneyl)propyl)azanediyl)bis(8-hydroxynonanoate) (GL-HEPES-E4-E7Ei3-DS-3-E9Ei5)

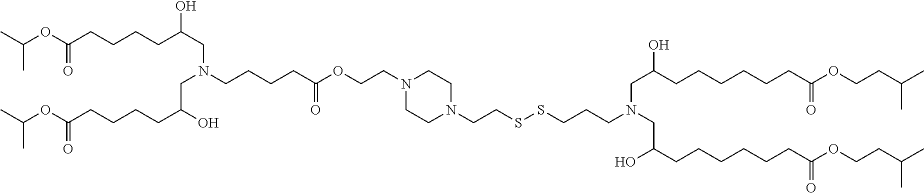 [SMILES: CC(C)CCOC(=O)CCCCCCC(O)CN(CCCSSCCN1CCN(CCOC(=O)CCCCN(CC(O)CCCCC(=O)OC(C)C)CC(O)CCCCC(=O)OC(C)C)CC1)CC(O)CCCCCCC(=O)OCCC(C)C]

¹H NMR (300 MHz, CDCl₃) δ 4.99 (hept, 2H), 4.19 (t, 2H), 4.08 (t, 8H), 3.63 (m, 4H), 2.85-2.24 (m, 40H), 1.95-1.27 (m, 40H), 1.22 (d, 12H), 0.91 (t, 12H).

APCI-MS analysis: Calculated C64H122N4O14S2 [M+H]=1235.8, Observed=1235.9.

### Diisopentyl 9,9′-((3-((2-(4-(2-((5-(bis(7-(2-ethylbutoxy)-2-hydroxy-7-oxoheptyl)amino)-pentanoyl)oxy)ethyl)piperazin-1-yl)ethyl)disulfaneyl)propyl)azanediyl)bis(8-hydroxynonanoate)) (GL-HEPES-E4-E7Es6-DS-3-E9Ei5)

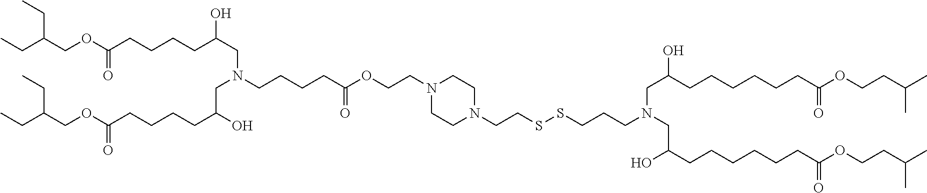 [SMILES: CCC(CC)COC(=O)CCCCC(O)CN(CCCCC(=O)OCCN1CCN(CCSSCCCN(CC(O)CCCCCCC(=O)OCCC(C)C)CC(O)CCCCCCC(=O)OCCC(C)C)CC1)CC(O)CCCCC(=O)OCC(CC)CC]

¹H NMR (300 MHz, CDCl₃) δ 4.19 (t, 2H), 4.08 (t, 4H), 3.98 (d, 4H), 3.63 (m, 4H), 2.85-2.25 (m, 40H), 1.95-1.24 (m, 52H), 0.91 (t, 12H), 0.88 (t, 12H).

APCI-MS analysis: Calculated C70H134N4O14S2 [M+H]=1319.9, Observed=1320.0.

### Dibutyl 7,7′-((4-(2-(4-(2-((3-(bis(2-hydroxy-7-isopropoxy-7-oxoheptyl)amino)propyl)-disulfaneyl)ethyl)piperazin-1-yl)ethoxy)-4-oxobutyl)azanediyl)bis(6-hydroxyheptanoate) (GL-HEPES-E3-E7E4-DS-4-E7Ei3)

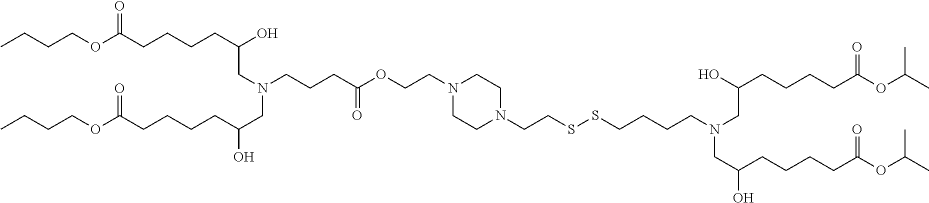 [SMILES: CCCCOC(=O)CCCCC(O)CN(CCCC(=O)OCCN1CCN(CCSSCCCCN(CC(O)CCCCC(=O)OC(C)C)CC(O)CCCCC(=O)OC(C)C)CC1)CC(O)CCCCC(=O)OCCCC]

¹H NMR (300 MHz, CDCl₃) δ 4.99 (hept, 2H), 4.19 (t, 2H), 4.06 (t, 4H), 3.63 (m, 4H), 2.85-2.22 (m, 38H), 1.85-1.28 (m, 38H), 1.22 (d, 12H), 0.92 (t, 6H).

APCI-MS analysis: Calculated C58H110N4O14S2 [M+H]=1151.6, Observed=1151.1.

### Dibutyl 7,7′-((5-(2-(4-(2-((4-(bis(2-hydroxy-7-isopropoxy-7-oxoheptyl)amino)butyl)-disulfaneyl)ethyl)piperazin-1-yl)ethoxy)-5-oxopentyl)azanediyl)bis(6-hydroxyheptanoate) (GL-HEPES-E4-E7E4-DS-4-E7Ei3)

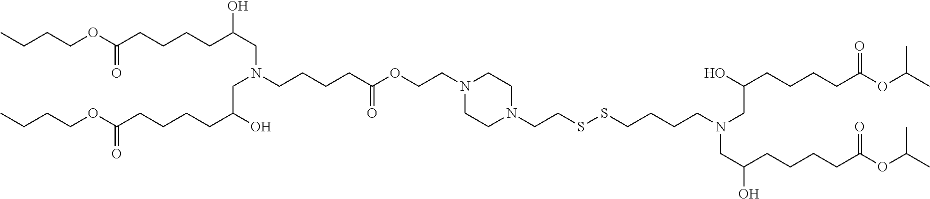 [SMILES: CCCCOC(=O)CCCCC(O)CN(CCCCC(=O)OCCN1CCN(CCSSCCCCN(CC(O)CCCCC(=O)OC(C)C)CC(O)CCCCC(=O)OC(C)C)CC1)CC(O)CCCCC(=O)OCCCC]

¹H NMR (300 MHz, CDCl₃) δ 4.99 (hept, 2H), 4.19 (t, 2H), 4.06 (t, 4H), 3.62 (m, 4H), 2.85-2.22 (m, 38H), 1.85-1.24 (m, 40H), 1.22 (d, 12H), 0.92 (t, 6H).

APCI-MS analysis: Calculated C59H112N4O14S2 [M+H]=1165.6, Observed=1165.2.

### Dibutyl 7,7′-((4-((2-(4-(2-((4-(bis(7-butoxy-2-hydroxy-7-oxoheptyl)amino)butanoyl)-oxy)ethyl)piperazin-1-yl)ethyl)disulfaneyl)butyl)azanediyl)bis(6-hydroxyheptanoate) (GL-HEPES-E3-E7E4-DS-4-E7E4)

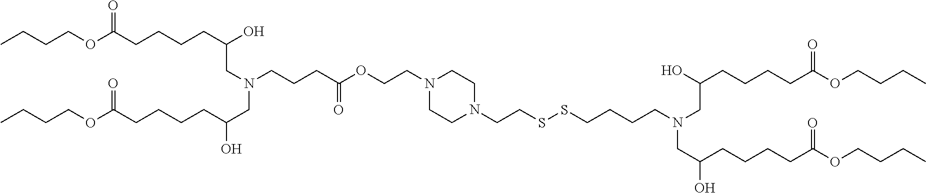 [SMILES: CCCCOC(=O)CCCCC(O)CN(CCCCSSCCN1CCN(CCOC(=O)CCCN(CC(O)CCCCC(=O)OCCCC)CC(O)CCCCC(=O)OCCCC)CC1)CC(O)CCCCC(=O)OCCCC]

¹H NMR (300 MHz, CDCl₃) δ 4.19 (t, 2H), 4.06 (t, 8H), 3.62 (m, 4H), 2.85-2.22 (m, 38H), 1.85-1.24 (m, 50H), 0.92 (t, 12H).

APCI-MS analysis: Calculated C60H114N4O14S2 [M+H]=1179.7, Observed=1179.0.

### Dibutyl 7,7′-((4-(2-(4-(2-((4-(bis(7-(2-ethyl butoxy)-2-hydroxy-7-oxoheptyl)amino)butyl)-disulfaneyl)ethyl)piperazin-1-yl)ethoxy)-4-oxobutyl)azanediyl)bis(6-hydroxyheptanoate) (GL-HEPES-E3-E7E4-DS-4-E7Es6)

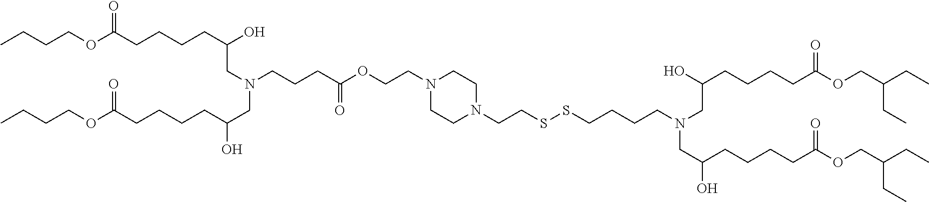 [SMILES: CCCCOC(=O)CCCCC(O)CN(CCCC(=O)OCCN1CCN(CCSSCCCCN(CC(O)CCCCC(=O)OCC(CC)CC)CC(O)CCCCC(=O)OCC(CC)CC)CC1)CC(O)CCCCC(=O)OCCCC]

¹H NMR (300 MHz, CDCl₃) δ 4.19 (t, 2H), 4.06 (t, 4H), 3.98 (d, 4H), 3.63 (m, 4H), 2.85-2.22 (m, 38H), 1.85-1.24 (m, 36H), 0.92 (t, 6H), 0.88 (t, 12H).

APCI-MS analysis: Calculated C64H122N4O14S2 [M+H]=1235.8, Observed=1235.0.

### Bis(2-ethylbutyl) 9,9′-((4-((2-(4-(2-((5-(bis(2-hydroxy-7-isopropoxy-7-oxoheptyl)amino)-pentanoyl)oxy)ethyl)piperazin-1-yl)ethyl)disulfaneyl)butyl)azanediyl)bis(8-hydroxynonanoate) (GL-HEPES-E4-E7Ei3-DS-4-E9Es6)

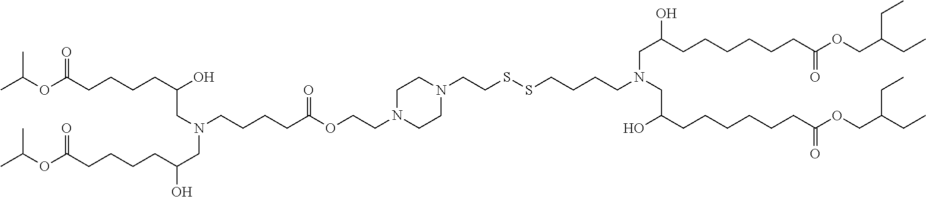 [SMILES: CCC(CC)COC(=O)CCCCCCC(O)CN(CCCCSSCCN1CCN(CCOC(=O)CCCCN(CC(O)CCCCC(=O)OC(C)C)CC(O)CCCCC(=O)OC(C)C)CC1)CC(O)CCCCCCC(=O)OCC(CC)CC]

¹H NMR (300 MHz, CDCl₃) δ 4.99 (hept, 2H), 4.19 (t, 2H), 3.98 (d, 4H), 3.64 (m, 4H), 2.85-2.24 (m, 40H), 1.78-1.29 (m, 48H), 1.21 (d, 12H), 0.88 (t, 12H).

APCI-MS analysis: Calculated C67H128N4O14S2 [M+H]=1277.9, Observed=1277.0.

### Bis(2-ethylbutyl) 9,9′-((4-((2-(4-(2-((5-(bis(7-(2-ethylbutoxy)-2-hydroxy-7-oxoheptyl)-amino)pentanoyl)oxy)ethyl)piperazin-1-yl)ethyl)disulfaneyl)butyl)azanediyl)bis(8-hydroxynonanoate) (GL-HEPES-E4-E7Es6-DS-4-E9Es6)

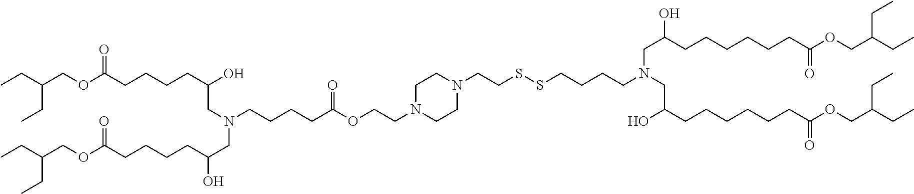 [SMILES: CCC(CC)COC(=O)CCCCCCC(O)CN(CCCCSSCCN1CCN(CCOC(=O)CCCCN(CC(O)CCCCC(=O)OCC(CC)CC)CC(O)CCCCC(=O)OCC(CC)CC)CC1)CC(O)CCCCCCC(=O)OCC(CC)CC]

¹H NMR (300 MHz, CDCl3) δ 4.19 (t, 2H), 3.98 (d, 8H), 3.63 (m, 4H), 2.85-2.25 (m, 40H), 1.90-1.24 (m, 62H), 0.88 (t, 24H).

APCI-MS analysis: Calculated C73H140N4O14S2 [M+H]=1362.0, Observed=1361.2.

### Dibutyl 9,9′-((3-((2-(4-(2-((5-(bis(2-hydroxy-9-(isopentyloxy)-9-oxononyl)amino)-pentanoyl)oxy)ethyl)piperazin-1-yl)ethyl)disulfaneyl)propyl)azanediyl)bis(8-hydroxynonanoate) (GL-HEPES-E4-E9Ei5-DS-3-E9E4)

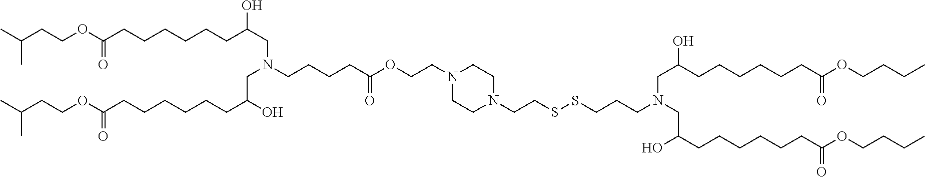 [SMILES: CCCCOC(=O)CCCCCCC(O)CN(CCCSSCCN1CCN(CCOC(=O)CCCCN(CC(O)CCCCCCC(=O)OCCC(C)C)CC(O)CCCCCCC(=O)OCCC(C)C)CC1)CC(O)CCCCCCC(=O)OCCCC]

¹H NMR (300 MHz, CDCl₃) δ 4.19 (t, 2H), 4.08 (t, 4H), 4.06 (t, 4H), 3.64 (m, 4H), 2.86-2.46 (m), 2.45-2.23 (m, 16H), 1.90-1.23 (m, 64H), 0.92 (t, 6H), 0.91 (d, 12H).

APCI-MS analysis: Calculated C70H134N4O14S2 [M+H]=1319.9, Observed=1319.0.

### Dibutyl 9,9′-((3-((2-(4-(2-((5-(bis(9-(2-ethylbutoxy)-2-hydroxy-9-oxononyl)amino)-pentanoyl)oxy)ethyl)piperazin-1-yl)ethyl)disulfaneyl)propyl)azanediyl)bis(8-hydroxynonanoate) (GL-HEPES-E4-E9Es6-DS-3-E9E4)

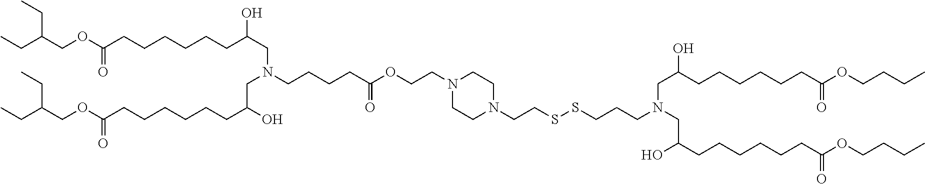 [SMILES: CCCCOC(=O)CCCCCCC(O)CN(CCCSSCCN1CCN(CCOC(=O)CCCCN(CC(O)CCCCCCC(=O)OCC(CC)CC)CC(O)CCCCCCC(=O)OCC(CC)CC)CC1)CC(O)CCCCCCC(=O)OCCCC]

¹H NMR (300 MHz, CDCl₃) δ 4.19 (t, 2H), 4.06 (t, 4H), 3.98 (d, 4H), 3.61 (m, 4H), 2.86-2.46 (m, 22H), 2.45-2.23 (m, 16H), 1.90-1.23 (m, 68H), 0.92 (t, 6H), 0.88 (d, 12H).

APCI-MS analysis: Calculated C72H138N4O14S2 [M+H]=1348.0, Observed=1346.9.

### Dibutyl 7,7′-((4-(2-(4-(2-((3-(bis(2-hydroxy-7-(isopentyloxy)-7-oxoheptyl)amino)propyl)-disulfaneyl)ethyl)piperazin-1-yl)ethoxy)-4-oxobutyl)azanediyl)bis(6-hydroxyheptanoate) (GL-HEPES-E3-E7E4-DS-3-E7Ei5)

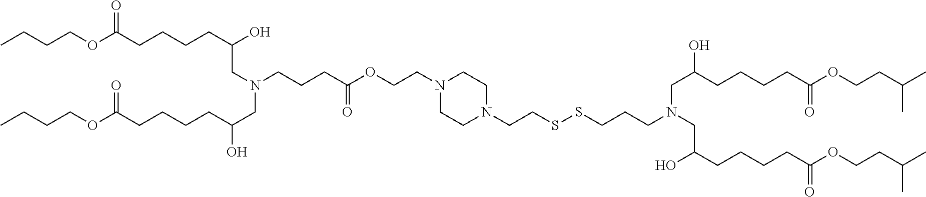 [SMILES: CCCCOC(=O)CCCCC(O)CN(CCCC(=O)OCCN1CCN(CCSSCCCN(CC(O)CCCCC(=O)OCCC(C)C)CC(O)CCCCC(=O)OCCC(C)C)CC1)CC(O)CCCCC(=O)OCCCC]

¹H NMR (300 MHz, CDCl₃) δ 4.19 (t, 2H), 4.08 (t, 4H), 4.06 (t, 4H), 3.64 (m, 4H), 2.87-2.46 (m, 22H), 2.45-2.26 (m, 16H), 1.91-1.31 (m, 46H), 0.92 (t, 6H), 0.91 (d, 12H).

APCI-MS analysis: Calculated C61H116N4O14S2 [M+H]=1193.7, Observed=1193.4.

### Dibutyl 7,7′-((4-(2-(4-(2-((4-(bis(2-hydroxy-7-(isopentyloxy)-7-oxoheptyl)amino)-butyl)disulfaneyl)ethyl)piperazin-1-yl)ethoxy)-4-oxobutyl)azanediyl)bis(6-hydroxyheptanoate) (GL-HEPES-E3-E7E4-DS-4-E7Ei5)

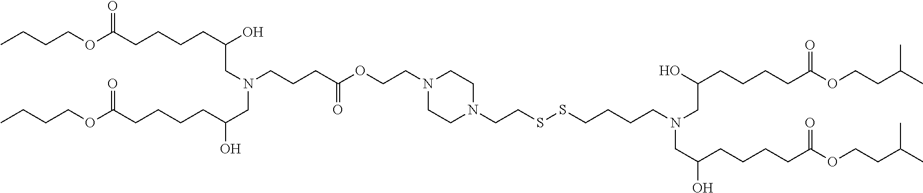 [SMILES: CCCCOC(=O)CCCCC(O)CN(CCCC(=O)OCCN1CCN(CCSSCCCCN(CC(O)CCCCC(=O)OCCC(C)C)CC(O)CCCCC(=O)OCCC(C)C)CC1)CC(O)CCCCC(=O)OCCCC]

¹H NMR (300 MHz, CDCl₃) δ 4.19 (t, 2H), 4.08 (t, 4H), 4.06 (t, 4H), 3.63 (m, 4H), 2.86-2.46 (m, 22H), 2.45-2.25 (m, 16H), 1.91-1.28 (m, 48H), 0.92 (t, 6H), 0.91 (d, 12H).

APCI-MS analysis: Calculated C62H118N4O14S2 [M+H]=1207.7, Observed=1207.4.

### Dibutyl 9,9′-((4-((2-(4-(2-((5-(bis(2-hydroxy-9-(isopentyloxy)-9-oxononyl)amino)-pentanoyl)oxy)ethyl)piperazin-1-yl)ethyl)disulfaneyl)butyl)azanediyl)bis(8-hydroxynonanoate) (GL-HEPES-E4-E9Ei5-DS-4-E9E4)

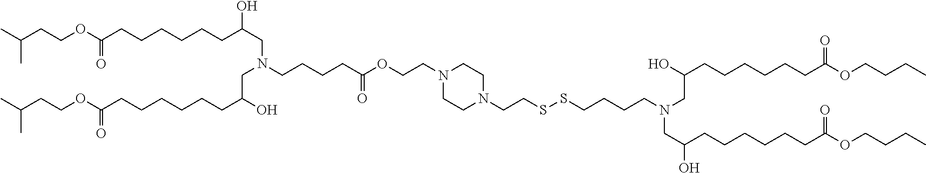 [SMILES: CCCCOC(=O)CCCCCCC(O)CN(CCCCSSCCN1CCN(CCOC(=O)CCCCN(CC(O)CCCCCCC(=O)OCCC(C)C)CC(O)CCCCCCC(=O)OCCC(C)C)CC1)CC(O)CCCCCCC(=O)OCCCC]

¹H NMR (300 MHz, CDCl₃) δ 4.19 (t, 2H), 4.08 (t, 4H), 4.05 (t, 4H), 3.64 (m, 4H), 2.86-2.23 (m, 40H), 1.75-1.23 (m, 64H), 0.92 (t, 6H), 0.91 (d, 12H).

APCI-MS analysis: Calculated C71H136N4O14S2 [M+H]=1334.0, Observed=1333.7.

### Dibutyl 9,9′-((3-((2-(4-(2-((5-(bis(9-(2-ethylbutoxy)-2-hydroxy-9-oxononyl)amino)-pentanoyl)oxy)ethyl)piperazin-1-yl)ethyl)disulfaneyl)propyl)azanediyl)bis(8-hydroxynonanoate) (GL-HEPES-E4-E9Es6-DS-4-E9E4)

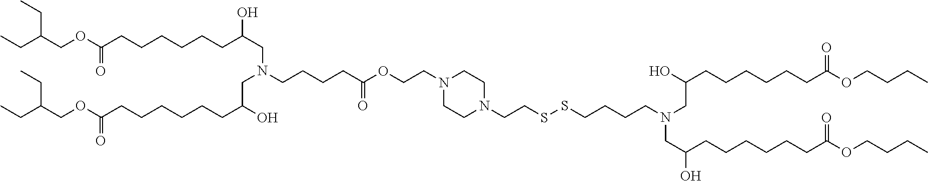 [SMILES: CCCCOC(=O)CCCCCCC(O)CN(CCCCSSCCN1CCN(CCOC(=O)CCCCN(CC(O)CCCCCCC(=O)OCC(CC)CC)CC(O)CCCCCCC(=O)OCC(CC)CC)CC1)CC(O)CCCCCCC(=O)OCCCC]

¹H NMR (300 MHz, CDCl₃) δ 4.19 (t, 2H), 4.06 (t, 4H), 3.98 (d, 4H), 3.63 (m, 4H), 2.86-2.23 (m, 38H), 1.70-1.23 (m, 62H), 0.92 (t, 6H), 0.88 (t, 12H).

APCI-MS analysis: Calculated C71H136N4O14S2 [M+H]=1362.0, Observed=1361.5.

### Dibutyl 7,7′-((3-((2-(4-(2-((5-(bis(7-butoxy-2-hydroxy-7-oxoheptyl)amino)-pentanoyl)oxy)ethyl)piperazin-1-yl)ethyl)disulfaneyl)propyl)azanediyl)bis(6-hydroxyheptanoate) (GL-HEPES-E4-E7E4-DS-3-E7E4)

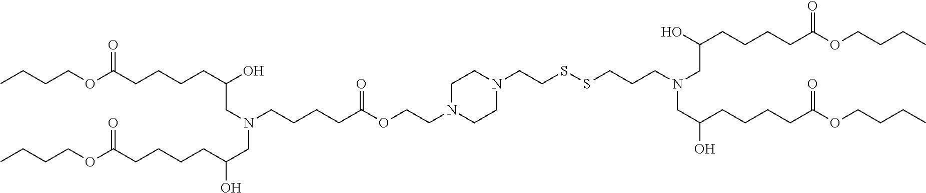 [SMILES: CCCCOC(=O)CCCCC(O)CN(CCCCC(=O)OCCN1CCN(CCSSCCCN(CC(O)CCCCC(=O)OCCCC)CC(O)CCCCC(=O)OCCCC)CC1)CC(O)CCCCC(=O)OCCCC]

¹H NMR (300 MHz, CDCl₃) δ 4.19 (t, 2H), 4.06 (t, 8H), 3.64 (m, 4H), 2.86-2.46 (m, 22H), 2.45-2.26 (m, 16H), 1.91-1.30 (m, 50H), 0.92 (t, 12H).

APCI-MS analysis: Calculated C60H114N4O14S2 [M+H]=1179.7, Observed=1179.4.

### Dibutyl 7,7′-((5-(2-(4-(2-((4-(bis(7-butoxy-2-hydroxy-7-oxoheptyl)amino)butyl)-disulfaneyl)ethyl)piperazin-1-yl)ethoxy)-5-oxopentyl)azanediyl)bis(6-hydroxyheptanoate) (GL-HEPES-E4-E7E4-DS-4-E7E4)

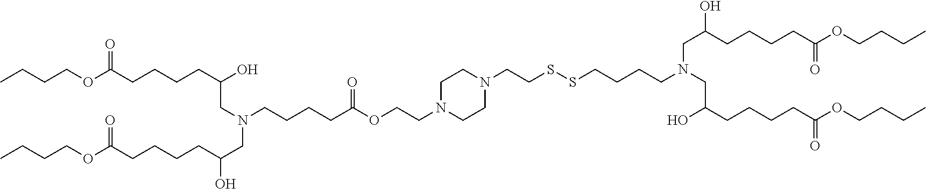 [SMILES: CCCCOC(=O)CCCCC(O)CN(CCCCSSCCN1CCN(CCOC(=O)CCCCN(CC(O)CCCCC(=O)OCCCC)CC(O)CCCCC(=O)OCCCC)CC1)CC(O)CCCCC(=O)OCCCC]

¹H NMR (300 MHz, CDCl₃) δ 4.19 (t, 2H), 4.06 (t, 8H), 3.63 (m, 4H), 2.86-2.46 (m, 22H), 2.45-2.26 (m, 16H), 1.81-1.30 (m, 52H), 0.92 (t, 12H).

APCI-MS analysis: Calculated C61H116N4O14S2 [M+H]=1193.7, Observed=1193.5.

### Bis(2-ethylbutyl) 9,9′-((5-(2-(4-(2-((3-(bis(2-hydroxy-7-isopropoxy-7-oxoheptyl)amino)-propyl)disulfaneyl)ethyl)piperazin-1-yl)ethoxy)-5-oxopentyl)azanediyl)bis(8-hydroxynonanoate) (GL-HEPES-E4-E9Es6-DS-3-E7Ei3)

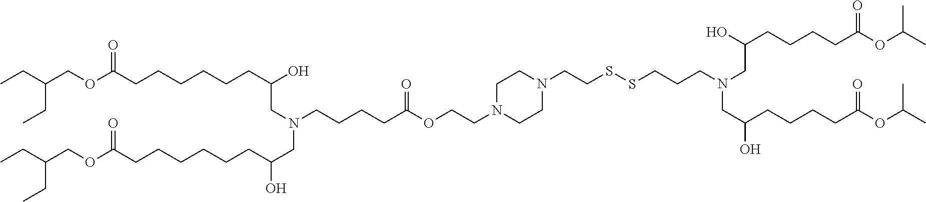 [SMILES: CCC(CC)COC(=O)CCCCCCC(O)CN(CCCCC(=O)OCCN1CCN(CCSSCCCN(CC(O)CCCCC(=O)OC(C)C)CC(O)CCCCC(=O)OC(C)C)CC1)CC(O)CCCCCCC(=O)OCC(CC)CC]

¹H NMR (300 MHz, CDCl₃) δ 4.99 (hept, 2H), 4.19 (t, 2H), 3.98 (d, 4H), 3.62 (m, 4H), 2.86-2.22 (m, 38H), 1.85-1.24 (m, 40H), 1.22 (d, 12H), 0.88 (t, 12H).

APCI-MS analysis: Calculated C66H126N4O14S2 [M+H]=1263.8, Observed=1263.9.

### Bis(2-ethylbutyl) 9,9′-((4-((2-(4-(2-((4-(bis(2-hydroxy-7-isopropoxy-7-oxoheptyl)amino)-butanoyl)oxy)ethyl)piperazin-1-yl)ethyl)disulfaneyl)butyl)azanediyl)bis(8-hydroxynonanoate) (GL-HEPES-E3-E7Ei3-DS-4-E9Es6)

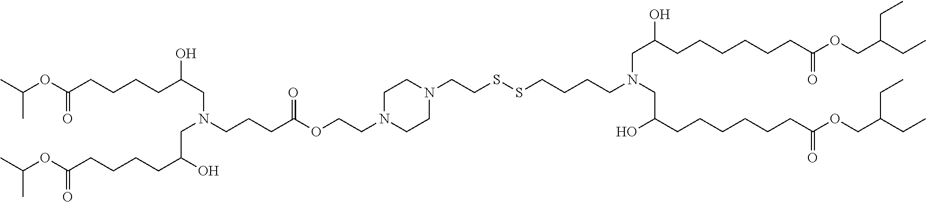 [SMILES: CCC(CC)COC(=O)CCCCCCC(O)CN(CCCCSSCCN1CCN(CCOC(=O)CCCN(CC(O)CCCCC(=O)OC(C)C)CC(O)CCCCC(=O)OC(C)C)CC1)CC(O)CCCCCCC(=O)OCC(CC)CC]

¹H NMR (300 MHz, CDCl₃) δ 4.99 (hept, 2H), 4.19 (t, 2H), 3.98 (d, 4H), 3.61 (m, 4H), 2.85-2.24 (m, 38H), 1.86-1.27 (m, 48H), 1.22 (d, 12H), 0.88 (t, 12H).

APCI-MS analysis: Calculated C66H126N4O14S2 [M+H]=1263.8, Observed=1263.9.

### Diisopentyl 7,7′-((3-((2-(4-(2-((5-(bis(2-hydroxy-7-isopropoxy-7-oxoheptyl)amino)-pentanoyl)oxy)ethyl)piperazin-1-yl)ethyl)disulfaneyl)propyl)azanediyl)bis(6-hydroxyheptanoate) (GL-HEPES-E4-E7Ei3-DS-3-E7Ei5)

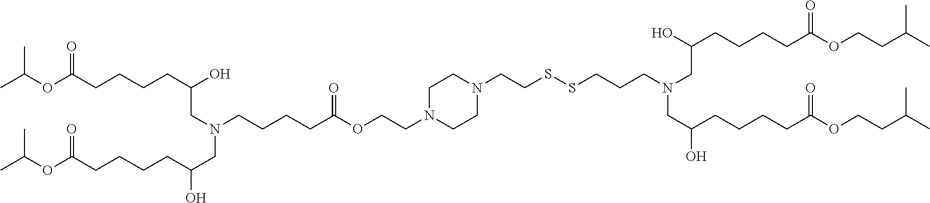 [SMILES: CC(C)CCOC(=O)CCCCC(O)CN(CCCSSCCN1CCN(CCOC(=O)CCCCN(CC(O)CCCCC(=O)OC(C)C)CC(O)CCCCC(=O)OC(C)C)CC1)CC(O)CCCCC(=O)OCCC(C)C]

¹H NMR (300 MHz, CDCl₃) δ 4.99 (hept, 2H), 4.19 (t, 2H), 4.08 (t, 8H), 3.62 (m, 4H), 2.85-2.24 (m, 40H), 1.95-1.27 (m, 34H), 1.21 (d, 12H), 0.91 (d, 12H).

APCI-MS analysis: Calculated C60H114N4O14S2 [M+H]=1179.7, Observed=1179.8.

### Diisopentyl 7,7′-((3-((2-(4-(2-((5-(bis(7-(2-ethylbutoxy)-2-hydroxy-7-oxoheptyl)amino)-pentanoyl)oxy)ethyl)piperazin-1-yl)ethyl)disulfaneyl)propyl)azanediyl)bis(6-hydroxyheptanoate)) (GL-HEPES-E4-E7Es6-DS-3-E7Ei5)

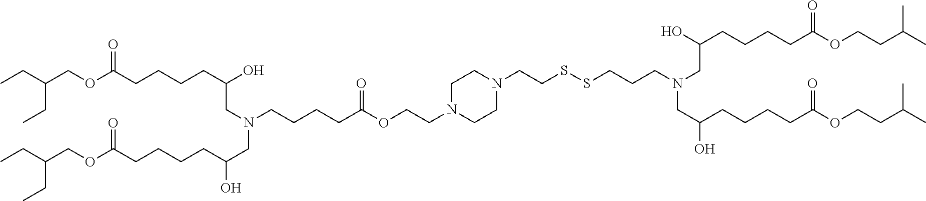 [SMILES: CCC(CC)COC(=O)CCCCC(O)CN(CCCCC(=O)OCCN1CCN(CCSSCCCN(CC(O)CCCCC(=O)OCCC(C)C)CC(O)CCCCC(=O)OCCC(C)C)CC1)CC(O)CCCCC(=O)OCC(CC)CC]

¹H NMR (300 MHz, CDCl₃) δ 4.19 (t, 2H), 4.08 (t, 4H), 3.98 (d, 4H), 3.63 (m, 4H), 2.85-2.25 (m, 40H), 1.95-1.24 (m, 48H), 0.91 (d, 12H), 0.88 (t, 12H).

APCI-MS analysis: Calculated C66H126N4O14S2 [M+H]=1263.8, Observed=1263.9.

### Diisopentyl 9,9′-((5-(2-(4-(2-((3-(bis(7-(2-ethylbutoxy)-2-hydroxy-7-oxoheptyl)amino)-propyl)disulfaneyl)ethyl)piperazin-1-yl)ethoxy)-5-oxopentyl)azanediyl)bis(8-hydroxynonanoate) (GL-HEPES-E4-E9Ei5-DS-3-E7Es6)

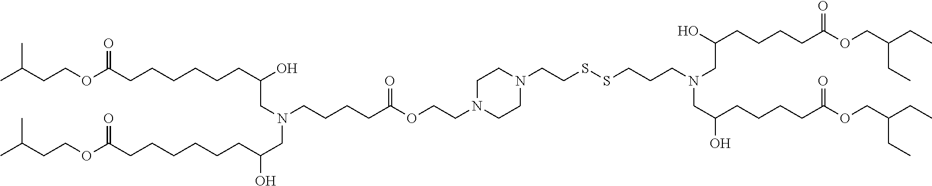 [SMILES: CCC(CC)COC(=O)CCCCC(O)CN(CCCSSCCN1CCN(CCOC(=O)CCCCN(CC(O)CCCCCCC(=O)OCCC(C)C)CC(O)CCCCCCC(=O)OCCC(C)C)CC1)CC(O)CCCCC(=O)OCC(CC)CC]

¹H NMR (300 MHz, CDCl₃) δ 4.19 (t, 2H), 4.08 (t, 4H), 3.98 (d, 4H), 3.62 (m, 4H), 2.85-2.23 (m, 38H), 1.79-1.25 (m, 58H), 0.91 (d, 12H), 0.88 (t, 12H).

APCI-MS analysis: Calculated C70H134N4O14S2 [M+H]=1319.9, Observed=1320.0.

### Bis(2-ethylbutyl) 9,9′-((5-(2-(4-(2-((3-(bis(7-(2-ethylbutoxy)-2-hydroxy-7-oxoheptyl)amino)-propyl)disulfaneyl)ethyl)piperazin-1-yl)ethoxy)-5-oxopentyl)azanediyl)bis(8-hydroxynonanoate) (GL-HEPES-E4-E9Es6-DS-3-E7Es6)

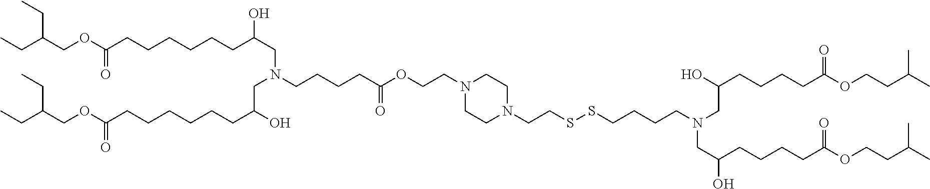 [SMILES: CCC(CC)COC(=O)CCCCCCC(O)CN(CCCCC(=O)OCCN1CCN(CCSSCCCCN(CC(O)CCCCC(=O)OCCC(C)C)CC(O)CCCCC(=O)OCCC(C)C)CC1)CC(O)CCCCCCC(=O)OCC(CC)CC]

¹H NMR (300 MHz, CDCl₃) δ 4.19 (t, 2H), 3.98 (d, 8H), 3.63 (m, 4H), 2.86-2.46 (m, 22H), 2.45-2.23 (m, 16H), 1.88-1.24 (m, 62H), 0.88 (t, 24H).

APCI-MS analysis: Calculated C72H138N4O14S2 [M+H]=1348.0, Observed=1348.0.

### Bis(2-ethylbutyl) 7,7′-((3-((2-(4-(2-((5-(bis(2-hydroxy-7-isopropoxy-7-oxoheptyl)amino)-pentanoyl)oxy)ethyl)piperazin-1-yl)ethyl)disulfaneyl)propyl)azanediyl)bis(6-hydroxyheptanoate) (GL-HEPES-E4-E7Ei3-DS-3-E7Es6)

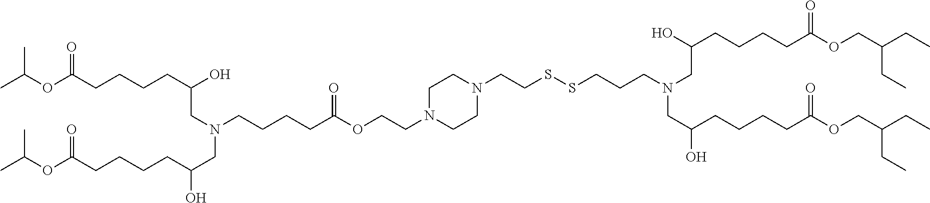 [SMILES: CCC(CC)COC(=O)CCCCC(O)CN(CCCSSCCN1CCN(CCOC(=O)CCCCN(CC(O)CCCCC(=O)OC(C)C)CC(O)CCCCC(=O)OC(C)C)CC1)CC(O)CCCCC(=O)OCC(CC)CC]

¹H NMR (300 MHz, CDCl₃) δ 4.99 (hept, 2H), 4.19 (t, 2H), 3.98 (d, 8H), 3.64 (m, 4H), 2.85-2.24 (m, 40H), 1.95-1.29 (m, 38H), 1.21 (d, 12H), 0.88 (t, 12H).

APCI-MS analysis: Calculated C62H118N4O14S2 [M+H]=1207.7, Observed=1207.8.

### Bis(2-ethylbutyl) 7,7′-((3-((2-(4-(2-((5-(bis(7-(2-ethylbutoxy)-2-hydroxy-7-oxoheptyl)-amino)pentanoyl)oxy)ethyl)piperazin-1-yl)ethyl)disulfaneyl)propyl)azanediyl)bis(6-hydroxyheptanoate) (GL-HEPES-E4-E7Es6-DS-3-E7Es6)

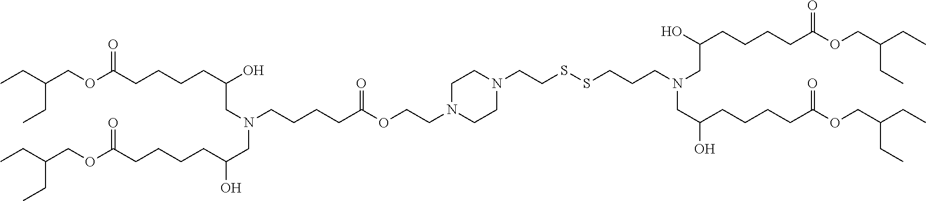 [SMILES: CCC(CC)COC(=O)CCCCC(O)CN(CCCCC(=O)OCCN1CCN(CCSSCCCN(CC(O)CCCCC(=O)OCC(CC)CC)CC(O)CCCCC(=O)OCC(CC)CC)CC1)CC(O)CCCCC(=O)OCC(CC)CC]

¹H NMR (300 MHz, CDCl₃) δ 4.19 (t, 2H), 3.98 (d, 8H), 3.63 (m, 4H), 2.85-2.25 (m, 40H), 1.95-1.24 (m, 52H), 0.88 (t, 24H).

APCI-MS analysis: Calculated C68H130N4O14S2 [M+H]=1291.9, Observed=1291.9.

### Diisopentyl 9,9′-((5-(2-(4-(2-((4-(bis(7-(2-ethylbutoxy)-2-hydroxy-7-oxoheptyl)amino)-butyl)disulfaneyl)ethyl)piperazin-1-yl)ethoxy)-5-oxopentyl)azanediyl)bis(8-hydroxynonanoate) (GL-HEPES-E4-E9Ei5-DS-4-E7Es6)

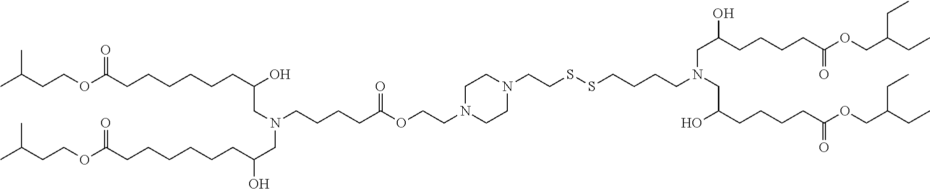 [SMILES: CCC(CC)COC(=O)CCCCC(O)CN(CCCCSSCCN1CCN(CCOC(=O)CCCCN(CC(O)CCCCCCC(=O)OCCC(C)C)CC(O)CCCCCCC(=O)OCCC(C)C)CC1)CC(O)CCCCC(=O)OCC(CC)CC]

¹H NMR (300 MHz, CDCl₃) δ 4.19 (t, 2H), 4.08 (t, 4H), 3.98 (d, 4H), 3.61 (m, 4H), 2.85-2.23 (m, 40H), 1.79-1.25 (m, 52H), 0.91 (d, 12H), 0.88 (t, 12H).

APCI-MS analysis: Calculated C71H136N4O14S2 [M+H]=1334.0, Observed=1334.0.

### Bis(2-ethylbutyl) 9,9′-((5-(2-(4-(2-((4-(bis(7-(2-ethylbutoxy)-2-hydroxy-7-oxoheptyl)amino)-butyl)disulfaneyl)ethyl)piperazin-1-yl)ethoxy)-5-oxopentyl)azanediyl)bis(8-hydroxynonanoate) (GL-HEPES-E4-E9Es6-DS-4-E7Es6)

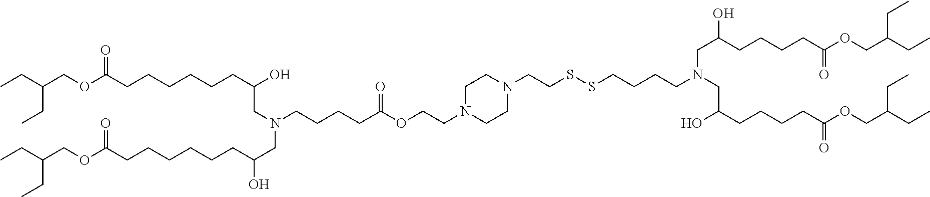 [SMILES: CCC(CC)COC(=O)CCCCCCC(O)CN(CCCCC(=O)OCCN1CCN(CCSSCCCCN(CC(O)CCCCC(=O)OCC(CC)CC)CC(O)CCCCC(=O)OCC(CC)CC)CC1)CC(O)CCCCCCC(=O)OCC(CC)CC]

¹H NMR (300 MHz, CDCl₃) δ 4.19 (t, 2H), 3.98 (d, 8H), 3.61 (m, 4H), 2.86-2.46 (m, 22H), 2.45-2.23 (m, 16H), 1.75-1.24 (m, 60H), 0.88 (t, 24H).

APCI-MS analysis: Calculated C73H140N4O14S2 [M+H]=1362.0, Observed=1362.0.

### Dibutyl 7,7′-((4-(2-(4-(2-((3-(bis(2-hydroxy-7-isopropoxy-7-oxoheptyl)amino)propyl)-disulfaneyl)ethyl)piperazin-1-yl)ethoxy)-4-oxobutyl)azanediyl)bis(6-hydroxyheptanoate) (GL-HEPES-E3-E7E4-DS-3-E7Ei3)

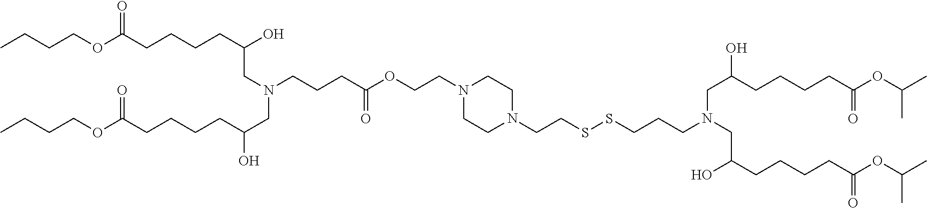 [SMILES: CCCCOC(=O)CCCCC(O)CN(CCCC(=O)OCCN1CCN(CCSSCCCN(CC(O)CCCCC(=O)OC(C)C)CC(O)CCCCC(=O)OC(C)C)CC1)CC(O)CCCCC(=O)OCCCC]

¹H NMR (300 MHz, CDCl₃) δ 4.99 (hept, 2H), 4.19 (t, 2H), 4.05 (t, 4H), 3.63 (m, 4H), 2.86-2.46 (m, 22H), 2.45-2.23 (m, 16H), 1.88-1.30 (m, 40H), 1.22 (d, 12H), 0.92 (t, 6H).

APCI-MS analysis: Calculated C57H108N4O14S2 [M+H]=1137.6, Observed=1137.7.

### Dibutyl 7,7′-((5-(2-(4-(2-((3-(bis(2-hydroxy-7-isopropoxy-7-oxoheptyl)amino)propyl)-disulfaneyl)ethyl)piperazin-1-yl)ethoxy)-5-oxopentyl)azanediyl)bis(6-hydroxyheptanoate) (GL-HEPES-E4-E7E4-DS-3-E7Ei3)

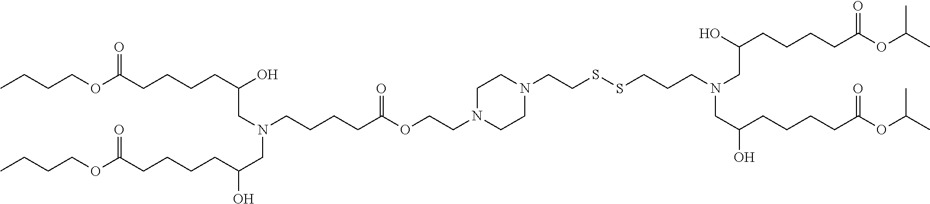 [SMILES: CCCCOC(=O)CCCCC(O)CN(CCCCC(=O)OCCN1CCN(CCSSCCCN(CC(O)CCCCC(=O)OC(C)C)CC(O)CCCCC(=O)OC(C)C)CC1)CC(O)CCCCC(=O)OCCCC]

¹H NMR (300 MHz, CDCl₃) δ 4.99 (hept, 2H), 4.19 (t, 2H), 4.05 (t, 4H), 3.63 (m, 4H), 2.86-2.46 (m, 22H), 2.45-2.23 (m, 16H), 1.88-1.30 (m, 42H), 1.22 (d, 12H), 0.92 (t, 6H).

APCI-MS analysis: Calculated C58H110N4O14S2 [M+H]=1151.6, Observed=1151.7.

HEPBS-based cationic lipids described herein may also be prepared according to Scheme 3:

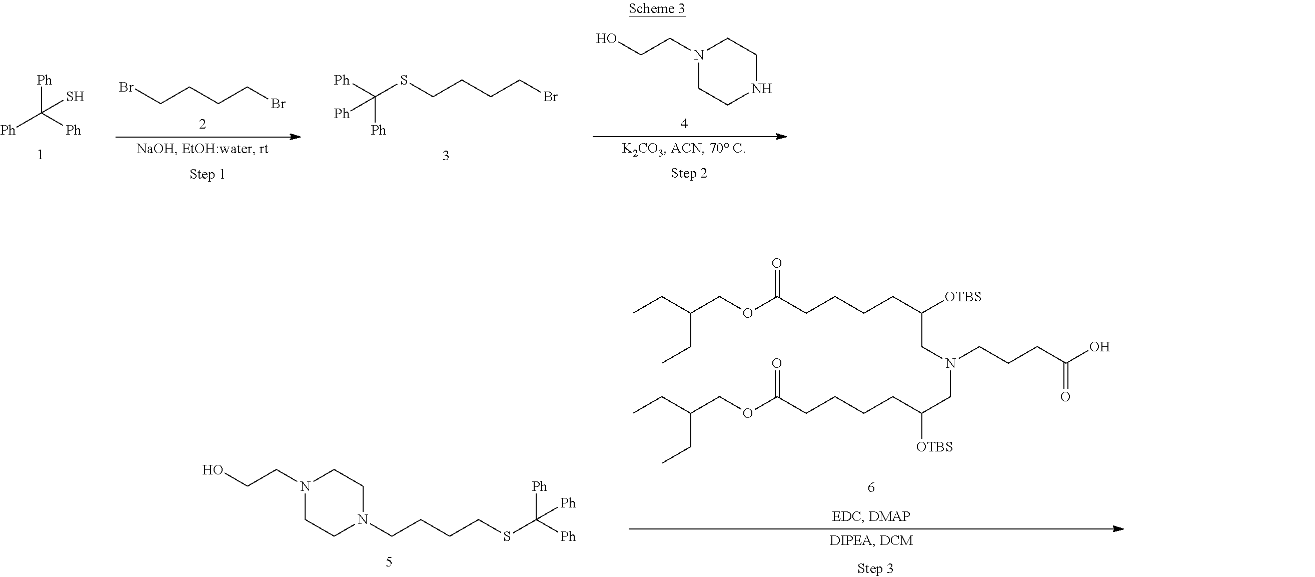 [SMILES: SC(c1ccccc1)(c1ccccc1)c1ccccc1 ; BrCCCCBr ; BrCCCCSC(c1ccccc1)(c1ccccc1)c1ccccc1 ; OCCN1CCNCC1 ; CCC(CC)COC(=O)CCCCC(CN(CCCC(=O)O)CC(CCCCC(=O)OCC(CC)CC)O[Si](C)(C)C(C)(C)C)O[Si](C)(C)C(C)(C)C ; OCCN1CCN(CCCCSC(c2ccccc2)(c2ccccc2)c2ccccc2)CC1]

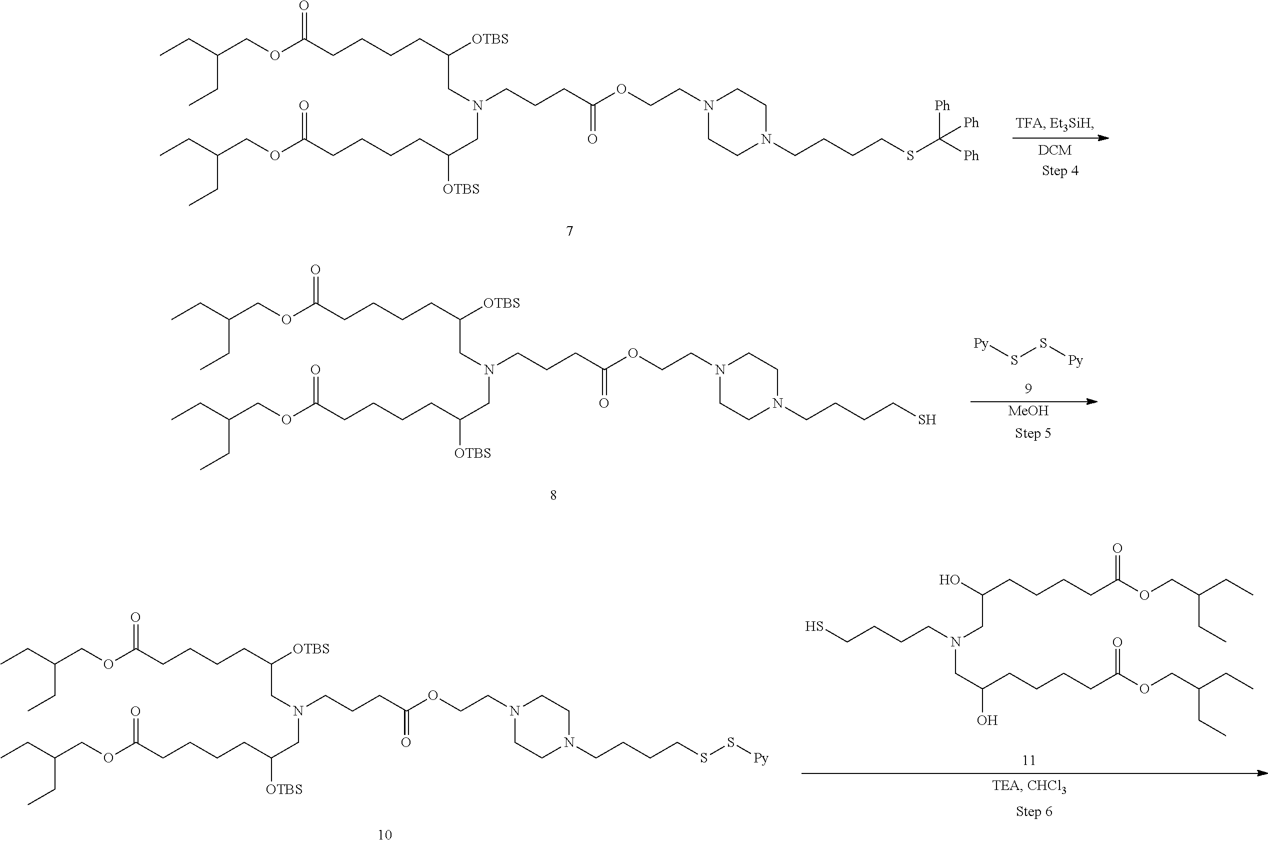

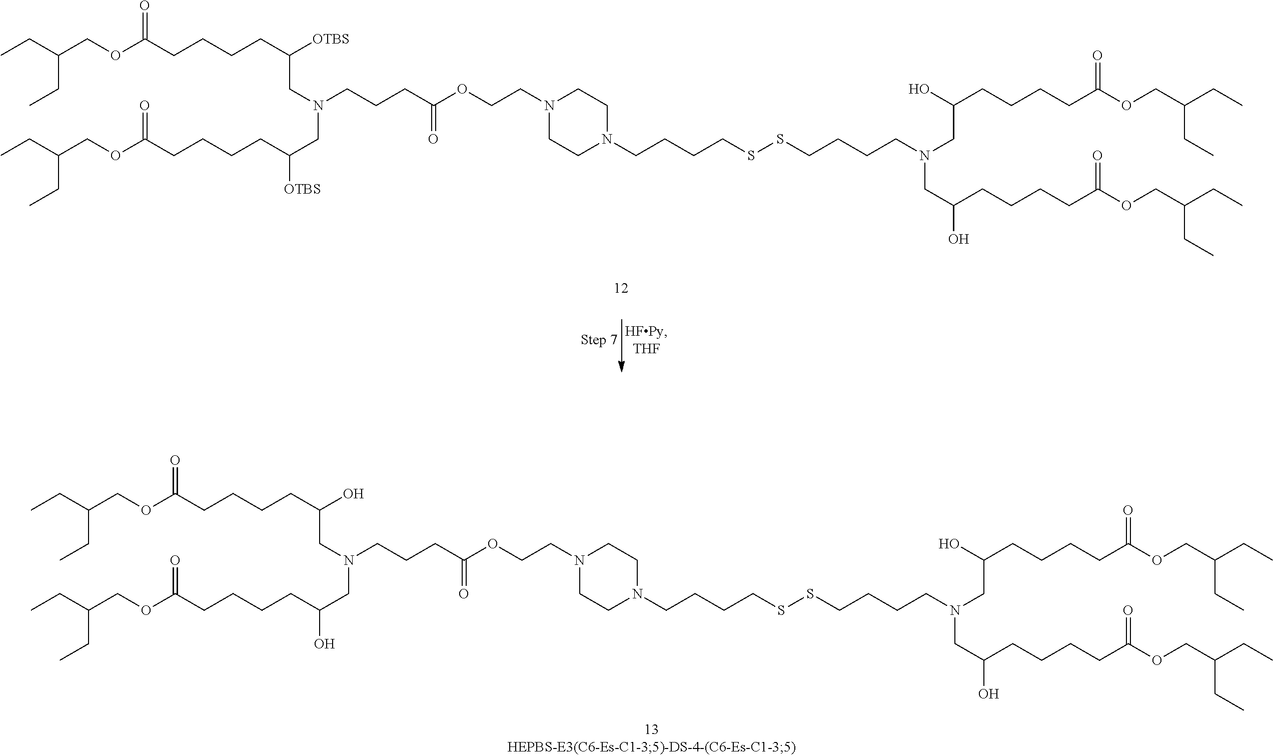

### Intermediate [3]

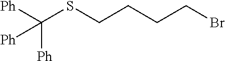 [SMILES: BrCCCCSC(c1ccccc1)(c1ccccc1)c1ccccc1]

To a solution of triphenylmethanethiol (5.0 g, 18.08 mmol) in EtOH (40 mL) and water (40 mL) was added a solution (in 40 mL water) of NaOH (1.44 g, 36.16 mmol). The reaction mixture was stirred for 10 min and added a solution (in 40 ml EtOH) of 1,4-dibromobutane (3.65 g, 18.08 mmol) to reaction mixture. The reaction mixture was stirred for 4 hours at room temperature. The progress of reaction was monitored by TLC (5% EtOAc/hexanes). The reaction mixture was diluted DCM and aqueous sodium bicarbonate solution, the organic layer was washed with brine. The organic layer was dried over sodium sulphate, concentrated under vacuum to give crude compound. To the crude was added MeOH (15 mL) and stirred for 15 min at 0-10° C., the solid compound was filtered and dried under vacuum to give [3](5.1 g, 69%) as a white solid.

### Results:

1H NMR (400 MHz, CDCl3): δ 7.42-7.39 (m, 6H), 7.30-7.26 (m, 6H), 7.23-7.19 (m, 3H), 3.24 (t, 2H), 2.17 (t, 2H), 1.82-1.77 (m, 2H), 1.55-1.50 (m, 2H). LCMS: Purity 84.99% (low ionization)

### Intermediate [5]

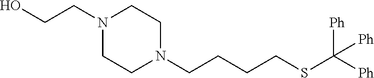 [SMILES: OCCN1CCN(CCCCSC(c2ccccc2)(c2ccccc2)c2ccccc2)CC1]

To a solution of [3](5.0 g, 12.16 mmol) and [4](3.16 g, 24.32 mmol) in ACN (75 mL) was added K2CO3 (6.72 g, 48.62 mmol). The reaction mixture was heated at 40° C. for 48 hours. The reaction progress was monitored by TLC (2.5% MeOH in DCM)). The reaction mixture was cooled to room temperature and filtered. The filtrate was concentrated under vacuum to give crude product. The crude was purified by flash chromatography (0 to 2.5% MeOH in DCM) to give [5](2.6 g, 46%) as a white solid.

### Results:

1H NMR (400 MHz, DMSO-d6): δ 7.41 (d, 6H), 7.28 (d, 6H), 7.20 (t, 3H), 3.59 (t, 2H), 2.73 (brs, 1H), 2.53-2.39 (m, 10H), 2.20-2.14 (m, 4H), 1.41 (brs, 4H). LCMS: Purity 98%

ESI-MS analysis: Calculated C29H37N20S, [M+H]=461.26, Observed=461.29

### Intermediate [7]

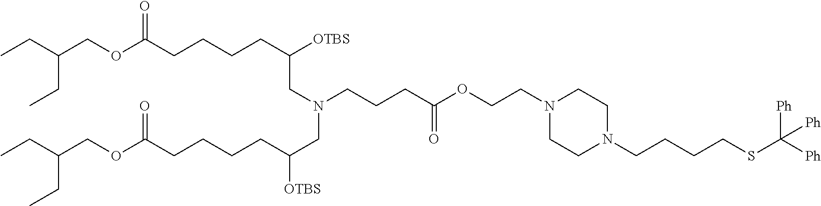 [SMILES: CCC(CC)COC(=O)CCCCC(CN(CCCC(=O)OCCN1CCN(CCCCSC(c2ccccc2)(c2ccccc2)c2ccccc2)CC1)CC(CCCCC(=O)OCC(CC)CC)O[Si](C)(C)C(C)(C)C)O[Si](C)(C)C(C)(C)C]

To a solution of [5](0.613 g, 1.33 mmol) in DCM (7 mL) were added [6](1.0 g, 1.26 mmol) in DCM (8 mL), EDC (0.364 g, 1.90 mmol), DMAP (31 mg, 0.253 mmol), DIPEA (0.442 mL, 2.54 mmol) and stirred at room temperature for 14 hours. After completion of the reaction as monitored by MS. The reaction mixture was diluted with DCM washed with NaHCO₃ solution, water and brine. The organic layer was dried over anhydrous Na₂SO₄, concentrated, and the crude compound was purified (eluent: 20% EtOAc in hexanes) to obtain pure compound [7] as a color less oil (0.77 g, 49%). It was confirmed by MS analysis.

### Results:

ESI-MS analysis: Calculated C₇₁H₁₁₉N₃O₈SSi₂, [M+H]=1230.98, Observed=1230.8

### Intermediate [8]

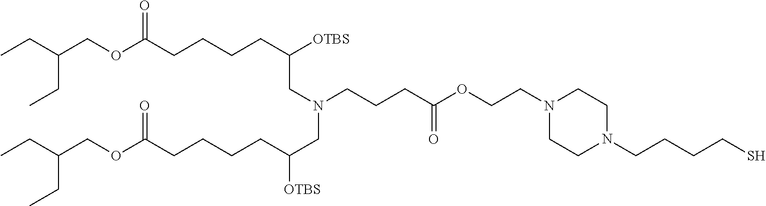 [SMILES: CCC(CC)COC(=O)CCCCC(CN(CCCC(=O)OCCN1CCN(CCCCS)CC1)CC(CCCCC(=O)OCC(CC)CC)O[Si](C)(C)C(C)(C)C)O[Si](C)(C)C(C)(C)C]

To a solution of [7](0.77 g, 0.625 mmol) in DCM (3 mL) was slowly added TFA (3 mL) at room temperature and stirred at room temperature for 0.5 hour. To that triethylsilane (0.124 mL, 0.782 mmol) was added slowly and stirred for 1 hour. After completion of the reaction as monitored by MS. The reaction mixture was concentrated to obtain crude product [8](quantitative). It was confirmed by MS analysis.

### Results:

ESI-MS analysis: Calculated C₅₂H₁₀₅N₃O₈SSi₂, [M+H]=988.66, Observed=988.66

### Intermediate [10]

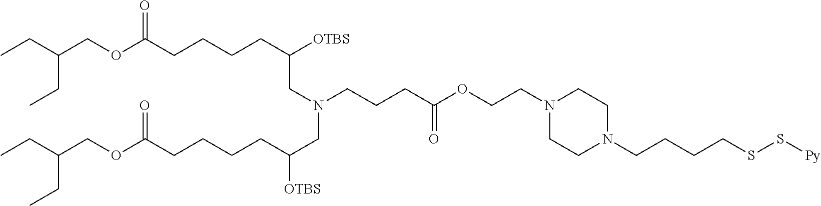

To a solution of [8](quantitative) in MeOH (4 mL) was added [9](0.234 g, 1.06 mmol) at room temperature and stirred for 2 hours. After completion of the reaction as monitored by MS. The reaction mixture was concentrated, and the crude compound was purified (eluent:100% Ethyl Acetate, then 0-20% Methanol in Ethyl Acetate) to obtain pure product [10](0.691 g, Quantitative Yield). It was confirmed by MS analysis.

### Results:

ESI-MS analysis: Calculated for C₅₇H₁₀₈N₄O₈S₂Si₂, [M+H]=1097.80; Observed=1097.8

### Intermediate [12]

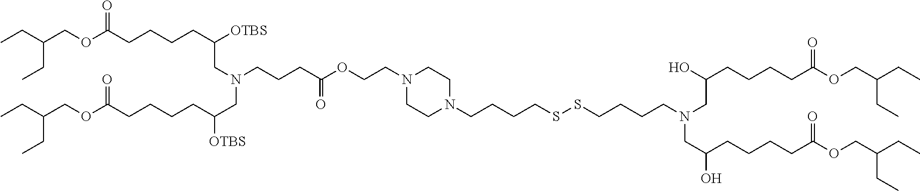 [SMILES: CCC(CC)COC(=O)CCCCC(O)CN(CCCCSSCCCCN1CCN(CCOC(=O)CCCN(CC(CCCCC(=O)OCC(CC)CC)O[Si](C)(C)C(C)(C)C)CC(CCCCC(=O)OCC(CC)CC)O[Si](C)(C)C(C)(C)C)CC1)CC(O)CCCCC(=O)OCC(CC)CC]

To a solution of [10](0.350 g, 0.319 mmol) and [11](0.322 g, 0.574 mmol) in chloroform was added triethylamine (0.266 ml, 1.91 mmol) and allowed to react at room temperature for 2.5 hours. After completion of the reaction, the reaction mixture was concentrated and taken to the next step without purification (0.800 g Crude Material).

ESI-MS analysis: Calculated for C82H₁₆₂N₄O₁₄S₂Si₂, [M+H]=1548.50; Observed=1548.8

### GL-HEPBS-E3(C6-Es-C1-3;5)-DS-4-(C6-Es-C1-3;5) [13]:

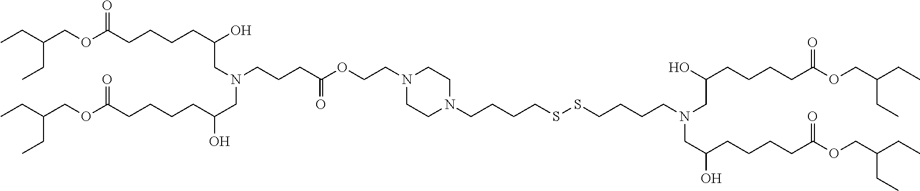 [SMILES: CCC(CC)COC(=O)CCCCC(O)CN(CCCCSSCCCCN1CCN(CCOC(=O)CCCN(CC(O)CCCCC(=O)OCC(CC)CC)CC(O)CCCCC(=O)OCC(CC)CC)CC1)CC(O)CCCCC(=O)OCC(CC)CC]

To a 20 ml polypropylene scintillation vial was added [12](Crude Material, 0.800 g) along with 4 mL of dry tetrahydrofuran. The vial was cooled to 0-5° C. and HF/pyridine (2.0 mL, 76.33 mmol) was added dropwise. After addition, the reaction vial was allowed to warm to room temperature and stirred for 18 hours. Afterwards, the reaction mixture was cooled back to 0° C. and neutralized with solid sodium bicarbonate solid, diluted with ethyl acetate, washed with NaHCO₃ solution, water and brine. The organic layer was dried over anhydrous Na₂SO₄ and concentrated. The crude product was purified to obtain compound [13](0.196 g, 46% Over Two Steps). It was confirmed by ¹H NMR and MS analysis.

### Results:

¹H NMR (400 MHz, CDCl₃) 4.19 (t, 2H), 3.97 (d, 8H), 3.64 (br, 4H), 2.76-2.22 (m, 36H), 1.86-1.74 (m, 2H), 1.73-1.56 (m, 15H), 1.55-1.44 (m, 9H), 1.43-1.26 (m, 28H), 0.87 (t, 24H).

ESI-MS analysis: Calculated for C₇₀H₁₃₄N₄O₁₄S₂, [M+H]=1319.98; Observed=1319.8

HEPBS-based cationic lipids described herein may also be prepared according to Scheme 4:

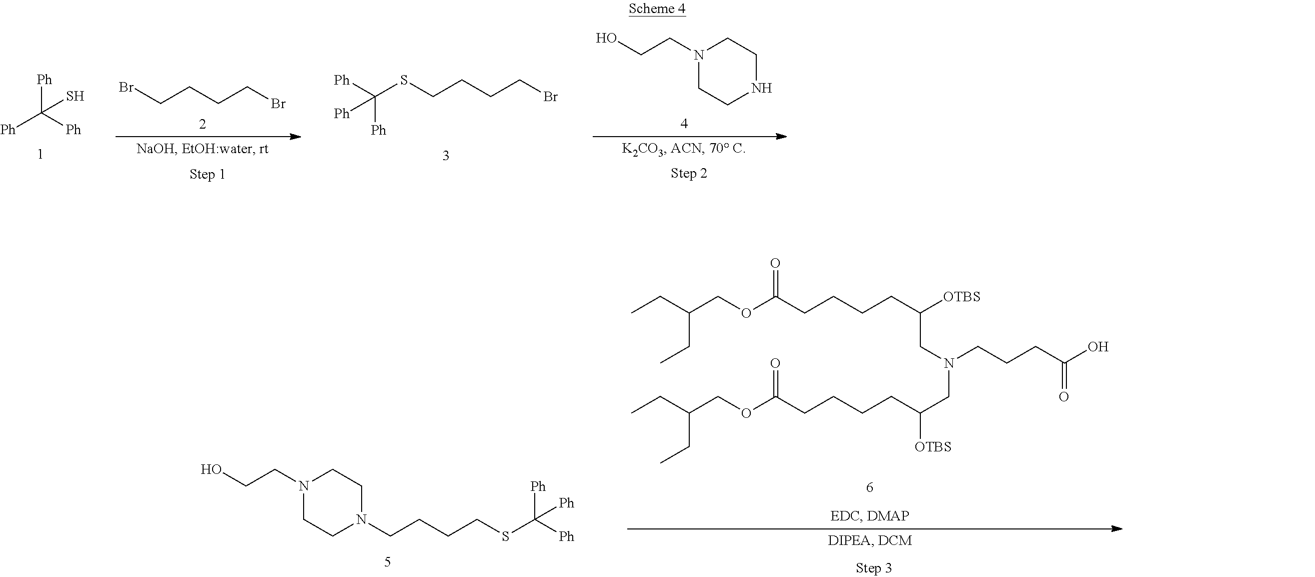 [SMILES: SC(c1ccccc1)(c1ccccc1)c1ccccc1 ; BrCCCCBr ; BrCCCCSC(c1ccccc1)(c1ccccc1)c1ccccc1 ; OCCN1CCNCC1 ; CCC(CC)COC(=O)CCCCC(CN(CCCC(=O)O)CC(CCCCC(=O)OCC(CC)CC)O[Si](C)(C)C(C)(C)C)O[Si](C)(C)C(C)(C)C ; OCCN1CCN(CCCCSC(c2ccccc2)(c2ccccc2)c2ccccc2)CC1]

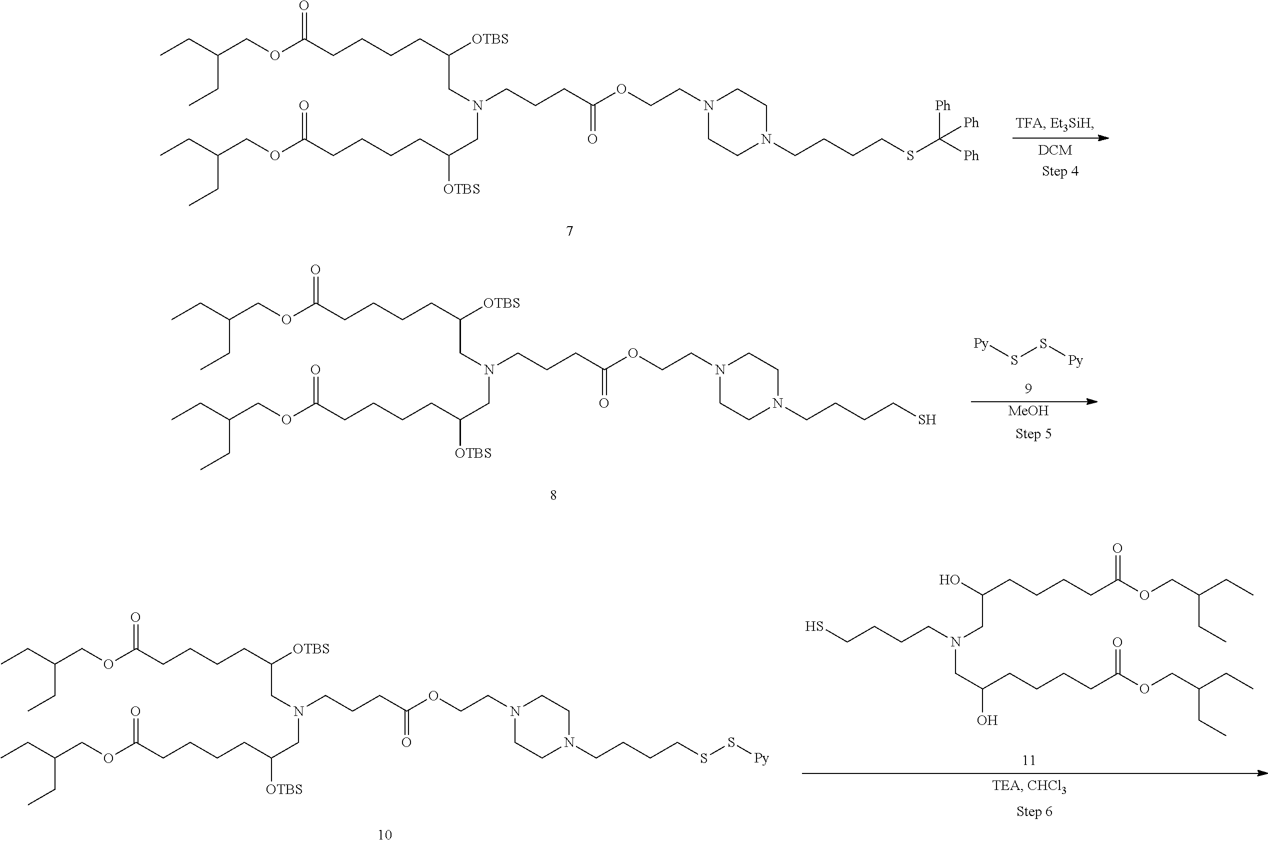

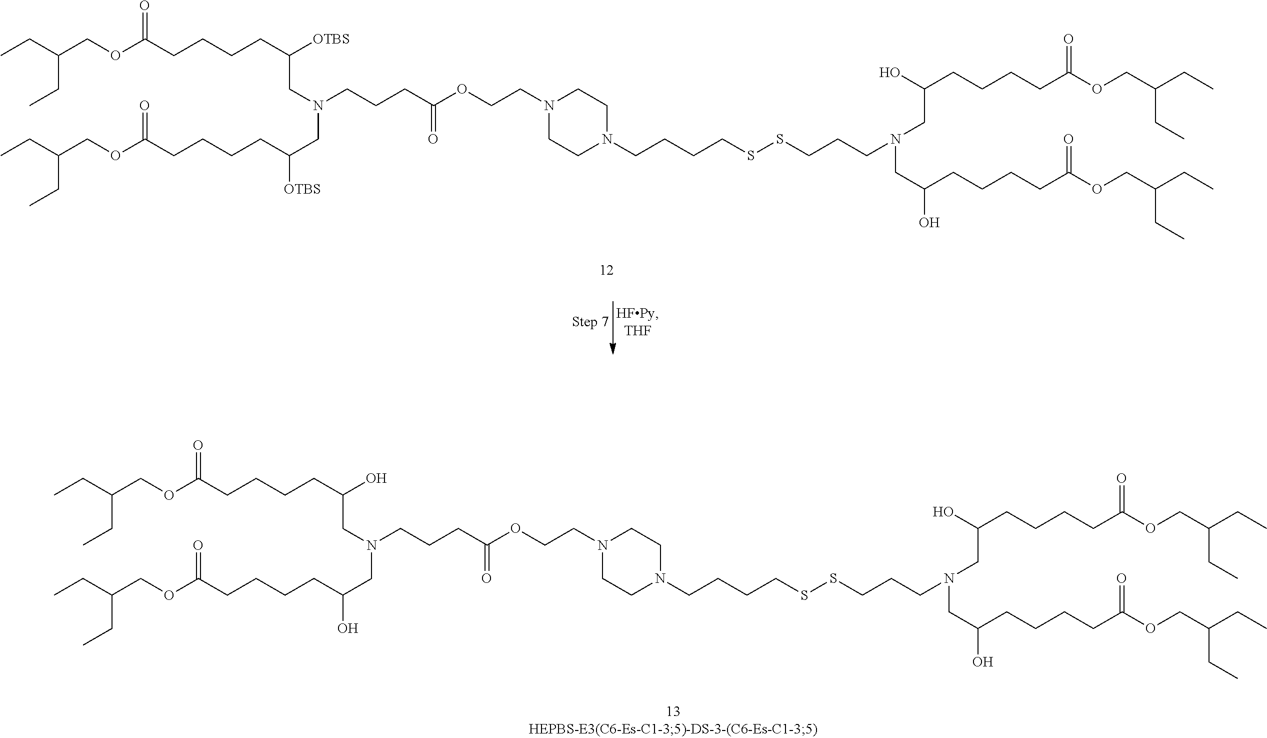

Intermediate 5 was synthesized using the same procedures as Scheme 3.

### Intermediate [7]

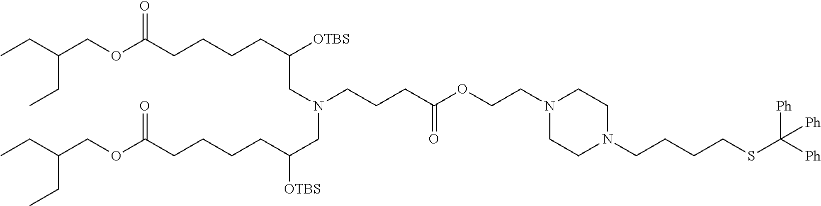 [SMILES: CCC(CC)COC(=O)CCCCC(CN(CCCC(=O)OCCN1CCN(CCCCSC(c2ccccc2)(c2ccccc2)c2ccccc2)CC1)CC(CCCCC(=O)OCC(CC)CC)O[Si](C)(C)C(C)(C)C)O[Si](C)(C)C(C)(C)C]

To a solution of [5](0.613 g, 1.33 mmol) in DCM (7 mL) were added [6](1.0 g, 1.26 mmol) in DCM (8 mL), EDC (0.364 g, 1.90 mmol), DMAP (31 mg, 0.253 mmol), DIPEA (0.442 mL, 2.54 mmol) and stirred at room temperature for 14 hours. After completion of the reaction as monitored by MS. The reaction mixture was diluted with DCM washed with NaHCO₃ solution, water and brine. The organic layer was dried over anhydrous Na₂SO₄, concentrated, and the crude compound was purified (eluent: 20% EtOAc in hexanes) to obtain pure compound [7] as a color less oil (0.77 g, 49%). It was confirmed by MS analysis.

### Results:

ESI-MS analysis: Calculated C₇₁H₁₁₉N₃O₈SSi₂, [M+H]=1230.98, Observed=1230.8

### Intermediate [8]

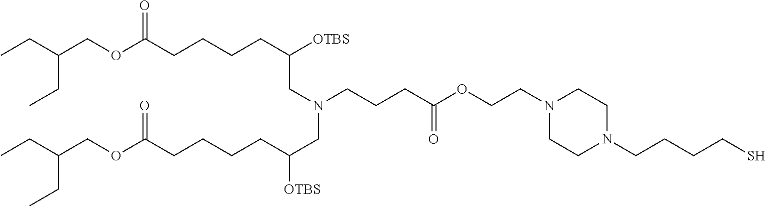 [SMILES: CCC(CC)COC(=O)CCCCC(CN(CCCC(=O)OCCN1CCN(CCCCS)CC1)CC(CCCCC(=O)OCC(CC)CC)O[Si](C)(C)C(C)(C)C)O[Si](C)(C)C(C)(C)C]

To a solution of [7](0.77 g, 0.625 mmol) in DCM (3 mL) was slowly added TFA (3 mL) at room temperature and stirred at room temperature for 0.5 hour. To that triethylsilane (0.124 mL, 0.782 mmol) was added slowly and stirred for 1 hour. After completion of the reaction as monitored by MS. The reaction mixture was concentrated to obtain crude product [8](quantitative). It was confirmed by MS analysis.

### Results:

ESI-MS analysis: Calculated C₅₂H₁₀₅N₃O₈SSi₂, [M+H]=988.66, Observed=988.66

### Intermediate [10]

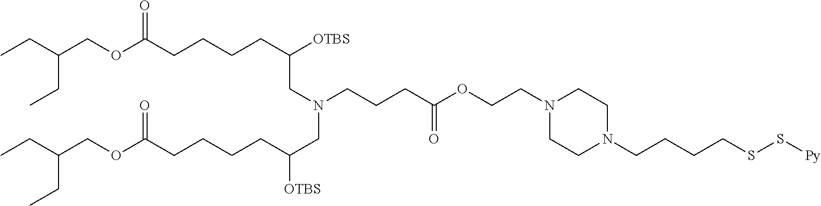

To a solution of [8](quantitative) in MeOH (4 mL) was added [9](0.234 g, 1.06 mmol) at room temperature and stirred for 2 hours. After completion of the reaction as monitored by MS. The reaction mixture was concentrated, and the crude compound was purified (eluent:100% Ethyl Acetate, then 0-20% Methanol in Ethyl Acetate) to obtain pure product [10](0.691 g, Quantitative Yield). It was confirmed by MS analysis.

### Results:

ESI-MS analysis: Calculated for C₅₇H₁₀₈N₄O₈S₂Si₂, [M+H]=1097.80; Observed=1097.8

### Intermediate [12]

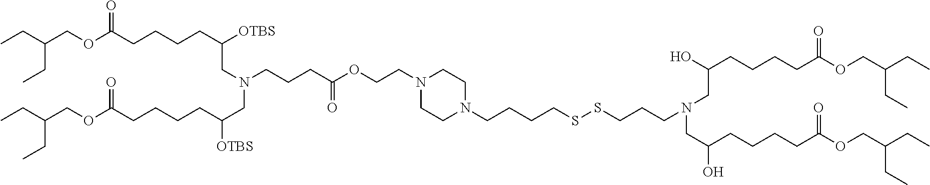 [SMILES: CCC(CC)COC(=O)CCCCC(O)CN(CCCSSCCCCN1CCN(CCOC(=O)CCCN(CC(CCCCC(=O)OCC(CC)CC)O[Si](C)(C)C(C)(C)C)CC(CCCCC(=O)OCC(CC)CC)O[Si](C)(C)C(C)(C)C)CC1)CC(O)CCCCC(=O)OCC(CC)CC]

To a solution of [10](0.320 g, 0.291 mmol) and [11](0.287 g, 0.525 mmol) in chloroform was added triethylamine (0.243 ml, 1.75 mmol) and allowed to react at room temperature for 2.5 hours. After completion of the reaction, the reaction mixture was concentrated and taken to the next step without purification (0.800 g Crude Material).

ESI-MS analysis: Calculated for C₈₁H₁₆₀N₄O₁₄S₂Si₂, [M+H]=1534.48; Observed=1534.8

### GL-HEPBS-E3(C6-Es-C1-3;5)-DS-3-(C6-Es-C1-3;5) [13]:

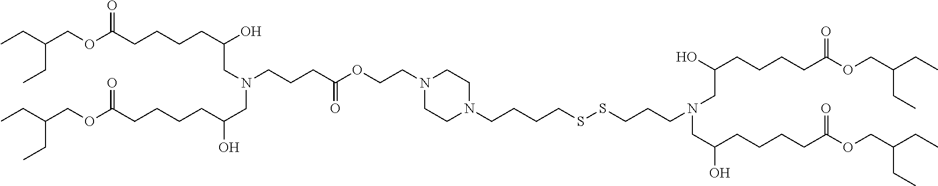 [SMILES: CCC(CC)COC(=O)CCCCC(O)CN(CCCSSCCCCN1CCN(CCOC(=O)CCCN(CC(O)CCCCC(=O)OCC(CC)CC)CC(O)CCCCC(=O)OCC(CC)CC)CC1)CC(O)CCCCC(=O)OCC(CC)CC]

To a 20 ml polypropylene scintillation vial was added [12](Crude Material, 0.800 g) along with 4 mL of dry tetra hydrofuran. The vial was cooled to 0-5° C. and HF/pyridine (2.0 mL, 77.03 mmol) was added dropwise. After addition, the reaction vial was allowed to warm to room temperature and stirred for 18 hours. Afterwards, the reaction mixture was cooled back to 0° C. and neutralized with solid sodium bicarbonate solid, diluted with ethyl acetate, washed with NaHCO₃ solution, water and brine. The organic layer was dried over anhydrous Na₂SO₄ and concentrated. The crude product was purified to obtain compound [13](0.211 g, 55% Over Two Steps). It was confirmed by 0H NMR and MS analysis.

### Results:

¹H NMR (400 MHz, CDCl₃) 4.19 (t, 2H), 3.97 (d, 8H), 3.64 (br, 4H), 2.85-2.23 (m, 36H), 1.89-1.74 (m, 4H), 1.73-1.55 (m, 12H), 1.55-1.44 (m, 8H), 1.43-1.28 (m, 30H), 0.87 (t, 24H).

ESI-MS analysis: Calculated for C₆₉H₁₃₂N₄O₁₄S₂, [M+H]=1305.95; Observed=1305.8

### Example 2: Lipid Nanoparticle Formulation

Cationic lipids described herein can be used in the preparation of lipid nanoparticles according to methods known in the art. For example, suitable methods include methods described in International Publication No. WO 2018/089801, which is hereby incorporated by reference in its entirety.

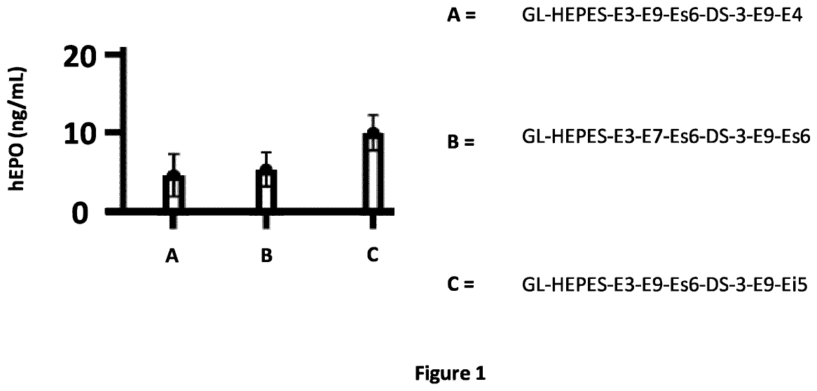

The lipid nanoparticles in the examples of the present invention were formulated using Process A of WO 2018/089801 (see, e.g., Example 1 and FIG. 1 of WO 2018/089801). Process A (“A”) relates to a conventional method of encapsulating mRNA by mixing mRNA with a mixture of lipids, without first pre-forming the lipids into lipid nanoparticles. In an exemplary process, an ethanolic solution of a mixture of lipids (cationic lipid, phosphatidylethanolamine, cholesterol, and polyethylene glycol-lipid) at a fixed lipid to mRNA ratio were combined with an aqueous buffered solution of target mRNA at a acidic under controlled conditions to yield a suspension of uniform LNPs. After ultrafiltration and concentrated. The e suitable diluent system, the resulting nanoparticle suspensions were diluted to final concentration, filtered, and stored frozen at −80° C. until use.

Lipid nanoparticle formulations of Table 3 were prepared by Process A. All of the lipid nanoparticle formulations comprised hEPO mRNA and the different lipids (Cationic Lipid: DMG-PEG2000: Cholesterol: DOPE/DSPC) in the mol % ratios specified in Table 3.

TABLE 3
Exemplary lipid nanoparticle characterizations

| Formu- | | Formulation Composition | | |
|---|---|---|---|---|
| lation | | Cationic Lipid:DMG- | | N/ |
| number | mRNA | PEG2000:Cholesterol:DOPE/DSPC | Process | P* |
| 1 | hEPO | Cationic Lipid:DMG- | A | 4 |
| | | PEG2000:Cholesterol:DOPE: | | |
| | | 40:1.5-3:27-28.5:30 | | |
| 2 | hEPO | Cationic Lipid:DMG- | A | 3 |
| | | PEG2000:Cholesterol:DSPC: | | |
| | | 50:1.5:38.5:10 | | |

*The N/P ratio is defined as the ratio of the number of nitrogen in cationic lipid to the number of phosphate in nucleic acid.

The cationic lipids of the present invention were evaluated with lipid nanoparticle formulation 1. MC3 was evaluated with lipid nanoparticle formulation 2, which is a typical MC3 formulation.

### Example 3: Delivery of hEPO mRNA by Intramuscular Administration

### Mouse Studies

In summary, lipid screening studies were conducted with female BALB/cJ mice 6-8 weeks of age. Mice were dosed with 0.1 μg in 30 μL of LNPs by a single intramuscular (IM) injection into the gastrocnemius leg muscle. Blood samples were taken 6 and 24 hours post injection and hEPO levels were measured in the blood serum of the mice using an ELISA assay according to the manufacture's protocol. WO2022/099003 A1 also describes an in vivo assay for intramuscular administration (e.g. on page 46, paragraph [00206]).

Further details of the intramuscular experiment performed in this application are provided below.

Study Design Table

| | | | Dose | | Dose | | |
|---|---|---|---|---|---|---|---|
| Group | No. of | | Levels | Conc. | Volume | Dosing Regimen | Terminal |
| No. | Animals | Test Article | (μg/An.) | (μg/mL) | (μL/An.) | ROA | Time Point |
| 1 | 64 | MC3 | 0.1 | 3.33 | 30 | Once on Day 1 | At 24 hours |
| 2 | 4 | 51 | 0.1 | 3.33 | | via IM injections | post dose on |
| 3 | 4 | 50 | 0.1 | 3.33 | | into the right | Day 2 |
| 4 | 4 | 57 | 0.1 | 3.33 | | gastrocnemius | |
| 5 | 4 | 92 | 0.1 | 3.33 | | muscle. | |
| 6 | 4 | 46 | 0.1 | 3.33 | | | |
| 7 | 4 | 29 | 0.1 | 3.33 | | | |
| 8 | 4 | 42 | 0.1 | 3.33 | | | |
| 9 | 4 | 53 | 0.1 | 3.33 | | | |
| 10 | 4 | 6 | 0.1 | 3.33 | | | |
| 11 | 4 | 45 | 0.1 | 3.33 | | | |
| 12 | 4 | 12 | 0.1 | 3.33 | | | |
| 13 | 4 | 78 | 0.1 | 3.33 | | | |

An. = animal; TA = test article; Conc. = concentration; ROA = route of administration; IM = intramuscular.

### Test Materials and Treatment Regimen

Test materials remained RNase free during loading into the syringe (as applicable).

### Test Article Class of Compound: Oligonucleotides

### ABSL-1

Treatment Regimen: On Day 1, animals from Groups 1-13 were dosed via intramuscular injection while under light isoflurane anesthesia according to the study design table above. Animals in Groups 1-13 were injected with EPO mRNA LNPs in the right leg only. Group 1 animals received MC3 control. The cationic lipid MC3 is the current gold standard for in vivo delivery of e.g. siRNA (see WO2010/144740).

### Study Animals

### Animals:

| | |
|---|---|
| Species/Sex | Mouse/Female |
| Strain | BALB/cJ (Jax #000651) |
| Number | N = 112 |
| Age | 6-8 Weeks |

Acclimation: Animals were acclimatised to the Test Facility for at least 24 hours.

Housing: All animals were socially housed in polycarbonate cages with contact bedding in an animal housing room.

Food and Water: Food (Envigo irradiated 2918 diet) and filtered tap water was provided to animals ad libitum.

### In-Life Observations and Measures

Animal Health Checks: At least once daily animals received a cage side health check observation.

Clinical Observations: Clinical observations were performed for all animals on Day 1 prior to dose administration and prior to euthanasia. Clinical observations were performed more often if abnormal clinical signs were exhibited by animals on study.

Body Weights: Body weights were recorded prior to test material administration. Body weights were rounded to the nearest 0.1 g.

Interim Sample Collections: Interim whole blood (^{˜}50 μL) was collected by tail snip or saphenous vein at 6 and 24 hours post dose administration (±5%). Blood samples were collected into serum separator tubes, allowed to clot at room temperature for at least 10 minutes, centrifuged at ambient temperature at minimum 1000 g for 10 minutes and the serum was extracted. All serum samples were stored at nominally −70° C. until analysis hEPO by the Testing Facility. The results of the EPO analysis were included in the Data Submission.

In-Life Sample Collection Table

| | In Life Sample Collection Time Points |
|---|---|
| Group No. | Whole Blood |
| 1-13 | T = 6 Hours Post Dose |
| Volume | ~50 μL |
| Additive | None |

No. = number

### Terminal Procedures

Euthanasia: On Day 2, 24 hours post dose, all animals were euthanized by CO₂ asphyxiation followed by thoracotomy and terminal blood collection.

Terminal Blood Collections: Whole blood was collected via cardiac puncture into serum separator tube, allowed to clot at room temperature for at least 10 minutes, centrifuged at ambient temperature at minimum 1000 g for 10 minutes and the serum was extracted. Serum samples were stored at nominally −70° C. until analyzed for hEPO by the Test Facility.

Terminal Sample Collection Table

| | Terminal Sample Collection Time Points |
|---|---|
| Group No. | Whole Blood |
| 1-13 | T = 24 Hours Post Dose |
| Volume | MOV |
| Additive | None |

No. = number; MOV = maximum obtainable volume.

### In-Vitro Assays:

ELISA Assay: Human erythropoietin (hEPO) levels in sera samples were determined by ELISA kit (R&D systems, Cat #DEP-00) according to the manufactory instruction and the results were included in the Data Submission. The “shaker” protocol was used. The serum samples were diluted between 1:40 and 1:100.

### Reporting and Data Retention

Data Submission: A tabulated data summary of animal assignment, individual and group means (as applicable) for times of dose administration and euthanasia, body weights, clinical observations in-vitro analysis and mortality (as applicable) were delivered for this study.

TABLE 4
Results of hEPO mRNA delivery studies - intramuscular
administration of hEPO mRNA lipid formulations
comprising the claimed cationic lipids.

| Com- | hEPO 6 Hr | | hEPO 24 Hr | | |
|---|---|---|---|---|---|
| pound | (ng/mL) | hEPO 6 Hr | (ng/mL) | hEPO 24 Hr | # of |
| # | Mean | (ng/mL) SD | Mean | (ng/mL) SD | mice |
| MC3 | 3.94 | 1.24 | 2.44 | 0.63 | 64 |
| 51 | 16.39 | 3.50 | 9.03 | 2.50 | 4 |
| 50 | 14.94 | 1.57 | 8.87 | 1.22 | 4 |
| 57 | 13.33 | 1.68 | 8.19 | 0.44 | 4 |
| 92 | 13.11 | 2.62 | 5.75 | 0.77 | 4 |
| 46 | 12.38 | 1.61 | 5.78 | 0.68 | 4 |
| 29 | 10.01 | 2.25 | 4.93 | 0.62 | 4 |
| 42 | 9.07 | 0.85 | 3.06 | 0.15 | 4 |
| 53 | 9.07 | 3.01 | 4.66 | 1.75 | 4 |
| 6 | 8.72 | 1.41 | 8.90 | 1.47 | 4 |
| 45 | 8.33 | 1.50 | 3.99 | 1.30 | 4 |
| 12 | 8.06 | 1.54 | 4.86 | 1.06 | 4 |
| 78 | 7.05 | 2.08 | 3.30 | 1.18 | 4 |

### Example 4: Laurdan Assay for Determining Generalized Polarization (GP) Values

The laurdan probe was used to compare the lipid packing in lipid nanoparticles comprising the second generation of cationic lipids derived from “Good” buffers of the present invention with lipid nanoparticles comprising other cationic lipids derived from “Good” buffers.

Formulations were diluted into buffer solutions at pH 4.5, 5.5, 6.5, or 7.5 and the laurdan molecule was added to a final laurdan concentration of 1 μM. Solutions were incubated at room temperature, protected from light, for three hours. The GP value was calculated based off fluorescence values to give an idea of formulation lipid membrane packing. Samples were analyzed using a SpectraMax M5 Multi-Mode microplate reader. A fluorescence excitation wavelength of 340 nm was used along with emission wavelengths of 440 and 490 nm. GP values were calculated using the following equation: GP=(AUC₄₄₀−AUC₄₉₀)/(AUC₄₄₀+AUC₄₉₀).

Further details of the Laurdan Assay for determining Generalized Polarization (GP) values are provided in 1) Koitabashi, K.; Nagumo, H.; Nakao, M.; Machida, T.; Yoshida, K.; Sakai-Kato, K. Acidic PH-Induced Changes in Lipid Nanoparticle Membrane Packing. Biochimica Et Biophysica Acta Bba—Biomembr 2021, 1863 (8), 183627, and 2) Parasassi, T.; Stasio, G. D.; Ravagnan, G.; Rusch, R. M.; Gratton, E. Quantitation of Lipid Phases in Phospholipid Vesicles by the Generalized Polarization of Laurdan Fluorescence. Biophys J 1991, 60 (1), 179-189, both of which are incorporated herein by reference.

The laurdan probe inserts itself homogeneously into the hydrophilic/hydrophobic interface of the lipid bilayer and is used to measure polarity changes in the bilayer environment which can be related to lipid membrane packing and orderliness. A generalized polarization (GP) value was calculated from a shift in fluorescence intensity of 440 nm to 490 nm when the laurdan probe interacts with water molecules in the lipid membrane. A lower GP value is associated with a hydrated and fluid membrane while a higher GP value typically means less water molecules and more ordered lipid packing. The GP value of the lipid nanoparticles (LNPs) was measured in pH 7.5, 6.5, 5.5, and 4.5 buffers to simulate endosomal pH shift that occurs when particles are taken up by cells. It is contemplated that lower pH levels (4.5 and 5.5) may result in lower GP values for all formulations tested compared to pH 6.5 and 7.5. This suggests that lipid nanoparticles (LNPs) are becoming more fluid and less orderly when the pH environment decreases. Lipid nanoparticles comprising the second generation of cationic lipids derived from “Good” buffers are contemplated to have overall higher GP values compared with lipid nanoparticles comprising other cationic lipids derived from “Good” buffers. The additional esters and/or carbon branches in the lipid tails of the second generation of cationic lipids derived from “Good” buffers are contemplated to result in tighter packed membranes compared to other cationic lipids derived from “Good” buffers. A positive trend is contemplated to be observed between GP value and amount of hEPO produced in mice at pH 6.5 for lipid nanoparticles comprising the second generation of cationic lipids derived from “Good” buffers. One hypothesis for the contemplated correlation between GP value and protein production is that particles with tighter bilayer packing may perform better in vivo by increasing lipid nanoparticle (LNP) stability under physiological pH conditions.

In summary, lipid nanoparticles comprising the second generation of cationic lipids derived from “Good” buffers of the present invention are contemplated to have higher overall Generalized Polarization (GP) values compared to other cationic lipids derived from “Good” buffers. A positive linear correlation is contemplated between the laurdan GP value and the amount of EPO produced at 6 hours in mice. An increase in GP value is contemplated to correlate with an increase in EPO protein for pH 6.5 solutions.

### Example 5: In Vitro Degradation Study

### Lipid Degradability by MOUSE/HUMAN Lung S9 In Vitro

Assay format—4 or 5 time points in triplicate.

### 1. Assay Procedure:

1) Plan experiment, compounds, and reagents.
2) Dissolve each lipid in DMSO or IPA to make 5 mM stock, then dilute by IPA to 200 μM work solution.
3) Thaw mouse and human lung S9.
4) Prepare pooled incubation mixture as in the reaction formulas below on ice.
5) Aliquot 495 μL incubation mixture prepared in step #4 to each well of a 2 mL 96-well plate.
6) Add 5 μL compound to each well to initiate the reaction. Take t0 samples (as in step #8).
7) Cover the plate with 2 layers of breathable seals and incubate the plate on an orbital shaker at 150 rpm in a 37° C. CO₂ incubator.
8) At each time point, pipette to mix the incubation mixture 5 times, then take 70 μL of incubation mixture to a fresh plate. Store in −20° C. freezer immediately.
9) Add 210 μL (3× volume) of the cold stop solution to each well of the sample plates collected. Mix at 600 rpm on an orbital shaker for 15 min.
10) Centrifuge the quenched plates at 3800 rpm for 10 min at 4° C. and transfer supernatant to fresh plates.
11) Load the supernatant on filter plates and centrifuge again at 3800 rpm for 5 min at 4° C. Collect final samples in fresh plates for LC/MS.
   Ii. Time Course and Stop Solutions:
4-5 Time points (hour): e.g. 0, 4, 8, 24, 48 hr
stop solution: 1:1:1 ACN/MeOH/IPA (v/v/v) with propranolol & MC3 as internal standard. Store at 4° C.

### III. Reaction Components and Formulas:

MOUSE/HUMAN lung S9

| Final conc. | Reaction component | Volume (μL) |
|---|---|---|
| | H₂O | 155.0 |
| 100 mM | 5x potassium phosphate buffer pH 7.4 | 100.0 |
| 2.4 mg/ml | each species lung S9 (5 mg/ml) | 240.0 |
| 2 μM | lipid (200 μM) | 5.0 |
| | Total | 500 |

### Example 6: RiboGreen Assay

The encapsulation efficiency of mRNA in lipid nanoparticles can be determined using Invitrogen RiboGreen assay kit. The unencapsulated mRNA was detected directly. The total mRNA was measured after lysis of lipid nanoparticles in the presence 0.45% w/v of Triton X-100. The encapsulation efficiency was calculated as (Total mRNA−unencapsulated mRNA)/Total mRNA×100%.

The RiboGreen Assay is a fluorescence-based method for the determination of mRNA concentration (Total and Free) and % encapsulation using Quant-iT™ RiboGreen® RNA reagent in mRNA containing lipid nanoparticles.

### Materials/Reagents

Triton-X, 98%, for molecular biology, DNAse, RNAse and Protease free, Acros Organics, Cat. AC327371000
UltraPure DNase/RNase-free Distilled Water Life Technologies, Cat. 10977-023
RNaseZap® RNase Decontamination Solution Life Technologies, Cat. AM9784
Quant-iT™ RiboGreen® RNA Reagent Life Technologies, Cat. R11491 or Quant-iT™
RiboGreen® RNA Assay Kit Life Technologies, Cat. R11490
RNase free 20×TE Buffer Life Technologies, Cat. T11493
RNaseZap® RNase Decontamination Solution Life Technologies, Cat. AM9784

### Equipment

Molecular Devices Gemini EM Microplate Reader
RNase Free Microcentrifuge Tubes (2.0 mL)
RNase Free Flacon Tubes (15 and 50 mL)
Vortex mixer
Corning® 96 Well Special Optics Microplate with Clear Background (Cat #3615)
   Preparation of mRNA Standards

mRNA Dilution

| | 50X | 2X |
|---|---|---|
| 10X TE buffer | 950 μL | 1920 μL |
| mRNA | 50 μL | 80 μL |

| | 0 | | 0.02 ug/mL | | 0.05 ug/mL | | 0.2 ug/mL | | 0.4 ug/mL | | 0.6 ug/mL | |
|---|---|---|---|---|---|---|---|---|---|---|---|---|
| Standards | blank | | mRNA-1 | | mRNA-2 | | mRNA-3 | | mRNA-4 | | mRNA-5 | |
| 10X TE buffer | 950 | μL | 930 | μL | 900 | μL | 750 | μL | 550 | μL | 350 | μL |
| 4% Triton | 50 | μL | 50 | μL | 50 | μL | 50 | μL | 50 | μL | 50 | μL |
| 2X mRNA dil. | 0 | μL | 20 | μL | 50 | μL | 200 | μL | 400 | μL | 600 | μL |
| 200-Fold RG | 1000 | μL | 1000 | μL | 1000 | μL | 1000 | μL | 1000 | μL | 1000 | μL |

### Sample Preparation

Sample Dilution

| | 100X |
|---|---|
| 10X TE buffer | 990 μL |
| LNP sample | 10 μL |

Samples

| | |
|---|---|
| Total mRNA | Free mRNA |

| | | | | |
|---|---|---|---|---|
| 10X TE buffer | 900 | μL | 750 | μL |
| 4% Triton | 50 | μL | 0 | μL |
| LNP sample dil. (100x) | 50 | μL | 250 | μL |
| 200-Fold RG | 1000 | μL | 1000 | μL |

### 200-Fold RiboGreen Dye Preparation

200-Fold RiboGreen

| | |
|---|---|
| # tubes*5 uL = Amount of RG | Ex. (10 tubes + 2extra) *5 μL = 60 |
| in (#tube)mL TE | μL RG in 12 mL TE |

### Procedure

To each of the standards (Blank, mRNA-1, mRNA-2. mRNA-3, mRNA-4, mRNA-5) and Samples (free mRNA and total mRNA), add 1.0 mL of 200-fold Ribogreen Reagent Solution and gently mix by inversion. This is a 2× Dilution.
Add 200 μL of each standard and sample in triplicate using the reverse pipetting technique in a 96-well Costar Black with Clear Background Plate. Ensure no bubbles are present in the plate before the fluorescence reading.
Read the fluorescence signal using the below instrument parameters:
Read Type: Fluorescence, Bottom Read
Excitation: 485 nm; Cut-off: 515 nm; Emission: 530 nm
Plate Type: 96-well Costar Black with Clear Background

### Data Analysis

The average fluorescence from each calibration standard is plotted against the concentration to generate a linear calibration curve using the MS Excel software. The coefficient of determination (R²) of calibration curve must be R²>0.99.

The linear equation generated can be interpreted as follows:

$$y = mx + c$$

Where,
Y=average fluorescence value
m: slope
x: concentration (μg/mL)
c: y-intercept
   Using the linear equation, calculate the concentration of free and total mRNA concentration in the test sample by replacing the y value in the equation with the average fluorescence value of each respective sample
   Once the concentration is determined, the actual concentration in the sample can be back-calculated by multiplying the concentration in the test sample with the dilution factor (DF) as follows:

$$Free mRNA Conc . = Conc . of Free mRNA in Test Sample \times 800 ( DF ) \\ Total mRNA Conc . = Conc . of Total mRNA in Test Sample \times 4000 ( DF )$$

Concentration of encapsulated mRNA can be determined by subtracting the concentration of free mRNA from the total mRNA.
% Encapsulation can then be calculated by taking the ratio of encapsulated mRNA over total mRNA and multiplying the result with 100.

### Example 7: Delivery of Human Erythropoietin (hEPO) mRNA by Intramuscular (IM) Administration

Lipid nanoparticle (LNP) formulations encapsulating hEPO mRNA were prepared by Process A as described above for IM administration. The LNP compositions administered comprised 1.5% PEG, 40% Cationic lipid, 28.5% Cholesterol, and 30% DOPE an N/P ratio of 4. After LNP formulation, the nanoparticles were initially buffer exchanged with 20% EtOH, and then with a final buffer exchange in 10% Trehalose. The LNPs were characterized for size, PDI, encapsulation, and mRNA concentration. For the hEPO animal dosing studies, the LNPs were diluted to 3.33 ug/mL in 10% trehalose. Mice were dosed intramuscularly with 0.1 ug in 30 uL volume into the right gastrocnemius muscle. Blood samples were collected 6 hours and 24 hours post injection to measure the amount of hEPO protein produced in the serum. The EPO protein amounts were detected using an ELISA assay from commercially available kits. FIG. 1 shows that lipid nanoparticles comprising lipids described herein are highly effective in delivering hEPO mRNA and show high levels of hEPO protein expression at 6 hours post-IM injection dose.

The Polydispersity Index (PdI) of lipid nanoparticles can be determined by diluting the formulation in 10% trehalose at about 0.1 mg/ml mRNA concentration and then measuring the size on Malvern zetasizer.

The lipid nanoparticle size can be obtained with Malvern Zetasizer Nano-ZS.

From the foregoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

### NUMBERED EMBODIMENTS

1. A compound having a structure according to Formula (I):

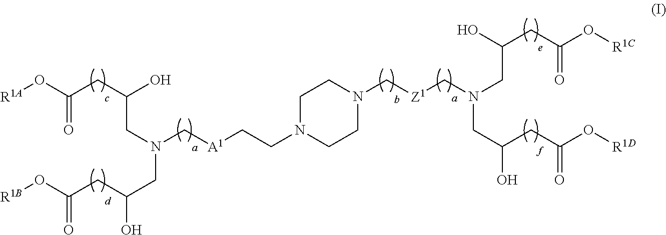

or a pharmaceutically acceptable salt thereof, wherein:
A¹ is selected from

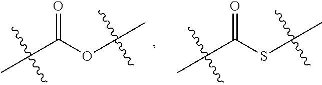 [SMILES: *OC(*)=O ; *SC(*)=O]

and —S—S—, wherein the left hand side of each depicted structure is bound to the —(CH₂)a-;
Z¹ is selected from

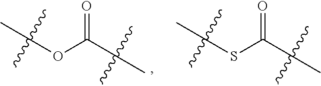 [SMILES: *OC(*)=O ; *SC(*)=O]

and —S—S—, wherein the right hand side of each depicted structure is bound to the —(CH₂)a-;
each a is independently selected from 3 or 4;
b is 1, 2, 3, 4 or 5;
each c, d, e and f is independently selected from 3, 4, 5 or 6; and
each R^{1A}, R^{1B}, R^{1C} and R^{1D} is independently selected from optionally substituted (C₃-C₆)alkyl.

2. The compound of numbered embodiment 1, wherein the compound has a structure according to Formula (Ia):

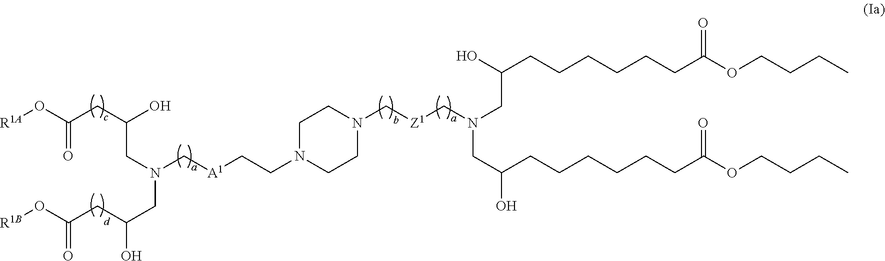

or a pharmaceutically acceptable salt thereof, optionally wherein:
(d) b is 2;
(e) b is 2, A¹ is

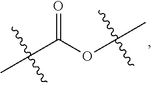 [SMILES: *OC(*)=O]

wherein the left hand side of the depicted structure is bound to the —(CH₂)a- and Z¹ is —S—S—; or
(f) b is 2, A¹ is

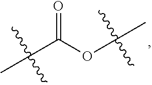 [SMILES: *OC(*)=O]

wherein the left hand side of the depicted structure is bound to the —(CH₂)a-, Z¹ is —S—S— and each c and d is independently selected from 3, 4, or 6.

3. The compound of numbered embodiment 1, wherein the compound has a structure according to Formula (Ib):

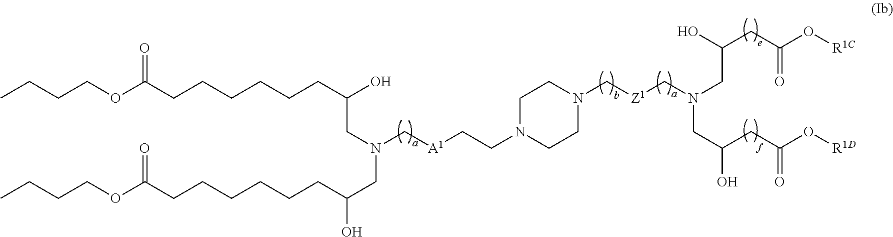

or a pharmaceutically acceptable salt thereof, optionally wherein:
(d) b is 2;
(e) b is 2, A¹ is

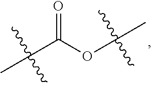 [SMILES: *OC(*)=O]

wherein the left hand side of the depicted structure is bound to the —(CH₂)a- and Z¹ is —S—S—; or
(f) b is 2, A¹ is

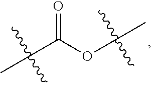 [SMILES: *OC(*)=O]

wherein the left hand side of the depicted structure is bound to the —(CH₂)a-, Z¹ is —S—S— and each e and f is independently selected from 3, 4, or 6.

4. The compound of numbered embodiment 1, wherein the compound has a structure according to Formula (Ic):

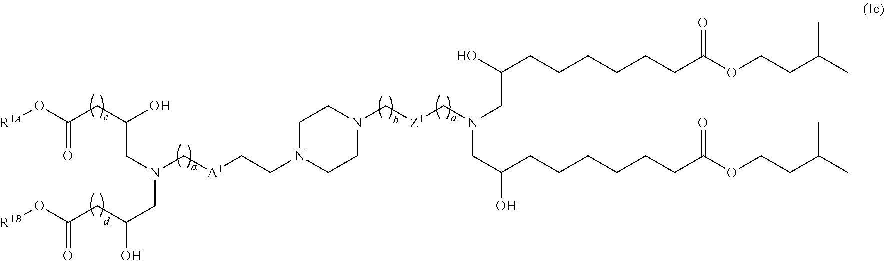

or a pharmaceutically acceptable salt thereof, optionally wherein:
(d) b is 2;
(e) b is 2, A¹ is

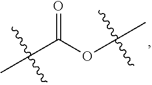 [SMILES: *OC(*)=O]

wherein the left hand side of the depicted structure is bound to the —(CH₂)a- and Z¹ is —S—S—; or
(f) b is 2, A¹ is

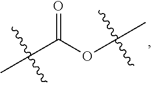 [SMILES: *OC(*)=O]

wherein the left hand side of the depicted structure is bound to the —(CH₂)a-, Z¹ is —S—S— and each c and d is independently selected from 3, 4, or 6.

5. The compound of numbered embodiment 1, wherein the compound has a structure according to Formula (Id):

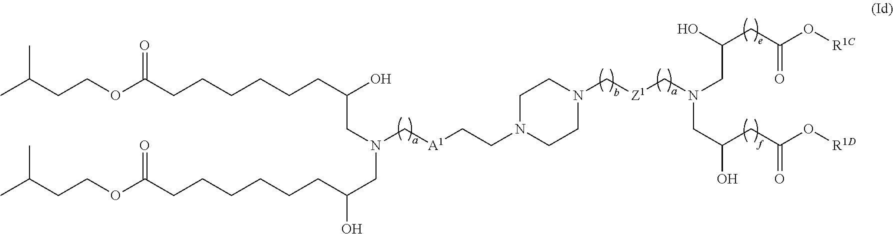

or a pharmaceutically acceptable salt thereof, optionally wherein:
(d) b is 2;
(e) b is 2, A¹ is

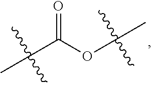 [SMILES: *OC(*)=O]

wherein the left hand side of the depicted structure is bound to the —(CH₂)a- and Z¹ is —S—S—; or
(f) b is 2, A¹ is

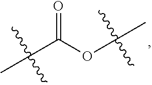 [SMILES: *OC(*)=O]

herein the left hand side of the depicted structure is bound to the —(CH₂)a-, Z¹ is —S—S— and each e and f is independently selected from 3, 4, or 6.

6. The compound of numbered embodiment 1, wherein the compound has a structure according to Formula (Ie):

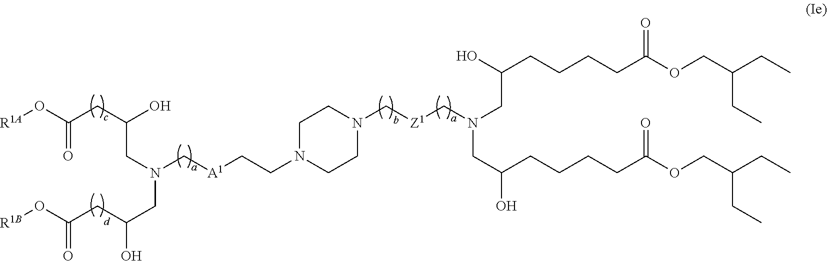

or a pharmaceutically acceptable salt thereof, optionally wherein:
(d) b is 2;
(e) b is 2, A¹ is

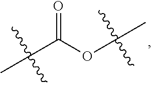 [SMILES: *OC(*)=O]

wherein the left hand side of the depicted structure is bound to the —(CH₂)a- and Z¹ is —S—S—; or
(f) b is 2, A¹ is

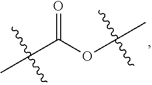 [SMILES: *OC(*)=O]

wherein the left hand side of the depicted structure is bound to the —(CH₂)a-, Z¹ is —S—S— and each c and d is independently selected from 3, 4, or 6.

7. The compound of numbered embodiment 1, wherein the compound has a structure according to Formula (if):

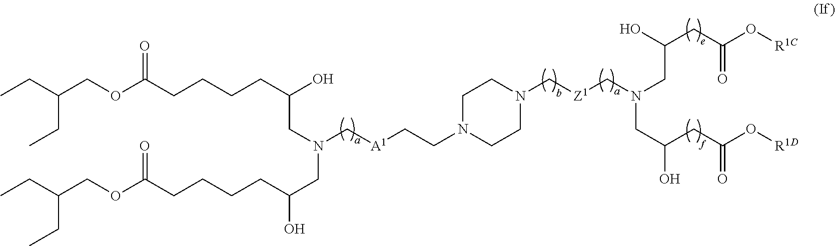

or a pharmaceutically acceptable salt thereof, optionally wherein:
(d) b is 2;
(e) b is 2, A¹ is

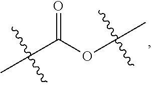 [SMILES: *OC(*)=O]

wherein the left hand side of the depicted structure is bound to the —(CH₂)a- and Z¹ is —S—S—; or
(f) b is 2, A¹ is

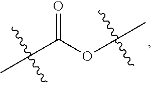 [SMILES: *OC(*)=O]

wherein the left hand side of the depicted structure is bound to the —(CH₂)a-, Z¹ is —S—S— and each e and f is independently selected from 3, 4, or 6.

8. The compound of numbered embodiment 1, wherein the compound has a structure according to Formula (Ig):

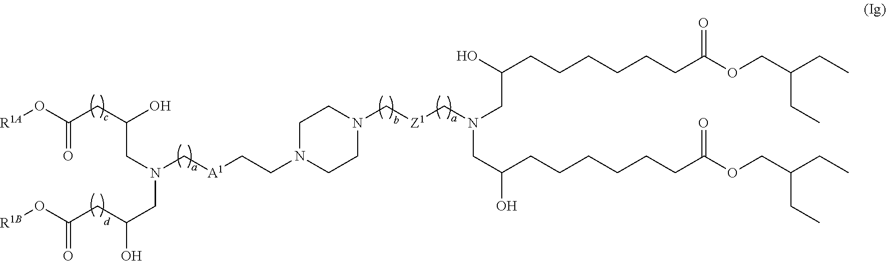

or a pharmaceutically acceptable salt thereof, optionally wherein:
(d) b is 2;
(e) b is 2, A¹ is

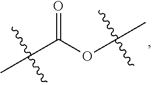 [SMILES: *OC(*)=O]

wherein the left hand side of the depicted structure is bound to the —(CH₂)a- and Z¹ is —S—S—; or
(f) b is 2, A¹ is

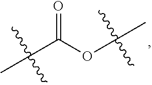 [SMILES: *OC(*)=O]

wherein the left hand side of the depicted structure is bound to the —(CH₂)a-, Z¹ is —S—S— and each c and d is independently selected from 3, 4, or 6.

9. The compound of numbered embodiment 1, wherein the compound has a structure according to Formula (Ih):

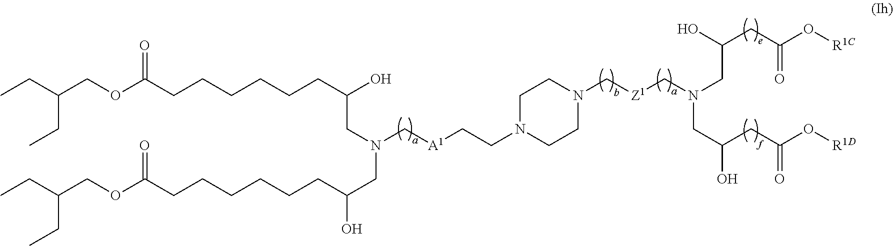

or a pharmaceutically acceptable salt thereof, optionally wherein:
(d) b is 2;
(e) b is 2, A¹ is

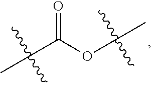 [SMILES: *OC(*)=O]

wherein the left hand side of the depicted structure is bound to the —(CH₂)a- and Z¹ is —S—S—; or
(f) b is 2, A¹ is

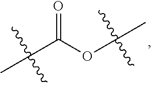 [SMILES: *OC(*)=O]

wherein the left hand side of the depicted structure is bound to the —(CH₂)a-, Z¹ is —S—S— and each e and f is independently selected from 3, 4, or 6.

10. The compound of numbered embodiment 1, wherein the compound has a structure according to Formula (Ii):

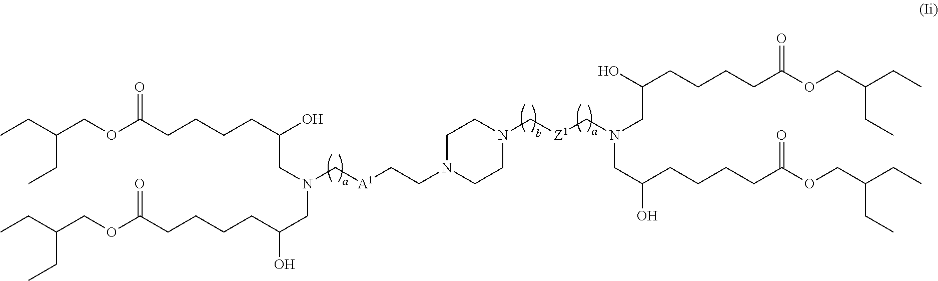

or a pharmaceutically acceptable salt thereof, optionally wherein:
(c) b is 2; or
(d) b is 2, A¹ is

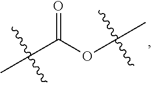 [SMILES: *OC(*)=O]

wherein the left hand side of the depicted structure is bound to the —(CH₂)a- and Z¹ is —S—S—.

11. The compound of numbered embodiment 1, wherein the compound has a structure according to Formula (Ij):

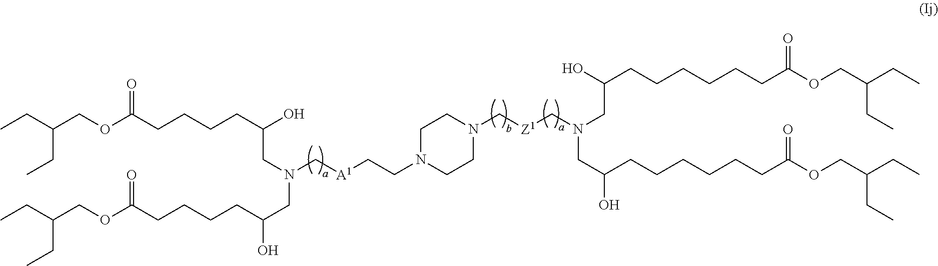

or a pharmaceutically acceptable salt thereof, optionally wherein:
(c) b is 2; or
(d) b is 2, A¹ is

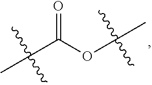 [SMILES: *OC(*)=O]

wherein the left hand side of the depicted structure is bound to the —(CH₂)a- and Z¹ is —S—S—.

12. The compound of numbered embodiment 1, wherein the compound has a structure according to Formula (Ik):

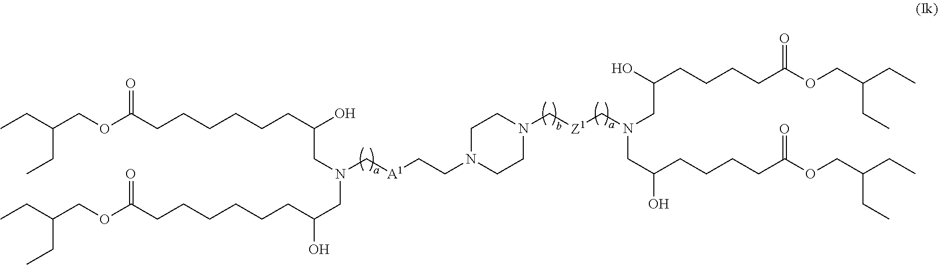

or a pharmaceutically acceptable salt thereof, optionally wherein:
(c) b is 2; or
(d) b is 2, A¹ is

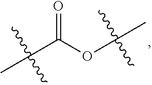 [SMILES: *OC(*)=O]

wherein the left hand side of the depicted structure is bound to the —(CH₂)a- and Z¹ is —S—S—.

13. The compound of numbered embodiment 1, wherein the compound has a structure according to Formula (Im):

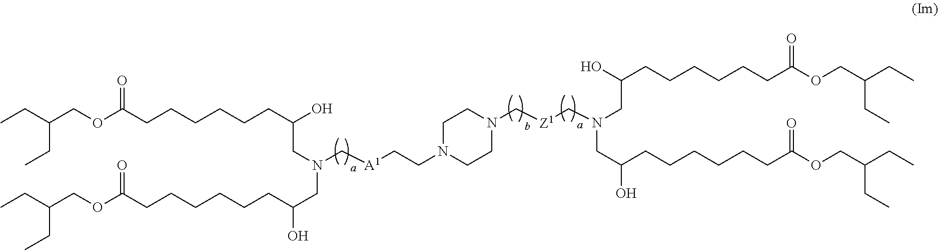

or a pharmaceutically acceptable salt thereof, optionally wherein:
(c) b is 2; or
(d) b is 2, A¹ is

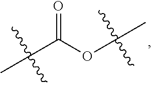 [SMILES: *OC(*)=O]

wherein the left hand side of the depicted structure is bound to the —(CH₂)a- and Z¹ is —S—S—.

14. The compound of numbered embodiment 1, wherein the compound has a structure according to Formula (In):

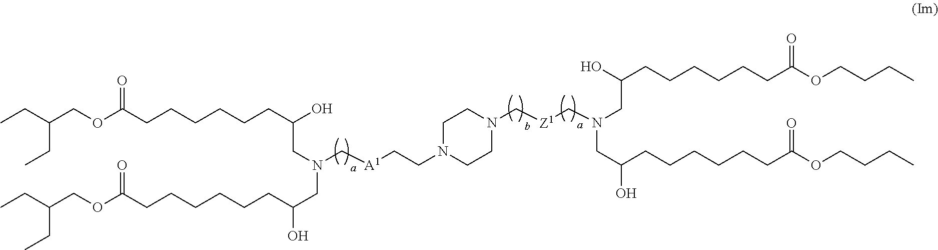

or a pharmaceutically acceptable salt thereof, optionally wherein:
(c) b is 2; or
(d) b is 2, A¹ is

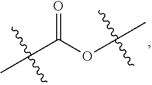 [SMILES: *OC(*)=O]

herein the left hand side of the depicted structure is bound to the —(CH₂)a- and Z¹ is —S—S—.

15. The compound of numbered embodiment 1, wherein the compound has a structure according to Formula (Io):

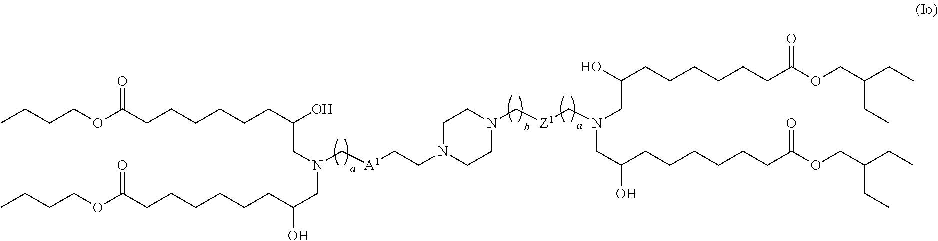

or a pharmaceutically acceptable salt thereof, optionally wherein:
(c) b is 2; or
(d) b is 2, A¹ is

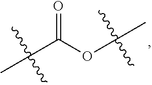 [SMILES: *OC(*)=O]

wherein the left hand side of the depicted structure is bound to the —(CH₂)a- and Z¹ is —S—S—.

16. The compound of numbered embodiment 1, wherein the compound has a structure according to Formula (Ip):

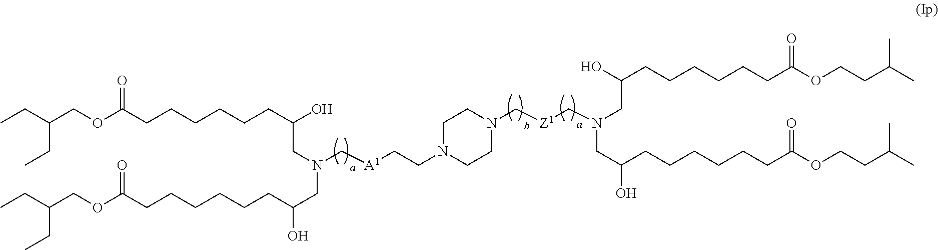

or a pharmaceutically acceptable salt thereof, optionally wherein:
(c) b is 2; or
(d) b is 2, A¹ is

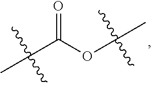 [SMILES: *OC(*)=O]

wherein the left hand side of the depicted structure is bound to the —(CH₂)a- and Z¹ is —S—S—.

17. The compound of numbered embodiment 1, wherein the compound has a structure according to Formula (Iq):

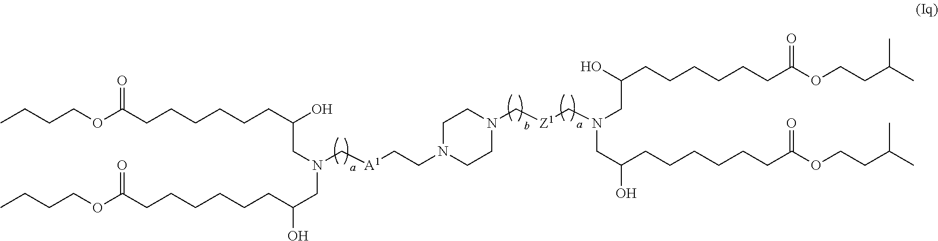

or a pharmaceutically acceptable salt thereof, optionally wherein:
(c) b is 2; or
(d) b is 2, A¹ is

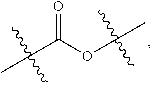 [SMILES: *OC(*)=O]

wherein the left hand side of the depicted structure is bound to the —(CH₂)a- and Z¹ is —S—S—.

18. The compound of any one of numbered embodiments 1-17 or a pharmaceutically acceptable salt thereof, wherein A¹ and Z¹ are the same.

193. The compound of any one of numbered embodiments 1-17 or a pharmaceutically acceptable salt thereof, wherein A¹ and Z¹ are different.

20. The compound of any one of numbered embodiments 1-19 or a pharmaceutically acceptable salt thereof, wherein A¹ is

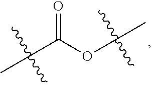 [SMILES: *OC(*)=O]

wherein the left hand side of the depicted structure is bound to the —(CH₂)a-.

21. The compound of any one of numbered embodiments 1-19 or a pharmaceutically acceptable salt thereof, wherein A¹ is

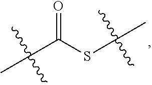 [SMILES: *SC(*)=O]

wherein the left hand side of the depicted structure is bound to the —(CH₂)a-.

22. The compound of any one of numbered embodiments 1-19 or a pharmaceutically acceptable salt thereof, wherein A¹ is-S—S—.

23. The compound of any one of numbered embodiments 1-22 or a pharmaceutically acceptable salt thereof, wherein Z¹ is

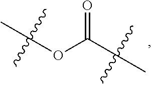 [SMILES: *OC(*)=O]

wherein the right hand side of the depicted structure is bound to the —(CH₂)a-.

24. The compound of any one of numbered embodiments 1-22 or a pharmaceutically acceptable salt thereof, wherein Z¹ is

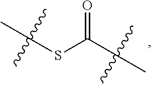 [SMILES: *SC(*)=O]

wherein the right hand side of the depicted structure is bound to the —(CH₂)a-.

25. The compound of any one of numbered embodiments 1-22 or a pharmaceutically acceptable salt thereof, wherein Z¹ is-S—S—.

26. The compound of any one of numbered embodiments 1-25 or a pharmaceutically acceptable salt thereof, wherein b is 2.

27. The compound of any one of numbered embodiments 1-25 or a pharmaceutically acceptable salt thereof, wherein b is 3.

28. The compound of any one of numbered embodiments 1-25 or a pharmaceutically acceptable salt thereof, wherein b is 4.

29. The compound of numbered embodiment 1, wherein the compound has a structure according to Formula (Ir):

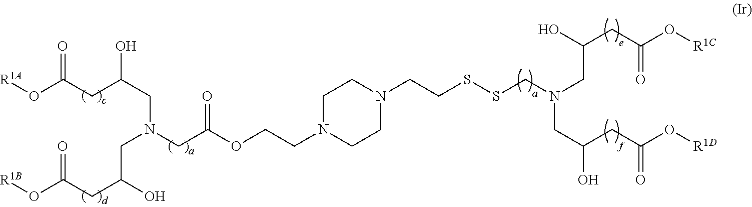

or a pharmaceutically acceptable salt thereof, optionally wherein each c, d, e and f is independently selected from 3, 4, or 6.

30. The compound of any one of numbered embodiments 1-29 or a pharmaceutically acceptable salt thereof, wherein each a is 3.

31. The compound of any one of numbered embodiments 1-29 or a pharmaceutically acceptable salt thereof, wherein each a is 4.

32. The compound of any one of numbered embodiments 1-29 or a pharmaceutically acceptable salt thereof, wherein the value for the a on the left hand side of the depicted Formula is 3 and the value for the a on the right hand side of the depicted Formula is 4.

33. The compound of any one of numbered embodiments 1-29 or a pharmaceutically acceptable salt thereof, wherein the value for the a on the left hand side of the depicted Formula is 4 and the value for the a on the right hand side of the depicted Formula is 3.

34. The compound of any one of numbered embodiments 1 or 18-33 or a pharmaceutically acceptable salt thereof, wherein in the compound of Formula (I) or (Ir) c, d, e and f are the same.

35. The compound of any one of numbered embodiments 1 or 18-34 or a pharmaceutically acceptable salt thereof, wherein in the compound of Formula (I) or (Ir) c, d, e and f are 3.

36. The compound of any one of numbered embodiments 1 or 18-34 or a pharmaceutically acceptable salt thereof, wherein in the compound of Formula (I) or (Ir) c, d, e and f are 4.

37. The compound of any one of numbered embodiments 1 or 18-34 or a pharmaceutically acceptable salt thereof, wherein in the compound of Formula (I) or (Ir) c, d, e and f are 5.

38. The compound of any one of numbered embodiments 1 or 18-34 or a pharmaceutically acceptable salt thereof, wherein in the compound of Formula (I) or (Ir) c, d, e and f are 6.

39. The compound of any one of numbered embodiments 1, 2, 4, 6, 8 or 18-33 or a pharmaceutically acceptable salt thereof, wherein in the compound of Formula (I), (Ia), (Ic), (Ie), (Ig), or (Ir) c and d are 3.

40. The compound of any one of numbered embodiments 1, 2, 4, 6, 8 or 18-33 or a pharmaceutically acceptable salt thereof, wherein in the compound of Formula (I), (Ia), (Ic), (Ie), (Ig), or (Ir) c and d are 4.

41. The compound of any one of numbered embodiments 1, 2, 4, 6, 8 or 18-33 or a pharmaceutically acceptable salt thereof, wherein in the compound of Formula (I), (Ia), (Ic), (Ie), (Ig), or (Ir) c and d are 5.

42. The compound of any one of numbered embodiments 1, 2, 4, 6, 8 or 18-33 or a pharmaceutically acceptable salt thereof, wherein in the compound of Formula (I), (Ia), (Ic), (Ie), (Ig), or (Ir) c and d are 6.

43. The compound of any one of numbered embodiments 1, 3, 5, 7, 9 or 18-33 or a pharmaceutically acceptable salt thereof, wherein in the compound of Formula (I), (Ib), (Id), (if), (Ih), or (Ir) e and f are 3.

44. The compound of any one of numbered embodiments 1, 3, 5, 7, 9 or 18-33 or a pharmaceutically acceptable salt thereof, wherein in the compound of Formula (I), (Ib), (Id), (if), (Ih), or (Ir) e and f are 4.

45. The compound of any one of numbered embodiments 1, 3, 5, 7, 9 or 18-33 or a pharmaceutically acceptable salt thereof, wherein in the compound of Formula (I), (Ib), (Id), (if), (Ih), or (Ir) e and f are 5.

46. The compound of any one of numbered embodiments 1, 3, 5, 7, 9 or 18-33 or a pharmaceutically acceptable salt thereof, wherein in the compound of Formula (I), (Ib), (Id), (if), (Ih), or (Ir) e and f are 6.

47. The compound of any one of numbered embodiments 1 or 18-33 or a pharmaceutically acceptable salt thereof, wherein in the compound of Formula (I) or (Ir) c and d are the same and e and f are the same, but wherein c and d are different to e and f.

48. The compound of any one of numbered embodiments 1 or 18-33 or 47 or a pharmaceutically acceptable salt thereof, wherein in the compound of Formula (I) or (Ir) c and d are 3 and e and f are 4.

49. The compound of any one of numbered embodiments 1 or 18-33 or 47 or a pharmaceutically acceptable salt thereof, wherein in the compound of Formula (I) or (Ir) c and d are 3 and e and f are 5.

50. The compound of any one of numbered embodiments 1 or 18-33 or 47 or a pharmaceutically acceptable salt thereof, wherein in the compound of Formula (I) or (Ir) c and d are 3 and e and f are 6.

51. The compound of any one of numbered embodiments 1 or 18-33 or 47 or a pharmaceutically acceptable salt thereof, wherein in the compound of Formula (I) or (Ir) c and d are 4 and e and f are 3.

52. The compound of any one of numbered embodiments 1 or 18-33 or 47 or a pharmaceutically acceptable salt thereof, wherein in the compound of Formula (I) or (Ir) c and d are 4 and e and f are 5.

53. The compound of any one of numbered embodiments 1 or 18-33 or 47 or a pharmaceutically acceptable salt thereof, wherein in the compound of Formula (I) or (Ir) c and d are 4 and e and f are 6.

54. The compound of any one of numbered embodiments 1 or 18-33 or 47 or a pharmaceutically acceptable salt thereof, wherein in the compound of Formula (I) or (Ir) c and d are 5 and e and f are 3.

55. The compound of any one of numbered embodiments 1 or 18-33 or 47 or a pharmaceutically acceptable salt thereof, wherein in the compound of Formula (I) or (Ir) c and d are 5 and e and f are 4.

56. The compound of any one of numbered embodiments 1 or 18-33 or 47 or a pharmaceutically acceptable salt thereof, wherein in the compound of Formula (I) or (Ir) c and d are 5 and e and f are 6.

57. The compound of any one of numbered embodiments 1 or 18-33 or 47 or a pharmaceutically acceptable salt thereof, wherein in the compound of Formula (I) or (Ir) c and d are 6 and e and f are 3.

58. The compound of any one of numbered embodiments 1 or 18-33 or 47 or a pharmaceutically acceptable salt thereof, wherein in the compound of Formula (I) or (Ir) c and d are 6 and e and f are 4.

59. The compound of any one of numbered embodiments 1 or 18-33 or 47 or a pharmaceutically acceptable salt thereof, wherein in the compound of Formula (I) or (Ir) c and d are 6 and e and f are 5.

60. The compound of any one of numbered embodiments 1 or 18-59 or a pharmaceutically acceptable salt thereof, wherein in the compound of Formula (I) or (Ir) R^{1A}, R^{1B}, R^{1C} and R^{1D} are the same.

61. The compound of any one of numbered embodiments 1 or 18-59 or a pharmaceutically acceptable salt thereof, wherein in the compound of Formula (I) or (Ir) R^{1A} and R^{1B} are the same and R^{1C} and R^{1D} are the same, but wherein R^{1A} and R^{1B} are different to R^{1C} and R^{1D}.

62. The compound of any one of numbered embodiments 1-61 or a pharmaceutically acceptable salt thereof, wherein each R^{1A}, R^{1B}, R^{1C} and R^{1D} when present is independently selected from:

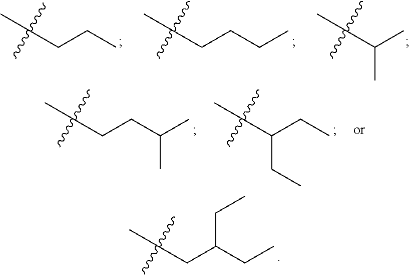

63. The compound of any one of numbered embodiments 1, 2, 4, 6, 8 or 18-62 or a pharmaceutically acceptable salt thereof, wherein in the compound of Formula (I), (Ia), (Ic), (Ie), (Ig), or (Ir) c and d are 4 and R^{1A} and R^{1B} are

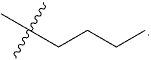 [SMILES: *CCCC]

64. The compound of any one of numbered embodiments 1, 2, 4, 6, 8 or 18-62 or a pharmaceutically acceptable salt thereof, wherein in the compound of Formula (I), (Ia), (Ic), (Ie), (Ig), or (Ir) c and d are 6 and R^{1A} and R^{1B} are

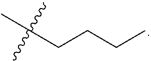 [SMILES: *CCCC]

65. The compound of any one of numbered embodiments 1, 2, 4, 6, 8 or 18-62 or a pharmaceutically acceptable salt thereof, wherein in the compound of Formula (I), (Ia), (Ic), (Ie), (Ig), or (Ir) c and d are 4 and R^{1A} and R^{1B} are

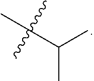 [SMILES: *C(C)C]

66. The compound of any one of numbered embodiments 1, 2, 4, 6, 8 or 18-62 or a pharmaceutically acceptable salt thereof, wherein in the compound of Formula (I), (Ia), (Ic), (Ie), (Ig), or (Ir) c and d are 6 and R^{1A} and R^{1B} are

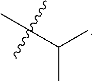 [SMILES: *C(C)C]

67. The compound of any one of numbered embodiments 1, 2, 4, 6, 8 or 18-62 or a pharmaceutically acceptable salt thereof, wherein in the compound of Formula (I), (Ia), (Ic), (Ie), (Ig), or (Ir) c and d are 4 and R^{1A} and R^{1B} are

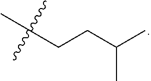 [SMILES: *CCC(C)C]

68. The compound of any one of numbered embodiments 1, 2, 4, 6, 8 or 18-62 or a pharmaceutically acceptable salt thereof, wherein in the compound of Formula (I), (Ia), (Ic), (Ie), (Ig), or (Ir) c and d are 6 and R^{1A} and R^{1B} are

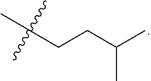 [SMILES: *CCC(C)C]

69. The compound of any one of numbered embodiments 1, 2, 4, 6, 8 or 18-62 or a pharmaceutically acceptable salt thereof, wherein in the compound of Formula (I), (Ia), (Ic), (Ie), (Ig), or (Ir) c and d are 3 and R^{1A} and R^{1B} are

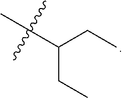 [SMILES: *C(CC)CC]

70. The compound of any one of numbered embodiments 1, 2, 4, 6, 8 or 18-62 or a pharmaceutically acceptable salt thereof, wherein in the compound of Formula (I), (Ia), (Ic), (Ie), (Ig), or (Ir) c and d are 4 and R^{1A} and R^{1B} are

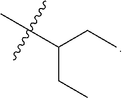 [SMILES: *C(CC)CC]

71. The compound of any one of numbered embodiments 1, 2, 4, 6, 8 or 18-62 or a pharmaceutically acceptable salt thereof, wherein in the compound of Formula (I), (Ia), (Ic), (Ie), (Ig), or (Ir) c and d are 6 and R^{1A} and R^{1B} are

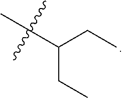 [SMILES: *C(CC)CC]

72. The compound of any one of numbered embodiments 1, 2, 4, 6, 8 or 18-62 or a pharmaceutically acceptable salt thereof, wherein in the compound of Formula (I), (Ia), (Ic), (Ie), (Ig), or (Ir) c and d are 3 and R^{1A} and R^{1B} are

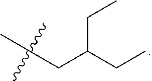 [SMILES: *CC(CC)CC]

73. The compound of any one of numbered embodiments 1, 2, 4, 6, 8 or 18-62 or a pharmaceutically acceptable salt thereof, wherein in the compound of Formula (I), (Ia), (Ic), (Ie), (Ig), or (Ir) c and d are 4 and R^{1A} and R^{1B} are

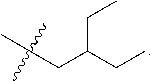 [SMILES: *CC(CC)CC]

74. The compound of any one of numbered embodiments 1, 2, 4, 6, 8 or 18-62 or a pharmaceutically acceptable salt thereof, wherein in the compound of Formula (I), (Ia), (Ic), (Ie), (Ig), or (Ir) c and d are 6 and R^{1A} and R^{1B} are

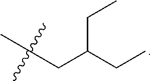 [SMILES: *CC(CC)CC]

75. The compound of any one of numbered embodiments 1, 3, 5, 7, 9 or 18-74 or a pharmaceutically acceptable salt thereof, wherein in the compound of Formula (I), (Ib), (Id), (If), (Ih), or (Ir) e and f are 4 and R^{1C} and R^{1D} are

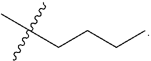 [SMILES: *CCCC]

76. The compound of any one of numbered embodiments 1, 3, 5, 7, 9 or 18-74 or a pharmaceutically acceptable salt thereof, wherein in the compound of Formula (I), (Ib), (Id), (If), (Ih), or (Ir) e and f are 6 and R^{1C} and R^{1D} are

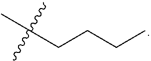 [SMILES: *CCCC]

77. The compound of any one of numbered embodiments 1, 3, 5, 7, 9 or 18-74 or a pharmaceutically acceptable salt thereof, wherein in the compound of Formula (I), (Ib), (Id), (If), (Ih), or (Ir) e and f are 4 and R^{1C} and R^{1D} are

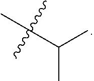 [SMILES: *C(C)C]

78. The compound of any one of numbered embodiments 1, 3, 5, 7, 9 or 18-74 or a pharmaceutically acceptable salt thereof, wherein in the compound of Formula (I), (Ib), (Id), (If), (Ih), or (Ir) e and f are 6 and R^{1C} and R^{1D} are

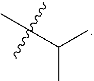 [SMILES: *C(C)C]

79. The compound of any one of numbered embodiments 1, 3, 5, 7, 9 or 18-74 or a pharmaceutically acceptable salt thereof, wherein in the compound of Formula (I), (Ib), (Id), (If), (Ih), or (Ir) e and f are 4 and R^{1C} and R^{1D} are

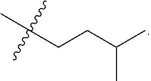 [SMILES: *CCC(C)C]

80. The compound of any one of numbered embodiments 1, 3, 5, 7, 9 or 18-74 or a pharmaceutically acceptable salt thereof, wherein in the compound of Formula (I), (Ib), (Id), (If), (Ih), or (Ir) e and f are 6 and R^{1C} and R^{1D} are

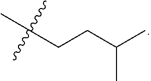 [SMILES: *CCC(C)C]

81. The compound of any one of numbered embodiments 1, 3, 5, 7, 9 or 18-74 or a pharmaceutically acceptable salt thereof, wherein in the compound of Formula (I), (Ib), (Id), (If), (Ih), or (Ir) e and f are 3 and R^{1C} and R^{1D} are

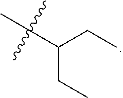 [SMILES: *C(CC)CC]

82. The compound of any one of numbered embodiments 1, 3, 5, 7, 9 or 18-74 or a pharmaceutically acceptable salt thereof, wherein in the compound of Formula (I), (Ib), (Id), (If), (Ih), or (Ir) e and f are 4 and R^{1C} and R^{1D} are

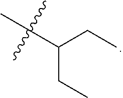 [SMILES: *C(CC)CC]

83. The compound of any one of numbered embodiments 1, 3, 5, 7, 9 or 18-74 or a pharmaceutically acceptable salt thereof, wherein in the compound of Formula (I), (Ib), (Id), (If), (Ih), or (Ir) e and f are 6 and R^{1C} and R^{1D} are

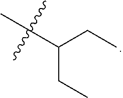 [SMILES: *C(CC)CC]

84. The compound of any one of numbered embodiments 1, 3, 5, 7, 9 or 18-74 or a pharmaceutically acceptable salt thereof, wherein in the compound of Formula (I), (Ib), (Id), (If), (Ih), or (Ir) e and f are 3 and R^{1C} and R^{1D} are

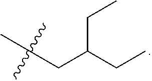 [SMILES: *CC(CC)CC]

85. The compound of any one of numbered embodiments 1, 3, 5, 7, 9 or 18-74 or a pharmaceutically acceptable salt thereof, wherein in the compound of Formula (I), (Ib), (Id), (If), (Ih), or (Ir) e and f are 4 and R^{1C} and R^{1D} are

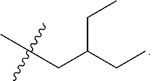 [SMILES: *CC(CC)CC]

86. The compound of any one of numbered embodiments 1, 3, 5, 7, 9 or 18-74 or a pharmaceutically acceptable salt thereof, wherein in the compound of Formula (I), (Ib), (Id), (If), (Ih), or (Ir) e and f are 6 and R^{1C} and R^{1D} are

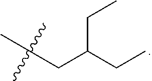 [SMILES: *CC(CC)CC]

87. The compound of any one of numbered embodiments 1 or 18-62 or a pharmaceutically acceptable salt thereof, wherein in the compound of Formula (I) or (Ir) each a is 4, c and d are 6, R^{1A} and R^{1B} are

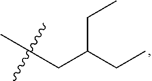 [SMILES: *CC(CC)CC]

e and f are 4 and R^{1C} and R^{1D} are

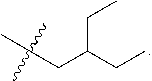 [SMILES: *CC(CC)CC]

88. A compound selected from those listed in Table A, Table B and/or Table C or a pharmaceutically acceptable salt thereof.

89. A composition comprising the cationic lipid of any one of numbered embodiments 1-88, and further comprising:
(i) one or more non-cationic lipids,
(ii) one or more cholesterol-based lipids, and
(iii) one or more PEG-modified lipids.

90. The composition of numbered embodiment 89, wherein the composition is a lipid nanoparticle, optionally a liposome.

91. The composition of numbered embodiment 90, wherein the one or more cationic lipid(s) constitute(s) about 30 mol %-60 mol % of the lipid nanoparticle.

92. The composition of numbered embodiment 90 or 91, wherein the one or more non-cationic lipid(s) constitute(s) about 10 mol %-50 mol % of the lipid nanoparticle.

93. The composition of any one of numbered embodiments 90-92, wherein the one or more PEG-modified lipid(s) constitute(s) about 1 mol %-10 mol % of the lipid nanoparticle.

94. The composition of any one of numbered embodiments 90-93, wherein the cholesterol-based lipid constitutes about 10 mol %-50 mol % of the lipid nanoparticle.

95. The composition of any one of numbered embodiments 90-94, wherein the lipid nanoparticle encapsulates a nucleic acid, optionally an mRNA encoding a peptide or protein.

96. The composition of any one of numbered embodiments 90-95, wherein the lipid nanoparticle encapsulates an mRNA encoding a peptide or protein, optionally for use in a vaccine.

97. The composition of numbered embodiment 96, wherein the lipid nanoparticles have an encapsulation percentage for mRNA of
(i) at least 50%;
(ii) at least 55%;
(iii) at least 60%;
(iv) at least 65%;
(v) at least 70%;
(vi) at least 75%;
(vii) at least 80%;
(viii) at least 85%;
(ix) at least 90%; or
(x) at least 95%.

98. The composition of numbered embodiment 96 or 97 for use in therapy.

99. The composition of numbered embodiment 96 or 97 for use in a method of treating or preventing a disease amenable to treatment or prevention by the peptide or protein encoded by the mRNA, optionally wherein the mRNA encodes an antigen and/or the disease is (a) a protein deficiency, optionally wherein the protein deficiency affects the liver, lung, brain or muscle, (b) an autoimmune disease, (c) an infectious disease, or (d) cancer.

100. The composition for use according to numbered embodiment 98 or 99, wherein the composition is administered intravenously, intrathecally or intramuscular, or by pulmonary delivery, optionally through nebulization.

101. A method for treating or preventing a disease wherein said method comprises administering to a subject in need thereof the composition of numbered embodiment 96 or 97 and wherein the disease is amenable to treatment or prevention by the peptide or protein encoded by the mRNA, optionally wherein the mRNA encodes an antigen and/or the disease is (a) a protein deficiency, optionally wherein the protein deficiency affects the liver, lung, brain or muscle, (b) an autoimmune disease, (c) an infectious disease, or (d) cancer.

102. The method of numbered embodiment 101, wherein the composition is administered intravenously, intrathecally or intramuscular, or by pulmonary delivery, optionally through nebulization.

What is claimed is:

1. A compound having a structure according to Formula (I): 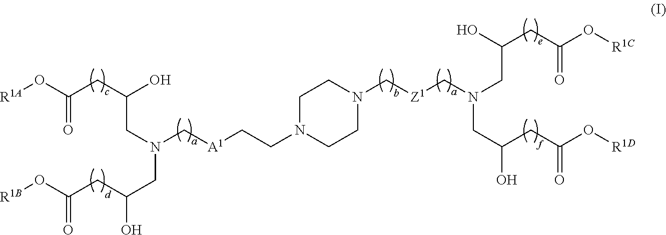
or a pharmaceutically acceptable salt thereof, wherein:
A¹ is selected from 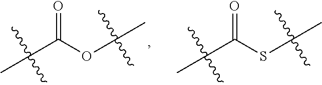 [SMILES: *OC(*)=O ; *SC(*)=O]
and —S—S—, wherein the left hand side of each depicted structure is bound to the —(CH₂)a-;
Z¹ is selected from 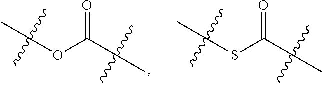 [SMILES: *OC(*)=O ; *SC(*)=O]
and —S—S—, wherein the right hand side of each depicted structure is bound to the —(CH₂)a-;
each a is independently selected from 3 or 4;
b is 1, 2, 3, 4 or 5;
each c, d, e and f is independently selected from 3, 4, 5 or 6; and
each R^{1A}, R^{1B}, R^{1C} and R^{1D} is independently selected from optionally substituted (C₃-C₆)alkyl.

2. The compound of claim 1 or a pharmaceutically acceptable salt thereof, wherein b is 2.

3. The compound of claim 1, wherein the compound has a structure according to Formula (Ir): 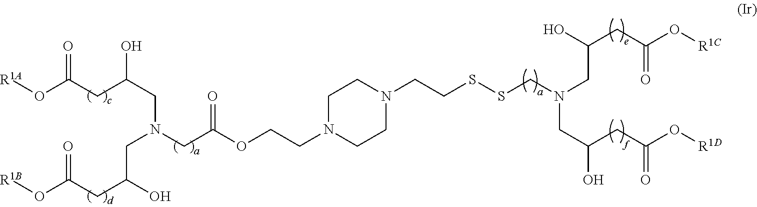
or a pharmaceutically acceptable salt thereof, optionally wherein each c, d, e and f is independently selected from 3, 4, or 6.

4. The compound of any one of claims 1-3 or a pharmaceutically acceptable salt thereof, wherein each a is 3.

5. The compound of any one of claims 1-3 or a pharmaceutically acceptable salt thereof, wherein each a is 4.

6. The compound of any one of claims 1-3 or a pharmaceutically acceptable salt thereof, wherein the value for the a on the left hand side of the depicted Formula is 3 and the value for the a on the right hand side of the depicted Formula is 4.

7. The compound of any one of claims 1-3 or a pharmaceutically acceptable salt thereof, wherein the value for the a on the left hand side of the depicted Formula is 4 and the value for the a on the right hand side of the depicted Formula is 3.

8. The compound of any one of claims 1-7 or a pharmaceutically acceptable salt thereof, wherein each R^{1A}, R^{1B}, R^{1C} and R^{1D} is independently selected from:

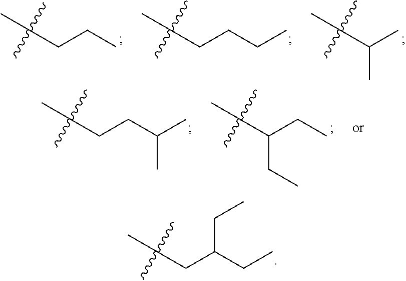

9. A composition comprising the cationic lipid of any one of claims 1-8, and further comprising:
(i) one or more non-cationic lipids,
(ii) one or more cholesterol-based lipids, and
(iii) one or more PEG-modified lipids.

10. The composition of claim 9, wherein the composition is a lipid nanoparticle, optionally a liposome.

11. The composition of claim 10, wherein the lipid nanoparticle encapsulates a nucleic acid, optionally an mRNA encoding a peptide or protein.

12. The composition of claim 10 or 11, wherein the lipid nanoparticle encapsulates an mRNA encoding a peptide or protein, optionally for use in a vaccine.

13. The composition of claim 12 for use in therapy.

14. The composition of claim 12 for use in a method of treating or preventing a disease amenable to treatment or prevention by the peptide or protein encoded by the mRNA, optionally wherein the mRNA encodes an antigen and/or the disease is (a) a protein deficiency, optionally wherein the protein deficiency affects the liver, lung, brain or muscle, (b) an autoimmune disease, (c) an infectious disease, or (d) cancer.

15. The composition for use according to claim 13 or 14, wherein the composition is administered intravenously, intrathecally or intramuscular, or by pulmonary delivery, optionally through nebulization.
